(19) United States
(12) Patent Application Publication
(10) Pub. No.: US 2024/0257926 A1
(43) Pub. Date: Aug. 1, 2024

(54) DIGITAL MEASUREMENT STACKS FOR CHARACTERIZING DISEASES, MEASURING INTERVENTIONS, OR DETERMINING OUTCOMES

(71) Applicant: Janssen Pharmaceutica NV, Beerse (BE)

(72) Inventors: Kai Langel, Beerse (BE); Bert Hartog, Beerse (BE); Erwin De Beuckelaer, Beerse (BE)

(21) Appl. No.: 18/558,925

(22) PCT Filed: May 6, 2022
(86) PCT No.: PCT/EP2022/062360

§ 371 Date: Nov. 3, 2023

Related U.S. Application Data
(60) Provisional application No. 63/184,907, filed on May 6, 2021.

(30) Foreign Application Priority Data
Nov. 16, 2021 (EP) 21383036.7

(51) Int. Cl. G16H 10/20; G16H 40/20

(52) U.S. Cl. CPC G16H 10/20; G16H 40/20

(57) ABSTRACT

Disclosed herein are standardized digital solutions, such as target solution profiles (TSPs) and digital measurement solutions (DMSs) that are useful for characterizing a disease for a subject. Generally, TSPs and DMSs are composed of a measurement stack comprising multiple components. The development of these standardized solutions for various diseases enables harmonization between various parties e.g., parties involved in clinical trials who are interested in characterizing diseases. Furthermore, standardized solutions enable improved life cycle management in view of the ever-developing landscape of new devices and software. Additionally, these digital measurement solutions represent novel solutions to characterizing disease.

### CROSS REFERENCE TO RELATED APPLICATIONS

This application claims the benefit of and priority to U.S. Provisional Patent Application No. 63/184,907 filed May 6, 2021 and EP21383036.7 filed Nov. 16, 2021, the entire disclosure of each of which is hereby incorporated by reference in its entirety for all purposes.

### BACKGROUND OF THE INVENTION

Digital health technologies show high potential in real-world evidence data generation. In the past decade, the number of clinical trials with digital health technologies involved showed a compound annual growth rate of 34.1%. However, to date, multiple limitations prevent the adoption of digital health technologies. Regularly named limitations include: (1) the lack of standardization, (2) concerns of how to choose the most appropriate digital measure, (3) how to collect, analyze and interpret the captured real-world evidence, (4) difficulty in maintaining integrity of solutions in light of everchanging technology, (5) how to prepare supporting materials for regulatory submission, and (6) the lack of translation from ideation to actual practice in clinical research and clinical care.

### SUMMARY OF THE INVENTION

Disclosed herein are methods, systems, and non-transitory computer readable media for building, implementing, and providing standardized digital solutions, such as target solution profiles (TSPs) and digital measurement solutions (DMSs). Generally, DMSs specify components of a full solution (e.g., particular devices, algorithms, and details for a measurement solution), and TSPs represent measurement methodologies that describe how the different components interact. These TSPs and DMSs are useful for characterizing a disease for a subject and can be provided to third parties to enable such third parties to characterize diseases. Generally, TSPs and DMSs are composed of a measurement stack comprising multiple layers, also referred to herein as components. Components are connected to adjacent components in the measurement stack, and each component is useful for the approved application of digital measurement solutions. In various embodiments, particular components of TSPs and DMSs are specifically developed for or are unique to a particular disease, or (sub)groups of patients suffering from a particular disease. In various embodiments, certain components of DMSs are interchangeable and can be swapped in and out of DMSs for various diseases.

Generally, digital measurement solutions (DMSs) are profiled into generic target solution profiles (TSPs). Therefore, various DMSs can be of a common class represented by a TSP. TSPs aim to fill the earlier mentioned gaps of standardization by describing agnostic classifications (e.g., device agnostic, device-software agnostic, algorithm-agnostic). Thus, TSPs represent a standardized class of solutions with aligned definitions and validated instrumentation.

Altogether, TSPs and DMSs, as described herein, represent standardized solutions for characterizing disease. Examples of characterizing disease include, but are not limited to, determining disease severity, determining likelihood of disease progression, and measuring treatment outcomes for a disease. A first specific benefit is that TSPs allow for harmonization between multiple assets and components, thereby improving standardization within the ecosystem. Second, the development time of standardized solutions (e.g., DMS) is significantly shortened, which allows for conservation of resources and reduction of unnecessary costs. For example, available components or assets can be repurposed for similar or identical conditions with ease. Third, TSPs allow for improved life cycle management. Qualification protocols are developed for individual TSPs which encompass various DMSs. In one scenario, this ensures that DMSs in a common class represented by a TSP perform in a similar or comparable manner. In a second scenario, when upgrades occur (e.g., when new instrumentations are developed or when new software algorithms are made available), DMSs can be efficiently evaluated using qualification protocols to ensure comparable solutions of DMSs within the class of solutions. Thus, introduction of TSPs and DMSs to the ecosystem accelerates the adoption of digital measures and long-term research interoperability (e.g., interoperability across different clinical trials) first in clinical research and additionally in clinical care.

Disclosed herein is a method for characterizing a disease of a subject, the method comprising: obtaining a measurement of interest from the subject; selecting a digital measurement solution from a plurality of digital measurement solutions, wherein the plurality of digital measurement solutions are of a common class that is represented by a target solution profile; and applying the selected digital measurement solution to the obtained measurement of interest to characterize the disease for the subject, wherein the digital measurement solution comprises: a measurement definition defining one or more concepts of interest relevant to the disease; an instrumentation asset that transforms the measurement of interest captured according to the measurement definition to a dataset that is informative for characterizing the disease, wherein the instrumentation asset of the digital measurement solution is specific for a device used to capture the measurement of interest; and optionally, an evidence asset for performing one or more validations on the dataset generated by the instrumentation asset, wherein the target solution profile is unchanged over time and enables efficient life-cycle management of the plurality of digital measurement solutions. In various embodiments, the target solution profile represents a generalization of the plurality of digital measurement solutions, wherein an instrumentation asset of the target solution profile is device technology agnostic. In various embodiments, performing the one or more validations comprises performing one or more of a technical validation, an analytical validation, or a clinical validation. In various embodiments, performing the technical validation comprises comparing the dataset generated by the instrumentation asset to specifications of one or more devices used to capture the measurement of interest. In various embodiments, performing the analytical validation comprises: determining any of reliability, specificity, or sensitivity metrics for the dataset; and comparing the reliability, specificity, or sensitivity metrics to a threshold value. In various embodiments, performing the clinical validation comprises: assessing treatment effects on measurements of interest for the disease.

In various embodiments, the digital measurement solution is previously validated by implementing one or more qualification protocols used to establish comparability of solutions across the digital measurement solutions of the target solution profile. In various embodiments, a qualification protocol comprises steps of: a) recruiting a N member participant group; b) capturing measurements of interest across the N member participant group according to a specification of the digital measurement solution; c) transforming the measurements of interest into a dataset according to the specification; and d) validating the dataset to determine whether the digital measurement solution achieves comparable solutions of the target solution profile. In various embodiments, validating the dataset comprises: determining whether a characteristic of the dataset satisfies a threshold value of the target solution profile; and responsive to the determination that the characteristic of the dataset satisfies the threshold value, validating the digital measurement solution as achieving comparability of solutions. In various embodiments, validating the dataset further comprises responsive to determining that the digital measurement solution achieves comparability of solutions, storing an indication of a successful validation in metadata of the digital measurement solution. In various embodiments, the metadata of the digital measurement solution is stored in a catalog accessible for inspection by third party users. In various embodiments, the specification of the digital measurement solution represents an upgraded capability in comparison to a prior version of the digital measurement solution. In various embodiments, the specification of the digital measurement solution represents an upgraded capability included in a newly released device used to capture the measurement of interest. In various embodiments, the upgraded capability is one of an upgraded battery, upgraded data storage, upgraded acquisition frequency, or upgraded data collection algorithm. In various embodiments, the common class of the plurality of digital measurement solutions represents a common method of measuring activity from an individual. In various embodiments, the common method of measuring activity uses a class of devices comprising one or more of wearable devices, devices including accelerometers, devices including gyroscopes, ingestibles, image and voice based devices, touchless sensors, and sensor based smart devices (e.g., scales, thermometers, and respirators). In various embodiments, the instrumentation asset comprises a machine learning algorithm that transforms data captured according to the measurement definition to the dataset.

Additionally disclosed herein is a method for building a digital measurement solution for characterizing a disease, the method comprising: generating a measurement definition of a target solution profile, the measurement definition defining one or more concepts of interest relevant to the disease; generating or selecting an instrumentation asset for the target solution profile, the instrumentation asset configured to transform data captured according to the measurement definition to a dataset, the instrumentation asset being device technology agnostic and is thereby interchangeable across different target solution profiles; generating an evidence asset of the target solution profile for performing one or more validations on the dataset generated by the instrumentation asset; generating a digital measurement solution by at least specifying a device for the instrumentation asset of target solution profile, wherein the digital measurement solution is of a common class that is represented by the target solution profile, wherein the target solution profile is unchanged over time and thereby enables efficient life-cycle management of the plurality of digital measurement solutions.

In various embodiments, the one or more concepts of interest relevant to the disease comprise medical measurements of the disease or measurable experiences of individuals suffering from the disease. In various embodiments, device technology agnostic comprises one or both of being device-agnostic and being device-version agnostic. In various embodiments, methods disclosed herein further comprise implementing a qualification protocol to validate the digital measurement solution, the qualification protocol used to establish comparability of solutions across the plurality of digital measurement solutions of the target solution profile. In various embodiments, a qualification protocol comprises steps of: a) recruiting a N member participant group; b) capturing measurements of interest across the N member participant group using a specification of the digital measurement solution; c) transforming the measurements of interest into a dataset according to the specification; and d) validating the dataset to determine whether the digital measurement solution achieves comparable solutions of the target solution profile. In various embodiments, validating the dataset comprises determining whether the dataset satisfies a threshold value of the target solution profile; and responsive to the determination that the dataset satisfies the threshold value, validating the digital measurement solution as achieving comparability of solutions. In various embodiments, validating the dataset further comprises responsive to determining that the digital measurement solution achieves comparability of solutions, storing an indication of a successful validation in metadata of the digital measurement solution. In various embodiments, the metadata of the digital measurement solution is stored in a catalog accessible for inspection by third party users. In various embodiments, the specification of the digital measurement solution represents an upgraded capability of a prior version of the digital measurement solution. In various embodiments, the specification of the digital measurement solution represents an upgraded capability included in a newly released device used to capture the measurement of interest. In various embodiments, the upgraded capability is one of an upgraded battery, upgraded data storage, upgraded acquisition frequency, or upgraded data collection algorithm.

In various embodiments, the measurement definition and evidence asset are fixed for the target solution profile and specific for the disease. In various embodiments, the instrumentation asset of the target solution profile is interchangeable across different target solution profiles for characterizing a same disease or different diseases. In various embodiments, the instrumentation asset is specific for a common method of measuring activity from an individual. In various embodiments, the common method of measuring activity uses a class of devices comprising one or more of wearable devices, devices including accelerometers, devices including gyroscopes, ingestibles, image and voice based devices, touchless sensors, and sensor based smart devices (e.g., scales, thermometers, and respirators). In various embodiments, the digital measurement solution comprises providing the digital measurement solution to a third party for regulatory approval. In various embodiments, the digital measurement solution comprises providing input to the third party on one or more assets of the digital measurement solution.

In various embodiments, methods disclosed herein further comprise providing the digital measurement solution to a third party for regulatory approval. In various embodiments, methods disclosed herein further comprise providing input to the third party on one or more assets of the digital measurement solution.

In various embodiments, the digital measurement solution is one of the digital measurement solutions shown in Table 5. In various embodiments, the target solution profile is one of the target solution profiles shown in Table 4. In various embodiments, the disease is a condition shown in Table 1. In various embodiments, the one or more concepts of interest are selected from a concept of interest shown in Table 3.

Additionally disclosed herein is a method for providing one or more digital measurement solutions useful for characterizing a disease, the method comprising: providing a catalogue comprising a plurality of target solution profiles, wherein each of one or more of the target solution profiles comprises: a measurement definition of the target solution profile defining one or more concepts of interest relevant to the disease; an instrumentation asset that transforms a measurement of interest captured according to the measurement definition to a dataset that is informative for characterizing the disease, the instrumentation asset being device technology agnostic and is thereby interchangeable across different target solution profiles; and an evidence asset for performing one or more validations on the dataset generated by the instrumentation asset; receiving, from a third party, a selection of one of the target solution profiles; and providing one or more digital measurement solutions useful for characterizing the disease to the third party, wherein the one or more digital measurement solutions are of a common class represented by the selected target solution profile.

In various embodiments, methods disclosed herein further comprise: receiving, from the third party, a search query; for each of the one or more target solution profiles in the plurality of target solution profiles, evaluating the target solution profile to determine whether the target solution profile satisfies the query; and returning a list of target solution profiles that satisfy the query. In various embodiments, evaluating the target solution profile comprises: evaluating one or more components of the measurement definition for a concept of interest that satisfies the query. In various embodiments, methods disclosed herein further comprise: replacing an instrumentation asset of one of the one or more digital measurement solutions with a second instrumentation asset to generate a revised digital measurement solution; and providing the revised digital measurement solution to the third party. In various embodiments, methods disclosed herein further comprise receiving, from a third party, a suggested target solution profile not present in the provided catalog; and further generating the suggested target solution profile for inclusion in the catalog.

In various embodiments, the target solution profile comprises a measurement stack comprising a plurality of components. In various embodiments, each of the measurement definition, the instrumentation asset, and the evidence asset are represented by one or more components in the plurality of components. In various embodiments, an order of the plurality of components comprises one or more components of the measurement definition, followed by one or more components of the instrumentation asset, and further followed by one or more components of the evidence asset. In various embodiments, a component of the measurement definition interfaces with a component of the instrumentation asset, and a component of the instrumentation asset interfaces with the evidence asset. In various embodiments, the one or more components of the measurement definition comprise one or more of a hypothesis of the disease and a measurable concept of interest. In various embodiments, the one or more components of the instrumentation asset comprise one or more of a measurement method, raw data, and a machine learning algorithm. In various embodiments, the one or more components of the evidence asset comprise one or more of a technical validation, an analytical validation, and a clinical validation. In various embodiments, the one or more components of the measurement definition comprise a hypothesis of the disease and a measurable concept of interest, wherein the one or more components of the instrumentation asset comprise a measurement method, raw data, and a machine learning algorithm, and wherein the one or more components of the evidence asset comprise a technical validation, an analytical validation, and a clinical validation. In various embodiments, the plurality of components of the measurement stack are a plurality of layers.

In various embodiments, the disease is dementia. In various embodiments, the hypothesis comprises an intervention that slows progression of dementia. In various embodiments, the measurable concept of interest comprises one or more of attention, language, or executive functioning. In various embodiments, the measurement method comprises a method for capturing speech. In various embodiments, the raw data comprises raw speech data captured from a subject or magnetic resonance imaging data. In various embodiments, the machine learning algorithm comprises one or more of a natural language processing algorithm, a clustering algorithm, and a clinical variable predictor. In various embodiments, the analytical validation comprises evidence validating performance of the machine learning algorithm. In various embodiments, the clinical interpretation comprises evidence supporting characterization of the disease according to a clinical dementia rating. In various embodiments, the disease is Parkinson's Disease. In various embodiments, the hypothesis comprises an intervention that reduces tremor. In various embodiments, the measurable concept of interest comprises ability to perform daily activities of moderate intensity. In various embodiments, the measurement method comprises methods for capturing physiological data using a biosensor. In various embodiments, the raw data comprises raw physiological data captured using a biosensor. In various embodiments, the machine learning algorithm transforms the raw data to the dataset. In various embodiments, the analytical validation comprises evidence validating performance of the machine learning algorithm. In various embodiments, the clinical interpretation comprises evidence supporting characterization of the disease in a Parkinson's Disease patient population.

In various embodiments, the disease is Atopic Dermatitis. In various embodiments, the hypothesis comprises an intervention that reduces nocturnal scratch. In various embodiments, the measurable concept of interest comprises nocturnal scratching. In various embodiments, the measurement method comprises methods for capturing physiological data using a wearable device. In various embodiments, the raw data comprises raw physiological data captured using the wearable device. In various embodiments, the machine learning algorithm transforms the raw data to the dataset. In various embodiments, the clinical validation comprises evidence supporting treatments effects on nocturnal scratching in an atopic dermatitis population.

In various embodiments, the disease is Pulmonary Arterial Hypertension. In various embodiments, the hypothesis comprises an intervention that improves patient ability to perform physical activities following treatment. In various embodiments, the measurable concept of interest comprises a performance of daily activities by patients affected by pulmonary arterial hypertension. In various embodiments, the measurement method comprises a wrist-worn device for capturing physiological data. In various embodiments, the raw data comprises raw physiological data measured from sensors of the wrist-worn device, wherein the sensors comprise one or more of an accelerometer, gyroscope, and magnetometer. In various embodiments, the machine learning algorithm transforms the raw data to the dataset. In various embodiments, the clinical validation comprises evidence of improvement in daily performance following an intervention.

Additionally disclosed herein is a non-transitory computer readable medium for characterizing a disease of a subject, the non-transitory computer readable medium comprising instructions that, when executed by a processor, cause the processor to: obtain a measurement of interest from the subject; select a digital measurement solution from a plurality of digital measurement solutions, wherein the plurality of digital measurement solutions are of a common class that is represented by a target solution profile; and apply the selected digital measurement solution to the obtained measurement of interest to characterize the disease for the subject, wherein the digital measurement solution comprises: a measurement definition defining one or more concepts of interest relevant to the disease; an instrumentation asset that transforms the measurement of interest captured according to the measurement definition to a dataset that is informative for characterizing the disease, wherein the instrumentation asset of the digital measurement solution is specific for a device used to capture the measurement of interest; and optionally, an evidence asset for performing one or more validations on the dataset generated by the instrumentation asset, wherein the target solution profile is unchanged over time and enables efficient life-cycle management of the plurality of digital measurement solutions. In various embodiments, the target solution profile represents a generalization of the plurality of digital measurement solutions, wherein an instrumentation asset of the target solution profile is device technology agnostic.

In various embodiments, the instructions that cause the processor to perform the one or more validations further comprises instructions that, when executed by the processor, cause the processor to: perform one or more of a technical validation, an analytical validation, or a clinical validation. In various embodiments, the instructions that cause the processor to perform the technical validation further comprises instructions that, when executed by the processor, cause the processor to compare the dataset generated by the instrumentation asset to specifications of one or more devices used to capture the measurement of interest. In various embodiments, the instructions that cause the processor to perform the analytical validation further comprises instructions that, when executed by the processor, cause the processor to perform any of reliability, specificity, or sensitivity metrics for the dataset; and compare the reliability, specificity, or sensitivity metrics to a threshold value. In various embodiments, the instructions that cause the processor to perform the clinical validation further comprises instructions that, when executed by the processor, cause the processor to assess treatment effects on measurements of interest for the disease. In various embodiments, the digital measurement solution is previously validated by implementing one or more qualification protocols used to establish comparability of solutions across the digital measurement solutions of the target solution profile. In various embodiments, a qualification protocol comprises steps of: a) recruiting a N member participant group; b) capturing measurements of interest across the N member participant group according to a specification of the digital measurement solution; c) transforming the measurements of interest into a dataset according to the specification; and d) validating the dataset to determine whether the digital measurement solution achieves comparable solutions of the target solution profile. In various embodiments, validating the dataset comprises: determining whether a characteristic of the dataset satisfies a threshold value of the target solution profile; and responsive to the determination that the characteristic of the dataset satisfies the threshold value, validating the digital measurement solution as achieving comparability of solutions. In various embodiments, validating the dataset further comprises responsive to determining that the digital measurement solution achieves comparability of solutions, storing an indication of a successful validation in metadata of the digital measurement solution. In various embodiments, the metadata of the digital measurement solution is stored in a catalog accessible for inspection by third party users. In various embodiments, the specification of the digital measurement solution represents an upgraded capability in comparison to a prior version of the digital measurement solution. In various embodiments, the specification of the digital measurement solution represents an upgraded capability included in a newly released device used to capture the measurement of interest. In various embodiments, the upgraded capability is one of an upgraded battery, upgraded data storage, upgraded acquisition frequency, or upgraded data collection algorithm. In various embodiments, the common class of the plurality of digital measurement solutions represents a common method of measuring activity from an individual. In various embodiments, the common method of measuring activity uses a class of devices comprising one or more of wearable devices, devices including accelerometers, devices including gyroscopes, ingestibles, image and voice based devices, touchless sensors, and sensor based smart devices (e.g., scales, thermometers, and respirators). In various embodiments, the instrumentation asset comprises a machine learning algorithm that transforms data captured according to the measurement definition to the dataset.

Additionally disclosed herein is a non-transitory computer readable medium for building a digital measurement solution for characterizing a disease, the non-transitory computer readable medium comprising instructions that, when executed by a processor, cause the processor to: generate a measurement definition of a target solution profile, the measurement definition defining one or more concepts of interest relevant to the disease; generate or select an instrumentation asset for the target solution profile, the instrumentation asset configured to transform data captured according to the measurement definition to a dataset, the instrumentation asset being device technology agnostic and is thereby interchangeable across different target solution profiles; generate an evidence asset of the target solution profile for performing one or more validations on the dataset generated by the instrumentation asset; generate a digital measurement solution by at least specifying a device for the instrumentation asset of target solution profile, wherein the digital measurement solution is of a common class that is represented by the target solution profile, wherein the target solution profile is unchanged over time and thereby enables efficient life-cycle management of the plurality of digital measurement solutions.

In various embodiments, the one or more concepts of interest relevant to the disease comprise medical measurements of the disease or measurable experiences of individuals suffering from the disease. In various embodiments, device technology agnostic comprises one or both of being device-agnostic and being device-version agnostic. In various embodiments, the non-transitory computer readable medium further comprises instructions that, when executed by the processor, cause the processor to implement a qualification protocol to validate the digital measurement solution, the qualification protocol used to establish comparability of solutions across the plurality of digital measurement solutions of the target solution profile. In various embodiments, a qualification protocol comprises steps of: a) recruiting a N member participant group; b) capturing measurements of interest across the N member participant group using a specification of the digital measurement solution; c) transforming the measurements of interest into a dataset according to the specification; and d) validating the dataset to determine whether the digital measurement solution achieves comparable solutions of the target solution profile. In various embodiments, validating the dataset comprises determining whether the dataset satisfies a threshold value of the target solution profile; and responsive to the determination that the dataset satisfies the threshold value, validating the digital measurement solution as achieving comparability of solutions. In various embodiments, validating the dataset further comprises responsive to determining that the digital measurement solution achieves comparability of solutions, storing an indication of a successful validation in metadata of the digital measurement solution. In various embodiments, the metadata of the digital measurement solution is stored in a catalog accessible for inspection by third party users. In various embodiments, the specification of the digital measurement solution represents an upgraded capability of a prior version of the digital measurement solution. In various embodiments, the specification of the digital measurement solution represents an upgraded capability included in a newly released device used to capture the measurement of interest. In various embodiments, the upgraded capability is one of an upgraded battery, upgraded data storage, upgraded acquisition frequency, or upgraded data collection algorithm.

In various embodiments, the measurement definition and evidence asset are fixed for the target solution profile and specific for the disease. In various embodiments, the instrumentation asset of the target solution profile is interchangeable across different target solution profiles for characterizing a same disease or different diseases. In various embodiments, the instrumentation asset is specific for a common method of measuring activity from an individual. In various embodiments, the common method of measuring activity uses a class of devices comprising one or more of wearable devices, devices including accelerometers, devices including gyroscopes, ingestibles, image and voice based devices, touchless sensors, and sensor based smart devices (e.g., scales, thermometers, and respirators). In various embodiments, the digital measurement solution is one of the digital measurement solutions shown in Table 5. In various embodiments, the target solution profile is one of the target solution profiles shown in Table 4. In various embodiments, the disease is a condition shown in Table 1. In various embodiments, the one or more concepts of interest are selected from a concept of interest shown in Table 3.

Additionally disclosed herein is a non-transitory computer readable medium for providing one or more digital measurement solutions useful for characterizing a disease, the non-transitory computer readable medium comprising instructions that, when executed by a processor, cause the processor to: provide a catalogue comprising a plurality of target solution profiles, wherein each of one or more of the target solution profiles comprises: a measurement definition of the target solution profile defining one or more concepts of interest relevant to the disease; an instrumentation asset that transforms a measurement of interest captured according to the measurement definition to a dataset that is informative for characterizing the disease, the instrumentation asset being device technology agnostic and is thereby interchangeable across different target solution profiles; and an evidence asset for performing one or more validations on the dataset generated by the instrumentation asset; receive, from a third party, a selection of one of the target solution profiles; and provide one or more digital measurement solutions useful for characterizing the disease to the third party, wherein the one or more digital measurement solutions are of a common class represented by the selected target solution profile. In various embodiments, non-transitory computer readable media disclosed herein further comprise instructions that, when executed by a processor, cause the processor to: receive, from the third party, a search query; for each of the one or more target solution profiles in the plurality of target solution profiles, evaluate the target solution profile to determine whether the target solution profile satisfies the query; and return a list of target solution profiles that satisfy the query. In various embodiments, the instructions that cause the processor to evaluate the target solution profile further comprises instructions that, when executed by the processor, cause the processor to evaluate one or more components of the measurement definition for a concept of interest that satisfies the query. In various embodiments, non-transitory computer readable media disclosed herein further comprise instructions that, when executed by a processor, cause the processor to replace an instrumentation asset of one of the one or more digital measurement solutions with a second instrumentation asset to generate a revised digital measurement solution; and provide the revised digital measurement solution to the third party.

In various embodiments, non-transitory computer readable media disclosed herein, further comprise instructions that, when executed by a processor, cause the processor to: receive, from a third party, a suggested target solution profile not present in the provided catalog; and further generate the suggested target solution profile for inclusion in the catalog.

In various embodiments, the target solution profile comprises a measurement stack comprising a plurality of components. In various embodiments, each of the measurement definition, the instrumentation asset, and the evidence asset are represented by one or more components in the plurality of components. In various embodiments, an order of the plurality of components comprises one or more components of the measurement definition, followed by one or more components of the instrumentation asset, and further followed by one or more components of the evidence asset. In various embodiments, a component of the measurement definition interfaces with a component of the instrumentation asset, and a component of the instrumentation asset interfaces with the evidence asset. In various embodiments, the one or more components of the measurement definition comprise one or more of a hypothesis of the disease and a measurable concept of interest. In various embodiments, the one or more components of the instrumentation asset comprise one or more of a measurement method, raw data, and a machine learning algorithm. In various embodiments, the one or more components of the evidence asset comprise one or more of a technical validation, an analytical validation, and a clinical validation. In various embodiments, the one or more components of the measurement definition comprise a hypothesis of the disease and a measurable concept of interest, wherein the one or more components of the instrumentation asset comprise a measurement method, raw data, and a machine learning algorithm, and wherein the one or more components of the evidence asset comprise a technical validation, an analytical validation, and a clinical validation. In various embodiments, the plurality of components of the measurement stack are a plurality of layers.

In various embodiments, the disease is dementia. In various embodiments, the hypothesis comprises an intervention that slows progression of dementia. In various embodiments, the measurable concept of interest comprises one or more of attention, language, or executive functioning. In various embodiments, the measurement method comprises a method for capturing speech. In various embodiments, the raw data comprises raw speech data captured from a subject or magnetic resonance imaging data. In various embodiments, the machine learning algorithm comprises one or more of a natural language processing algorithm, a clustering algorithm, and a clinical variable predictor. In various embodiments, the analytical validation comprises evidence validating performance of the machine learning algorithm. In various embodiments, the clinical interpretation comprises evidence supporting characterization of the disease according to a clinical dementia rating.

In various embodiments, the disease is Parkinson's Disease. In various embodiments, the hypothesis comprises an intervention that reduces tremor. In various embodiments, the measurable concept of interest comprises ability to perform daily activities of moderate intensity. In various embodiments, the measurement method comprises methods for capturing physiological data using a biosensor. In various embodiments, the raw data comprises raw physiological data captured using a biosensor. In various embodiments, the machine learning algorithm transforms the raw data to the dataset. In various embodiments, the analytical validation comprises evidence validating performance of the machine learning algorithm. In various embodiments, the clinical interpretation comprises evidence supporting characterization of the disease in a Parkinson's Disease patient population

In various embodiments, the disease is Atopic Dermatitis. In various embodiments, the hypothesis comprises an intervention that reduces nocturnal scratch. In various embodiments, the measurable concept of interest comprises nocturnal scratching. In various embodiments, the measurement method comprises methods for capturing physiological data using a wearable device. In various embodiments, the raw data comprises raw physiological data captured using the wearable device. In various embodiments, the machine learning algorithm transforms the raw data to the dataset. In various embodiments, the clinical validation comprises evidence supporting treatments effects on nocturnal scratching in an atopic dermatitis population.

In various embodiments, the disease is Pulmonary Arterial Hypertension. In various embodiments, the hypothesis comprises an intervention that improves patient ability to perform physical activities following treatment. In various embodiments, the measurable concept of interest comprises a performance of daily activities by patients affected by pulmonary arterial hypertension. In various embodiments, the measurement method comprises a wrist-worn device for capturing physiological data. In various embodiments, the raw data comprises raw physiological data measured from sensors of the wrist-worn device, wherein the sensors comprise one or more of an accelerometer, gyroscope, and magnetometer. In various embodiments, the machine learning algorithm transforms the raw data to the dataset. In various embodiments, the clinical validation comprises evidence of improvement in daily performance following an intervention.

### BRIEF DESCRIPTION OF THE DRAWINGS

These and other features, aspects, and advantages of the present invention will become better understood with regard to the following description and accompanying drawings. It is noted that wherever practicable similar or like reference numbers may be used in the figures and may indicate similar or like functionality. For example, a letter after a reference numeral, such as “third party entity 110A,” indicates that the text refers specifically to the element having that particular reference numeral. A reference numeral in the text without a following letter, such as “third party entity 110,” refers to any or all of the elements in the figures bearing that reference numeral (e.g., “third party entity 110” in the text refers to reference numerals “third party entity 110A” and/or “third party entity 110A” in the figures).

FIG. 1A is a system overview including the digital solution system and one or more third party entities, in accordance with an embodiment.

FIG. 1B is a block diagram of the digital solution system, in accordance with an embodiment.

FIG. 2A is an example measurement stack, in accordance with an embodiment.

FIG. 2B is an example measurement stack showing individual components, in accordance with an embodiment.

FIG. 2C is an example measurement stack indicating one or more components that form a target solution profile (TSP), target instrumentation profile (TIP), or target component profile (TCP), in accordance with an embodiment.

FIG. 2D shows an example target solution profile, in accordance with an embodiment.

FIG. 2E shows an example digital measurement solution, in accordance with a first embodiment.

FIG. 2F shows an example digital measurement solution, in accordance with a second embodiment.

FIG. 3A is an example flow process for building a digital measurement solution, in accordance with an embodiment.

FIG. 3B is an example flow process for characterizing a disease for a subject using a digital measurement solution (DMS), in accordance with an embodiment.

FIG. 3C is an example flow process for providing a target solution profile or one or more digital measurement solutions, in accordance with an embodiment.

FIG. 4 illustrates an example computing device for implementing system and methods described in FIGS. 1A-1B, 2A-2F, and 3A-3C.

FIG. 5A depicts an example target solution profile for atopic dermatitis.

FIG. 5B depicts an example digital measurement solution for atopic dermatitis.

FIG. 5C depicts the interchangeability of assets of different digital measurement solutions for atopic dermatitis.

FIG. 6A depicts an example target solution profile for pulmonary arterial hypertension.

FIG. 6B depicts a first example digital measurement solution for pulmonary arterial hypertension.

FIG. 6C depicts a second example digital measurement solution for pulmonary arterial hypertension.

FIG. 6D depicts the repurposing of at least the instrumentation asset of digital measurement solutions.

FIG. 7 depicts an example digital measurement solution for Parkinson's Disease.

FIG. 8A depicts a high level overview involving collaborative efforts for developing standardized solutions.

FIG. 8B depicts an example flow process involving various parties for enabling dynamic regulatory assessment of standardized solutions.

### DETAILED DESCRIPTION OF THE INVENTION

### Definitions

Terms used in the claims and specification are defined as set forth below unless otherwise specified.

The term “subject” or “patient” are used interchangeably and encompass a cell, tissue, organism, human or non-human, mammal or non-mammal, male or female, whether in vivo, ex vivo, or in vitro.

The term “disease” or “condition” are used interchangeably and generally refer to a diseased status of a subject. Generally, a standardized solution, such as a digital measurement solution, is implemented to characterize the disease for the subject.

The phrase “measurement stack” refers to an organization of one or more assets that are composed of components. In particular embodiments, the measurement stack is composed of two or more assets. In particular embodiments, the measurement stack is composed of three or more assets. For example, the measurement stack includes a measurement definition asset, an instrumentation asset, and an evidence asset. The measurement stack provides a structure for standardized solutions, such as a target solution profile or a digital measurement solution.

The phrases “target solution profile” or “TSP” refer to a measurement stack in which generic descriptions are incorporated to provide a device technology agnostic profile (e.g., a profile that is independent of a particular hardware device and/or independent of particular software). In various embodiments, a target solution profile includes each of a measurement definition asset, an instrumentation asset, and an evidence asset. In various embodiments, the instrumentation asset of the target solution profile describes general methods of capturing and transforming raw data of interest but does not specify particular devices or algorithms for capturing and transforming the raw data. Target solution profiles represent a common class of digital measurement solutions. Target solution profiles may specify performance requirements and/or standards such that digital measurement solutions of the common class represented by the target solution profile are evaluated and confirmed to perform within the performance requirements and/or standards.

The phrases “digital measurement solution” or “DMS” refer to a specific digital solution built upon a measurement stack. In various embodiments, a DMS specifies all of the components of a full solution, which can include devices, algorithms, external data, definition, and/or evidence. For example, a digital measurement solution identifies specific devices or software for capturing raw data. In various embodiments, a digital measurement solution identifies a specific algorithm for transforming the raw data into meaningful health data. Thus, implementation of a digital measurement solution is useful for characterizing a disease for a subject.

The phrase “standardized solution” refers to standard digital solutions useful for characterizing disease. Examples of standardized solutions include digital measurement solutions and target solution profiles.

### Overview

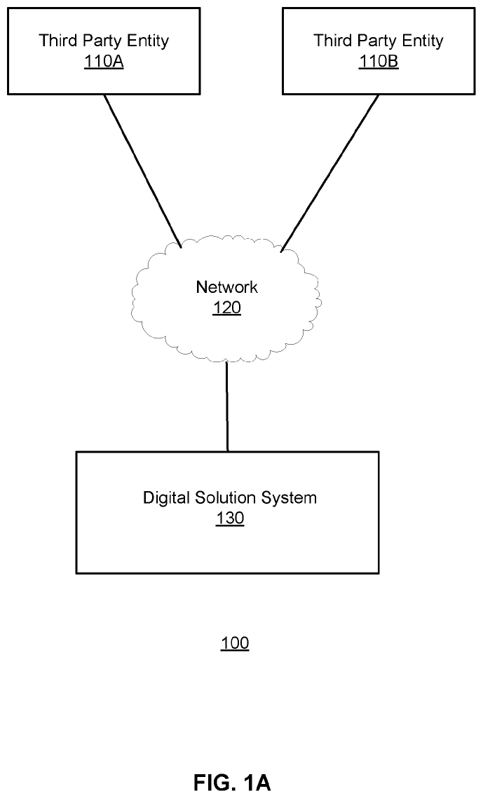

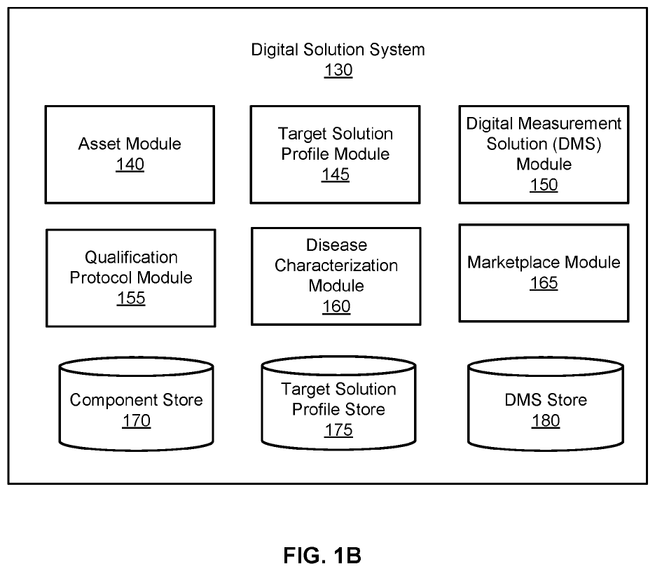

FIG. 1A is a system overview including the digital solution system 130 and one or more third party entities 110, in accordance with an embodiment. Specifically, FIG. 1A introduces a digital solution system 130 connected to one or more third party entities 110 via a network 120. Although FIG. 1A depicts a digital solution system 130 connected to two separate third party entities 110A and 110B, in various embodiments, the digital solution system 130 can be connected to additional third party entities (e.g., tens, hundreds, or even thousands of third party entities).

Generally, the digital solution system 130 builds and/or maintains standardized solutions, examples of which include digital measurement solutions (DMSs) and target solution profiles (TSPs), that are built on measurement stacks. Standardized solutions are useful for characterizing diseases for subjects and furthermore, enables efficient life cycle management of the various solutions. In various embodiments, the digital solution system 130 interacts with various third party entities (e.g., third party entities 110A and/or 110B) to build and maintain DMSs and TSPs. In various embodiments, the digital solution system 130 represents a centralized marketplace incorporating these standardized DMSs and TSPs. Thus, the digital solution system 130 provides standardized DMSs and TSPs to third party entities (e.g., third party entities 110A and/or 110B) via the centralized marketplace such that the third party entities can use the standardized solutions e.g., for characterize a disease or condition.

### Third Party Entity

In various embodiments, the third party entity 110 represents a partner entity of the digital solution system 130. In some embodiments, the third party entity 110 is a partner entity that collaborates with the digital solution system 130 for building TSPs and/or DMSs. In some scenarios, the third party entity 110 represents an asset developer. As one example, the third party entity 110 can develop components that can be provided to the digital solution system 130 for incorporation into standardized solutions (e.g., DMSs or TSPs). As another example, the third party entity 110 can provide feedback to the digital solution system 130. For example, the third party entity 110 can provide suggestions as to valuable standardized solutions (e.g., DMSs or TSPs). These standardized solutions may be currently missing (e.g., not present in the catalog or available in the marketplace). Thus, the digital solution system 130 can generate these suggested standardized solutions, perform the appropriate validation, and include them in the marketplace.

In some embodiments, the third party entity 110 represents a regulatory specialist. Here, the third party entity 110 can interact with the digital solution system 130 to verify the standardized solutions (e.g., DMSs and TSPs) and approve them as standard solutions for clinical trials. As described in further detail herein, DMSs and TSPs may include components that provide specific guidelines for regulatory specialists, which can lead to improved standardization and adoption of these solutions.

In various embodiments, multiple third party entities 110 collaborate together to build standardized solutions and to achieve regulatory acceptance of the standardized solutions. For example, the multiple third party entities 110 collaborate together to enable dynamic regulatory assessment of standardized solutions (e.g., DMSs and/or TSPs). In various embodiments, the multiple third party entities 110 includes stakeholders who are interested in building the standardized solutions. Such stakeholders can include asset developers (e.g., entities that build and/or provide components and/or assets), pharmaceutical companies, observers, service providers, and/or customers (e.g, entities interested in using standardized solutions). Thus, these stakeholders can provide feedback in working together to build the standardized solutions. In various embodiments, the multiple third party entities 110 further includes regulatory individuals who perform the regulatory assessment of the standardized solutions. Thus, the regulatory individuals can provide regulatory acceptance of standardized solutions. In various embodiments, the regulatory individuals can interact with other multiple third party entities 110 (e.g., stakeholders) to enable dynamic regulatory assessment. For example, regulatory individuals can correspond with stakeholders in understanding the context and use cases of the standardized solutions, thereby ensuring more rapid regulatory approval.

In some embodiments, the third party entity 110 represents a customer who is interested in accessing and using the standardized solutions, such as DMSs, to characterize diseases for subjects. Example customers include any of a sponsor (e.g., clinical trial sponsor), a clinical researcher, a health care specialist, a physician, a vendor, or a supplier. In such embodiments, the third party entity 110 can interact with the digital solution system 130 to access and use the standardized solutions. For example, the digital solution system 130 may provide TSPs and/or DMSs to the third party entity 110 that suits the needs of the third party entity 110. For example, as described in further detail herein, DMSs and TSPs may identify particular specifications (e.g., device specifications or software specifications) that establish the measurements of interest that are captured for a particular disease or condition, e.g., captured from subjects with or without the disease or condition. Thus, a third party entity 110 who is interested in characterizing the particular disease or condition can evaluate the required specifications and identify the appropriate DMSs or TSPs that best suit their need. The digital solution system 130 can provide the appropriate DMSs or TSPs. Using the appropriate DMS, the third party entity 110 characterizes a disease for one or more subjects. For example, the third party entity 110 can capture a measurement of interest from a subject according to measurement methods described in a DMS. The third party entity 110 can further transform the measurement of interest into meaningful health data using an algorithm specified in the DMS. Then, the third party entity 110 interprets the meaningful health data and characterizes the disease.

### Network

This disclosure contemplates any suitable network 120 that enables connection between the digital solution system 130 and third party entities 702. The network 120 may comprise any combination of local area and/or wide area networks, using both wired and/or wireless communication systems. In one embodiment, the network 120 uses standard communications technologies and/or protocols. For example, the network 120 includes communication links using technologies such as Ethernet, 802.11, worldwide interoperability for microwave access (WiMAX), 3G, 4G, code division multiple access (CDMA), digital subscriber line (DSL), etc. Examples of networking protocols used for communicating via the network 120 include multiprotocol label switching (MPLS), transmission control protocol/Internet protocol (TCP/IP), hypertext transport protocol (HTTP), simple mail transfer protocol (SMTP), and file transfer protocol (FTP). Data exchanged over the network 120 may be represented using any suitable format, such as hypertext markup language (HTML) or extensible markup language (XML). In some embodiments, all or some of the communication links of the network 120 may be encrypted using any suitable technique or techniques.

### Digital Solution System

FIG. 1B is a block diagram of the digital solution system 130, in accordance with an embodiment. FIG. 1B is presented to introduce components of the digital solution system 130 including an asset module 140, a target solution profile module 145, a digital measurement solution (DMS) module 150, a qualification protocol module 155, a disease characterization module 160, and a marketplace module 165. The digital solution system 130 may further include data stores, such as a component store 170, target solution profile store 175, and digital measurement solution (DMS) store 180. In various embodiments, the digital solution system 130 may include additional components or need not include all of the components as shown in FIG. 1B. For example, the disease characterization module 160 may be implemented by a different party (e.g., a third party entity 110 as shown in FIG. 1A). Therefore, the steps of characterizing a disease performed by the disease characterization module 160 may be additionally or alternatively performed, in some embodiments, by a third party entity.

Referring first to the asset module 140, it generates or obtains individual components and constructs assets composed of two or more components. The asset module 140 may store components and/or assets in the component store 170. Examples of components include 1) an aspect of health component relevant to the disease, 2) a hypothesis component, 3) a concept of interest component which defines a measurable unit that informs the aspect of health of the disease, 4) a measurement method component that defines how raw data is captured, 5) a raw data component specifying characteristics of the raw data, 6) an algorithm component for implementing an algorithm that transforms the raw data, 7) a health data component describing meaningful interpretation of data relevant for the disease, 8) an analytical validation component, 9) clinical validation component, and 10) a regulatory intelligence component. Further description of these example components is included herein.

In various embodiments, the asset module 140 may organize individual component into assets that are composed of two or more components. As an example, the asset module 140 may organize A) an aspect of health component relevant to the disease, B) a hypothesis component, and C) a concept of interest component into an asset, hereafter referred to as a measurement definition asset. As another example, the asset module 140 can organize A) a measurement method component that defines how raw data is captured, B) raw data component specifying characteristics of the raw data, C) algorithm component for implementing an algorithm that transforms the raw data, and D) health data component describing meaningful interpretation of data relevant for the disease into an asset, hereafter referred to as an instrumentation asset. As yet another example, the asset module 140 can organize A) analytical validation component, B) clinical validation component, and C) regulatory intelligence component into an asset, hereafter referred to as an evidence asset. Further details of these example assets are described herein.

In various embodiments, the asset module 140 generates components and constructs assets through de novo methods. For example, the asset module 140 identifies a particular disease and generates components and constructs assets that are useful for characterizing the particular disease. In various embodiments, the asset module 140 may receive components and/or assets from third party entities (e.g., third party entities 110 shown in FIG. 1A). In such embodiments, the third party entities may be asset developers who create and provide their own components and/or assets to the digital solution system 130. Thus, the asset module 140 can organize components received from third party entities into assets. In various embodiments, the asset module 140 can organize a mix of components that are generated de novo and components received from third party entities into assets.

The target solution profile module 145 generates target solution profiles (TSPs) using components and/or assets e.g., components and/or assets generated de novo by the asset module 140 or components and/or assets obtained by the asset module 140 from third party entities. In various embodiments, a TSP includes a measurement definition asset, instrumentation asset, and/or evidence asset. In particular embodiments, a TSP includes each of a measurement definition asset, instrumentation asset, and evidence asset. Generally, a TSP represents a measurement stack in which generic descriptions are incorporated to provide a device technology agnostic profile (e.g., a profile that is independent of a specific hardware device and independent of specific software). The generic descriptions are valuable to ensure that assets of the TSP can be readily interchangeable. For example, the instrumentation asset of a TSP can specify a class of devices for capturing measurements. Examples of a class of devices include, but are not limited to: wearable devices (e.g., wrist-worn device), ingestibles, image and voice based devices, touchless sensors, and sensor based smart devices (e.g., scales, thermometers, and respirators).

In various embodiments, the TSP module 145 builds a TSP using a condition-focused approach (e.g., bottom-up approach). Here, the TSP is built by first identifying a condition or disease of interest. Thus, the components of the TSP are assembled for the purpose of characterizing the disease of interest. In various embodiments, the TSP module 145 builds a TSP an instrumentation-focused approach (e.g., top-down approach). Here, the TSP is built by identifying the components and assets that are available for use (e.g., components and assets stored in component store 170). This ensures that components and assets that have previously been generated and/or validated can be easily repurposed. Thus, in various embodiments, building a TSP can involve repurposing components and assets from other TSPs such that new components and assets need not be generated. In particular embodiments, instrumentation assets of other TSPs can be repurposed for building a new TSP, even in scenarios where the other TSPs and the new TSP are developed for different diseases. The TSP module 145 can store the generated TSPs in the TSP store 175. Further details of example TSPs are described herein.

The digital measurement solution (DMS) module 150 builds one or more DMSs. In various embodiments, the DMS module 150 builds one or more DMSs by incorporating specific information into a TSP. Here, the TSP represents a class of solutions for the one or more DMSs. For example, the DMS module 150 can incorporate specific device hardware into a component of a TSP. Thus, a DMS specifies the particular device that is to be used to capture raw measurements. As another example, the DMS module 150 can incorporate specific algorithms into a component of a TSP. Thus, a DMS specifies the particular algorithm that is used to transform raw measurements into a meaningful health dataset that can be interpreted to characterize the disease.

In various embodiments, the DMS module 150 builds two or more DMSs of a common class represented by a TSP. In various embodiments, the DMS module 150 builds three or more DMSs, four or more DMSs, five or more DMSs, six or more DMSs, seven or more DMSs, eight or more DMSs, nine or more DMSs, ten or more DMSs, eleven or more DMSs, twelve or more DMSs, thirteen or more DMSs, fourteen or more DMSs, fifteen or more DMSs, sixteen or more DMSs, seventeen or more DMSs, eighteen or more DMSs, nineteen or more DMSs, twenty or more DMSs, twenty five or more DMSs, fifty or more DMSs, a hundred or more DMSs, two hundred or more DMSs, three hundred or more DMSs, four hundred or more DMSs, five hundred or more DMSs, six hundred or more DMSs, seven hundred or more DMSs, eight hundred or more DMSs, nine hundred or more DMSs, or a thousand or more DMSs of a common class represented by a TSP. The DMS module 150 can store the generated DMSs in the DMS store 180. Further details of example DMSs are described herein.

The qualification protocol module 155 performs qualification protocols that enable rapid onboarding of upgraded DMSs (e.g., in view of upgraded devices and/or upgraded software releases) by validating comparability of results across multiple DMSs of a common class. For example, when a new device or software package is released, the new device or software package can be incorporated in an updated or upgraded DMS. Here, the qualification protocol module 155 implements a qualification protocol to validate the new DMS incorporating the new device or new software package. This ensures that the new DMS achieves comparable results to other DMSs of the same common class. Further details of the implementation of qualification protocols are described herein.

In various embodiments, DMSs that have undergone successful validation using a qualification protocol can be identified as successfully validated. For example, metadata associated with a successfully validated DMS can be annotated. For example, the metadata can identify the qualification protocol that was used, as well as the fact that the DMS was successfully validated. In various embodiments, the metadata including the annotation can be available for inspection by a third party. Therefore, a third party, such as a customer who is interested in using a DMS to characterize a disease, can select a DMS that has been successfully validated.

The disease characterization module 160 implements a DMS to characterize a disease. In various embodiments, the disease characterization module 160 can be employed by a third party entity (e.g., third party entity 110 shown in FIG. 1A). For example, the third party entity may be a customer interested in characterizing a disease. Thus, the third party entity can employ the disease characterization module 160 to implement a selected DMS to characterize a disease. In various embodiments, the disease characterization module 160 can obtain a measurement of interest. For example, the measurement of interest can be raw data that is obtained according to the measurement method specified by the DMS. Furthermore, implementing the DMS involves transforming the measurement of interest into meaningful health data using an algorithm specified in the DMS. The disease characterization module 160 can interpret the meaningful health data to characterize the disease.

The marketplace module 165 implements a marketplace of the standardized solutions (e.g., DMSs and TSPs) and enables third party entities to access the DMSs and TSPs for their uses. In various embodiments, the marketplace module 165 provides an interface to third party entities that depicts the various DMSs and TSPs that are available for access. Such an interface can be organized as a catalog for ease of access.

In various embodiments, the marketplace module 165 provides a catalog of TSPs that are useful for characterizing various diseases. The marketplace module 165 may receive a selection of one of the TSPs. For example, a third party may select a TSP for characterizing a disease that is of interest for the third party. Furthermore, the third party may select the TSP because it includes specifications that align with the capabilities of the third party. In one scenario, the marketplace module 165 can provide the selected TSP to the third party. In one scenario, the marketplace module 165 can identify one or more DMSs that are of a common class represented by the selected TSP. Here, the marketplace module 165 provides the one or more DMSs of the common class to the third party.

In various embodiments, the marketplace module 165 may provide recommendations to third parties that are accessing the marketplace. For example, the marketplace module 165 can provide a recommendation identifying one or more components, one or more assets, one or more TSPs, or one or more DMSs to a third party. This can be useful for third parties that may need additional guidance as to the best standardized solution that will fit their needs.

In various embodiments, the marketplace module 165 receives suggestions as to additional standardized solutions that would be of value. For example, the marketplace module 165 may receive a suggestion from a third party for a particular DMS or TSP that is not present in the marketplace. Such a third party may be an asset developer or a customer who identifies a gap that is not satisfied by the current offerings of standardized solutions. For example, the suggestion may identify that specifications of a particular device exceed the specifications of available TSPs and DMSs. Therefore, the marketplace module 165 can provide the suggestion to any of the asset module 140, TSP module 145, and/or DMS module 150 to generate additional standardized solutions that can be included in the marketplace.

In various embodiments, the marketplace module 165 provides a catalog of target solution profiles and receives a search query. For example, a third party presented with the catalog of target solution profiles my provide a search query for a particular component or asset in a target solution profile. In various embodiments, the third party provides a search query for a concept of interest or for a particular disease. The marketplace module 165 queries the available TSPs (e.g., TSPs stored in the target solution profile store 175) according to the search query, and returns a list of TSPs that satisfy the search query. For example, if the search query specifies a particular concept of interest the marketplace module 165 evaluates the components of the TSPs for a concept of interest that satisfies the search query. Thus, the marketplace module 165 can provide the list of TSPs that satisfy the search query (e.g., to the third party).

### Example Measurement Stack

Embodiments disclosed herein involve the building of TSPs and DMSs, as well as the implementation of TSPs and DMSs for characterizing disease. Generally, TSPs and DMSs are built on a measurement stack comprised of one or more components (also referred to herein as layers). Namely, a measurement stack provides a structure or framework for a TSP or DMS. The components and/or assets of a measurement stack can be generated and/or maintained by the asset module 140, as described above in reference to FIG. 1B.

The goal of the measurement stack is to fulfill the earlier mentioned gaps as, for example, the lack of standardization and concerns about the collection, analysis, and interpretation of data. First, the measurement stack provides a standardized structure that represents a universal way of describing a solution, thereby allowing for standardization. Second, the measurement stack initiates and allows for harmonization between multiple assets and components. Third, the measurement stack model will enable assets to transition between diseases and use-cases, enabling component level reusability.

In various embodiments, the measurement stack includes one or more assets. Examples of assets include a measurement definition asset, an instrumentation asset, or an evidence asset. An asset refers to one or more components of the stack. In various embodiments, an asset refers to two or more components. In various embodiments, an asset refers to three or more components. In various embodiments, an asset refers to three or more components.

In various embodiments, the measurement definition asset includes two components. In various embodiments, the measurement definition asset includes three components. In various embodiments, the measurement definition asset includes four components. In various embodiments, the instrumentation asset includes two components. In various embodiments, the instrumentation asset includes three components. In various embodiments, the instrumentation asset includes four components. In various embodiments, the evidence asset includes two components. In various embodiments, the evidence asset includes three components. In various embodiments, the evidence asset includes four components.

In various embodiments, the measurement stack includes two assets. For example, the measurement stack may include a measurement definition asset related to a particular disease and an instrumentation asset that describes the capturing of data that is useful for characterizing the condition. In particular embodiments, the measurement stack includes three assets. For example, the measurement stack may include a measurement definition asset related to a particular disease, an instrumentation asset that describes the capturing of data that is useful for characterizing the disease, and an evidence asset for validating meaningful datasets of the disease.

In various embodiments, the components of an asset are connected to one another. For example, the components of an asset are configured to communicate with at least one another component of the same asset. For example, within an asset, the components are organized as layers, and therefore, a first component is configured to communicate with a second component that is adjacent to the first component. This enables the transfer of information from one component to the next component.

In various embodiments, a component of a first asset is connected to a component of a second asset. Thus, the component of the first asset can communicate with the component of the second asset. As an example, within a measurement stack, a first asset may be located lower in the measurement stack in relation to a second asset. Here, a component of the first asset can be connected to a component of the second asset, thereby enabling the first asset and second asset to interface with each other.

In various embodiments, the assets of the measurement stack are ordered as follows (from bottom to top of the stack): 1) measurement definition asset and 2) instrumentation asset. In particular embodiments, the assets of the measurement stack are ordered as follows (from bottom to top of the stack): 1) measurement definition asset, 2) instrumentation asset, and evidence asset.

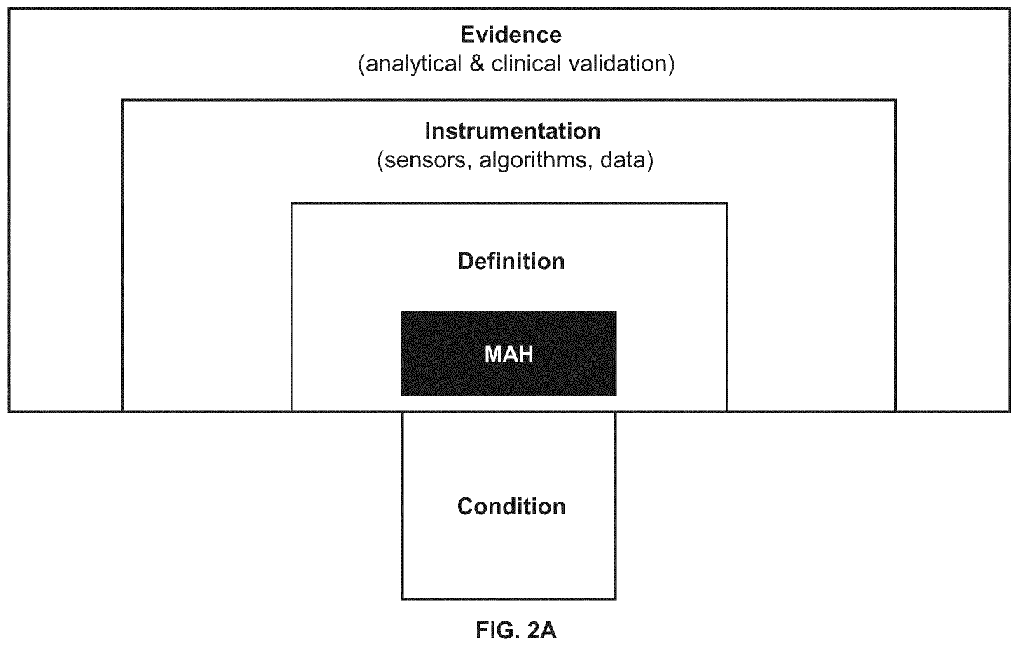

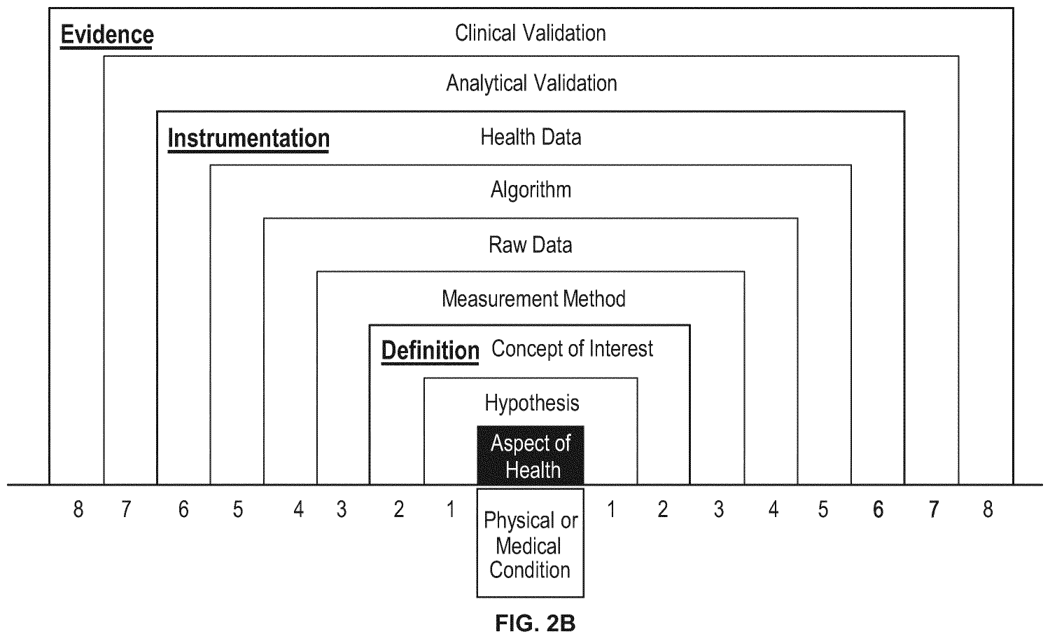

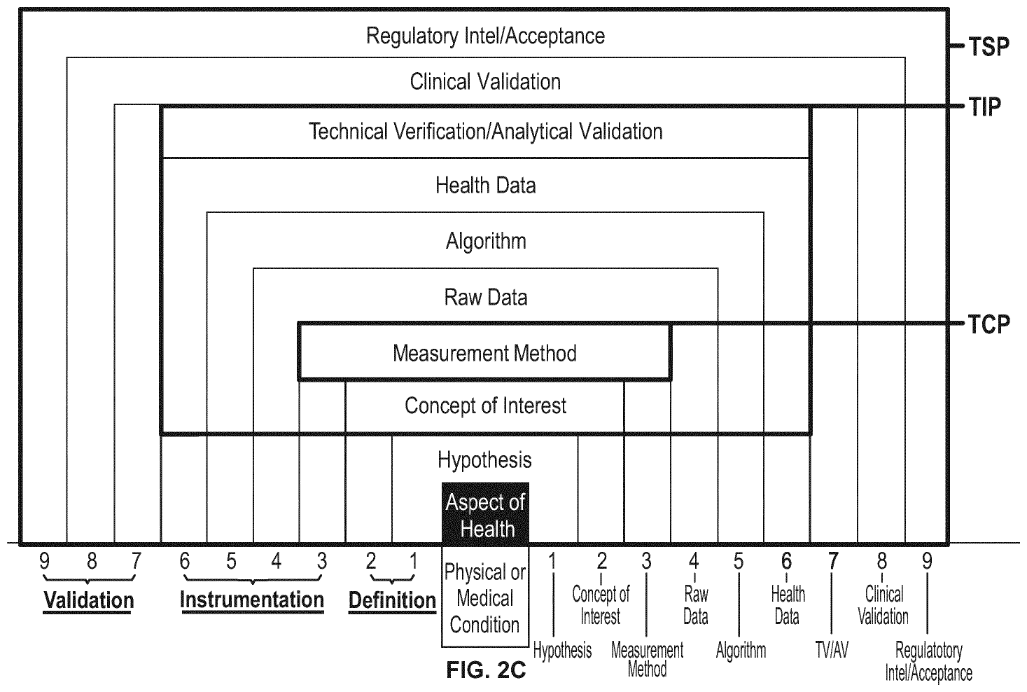

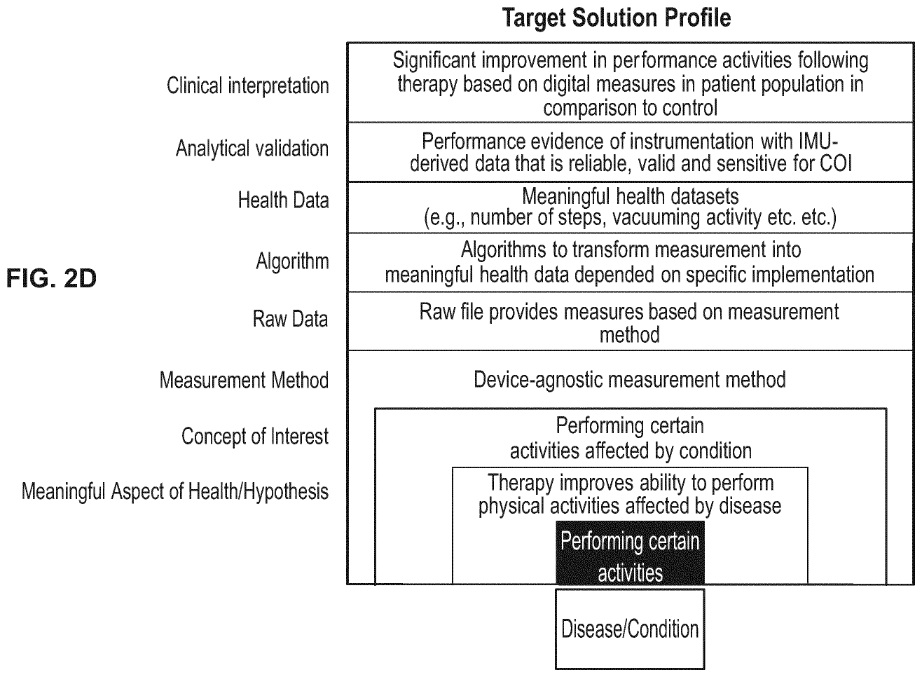

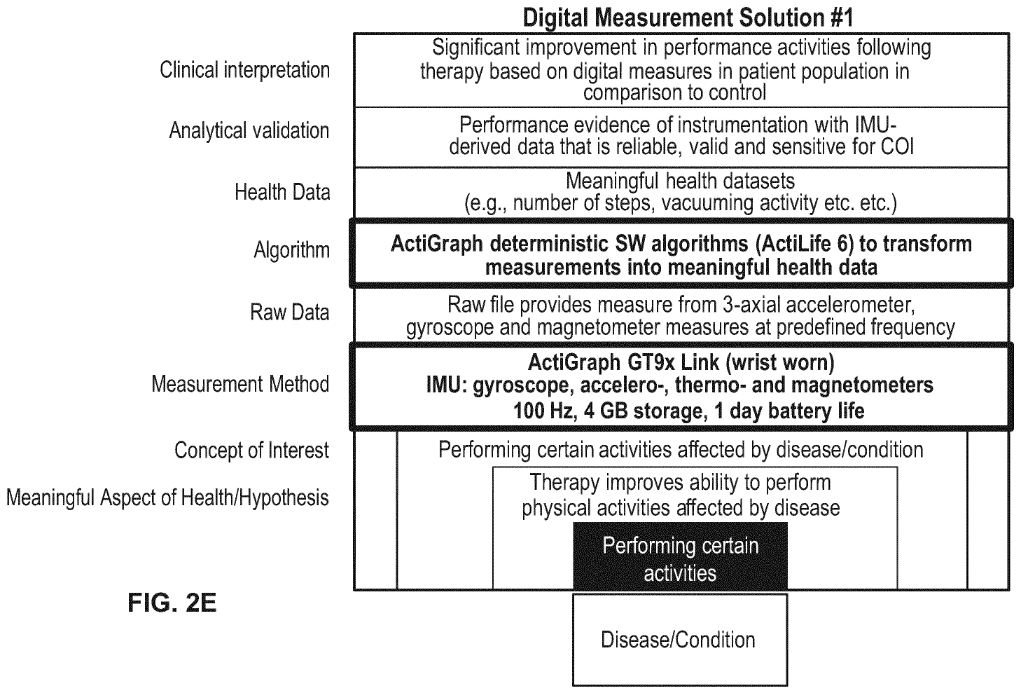

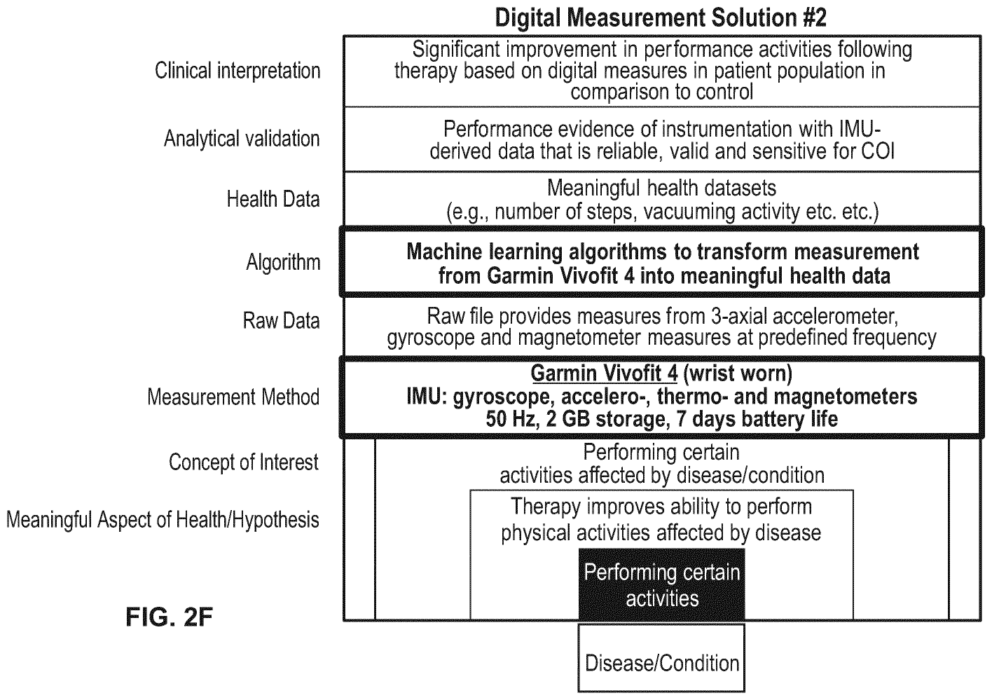

Reference is now made to FIG. 2A, which shows an example measurement stack, in accordance with an embodiment. As shown in FIG. 2A, the example measurement stack includes a particular disease (referred to as “Condition” in FIG. 2A), a measurement definition asset (labeled as “Definition” in FIG. 2A) that includes a meaningful aspect of health (MAH), an instrumentation asset (labeled as “Instrumentation” in FIG. 2A) which includes components related to the capturing of data, algorithms, and datasets, and an evidence asset (labeled as “Evidence” in FIG. 2A) that describes one or more validations for validating the generated datasets. Example conditions are further detailed in Table 1. Example meaningful aspects of health (MAH) are detailed in Table 2.

As shown in FIG. 2A, the particular disease is located at the bottom of the measurement stack. Here, the disease can govern the generation or selection of one or more of the assets above in the measurement stack. For example, the disease governs the generation or selection of the measurement definition asset.

Generally, the measurement definition asset defines measurable concepts related to the disease. Thus, the measurable concept is informative for characterizing a disease (e.g., presence of a disease, severity of a disease, progression of a disease, etc.). For example, for a condition of atopic dermatitis, the measurement definition asset may define a concept related to atopic dermatitis to be nocturnal scratching. Thus, nocturnal scratching can be measured for a subject to characterize the disease for the subject (e.g., higher quantity of nocturnal scratching can be indicative of more severe atopic dermatitis as opposed to lower quantity of nocturnal scratching). Examples of individual components of the measurement definition asset are described in further detail herein.

The instrumentation asset defines how the measurement concepts related to the disease are captured, and further defines how the captured raw data is transformed into an interpretable, meaningful health dataset. For example, the instrumentation asset can describe device specifications that influence the capture of the raw data. Furthermore, the instrumentation asset can transform the raw data into the health dataset that is more meaningful for the particular disease. The meaningful health dataset can be measurements of the concept related to the disease (as described in relation to the measurement definition asset) or can be readily interpreted to obtain measurements of the concept related to the disease. Returning to the atopic dermatitis example above, the meaningful health dataset can include a measure of nocturnal scratching (e.g., scratching events per hour, scratching duration per hour, total number of scratching events). Alternatively, the meaningful health dataset can be a dataset from which the measure of nocturnal scratching (e.g., scratching events per hour, scratching duration per hour, total number of scratching events) can be readily extracted. Examples of individual components of the instrumentation asset are described in further detail herein.

The evidence asset includes one or more validations that validate the dataset generated by the instrumentation asset. This ensures that the dataset (e.g., health dataset) generated by the instrumentation asset is accurate and can be used to accurately characterize the disease. Examples of validations included in the evidence asset can include technical validations, analytical validations, and/or clinical validations. In various embodiments, the evidence asset includes two or more validations. In various embodiments, the evidence asset includes three or more validations. Generally, performing the validations of the evidence asset ensures that measurements are accurate, and therefore, can be recognized as eligible (e.g., as a standard) for clinical trial use and approval. Examples of individual components of the evidence asset are described in further detail herein.

In various embodiments, the different assets of the measurement stack are selected or generated specifically for the particular condition. For example, the measurement definition asset may describe concepts particularly relevant to the condition, and therefore, the measurement definition asset may be specific for the condition. In some embodiments, the different assets in a measurement stack are interchangeable and can be used for measurement stacks of various diseases. For example, the instrumentation asset can be interchangeable, such that the instrumentation asset can be included in a first measurement stack for a first condition, and can further be included in a second measurement stack for a second condition. As such, interchangeable or reusable assets enables the more efficient generation and building of measurement stacks.

FIG. 2B is an example measurement stack showing individual components, in accordance with an embodiment. As shown in FIG. 2B, the measurement stack includes three assets (e.g., measurement definition asset, instrumentation asset, and evidence asset). Each of the three assets is composed of two or more components. Specifically, the measurement definition asset includes: 1) aspect of health component, 2) hypothesis component, and 3) concept of interest component. The instrumentation asset includes: 1) measurement method component, 2) raw data component, 3) algorithm component, 4) and health data component. The evidence asset includes: 1) analytical validation component, and 2) clinical validation component. As shown in FIG. 2B, there are a total of 8 components in the measurement stack.

In various embodiments, the measurement stack can be differently arranged such that additional or fewer components are included. In various embodiments, although not shown, the measurement stack can further include a regulatory component, which is valuable for aligning regulatory experts with the measurement endpoints. Such a regulatory component may be included in the evidence asset. Thus, in such embodiments, there are a total of 9 components in the measurement stack.

In various embodiments, functionalities of two or more components in an asset can be combined into a single component. Thus, there may be fewer components in the measurement stack than the 8 components that are explicitly shown in FIG. 2A. For example, the hypothesis component and the concept of interest component can be combined into a single component. As another example, the aspect of health component, the hypothesis component, and the concept of interest component can be combined into a single component.

Referring first to the condition (e.g., physical or medical condition shown in FIG. 2B), it can refer to any disease. Examples of conditions or diseases include atopic dermatitis, Parkinson's Disease, Alzheimer's Disease, chronic obstructive pulmonary disease (COPD), pulmonary arterial hypertension (PAH), asthma, retinal disease, major depressive disorder (MDD), or cancer. Additional examples of conditions are described herein and are shown in Table 1.

The meaningful aspect of health (MAH) (referred to as aspect of health in FIG. 2B), generally defines an aspect of the disease for improvement. Examples of meaningful aspects of health (MAH) are shown in Table 2. The hypothesis refers to a manner of improving the meaningful aspect of health. For example, the hypothesis can involve an intervention (e.g., a therapeutic intervention, a surgical intervention, or a change in lifestyle) that is predicted to improve the meaningful aspect of health relevant to the disease. Generally, improvement of the meaningful aspect of health correlates with improvement of the disease. In various embodiments, for a particular disease, there may be multiple available meaningful aspects of health (e.g., multiple aspects of the disease for improvement).

The concept of interest describes a measurable unit that informs the meaningful aspect of health. For example, the concept of interest is a measurable unit that can be used to inform the meaningful aspect of health relevant to the disease, which therefore informs the severity of the disease. Examples of concepts of interest (COI) are shown in Table 3. In various embodiments, the concept of interest describes a medical measurement of the disease (e.g., a measurable unit that the health care community would measure for determining severity of the disease). For example, in the context of Parkinson's disease, a medical measurement of Parkinson's is tremors. Here, the quantity of tremors can be a measure of the severity of the disease. In various embodiments, the concept of interest describes a measurable experience of individuals suffering from the disease. Here, the measurable experience may not be the medically relevant measurement unit, but may nonetheless have significant impact on patients afflicted with the disease. In such embodiments, the concept of interest can be a symptom of the disease that the patient would like to modify. For example, again in the context of Parkinson's disease, a measurable experience for individuals suffering from Parkinson's may be sleep deprivation. Although sleep deprivation is not the medical measurement unit of Parkinson's Disease, it is nonetheless a measure that can be informative of the severity of the disease.

Referring next to the measurement method component, it generally describes the solutions that are implemented for capturing data of the concept of interest. For example, solutions of the measurement method component include hardware, software, or firmware solutions. Example solutions of the measurement method component include sensors, devices such as computational devices, cellular devices or wearables, as well as mobile applications. In various embodiments, sensors can be built into devices, such as a wearable device or a cellular device.

In various embodiments, the measurement method component identifies the specifications of the measurement method. For example, for a wearable device, the measurement method component identifies the operating specifications of the wearable device (e.g., frequency or a frequency range at which the device captures data (e.g., 10-100 Hz), time intervals during which the device captures data (e.g., 24 hours a day, or in response to a command), presence of one or more sensors of the wearable device that capture data, storage capacity of the wearable device, and/or estimated battery life). In various embodiments, the measurement method employs products that process data captured by (mobile) sensors using algorithms to generate measures of behavioral and/or physiological function. This includes novel measures and indices of characteristics for which the underlying biological processes are not yet understood. Like other digital medicine products, these may be characterized by a body of evidence to support their quality, safety, and effectiveness as indicated in their performance requirements.

Referring next to the raw data, this component represents the raw datasets which are captured according to the particular methods of the measurement method component. For example, if the measurement method component identifies a wearable device (and the corresponding specifications), the raw data represents the dataset captured by the wearable device according to the specifications. In various embodiments, raw data by itself does not provide for interpretable, meaningful data. As a specific example, a raw file may include data captured at 10-100 Hz accelerations. This is captured in 3D SI units (XYZ g-force) with 28 days of continuous data collection. In short, this example describes what raw data is captured (accelerations in 3D SI units), its frequency (10-100 Hz), and the amount of data that is captured (28 days).

Referring next to the algorithm, it transforms the raw data from the raw data component into meaningful datasets (e.g., meaningful health data relevant for measuring the concept of interest). Returning again to the example of atopic dermatitis, an algorithm interprets raw measurement device data captured during sleep and transforms the raw data into meaningful health data (e.g., scratching events). In some scenarios, an algorithm is specific for a particular measurement method. Therefore, a particular algorithm in the algorithm component can only translate raw dataset outcomes that are captured from a particular measurement method.

Referring next to the health data component, it includes health data, also referred to herein as meaningful health data or meaningful health dataset. The health data is transformed by the algorithm from the raw data and represents an interpretable dataset that is informative for the particular concept of interest. Returning again to the atopic dermatitis example, health data can include, or be readily interpreted to include any of total sleep time, scratching events per hour, and the total number of scratching events. Here, health data is the outcome of algorithms/other processing to convert “raw data” into its final health-related data. One example may include converting accelerometer data into number of steps. There may be intermediary stages of this, for example identifying each episode of severe symptoms during the day could be one step, then a further refinement is the calculation of average time of all of these. Both of those could be classified as health data.

Referring next to the analytical validation component, it involves validating one or more of the other components in the measurement stack. In various embodiments, the input to the analytical validation include the components of the measurement definition asset, and components of the instrumentation asset. The output of the analytical validation includes supporting evidence of a successful or failed validation of the corresponding solution incorporating the components of the measurement definition asset and components of the instrumentation asset. Generally, a digital measurement solution is incomplete unless the results it generates are proven to be analytically valid to support clinical interpretation. During the analytical validation, a digital measurement solution is exposed to a series of test conditions and procedural stress to generate sample data and the results are documented for statistical analysis. The results either validate or redefine the functional range outside of which the reliability of measurements may be questionable. A successful analytical validation would mean solutions that fit the profile can support precise labelling claims without unanticipated risks or consequences.

In various embodiments, the analytical validation component may perform an analytical validation of device specifications, algorithms, and health data output. In various embodiments, the analytical validation involves comparing data to an appropriate measurement standard. Example measurement standards for various diseases can be established by third parties or in the community. For example, example measurement standards for different diseases can be standards established by ICHOM Conect.

For example, analytical validation ensures that the meaningful health data meets requisite sensitivity, specificity, and/or reliability requirements. In various embodiments, the requisite sensitivity requirements is any of at least 50% sensitivity, at least 60% sensitivity, at least 70% sensitivity, at least 75% sensitivity, at least 80% sensitivity, at least 85% sensitivity, at least 90% sensitivity, at least 91% sensitivity, at least 92% sensitivity, at least 93% sensitivity, at least 94% sensitivity, at least 95% sensitivity, at least 96% sensitivity, at least 97% sensitivity, at least 98% sensitivity, or at least 99% sensitivity. In various embodiments, the requisite specificity requirements is any of at least 50% specificity, at least 60% specificity, at least 70% specificity, at least 75% specificity, at least 80% specificity, at least 85% specificity, at least 90% specificity, at least 91% specificity, at least 92% specificity, at least 93% specificity, at least 94% specificity, at least 95% specificity, at least 96% specificity, at least 97% specificity, at least 98% specificity, or at least 99% specificity. In various embodiments, the requisite reliability requirements is any of at least 50% reliability, at least 60% reliability, at least 70% reliability, at least 75% reliability, at least 80% reliability, at least 85% reliability, at least 90% reliability, at least 91% reliability, at least 92% reliability, at least 93% reliability, at least 94% reliability, at least 95% reliability, at least 96% reliability, at least 97% reliability, at least 98% reliability, or at least 99% reliability.

In various embodiments, the analytical validation enables the comparison of the digital solution offered by the measurement stack to a reference measure that is currently employed or was previously developed for characterizing the disease. For example, returning to the example of atopic dermatitis, the analytical validation can establish that the digital solution offered by the measurement stack appropriately measures nocturnal scratching according to appropriate reliability, specificity, and sensitivity requirements. Here, the digital solution can be comparable to, or better than a reference measure (e.g., infrared observation to monitor nocturnal scratching).

In various embodiments, the analytical validation component includes an analytical validation and additionally or alternatively includes a technical validation. In various embodiments, the technical validation verifies that the datasets (e.g., raw data from the raw data component and the health data from the health data component) are appropriate. As an example, the technical validation can evaluate whether the captured raw data is in accordance with firmware and/or software protocols that are specific to the device. As another example, the technical validation can evaluate where the raw data captured by the measurement method is according to the specifications identified in the measurement method. For example, the specifications can include battery life, data storage, available measure frequencies. Therefore, the technical validation determines whether the raw data captured by the measurement method aligns with the specifications. As an example, if the raw dataset indicates that the data was captured at a frequency that exceeds the specifications identified in the measurement method, the technical validation can flag the issue and the validation process fails. Alternatively, if the raw dataset indicates that the data was captured at a frequency that is within the specifications identified in the measurement method, the technical validation can be deemed a success. Further details and examples of technical validations are described in Goldsack, J. C., et al. Verification, analytical validation, and clinical validation (V3): the foundation of determining fit-for-purpose for Biometric Monitoring Technologies (BioMeTs). npj Digit. Med. 3, 55 (2020), which is hereby incorporated by reference in its entirety.

Referring next to the clinical validation component, it involves a clinical validation of the digital solution. Clinical validation is the process that evaluates whether the measurement solution acceptably identifies, measures, or predicts a meaningful clinical, biological, physical, functional state, or experience in the specified context of use. An understanding of what level of accuracy, precision, and reliability is valuable for a solution to be useful in a specific clinical research setting. Clinical validation is intended to take a measurement that has undergone verification and analytical validation steps and evaluate whether it can answer a specific clinical question. Generally, a digital measurement solution is incomplete unless the results it generates are interpretable from a clinical perspective and sufficiently relevant to the meaningful aspects of health for the disease. Here the clinical validation component provides the guidelines to clinically interpret the measurements.

For example, the clinical validation can involve analyzing whether the digital solution identifies, measures, and predicts the meaningful clinical, biological, physical, functional state, or experience relevant for the disease.

As an example of a clinical validation, it may include guidelines identifying that a temperature measurement with a delta of 0.00001% is irrelevant for clinical decision making. Furthermore, it may identify that the standard for temperature is a delta of 0.1 degrees. In various embodiments, clinical validation is an in vivo validation that is performed in a specific target population. Thus, clinical validation represents a check as to whether the measurement stack is valid to answer clinical questions relevant to the disease. Returning again to the example of atopic dermatitis, the clinical validation can involve assessing the treatment effects of an intervention on nocturnal scratching within a patient population. Here, the intervention is expected to reduce the quantity of nocturnal scratching. The specific procedure identified in the clinical validation can be performed during or after the clinical trial to ensure that changes in the patient population are accurately evaluated, which provides an accurate evaluation of the impact of the intervention. Further details of clinical validation is described in Goldsack, J. C., et al. Verification, analytical validation, and clinical validation (V3): the foundation of determining fit-for-purpose for Biometric Monitoring Technologies (BioMeTs). npj Digit. Med. 3, 55 (2020), which is hereby incorporated by reference in its entirety.

In various embodiments, the measurement stack further includes a regulatory component for aligning regulatory experts with the measurement endpoints. Generally, the regulatory component may include regulatory guidelines. In various embodiments, the regulatory component includes scientific advice from third parties, such as third party regulators. For example, for a “nocturnal scratch” measure, there is a need for standardizing what “nocturnal” refers to. Thus, the regulatory component can identify guidelines for defining “nocturnal” (e.g., the start time when a person tries to go to sleep), an example of which can be a measure defined as what % of time patient is aware out of their Total Sleep Opportunity (TSO) time. Additional examples of regulatory guidelines may be standard guidelines (e.g., guidelines promulgated by the Food and Drug Administration (FDA) such as FDA patient-reported outcome (PRO) guidance or FDA's Patient Focused Drug Development (PFDD) guidance series).

In various embodiments, the regulatory component includes guidelines that are helpful for achieving regulatory acceptance. For example, different regulatory pathways involve different requirements to achieve regulatory acceptance. In some scenarios, requests can be submitted through FDA CPIM meetings, within an IND, or through the formal qualification procedure. In particular embodiments, the regulatory component assesses one or more other components of the measurement stack, such as components of the measurement definition asset and/or other components of the evidence asset. Thus, the regulatory component enables the saving of resources by involving the regulators early on as the context of use (COU), digital measures (medical device, digital biomarker, clinical outcome assessment), and all validations (e.g., technical, analytical, and clinical validation) can be approved.

In various embodiments, the regulatory component can be made available to third parties, such as regulators, who can further collaborate on co-developing and/or proposing improvements to standardized solutions. This enables a dynamic regulatory evaluation of standardized solutions. For example, regulators can provide new evidence requests and questions in more real time. In response, new evidence, comments, and additional context can be provided to the regulators. In various embodiments, the dynamic regulatory evaluation involves multiple stakeholders (e.g., involving customers, asset developers, pharmaceutical companies, regulators, etc.) and therefore, the regulatory component can be made available to the multiple stakeholders to enable a collaborative approach towards achieving regulatory approval of the standardized solution. An example of dynamic regulatory evaluation is described below in Example 5.

In various embodiments, regulators may evaluate the standardized solutions (e.g., DMSs) for tolerance and/or bias. Here, the regulatory component can provide guidelines for understanding the size of the expected treatment effect. If the effect is massive, tolerance can be greater, if the effect is minuscule, the measure also needs to be more precise. In various embodiments, the regulatory component can involve regulatory advice that is given independent of the intervention (e.g., which measures are meaningful).

FIG. 2C is an example measurement stack indicating one or more components that form a target solution profile (TSP), target instrumentation profile (TIP), or target component profile (TCP), in accordance with an embodiment. In various embodiments, the TSP encompasses the full measurement stack (e.g., all 9 layers as shown in FIG. 2C). In contrast, TIPs and TCPs include fewer than all the components of the measurement stack. For example, TIPs include six components (from the concept of interest up to the technical/analytical validation). TCPs refer to individual components of the measurement stack.

Generally, TSPs are considered solution-agnostic (e.g., no specific brands and versions are named). TIPs are instrumentation-centered and agnostic of certain components of the measurement definition asset (e.g., condition, meaningful aspect of health, hypothesis) as well as certain components of the evidence asset (e.g., clinical validation and regulatory intel). In addition, TIPs are considered condition-agnostic as no components of the TIP layers are associated with a specific condition, meaningful aspect of health, or patient population. This adds new value to the available assets provided by stakeholders and fitting these TIPs. For example, TIPs can be interchangeable across different TSPs that are designed for specific conditions. Therefore, developers (e.g., developers of individual components or assets) can develop functional assets covering multiple conditions. This is in contrast to having developers develop new assets for every specific condition and study design. Novel developments of individual assets may be a waste of resources as often the desired assets might already be available as off-the-shelf solutions. Furthermore, assets included in TIPs can readily be repurposed for multiple conditions. By implementing clinical validation for a specific condition, TIPs are applicable across various conditions, thereby allowing for improved reusability and sustainability of available assets. For example, a class of actigraphy solutions is validated to measure daily life physical activity (DLPA) for a first condition of pulmonary arterial hypertension. Analytical validation has validated the ability to measure DLPA parameters by devices included in this TIP. Additionally, DLPA can also be a concept of interest in a second condition of Parkinson's Disease (PD). Therefore, the validated TIP for measuring DLPA can be repurposed for the second condition of PD without the need to re-perform the technical and analytical validation steps, as these are already included in the TIP. Additionally, TIPs provide a structure to available assets, preventing stakeholders from being overwhelmed by the countless TSPs, devices, and algorithms accessible for digital measures. As a result, solutions within one TIP can be easily compared to deliver the best fit-for-purpose solution for the novel study design. This improves the likelihood that the best instrumentation is picked for specific use cases.

As shown in FIG. 2C, TCPs define a single generic component. TCPs thus include generic descriptions of technical details of the individual components. For example, the TCP identified in FIG. 2C can include a generic description of a measurement method (e.g., 3-axis accelerometer (wrist-worn), 10-100 Hz, 18+ hours battery life, 500+MB data storage). Generally, TCPs allow for the ease of substituting assets of the same or similar TCPs into multiple TIPs and TSPs. For example, a TSP includes a measurement method with a battery life of 18+ hours (Apple Watch 6), but a battery life of at least 96 hours is required for a specific study design. Therefore, the TCP describing 18+ hours can be substituted with a different TCP with at least 96 hours of battery life. This excludes the availability of the Apple Watch 6 in the TIP/TSP, and only components meeting the requirements will be included. TCPs thus allow researchers to best fit their specific needs with more ease.

### Example Target Solution Profile

As described herein, a TSP encompasses a full measurement stack. The generation and maintenance of TSPs can be performed by the TSP module 145, as described above in FIG. 1B. Individual components of a TSP are described using generic descriptions (in contrast to DMSs which describe specific solutions), thereby providing a device technology agnostic profile. Device technology agnostic refers to both hardware agnostic (e.g., agnostic of a particular device) as well as software agnostic (e.g., agnostic as to software version, such as software for an application or software for a device). This prevents any associations with a specific brand, model, or technology and allows for smooth emulation of specific DMSs into generic TSPs. Thus, a TSP is a generic profile that defines a class of DMSs, each of which provides further specificity to the generic profile. Since TSPs are device technology agnostic, they provide novel solutions for the same or even different conditions with more ease. This allows for the improved development of future-proof and sustainable solutions. Furthermore, TSPs provide harmonization in the ecosystem and show high potential to shorten the lifecycle of solution developments as earlier generated evidence can be repurposed. Taken together, TSPs show the potential to ultimately accelerate the adoption of digital measures in clinical research. Example TSPs are further detailed in Table 4.

TSPs provide a generic description that covers multiple DMSs within the same solution class. The DMSs that fit in the class represented by the TSP can be considered fit-for-purpose for the same use case. As a result of TSPs being device technology agnostic, included DMSs show improved reusability of assets. Instead of being solely purposed for one study, TSPs accelerate the repurposing of available assets and increase the value of all assets included in DMSs. Also, TSP-classes can be leveraged to compare slight differences between similar TSPs. This allows stakeholders to compare multiple TSPs (and DMSs in the class represented by a TSP) with more ease to select the best preferences for their specific use-case (e.g., costs, the weight of the device, or battery life). In various embodiments, a DMS can also fit the generic description of various TSPs.

Furthermore, TSPs allow for versioning (life cycle management of digital measurement solutions). In time, available devices, algorithms, and technologies evolve. In various embodiments, the TSPs can be edited and modified, resulting in updated versions (which can co-exist with older versions). This allows for components of the solution to be upgraded if the solution overall still meets the TSP criteria. The validation of evolving TSPs is assessed by qualification protocols (QPs). QPs validate versioning and ensure TSPs are considered future-proof In various embodiments, QPs allow for the versioning of specific assets and the ability to validate comparability between multiple DMSs. Qualification protocols are further described in.

FIG. 2D shows an example target solution profile, in accordance with an embodiment. Here, the components of the TSP are described in generic terms. However, although the TSP shown in FIG. 2D identifies a generic disease or condition, in various embodiments, the TSP identifies a specific disease or condition.

Referring to the measurement method component, it describes a device agnostic measurement method. For example, the measurement method component can specify a particular class of devices. Examples of a class devices can include, but are not limited to: any of wearable devices, ingestibles, image and voice based devices, touchless sensors, and sensor based smart devices (e.g., scales, thermometers, and respirators). The measurement method component can further include specifications of the measurement method e.g., battery life, data storage, available measure frequencies). Thus, a developer can determine whether the TSP is appropriate for their digital solution based on the specifications of the measurement method (e.g., if the developer needs to capture data for at least 96 hours, but the TSP measurement method specifies a battery life of 18 hours, then the developer determines that a different TSP is needed).

Referring to the raw data component of the TSP, it describes the raw file that is captured according to the measurement method. For example, the raw data according to the specifications of the measurement method. Therefore, if the measurement method indicates a measurement frequency of 100 Hz, the raw data component describes a raw file that includes data captured at the 100 Hz measurement frequency. In various embodiments, digital measurements reported by measurement methods are derived through a data supply chain, which includes hardware, firmware, and software components. The term “raw data” is used to describe data existing in an early stage of the data supply chain. Sensor output data at the sample level (for example, a 50 Hz accelerometer signal or a 250 Hz ECG signal) would be raw data. Although signal processing methods may have been applied to this data (e.g., down sampling, filtering, interpolation, smoothing, etc.), the data are still considered “raw” because it is a direct representation of the original signal produced by the sensor.

Referring to the algorithm component of the TSP, it identifies one or more algorithms that can appropriately transform the raw data into meaningful health data. Here, the algorithm is designed according to the specific measurement method that was used to capture the raw data. As an example, if the measurement method indicates a measurement frequency of 100 Hz, then the algorithm is designed to transform the data that was specifically captured at a frequency of Hz. In various embodiments, the algorithm component represents a range of data manipulation processes embedded in firmware and software, including but not limited to signal processing, data compression and decompression, artificial intelligence, and machine learning. An algorithm is a calculation that transforms the data from the sensor into meaningful information. The algorithms may be part of the sensor directly, or may be operated by a party to conduct additional data science to create a derived measure.

Referring to the analytical validation component, it enables the validation of the components of the instrumentation asset (e.g., measurement method, raw data, algorithm, and health data) to ensure that the raw data and/or health data is reliable, valid, and sensitive to meet appropriate standards. In various embodiments, the analytical validation occurs at the intersection of engineering and clinical expertise. It involves evaluation of the processed data and requires testing with human subjects. After verified sample-level data have been generated by a measurement method, algorithms are applied to these data in order to create behaviorally or physiologically meaningful metrics. This process begins at the point at which verified output data (sample-level data), becomes the data input for algorithmic processing. Therefore, the first step of analytical validation requires a defined data capture protocol and a specified test subject population. During the process of analytical validation, the metric produced by the algorithm is evaluated against an appropriate reference standard.

In various embodiments, a TSP can be built using a condition-focused approach (e.g., bottom-up approach). In various embodiments, a TSP can be built using an instrumentation-focused approach (e.g., top-down approach). Regardless of the approach (e.g., bottom up or top down), the final TSP and DMS(s) of the class can be identical.

Referring first to the condition-focused approach, a specific condition is identified. Here, a measurement definition meaningful for patients with the condition is determined. This includes determining the concept of interest that will be measured. Next, suitable instrumentation is developed, or, if available, off-the-shelf solutions could be selected. For example, an instrumentation asset of a different TSP could be selected and repurposed for this current TSP. Given that the instrumentation asset of TSPs is generally described in generic terms, the repurposing of the instrumentation asset for the current TSP can require little or no additional work. Next, an evidence asset is generated for the TSP. In various embodiments, generating an evidence asset involves determining technical and analytical validations that are appropriate for the instrumentation of the TSP. In various embodiments, components of the evidence asset, such as components for performing technical and analytical validations, can be repurposed from another TSP. Given that technical and analytical validations may have previously been performed for a generic instrumentation asset of another TSP, the current TSP need not re-perform the same technical and analytical validations again. In various embodiments, a component of the evidence asset includes a clinical validation component. Here, the clinical validation component verifies whether results are clinically valid. Thus, generating an evidence asset includes generating the clinical validation component that is valuable for ensuring the results of the TSP are of clinical value.

Referring to the instrumentation-focused approach, it begins with the discovery of available components. Components with similar instrumentation are identified and thereafter profiled into classes of solutions. These individual components are profiled into individual component classes known as TCPs (solely one generically described layer of the measurement stack, e.g., measurement method or algorithm). Multiple TCPs of different layers can be united into TIPs if completed with the concept of interest, measurement method, raw data, algorithm, health data, and technical verification/analytical validation. TSPs are built on top of these TIPs with aligned definitions and validated instrumentation by completing them with the condition-related layers. For example, TSPs are built by generating the meaningful aspect of health component and clinical validation component on top of the components of the TIPs.

The instrumentation-focused approach eases the development of TCPs, multiple TIPs, and numerous TSPs. In the instrumentation-focused approach, smaller stakeholders could easier contribute to the development of assets, as they often have assets as measurement methods and algorithms within their digital portfolios. Asset providers can now focus more on the development of one component, which can be useful for multiple studies.

In various embodiments, given a full TSP, assets and components of the TSP can be quickly drafted. For example, TIPs and TCPs can quickly be drafted from a complete TSP by excluding the definition-related layers or by picking one individual layer, respectively.

As described herein, assets and components of TSPs can be interchangeable and substituted. For example, a TIP from a first TSP can be substituted for in place of a second TIP in a second TSP. Here, substituting a TIP can be beneficial as it minimizes the validations that are required in view of the substitution. For example, only a technical validation/analytical validation for TSP including the now substituted TSP is re-assessed. As another example, only a clinical validation for the TSP including the now substituted TSP is re-assessed (e.g., technical/analytical validation need not be performed).

### Example Digital Measurement Solution

FIG. 2E shows an example digital measurement solution, in accordance with a first embodiment. FIG. 2F shows an example digital measurement solution, in accordance with a second embodiment. Although not explicitly identified in FIGS. 2E and 2F, each DMS can be developed for a specific disease or condition. As an example, the DMS in FIGS. 2E and 2F can be developed for characterizing pulmonary arterial hypertension. The generation and maintenance of DMSs can be performed by the DMS module 150, as described above in FIG. 1B.

Referring first to the DMS shown in FIG. 2E (referred to as DMS #1), this DMS is not device-technology agnostic as was the case for the TSP shown in FIG. 2D. Rather, DMS #1 specifically identifies the particular device that is used to capture the raw data. Here, the measurement method component of DMS #1 identifies an ActiGraph GT9X Link device that captures the raw data. The Actigraph GT9X link device include particular specifications, including the presence of a gyroscope, accelerometer, thermometer, and magnetometer, a measurement frequency of 100 Hz, a 4 GB storage, and a 1-day battery life. These specifications of the Actigraph GT9X Link device may satisfy the specifications of a measurement method of the corresponding TSP that represents the class in which the DMS #1 is a part of. Although not explicitly shown, the measurement method component of DMS #1 can further specify software that is used to capture the raw data. For example, the measurement method component of DMS #1 can specify a software version that the Actigraph GT9X Link device is operating on (e.g., an operating system version).

The algorithm component of DMS #1 identifies a specific algorithm (e.g., an ActiGraph deterministic algorithm such as ActiLife 6) that can transform raw data captured by the Actigraph GT9X Link device into meaningful health datasets. Again, in contrast to the corresponding TSP which generically described algorithms, the algorithm component of DMS #1 identifies a specific algorithm that can be executed.

Referring next to the DMS shown in FIG. 2F (referred as DMS #2), it also is no longer device-technology agnostic as was the case for TSPs. Here, DMS #2 specifically identifies a Garmin Vivofit 4 device in the measurement method component. Thus, the Garmin Vivofit 4 device can be used to capture raw data according to the specifications of the device. The measurement method component identifies those specifications including the presence of a gyroscope, accelerometer, thermometer, and magnetometer, a measurement frequency of 50 Hz, a 2 GB storage, and a 7 day battery life. The algorithm component of DMS #2 identifies a specific algorithm (e.g., a machine learning algorithm) that transforms raw data captured by the Garmin Vivofit 4 device into meaningful health datasets. Again, in contrast to the corresponding TSP which generically described algorithms, the algorithm component of DMS #1 identifies a specific algorithm that can be executed. Although not explicitly shown, the measurement method component of DMS #2 can further specify software that is used to capture the raw data. For example, the measurement method component of DMS #2 can specify a software version that the Garmin Vivofit 4 device is operating on (e.g., an operating system version).

Here, the DMSs (e.g., DMS #1 and DMS #2) shown in FIGS. 2E and 2F may be of a common class represented by a target solution profile. The device specifications of the measurement method components of DMS #1 and DMS #2 may satisfy the specifications identified in the measurement method component of the target solution profile. For example, the measurement method component of the target solution profile may identify one or more of the following device specifications: 1) availability of gyroscope, accelerometer, thermometer, and magnetometer, 2) measurement frequency between 1 Hz and 100 Hz, 3) storage between 1 GB and 4 GB, and 4) battery life between 1 and 7 days.

In various embodiments, a component of DMS #1 and DMS #2 can be interchangeable. For example, the measurement method component of DMS #1 can replace the measurement method component of DMS #2. As another example, the algorithm component of DMS #1 can replace the algorithm component of DMS #2. In various embodiments, a set of components of DMS #1 and DMS #2 can be interchangeable. For example, the measurement method component and algorithm component of DMS #1 can replace the measurement method component and algorithm component of DMS #2, respectively. As another example, the instrumentation asset of DMS #1 (e.g., measurement method component, raw data component, algorithm component, and health data component) can replace the corresponding instrumentation asset of DMS #2. As another example, the target instrumentation profile of DMS #1 (e.g., concept of interest, measurement method component, raw data component, algorithm component, health data component, and analytical validation component) can replace the corresponding target instrumentation profile of DMS #2.

Further examples of digital measurement solutions (DMSs) are further detailed in Table 5.

### Life-Cycle Management of TSPs and DMSs

Digital measurement solutions are subject to a rapidly evolving lifecycle as the components of the instrumentation asset are always facing the possibility of being upgraded (e.g., due to new device release or new software release). Managing the rapid technological evolution while maintaining equivalency between different measurement solutions and versions is a new challenge. These upgrades, for example, can be bug fixes or add novel features to available devices.

In various embodiments, qualification protocols are implemented to improve the life cycle management of rapidly evolving components in relation to their TSPs. The implementation of qualification protocols can be performed by the qualification protocol module 155 (see FIG. 1). Generally, qualification protocols (QPs) confirm the functionality of the upgrades without the need to re-assess the full digital measurement solution or full target solution profile. For example, QPs can be implemented to ensure that digital measurement solutions that incorporate an upgraded version (due to new device release or new software release) achieves comparable solutions in comparison to a prior version.

In various embodiments, a QP evaluates a new TSP and/or new DMS in view of an upgraded device or software release, and upon a successful validation, the new TSP or DMS can be stored e.g., as part of the marketplace or catalog. Here, the new TSP or DMS can replace the prior solution (e.g., prior TSP or prior DMS). In various embodiments, this can involve replacing hardware components and/or delivering software upgrades. In various embodiments, the new TSP or DMS that has been validated using a QP can represent an additional asset e.g., for inclusion in the marketplace or catalog. For example, the new DMS can include the upgraded device or upgraded software and therefore, can be used to characterize a disease. This can be in addition to the prior DMS that includes a prior version of the device or software, which continues to be a solution for characterizing the disease, albeit with older hardware/software.

QPs can involve an evidence-based validation process that enables improved life cycle management of TSPs. These QPs represent standardized experiments that generate evidence that the overall solution from a TSP performs at a sufficient level for its intended purpose. In various embodiments, QPs are fully automated for validating, for example, upgraded algorithms. Thus, results of the upgraded algorithms can be referenced against available reference datasets. In various embodiments, QPs involve controlled experiments. For example, if a new, upgraded device is released, a QP can involve evaluating the results of the upgraded device in comparison to a prior version of the device by using both devices on a patient population.

In particular embodiments, QPs are implemented to ensure that the upgraded TSP or DMS (e.g., due to incorporation of upgraded device or software) achieves comparable results in comparison to prior versions of the TSP or DMS. In various embodiments, raw data captured by a device of an older version of a DMS and a raw data captured by a new device of a newer version of a DMS are comparable if the difference between the raw data are less than a threshold number. In various embodiments, the threshold number is 1%, 2%, 3%, 4%, 5%, 6%, 7%, 8%, 9%, 10%, 11%, 12%, 13%, 14%, 15%, 16%, 17%, 18%, 19%, or 20%. In particular embodiments, the threshold number is 10%. In particular embodiments, the threshold number is 5%. In particular embodiments, the threshold number is 2%. In various embodiments, meaningful health data transformed from raw data captured by a device of an older version of a DMS and meaningful health data transformed from raw data captured by a new device of a newer version of a DMS are comparable if the difference between the different meaningful health data are less than a threshold number. In various embodiments, the threshold number is 1%, 2%, 3%, 4%, 5%, 6%, 7%, 8%, 9%, 10%, 11%, 12%, 13%, 14%, 15%, 16%, 17%, 18%, 19%, or 20%. In particular embodiments, the threshold number is 10%. In particular embodiments, the threshold number is 5%. In particular embodiments, the threshold number is 2%.

In various embodiments, upon a successful validation using a QP, the upgraded TSP or upgraded DMS can be annotated accordingly. For example, the metadata of the TSP or DMS can be annotated with an indication that a validation using a QP was successfully performed. In various embodiments, the metadata may be available for inspection by third parties (e.g., through a catalog or marketplace). Thus, a third party can readily be informed of TSPs and/or DMSs that have been successfully validated using a QP.

To provide an example, an actigraphy-assessed TSP with a daily life physical activity-endpoint (COI=gait speed), covers numerous smartwatch devices (e.g., the Apple Watch 5, GENEActiv Original watch, ActiGraph GT9X Link). Over time, a new smartwatch device, such as the Apple Watch 6, is released. The QP is implemented to ensure that the outcomes measured by DMSs due to the upgraded device remain valid. An example how this could be assessed using a qualification protocol is as follows:

1. Participants of a small group (N=20) wear both the old and the new device (e.g., on either wrist—if wrist-worn devices). Participants can be healthy individuals or, alternatively, can include patient populations. For example, if there is no difference in measuring gait speed between healthy participants and patient populations, the patient participants can be included as participants.

2. With a concept of interest being gait speed, participants are asked to perform physical activity related tasks as walking, running and walking the stairs while wearing both the old and new device.

3. Raw data is continuously captured for a prolonged period of time (e.g., 5 days) and corresponding algorithms (same or new) translate the raw dataset into meaningful health data sets with gait speed evidence.

4. If the translated health datasets are within a comparable range (e.g., <2%), both devices are validated as comparable and the Apple Watch 6 can now be considered for the same research purposes as its older device. Conversely, if the QP cannot validate the new device as generating comparable results, the manufacturer can decide to re-assess the new device to ensure that it will be validated by the QP in a second assessment.

In various embodiments, a new device release or new software release can exceed the specifications of a TSP. For example, assume that the newly released Apple Watch 6 acquires raw data at a frequency range between 32-256 Hz. This may exceed the specifications of the TSP (e.g., 1-100 Hz frequencies). Thus, a new TSP or upgraded TSP that incorporates the broader device specification (e.g., broader frequencies) can be generated and validated using the QPs.

### Example Methods

### Building a TSP or DMS

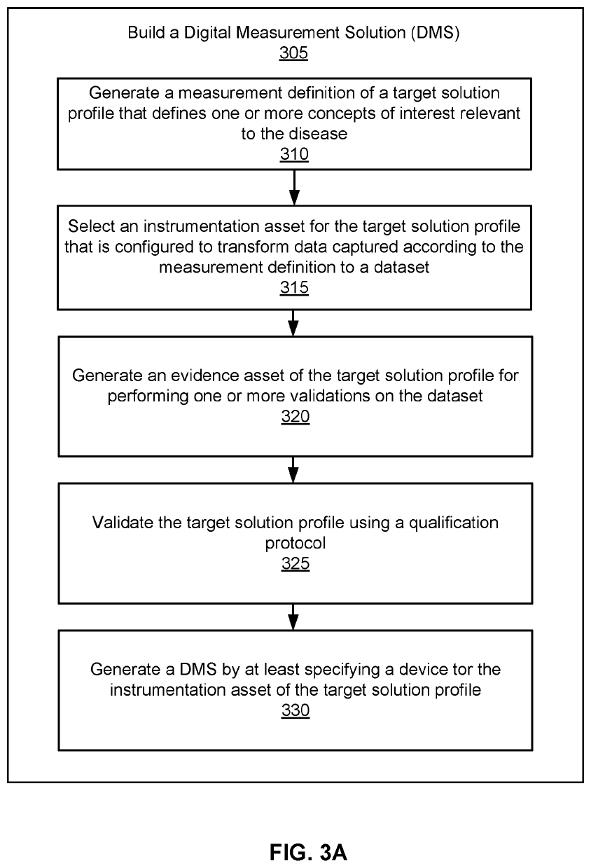

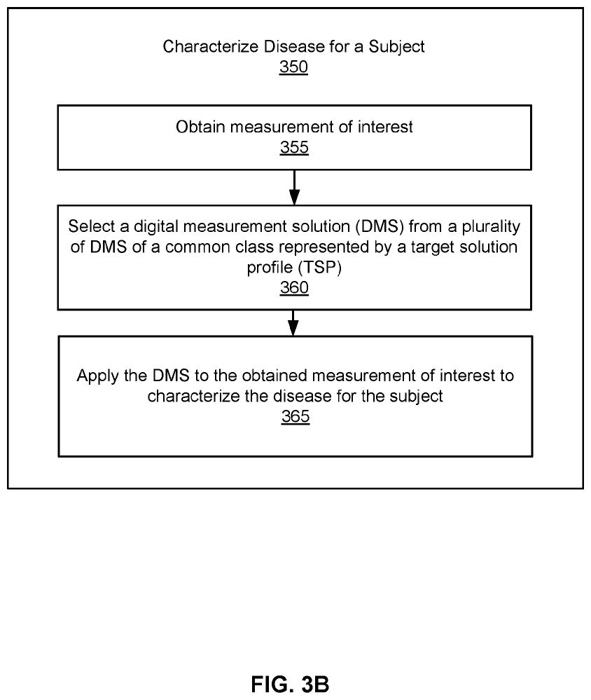

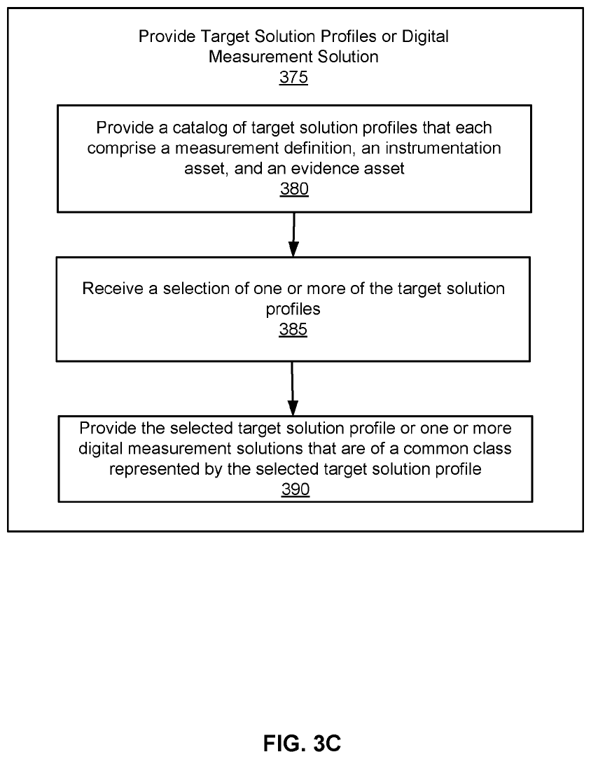

FIG. 3A is an example flow process 305 for building a digital measurement solution, in accordance with an embodiment. Step 310 involves generating a measurement definition of a target solution profile that defines one or more concepts of interest relevant to a disease. Step 315 involves generating or selecting an instrumentation asset for the target solution profile that is configured to transform data captured according to the measurement definition to a dataset, such as a meaningful health dataset. In one scenario, an instrumentation asset in a different TSP was previously generated and therefore, the instrumentation asset can be selected and repurposed here. In another scenario, an instrumentation asset is generated de novo. Step 320 involves generating an evidence asset for the target solution profile for performing one or more validations on the dataset, such as the meaningful health dataset. Step 325 involves validating the target solution profile using a qualification protocol. Step 330 involves generating a DMS by at least specifying a device for the instrumentation asset of the target solution profile. In various embodiments, step 330 further includes specifying a particular algorithm that transforms raw data captured by the device to a meaningful health dataset.

### Characterizing a Disease Using a DMS

FIG. 3B is an example flow process 350 for characterizing a disease for a subject using a digital measurement solution (DMS), in accordance with an embodiment. Although the flow diagram in FIG. 3B shows the steps of 355, 360, and 365 in that order, in various embodiments, the steps 355, 360, and 365 can be differently ordered. For example, a DMS can be first selected at step 360 before obtaining a measurement of interest at step 355.

Step 355 involves obtaining a measurement of interest. Here, the measurement of interest can be captured using a measurement method specified by a digital measurement solution. For example, the measurement of interest can be captured using a particular device having specifications (e.g., data storage, battery life, measurement frequency) that are specified in the digital measurement solution (e.g., in the measurement method component).

Step 360 involves selecting a DMS from a plurality of DMS of a common class represented by a target solution profile. Here, the selected DMS specifies the measurement method by which the measurement of interest was captured in step 355.

Step 365 involves applying one or more components of the DMS to the obtained measurement of interest to characterize the disease for the subject. For example, step 365 can involve applying an algorithm specified in the algorithm component of the DMS. The algorithm transforms raw data of the measurement of interest to a meaningful health dataset. Thus, the disease of the subject can be characterized according to the meaningful health dataset.

### Providing a TSP or DMS

FIG. 3C is an example flow process 375 for providing a target solution profile or one or more digital measurement solutions, in accordance with an embodiment. Step 380 involves providing a catalog of TSPs. For example, step 380 can involve presenting a catalog of TSPs in a marketplace to a third party, such that the third party can access the catalog of TSPs. In various embodiments, each TSP includes a measurement definition asset, an instrumentation asset, and an evidence asset.

Step 385 involves receiving a selection of one or more of the TSPs in the catalog. For example, a third party may select a TSP that suit their needs.

Step 390 involves providing the selected TSP or one or more digital measurement solutions that are of a common class represented by the selected TSP. In various embodiments, the selected TSP is provided. In various embodiments, the one or more digital measurement solutions is provided.

### Diseases and Conditions

Disclosed herein are TSPs and DMSs that are built and implemented for specific diseases or conditions. In various embodiments, the disease can be, for example, a cancer, inflammatory disease, neurodegenerative disease, neurological disease, autoimmune disorder, neuromuscular disease, metabolic disorder (e.g., diabetes), cardiac disease, or fibrotic disease.

In various embodiments, the cancer can be any one of lung bronchioloalveolar carcinoma (BAC), bladder cancer, a female genital tract malignancy (e.g., uterine serous carcinoma, endometrial carcinoma, vulvar squamous cell carcinoma, and uterine sarcoma), an ovarian surface epithelial carcinoma (e.g., clear cell carcinoma of the ovary, epithelial ovarian cancer, fallopian tube cancer, and primary peritoneal cancer), breast carcinoma, non-small cell lung cancer (NSCLC), a male genital tract malignancy (e.g., testicular cancer), retroperitoneal or peritoneal carcinoma, gastroesophageal adenocarcinoma, esophagogastric junction carcinoma, liver hepatocellular carcinoma, esophageal and esophagogastric junction carcinoma, cervical cancer, cholangiocarcinoma, pancreatic adenocarcinoma, extrahepatic bile duct adenocarcinoma, a small intestinal malignancy, gastric adenocarcinoma, cancer of unknown primary (CUP), colorectal adenocarcinoma, esophageal carcinoma, prostatic adenocarcinoma, kidney cancer, head and neck squamous carcinoma, thymic carcinoma, non-melanoma skin cancer, thyroid carcinoma (e.g., papillary carcinoma), a head and neck cancer, anal carcinoma, non-epithelial ovarian cancer (non-EOC), uveal melanoma, malignant pleural mesothelioma, small cell lung cancer (SCLC), a central nervous system cancer, a neuroendocrine tumor, and a soft tissue tumor. For example, in certain embodiments, the cancer is breast cancer, non-small cell lung cancer, bladder cancer, kidney cancer, colon cancer, and melanoma.

In various embodiments, the inflammatory disease can be any one of acute respiratory distress syndrome (ARDS), acute lung injury (ALI), alcoholic liver disease, allergic inflammation of the skin, lungs, and gastrointestinal tract, allergic rhinitis, ankylosing spondylitis, asthma (allergic and non-allergic), atopic dermatitis (also known as atopic eczema), atherosclerosis, celiac disease, chronic obstructive pulmonary disease (COPD), pulmonary arterial hypertension (PAH), chronic respiratory distress syndrome (CRDS), colitis, dermatitis, diabetes, eczema, endocarditis, fatty liver disease, fibrosis (e.g., idiopathic pulmonary fibrosis, scleroderma, kidney fibrosis, and scarring), food allergies (e.g., allergies to peanuts, eggs, dairy, shellfish, tree nuts, etc.), gastritis, gout, hepatic steatosis, hepatitis, inflammation of body organs including joint inflammation including joints in the knees, limbs or hands, inflammatory bowel disease (IBD) (including Crohn's disease or ulcerative colitis), intestinal hyperplasia, irritable bowel syndrome, juvenile rheumatoid arthritis, liver disease, metabolic syndrome, multiple sclerosis, myasthenia gravis, neurogenic lung edema, nephritis (e.g., glomerular nephritis), non-alcoholic fatty liver disease (NAFLD) (including non-alcoholic steatosis and non-alcoholic steatohepatitis (NASH)), obesity, prostatitis, psoriasis, psoriatic arthritis, rheumatoid arthritis (RA), sarcoidosis sinusitis, splenitis, seasonal allergies, sepsis, systemic lupus erythematosus, uveitis, and UV-induced skin inflammation.

In various embodiments, the neurodegenerative disease can be any one of Alzheimer's disease, Parkinson's disease, traumatic CNS injury, Down Syndrome (DS), glaucoma, amyotrophic lateral sclerosis (ALS), frontotemporal dementia (FTD), and Huntington's disease. In addition, the neurodegenerative disease can also include Absence of the Septum Pellucidum, Acid Lipase Disease, Acid Maltase Deficiency, Acquired Epileptiform Aphasia, Acute Disseminated Encephalomyelitis, ADHD, Adie's Pupil, Adie's Syndrome, Adrenoleukodystrophy, Agenesis of the Corpus Callosum, Agnosia, Aicardi Syndrome, AIDS, Alexander Disease, Alper's Disease, Alternating Hemiplegia, Anencephaly, Aneurysm, Angelman Syndrome, Angiomatosis, Anoxia, Antiphosphipid Syndrome, Aphasia, Apraxia, Arachnoid Cysts, Arachnoiditis, Arnold-Chiari Malformation, Arteriovenous Malformation, Asperger Syndrome, Ataxia, Ataxia Telangiectasia, Ataxias and Cerebellar or Spinocerebellar Degeneration, Autism, Autonomic Dysfunction, Barth Syndrome, Batten Disease, Becker's Myotonia, Behcet's Disease, Bell's Palsy, Benign Essential Blepharospasm, Benign Focal Amyotrophy, Benign Intracranial Hypertension, Bernhardt-Roth Syndrome, Binswanger's Disease, Blepharospasm, Bloch-Sulzberger Syndrome, Brachial Plexus Injuries, Bradbury-Eggleston Syndrome, Brain or Spinal Tumors, Brain Aneurysm, Brain injury, Brown-Sequard Syndrome, Bulbospinal Muscular Atrophy, Cadasil, Canavan Disease, Causalgia, Cavernomas, Cavernous Angioma, Central Cord Syndrome, Central Pain Syndrome, Central Pontine Myelinolysis, Cephalic Disorders, Ceramidase Deficiency, Cerebellar Degeneration, Cerebellar Hypoplasia, Cerebral Aneurysm, Cerebral Arteriosclerosis, Cerebral Atrophy, Cerebral Beriberi, Cerebral Gigantism, Cerebral Hypoxia, Cerebral Palsy, Cerebro-Oculo-Facio-Skeletal Syndrome, Charcot-Marie-Tooth Disease, Chiari Malformation, Chorea, Chronic Inflammatory Demyelinating Polyneuropathy (CIDP), Coffin Lowry Syndrome, Colpocephaly, Congenital Facial Diplegia, Congenital Myasthenia, Congenital Myopathy, Corticobasal Degeneration, Cranial Arteritis, Craniosynostosis, Creutzfeldt-Jakob Disease, Cumulative Trauma Disorders, Cushing's Syndrome, Cytomegalic Inclusion Body Disease, Dancing Eyes-Dancing Feet Syndrome, Dandy-Walker Syndrome, Dawson Disease, Dementia, Dementia With Lewy Bodies, Dentate Cerebellar Ataxia, Dentatorubral Atrophy, Dermatomyositis, Developmental Dyspraxia, Devic's Syndrome, Diabetic Neuropathy, Diffuse Sclerosis, Dravet Syndrome, Dysautonomia, Dysgraphia, Dyslexia, Dysphagia, Dyssynergia Cerebellaris Myoclonica, Dystonias, Early Infantile Epileptic Encephalopathy, Empty Sella Syndrome, Encephalitis, Encephalitis Lethargica, Encephaloceles, Encephalopathy, Encephalotrigeminal Angiomatosis, Epilepsy, Erb-Duchenne and Dejerine-Klumpke Palsies, Erb's Palsy, Essential Tremor, Extrapontine Myelinolysis, Fabry Disease, Fahr's Syndrome, Fainting, Familial Dysautonomia, Familial Hemangioma, Familial Periodic Paralyzes, Familial Spastic Paralysis, Farber's Disease, Febrile Seizures, Fibromuscular Dysplasia, Fisher Syndrome, Floppy Infant Syndrome, Foot Drop, Friedreich's Ataxia, Frontotemporal Dementia, Gangliosidoses, Gaucher's Disease, Gerstmann's Syndrome, Gerstmann-Straussler-Scheinker Disease, Giant Cell Arteritis, Giant Cell Inclusion Disease, Globoid Cell Leukodystrophy, Glossopharyngeal Neuralgia, Glycogen Storage Disease, Guillain-Barre Syndrome, Hallervorden-Spatz Disease, Head Injury, Hemicrania Continua, Hemifacial Spasm, Hemiplegia Alterans, Hereditary Neuropathy, Hereditary Spastic Paraplegia, Heredopathia Atactica Polyneuritiformis, Herpes Zoster, Herpes Zoster Oticus, Hirayama Syndrome, Holmes-Adie syndrome, Holoprosencephaly, HTLV-1 Associated Myelopathy, Hughes Syndrome, Huntington's Disease, Hydranencephaly, Hydrocephalus, Hydromyelia, Hypernychthemeral Syndrome, Hypersomnia, Hypertonia, Hypotonia, Hypoxia, Immune-Mediated Encephalomyelitis, Inclusion Body Myositis, Incontinentia Pigmenti, Infantile Hypotonia, Infantile Neuroaxonal Dystrophy, Infantile Phytanic Acid Storage Disease, Infantile Refsum Disease, Infantile Spasms, Inflammatory Myopathies, Iniencephaly, Intestinal Lipodystrophy, Intracranial Cysts, Intracranial Hypertension, Isaac's Syndrome, Joubert syndrome, Kearns-Sayre Syndrome, Kennedy's Disease, Kinsbourne syndrome, Kleine-Levin Syndrome, Klippel-Feil Syndrome, Klippel-Trenaunay Syndrome (KTS), Kluver-Bucy Syndrome, Korsakoff s Amnesic Syndrome, Krabbe Disease, Kugelberg-Welander Disease, Kuru, Lambert-Eaton Myasthenic Syndrome, Landau-Kleffner Syndrome, Lateral Medullary Syndrome, Learning Disabilities, Leigh's Disease, Lennox-Gastaut Syndrome, Lesch-Nyhan Syndrome, Leukodystrophy, Levine-Critchley Syndrome, Lewy Body Dementia, Lipid Storage Diseases, Lipoid Proteinosis, Lissencephaly, Locked-In Syndrome, Lou Gehrig's Disease, Lupus, Lyme Disease, Machado-Joseph Disease, Macrencephaly, Melkersson-Rosenthal Syndrome, Meningitis, Menkes Disease, Meralgia Paresthetica, Metachromatic Leukodystrophy, Microcephaly, Migraine, Miller Fisher Syndrome, Mini-Strokes, Mitochondrial Myopathies, Motor Neuron Diseases, Moyamoya Disease, Mucolipidoses, Mucopolysaccharidoses, Multiple sclerosis (MS), Multiple System Atrophy, Muscular Dystrophy, Myasthenia Gravis, Myoclonus, Myopathy, Myotonia, Narcolepsy, Neuroacanthocytosis, Neurodegeneration with Brain Iron Accumulation, Neurofibromatosis, Neuroleptic Malignant Syndrome, Neurosarcoidosis, Neurotoxicity, Nevus Cavernosus, Niemann-Pick Disease, Non 24 Sleep Wake Disorder, Normal Pressure Hydrocephalus, Occipital Neuralgia, Occult Spinal Dysraphism Sequence, Ohtahara Syndrome, Olivopontocerebellar Atrophy, Opsoclonus Myoclonus, Orthostatic Hypotension, O'Sullivan-McLeod Syndrome, Overuse Syndrome, Pantothenate Kinase-Associated Neurodegeneration, Paraneoplastic Syndromes, Paresthesia, Parkinson's Disease, Paroxysmal Choreoathetosis, Paroxysmal Hemicrania, Parry-Romberg, Pelizaeus-Merzbacher Disease, Perineural Cysts, Periodic Paralyzes, Peripheral Neuropathy, Periventricular Leukomalacia, Pervasive Developmental Disorders, Pinched Nerve, Piriformis Syndrome, Plexopathy, Polymyositis, Pompe Disease, Porencephaly, Postherpetic Neuralgia, Postinfectious Encephalomyelitis, Post-Polio Syndrome, Postural Hypotension, Postural Orthostatic Tachyardia Syndrome (POTS), Primary Lateral Sclerosis, Prion Diseases, Progressive Multifocal Leukoencephalopathy, Progressive Sclerosing Poliodystrophy, Progressive Supranuclear Palsy, Prosopagnosia, Pseudotumor Cerebri, Ramsay Hunt Syndrome I, Ramsay Hunt Syndrome II, Rasmussen's Encephalitis, Reflex Sympathetic Dystrophy Syndrome, Refsum Disease, Refsum Disease, Repetitive Motion Disorders, Repetitive Stress Injuries, Restless Legs Syndrome, Retrovirus-Associated Myelopathy, Rett Syndrome, Reye's Syndrome, Rheumatic Encephalitis, Riley-Day Syndrome, Saint Vitus Dance, Sandhoff Disease, Schizencephaly, Septo-Optic Dysplasia, Shingles, Shy-Drager Syndrome, Sjogren's Syndrome, Sleep Apnea, Sleeping Sickness, Sotos Syndrome, Spasticity, Spinal Cord Infarction, Spinal Cord Injury, Spinal Cord Tumors, Spinocerebellar Atrophy, Spinocerebellar Degeneration, Stiff-Person Syndrome, Striatonigral Degeneration, Stroke, Sturge-Weber Syndrome, SUNCT Headache, Syncope, Syphilitic Spinal Sclerosis, Syringomyelia, Tabes Dorsalis, Tardive Dyskinesia, Tarlov Cysts, Tay-Sachs Disease, Temporal Arteritis, Tethered Spinal Cord Syndrome, Thomsen's Myotonia, Thoracic Outlet Syndrome, Thyrotoxic Myopathy, Tinnitus, Todd's Paralysis, Tourette Syndrome, Transient Ischemic Attack, Transmissible Spongiform Encephalopathies, Transverse Myelitis, Traumatic Brain Injury, Tremor, Trigeminal Neuralgia, Tropical Spastic Paraparesis, Troyer Syndrome, Tuberous Sclerosis, Vasculitis including Temporal Arteritis, Von Economo's Disease, Von Hippel-Lindau Disease (VHL), Von Recklinghausen's Disease, Wallenberg's Syndrome, Werdnig-Hoffman Disease, Wernicke-Korsakoff Syndrome, West Syndrome, Whiplash, Whipple's Disease, Williams Syndrome, Wilson's Disease, Wolman's Disease, X-Linked Spinal and Bulbar Muscular Atrophy, and Zellweger Syndrome.

In various embodiments, the autoimmune disease or disorder can be any one of: arthritis, including rheumatoid arthritis, acute arthritis, chronic rheumatoid arthritis, gout or gouty arthritis, acute gouty arthritis, acute immunological arthritis, chronic inflammatory arthritis, degenerative arthritis, type II collagen-induced arthritis, infectious arthritis, Lyme arthritis, proliferative arthritis, psoriatic arthritis, Still's disease, vertebral arthritis, juvenile-onset rheumatoid arthritis, osteoarthritis, arthritis deformans, polyarthritis chronica primaria, reactive arthritis, and ankylosing spondylitis; inflammatory hyperproliferative skin diseases; psoriasis, such as plaque psoriasis, pustular psoriasis, and psoriasis of the nails; atopy, including atopic diseases such as hay fever and Job's syndrome; dermatitis, including contact dermatitis, chronic contact dermatitis, exfoliative dermatitis, allergic dermatitis, allergic contact dermatitis, dermatitis herpetiformis, nummular dermatitis, seborrheic dermatitis, non-specific dermatitis, primary irritant contact dermatitis, and atopic dermatitis; x-linked hyper IgM syndrome; allergic intraocular inflammatory diseases; urticaria, such as chronic allergic urticaria, chronic idiopathic urticaria, and chronic autoimmune urticaria; myositis; polymyositis/dermatomyositis; juvenile dermatomyositis; toxic epidermal necrolysis; scleroderma, including systemic scleroderma; sclerosis, such as systemic sclerosis, multiple sclerosis (MS), spino-optical MS, primary progressive MS (PPMS), relapsing remitting MS (RRMS), progressive systemic sclerosis, atherosclerosis, arteriosclerosis, sclerosis disseminata, and ataxic sclerosis; neuromyelitis optica (NMO); inflammatory bowel disease (IBD), including Crohn's disease, autoimmune-mediated gastrointestinal diseases, colitis, ulcerative colitis, colitis ulcerosa, microscopic colitis, collagenous colitis, colitis polyposa, necrotizing enterocolitis, transmural colitis, and autoimmune inflammatory bowel disease; bowel inflammation; pyoderma gangrenosum; erythema nodosum; primary sclerosing cholangitis; respiratory distress syndrome, including adult or acute respiratory distress syndrome (ARDS); meningitis; inflammation of all or part of the uvea; iritis; choroiditis; an autoimmune hematological disorder; rheumatoid spondylitis; rheumatoid synovitis; hereditary angioedema; cranial nerve damage, as in meningitis; herpes gestationis; pemphigoid gestationis; pruritis scroti; autoimmune premature ovarian failure; sudden hearing loss due to an autoimmune condition; IgE-mediated diseases, such as anaphylaxis and allergic and atopic rhinitis; encephalitis, such as Rasmussen's encephalitis and limbic and/or brainstem encephalitis; uveitis, such as anterior uveitis, acute anterior uveitis, granulomatous uveitis, nongranulomatous uveitis, phacoantigenic uveitis, posterior uveitis, or autoimmune uveitis; glomerulonephritis (GN) with and without nephrotic syndrome, such as chronic or acute glomerulonephritis, primary GN, immune-mediated GN, membranous GN (membranous nephropathy), idiopathic membranous GN or idiopathic membranous nephropathy, membrano- or membranous proliferative GN (MPGN), including Type I and Type II, and rapidly progressive GN; proliferative nephritis; autoimmune polyglandular endocrine failure; balanitis, including balanitis circumscripta plasmacellularis; balanoposthitis; erythema annulare centrifugum; erythema dyschromicum perstans; eythema multiform; granuloma annulare; lichen nitidus; lichen sclerosus et atrophicus; lichen simplex chronicus; lichen spinulosus; lichen planus; lamellar ichthyosis; epidermolytic hyperkeratosis; premalignant keratosis; pyoderma gangrenosum; allergic conditions and responses; allergic reaction; eczema, including allergic or atopic eczema, asteatotic eczema, dyshidrotic eczema, and vesicular palmoplantar eczema; asthma, such as asthma bronchiale, bronchial asthma, and auto-immune asthma; conditions involving infiltration of T cells and chronic inflammatory responses; immune reactions against foreign antigens such as fetal A-B-O blood groups during pregnancy; chronic pulmonary inflammatory disease; autoimmune myocarditis; leukocyte adhesion deficiency; lupus, including lupus nephritis, lupus cerebritis, pediatric lupus, non-renal lupus, extra-renal lupus, discoid lupus and discoid lupus erythematosus, alopecia lupus, systemic lupus erythematosus (SLE), cutaneous SLE, subacute cutaneous SLE, neonatal lupus syndrome (NLE), and lupus erythematosus disseminatus; juvenile onset (Type I) diabetes mellitus, including pediatric insulin-dependent diabetes mellitus (IDDM), adult onset diabetes mellitus (Type II diabetes), autoimmune diabetes, idiopathic diabetes insipidus, diabetic retinopathy, diabetic nephropathy, and diabetic large-artery disorder; immune responses associated with acute and delayed hypersensitivity mediated by cytokines and T-lymphocytes; tuberculosis; sarcoidosis; granulomatosis, including lymphomatoid granulomatosis; Wegener's granulomatosis; agranulocytosis; vasculitides, including vasculitis, large-vessel vasculitis, polymyalgia rheumatica and giant-cell (Takayasu's) arteritis, medium-vessel vasculitis, Kawasaki's disease, polyarteritis nodosa/periarteritis nodosa, microscopic polyarteritis, immunovasculitis, CNS vasculitis, cutaneous vasculitis, hypersensitivity vasculitis, necrotizing vasculitis, systemic necrotizing vasculitis, ANCA-associated vasculitis, Churg-Strauss vasculitis or syndrome (CSS), and ANCA-associated small-vessel vasculitis; temporal arteritis; aplastic anemia; autoimmune aplastic anemia; Coombs positive anemia; Diamond Blackfan anemia; hemolytic anemia or immune hemolytic anemia, including autoimmune hemolytic anemia (AIHA), pernicious anemia (anemia perniciosa); Addison's disease; pure red cell anemia or aplasia (PRCA); Factor VIII deficiency; hemophilia A; autoimmune neutropenia; pancytopenia; leukopenia; diseases involving leukocyte diapedesis; CNS inflammatory disorders; multiple organ injury syndrome, such as those secondary to septicemia, trauma or hemorrhage; antigen-antibody complex-mediated diseases; anti-glomerular basement membrane disease; anti-phospholipid antibody syndrome; allergic neuritis; Behcet's disease/syndrome; Castleman's syndrome; Goodpasture's syndrome; Reynaud's syndrome; Sjogren's syndrome; Stevens-Johnson syndrome; pemphigoid, such as pemphigoid bullous and skin pemphigoid, pemphigus, pemphigus vulgaris, pemphigus foliaceus, pemphigus mucus-membrane pemphigoid, and pemphigus erythematosus; autoimmune polyendocrinopathies; Reiter's disease or syndrome; thermal injury; preeclampsia; an immune complex disorder, such as immune complex nephritis, and antibody-mediated nephritis; polyneuropathies; chronic neuropathy, such as IgM polyneuropathies and IgM-mediated neuropathy; thrombocytopenia (as developed by myocardial infarction patients, for example), including thrombotic thrombocytopenic purpura (TTP), post-transfusion purpura (PTP), heparin-induced thrombocytopenia, autoimmune or immune-mediated thrombocytopenia, idiopathic thrombocytopenic purpura (ITP), and chronic or acute ITP; scleritis, such as idiopathic cerato-scleritis, and episcleritis; autoimmune disease of the testis and ovary including, autoimmune orchitis and oophoritis; primary hypothyroidism; hypoparathyroidism; autoimmune endocrine diseases, including thyroiditis, autoimmune thyroiditis, Hashimoto's disease, chronic thyroiditis (Hashimoto's thyroiditis), or subacute thyroiditis, autoimmune thyroid disease, idiopathic hypothyroidism, Grave's disease, polyglandular syndromes, autoimmune polyglandular syndromes, and polyglandular endocrinopathy syndromes; paraneoplastic syndromes, including neurologic paraneoplastic syndromes; Lambert-Eaton myasthenic syndrome or Eaton-Lambert syndrome; stiff-man or stiff-person syndrome; encephalomyelitis, such as allergic encephalomyelitis, encephalomyelitis allergica, and experimental allergic encephalomyelitis (EAE); myasthenia gravis, such as thymoma-associated myasthenia gravis; cerebellar degeneration; neuromyotonia; opsoclonus or opsoclonus myoclonus syndrome (OMS); sensory neuropathy; multifocal motor neuropathy; Sheehan's syndrome; hepatitis, including autoimmune hepatitis, chronic hepatitis, lupoid hepatitis, giant-cell hepatitis, chronic active hepatitis, and autoimmune chronic active hepatitis; lymphoid interstitial pneumonitis (LIP); bronchiolitis obliterans (non-transplant) vs NSIP; Guillain-Barre syndrome; Berger's disease (IgA nephropathy); idiopathic IgA nephropathy; linear IgA dermatosis; acute febrile neutrophilic dermatosis; subcorneal pustular dermatosis; transient acantholytic dermatosis; cirrhosis, such as primary biliary cirrhosis and pneumonocirrhosis; autoimmune enteropathy syndrome; Celiac or Coeliac disease; celiac sprue (gluten enteropathy); refractory sprue; idiopathic sprue; cryoglobulinemia; amylotrophic lateral sclerosis (ALS; Lou Gehrig's disease); coronary artery disease; autoimmune ear disease, such as autoimmune inner ear disease (AIED); autoimmune hearing loss; polychondritis, such as refractory or relapsed or relapsing polychondritis; pulmonary alveolar proteinosis; Cogan's syndrome/nonsyphilitic interstitial keratitis; Bell's palsy; Sweet's disease/syndrome; rosacea autoimmune; zoster-associated pain; amyloidosis; a non-cancerous lymphocytosis; a primary lymphocytosis, including monoclonal B cell lymphocytosis (e.g., benign monoclonal gammopathy and monoclonal gammopathy of undetermined significance, MGUS); peripheral neuropathy; channelopathies, such as epilepsy, migraine, arrhythmia, muscular disorders, deafness, blindness, periodic paralysis, and channelopathies of the CNS; autism; inflammatory myopathy; focal or segmental or focal segmental glomerulosclerosis (FSGS); endocrine opthalmopathy; uveoretinitis; chorioretinitis; autoimmune hepatological disorder; fibromyalgia; multiple endocrine failure; Schmidt's syndrome; adrenalitis; gastric atrophy; presenile dementia; demyelinating diseases, such as autoimmune demyelinating diseases and chronic inflammatory demyelinating polyneuropathy; Dressler's syndrome; alopecia areata; alopecia totalis; CREST syndrome (calcinosis, Raynaud's phenomenon, esophageal dysmotility, sclerodactyly, and telangiectasia); male and female autoimmune infertility (e.g., due to anti-spermatozoan antibodies); mixed connective tissue disease; Chagas' disease; rheumatic fever; recurrent abortion; farmer's lung; erythema multiforme; post-cardiotomy syndrome; Cushing's syndrome; bird-fancier's lung; allergic granulomatous angiitis; benign lymphocytic angiitis; Alport's syndrome; alveolitis, such as allergic alveolitis and fibrosing alveolitis; interstitial lung disease; transfusion reaction; leprosy; malaria; Samter's syndrome; Caplan's syndrome; endocarditis; endomyocardial fibrosis; diffuse interstitial pulmonary fibrosis; interstitial lung fibrosis; pulmonary fibrosis; idiopathic pulmonary fibrosis; cystic fibrosis; endophthalmitis; erythema elevatum et diutinum; erythroblastosis fetalis; eosinophilic fasciitis; Shulman's syndrome; Felty's syndrome; flariasis; cyclitis, such as chronic cyclitis, heterochronic cyclitis, iridocyclitis (acute or chronic), or Fuch's cyclitis; Henoch-Schonlein purpura; sepsis; endotoxemia; pancreatitis; thyroxicosis; Evan's syndrome; autoimmune gonadal failure; Sydenham's chorea; post-streptococcal nephritis; thromboangitis ubiterans; thyrotoxicosis; tabes dorsalis; choroiditis; giant-cell polymyalgia; chronic hypersensitivity pneumonitis; keratoconjunctivitis sicca; epidemic keratoconjunctivitis; idiopathic nephritic syndrome; minimal change nephropathy; benign familial and ischemia-reperfusion injury; transplant organ reperfusion; retinal autoimmunity; joint inflammation; bronchitis; chronic obstructive airway/pulmonary disease; silicosis; aphthae; aphthous stomatitis; arteriosclerotic disorders; aspermiogenese; autoimmune hemolysis; Boeck's disease; cryoglobulinemia; Dupuytren's contracture; endophthalmia phacoanaphylactica; enteritis allergica; erythema nodo sum leprosum; idiopathic facial paralysis; febris rheumatica; Hamman-Rich's disease; sensoneural hearing loss; haemoglobinuria paroxysmatica; hypogonadism; ileitis regionalis; leucopenia; mononucleosis infectiosa; traverse myelitis; primary idiopathic myxedema; nephrosis; ophthalmia symphatica; orchitis granulomatosa; pancreatitis; polyradiculitis acuta; pyoderma gangrenosum; Quervain's thyreoiditis; acquired splenic atrophy; non-malignant thymoma; vitiligo; toxic-shock syndrome; food poisoning; conditions involving infiltration of T cells; leukocyte-adhesion deficiency; immune responses associated with acute and delayed hypersensitivity mediated by cytokines and T-lymphocytes; diseases involving leukocyte diapedesis; multiple organ injury syndrome; antigen-antibody complex-mediated diseases; antiglomerular basement membrane disease; allergic neuritis; autoimmune polyendocrinopathies; oophoritis; primary myxedema; autoimmune atrophic gastritis; sympathetic ophthalmia; rheumatic diseases; mixed connective tissue disease; nephrotic syndrome; insulitis; polyendocrine failure; autoimmune polyglandular syndrome type I; adult-onset idiopathic hypoparathyroidism (AOIH); cardiomyopathy such as dilated cardiomyopathy; epidermolisis bullosa acquisita (EBA); hemochromatosis; myocarditis; nephrotic syndrome; primary sclerosing cholangitis; purulent or nonpurulent sinusitis; acute or chronic sinusitis; ethmoid, frontal, maxillary, or sphenoid sinusitis; an eosinophil-related disorder such as eosinophilia, pulmonary infiltration eosinophilia, eosinophilia-myalgia syndrome, Loffler's syndrome, chronic eosinophilic pneumonia, tropical pulmonary eosinophilia, bronchopneumonic aspergillosis, aspergilloma, or granulomas containing eosinophils; anaphylaxis; seronegative spondyloarthritides; polyendocrine autoimmune disease; sclerosing cholangitis; chronic mucocutaneous candidiasis; Bruton's syndrome; transient hypogammaglobulinemia of infancy; Wiskott-Aldrich syndrome; ataxia telangiectasia syndrome; angiectasis; autoimmune disorders associated with collagen disease, rheumatism, neurological disease, lymphadenitis, reduction in blood pressure response, vascular dysfunction, tissue injury, cardiovascular ischemia, hyperalgesia, renal ischemia, cerebral ischemia, and disease accompanying vascularization; allergic hypersensitivity disorders; glomerulonephritides; reperfusion injury; ischemic reperfusion disorder; reperfusion injury of myocardial or other tissues; lymphomatous tracheobronchitis; inflammatory dermatoses; dermatoses with acute inflammatory components; multiple organ failure; bullous diseases; renal cortical necrosis; acute purulent meningitis or other central nervous system inflammatory disorders; ocular and orbital inflammatory disorders; granulocyte transfusion-associated syndromes; cytokine-induced toxicity; narcolepsy; acute serious inflammation; chronic intractable inflammation; pyelitis; endarterial hyperplasia; peptic ulcer; valvulitis; and endometriosis. In particular embodiments, the autoimmune disorder in the subject can include one or more of: systemic lupus erythematosus (SLE), lupus nephritis, chronic graft versus host disease (cGVHD), rheumatoid arthritis (RA), Sjogren's syndrome, vitiligo, inflammatory bowed disease, and Crohn's Disease. In particular embodiments, the autoimmune disorder is systemic lupus erythematosus (SLE). In particular embodiments, the autoimmune disorder is rheumatoid arthritis.

Exemplary metabolic disorders include, for example, diabetes, insulin resistance, lysosomal storage disorders (e.g., Gauchers disease, Krabbe disease, Niemann Pick disease types A and B, multiple sclerosis, Fabry's disease, Tay Sachs disease, and Sandhoff Variant A, B), obesity, cardiovascular disease, and dyslipidemia. Other exemplary metabolic disorders include, for example, 17-alpha-hydroxylase deficiency, 17-beta hydroxysteroid dehydrogenase 3 deficiency, 18 hydroxylase deficiency, 2-hydroxyglutaric aciduria, 2-methylbutyryl-CoA dehydrogenase deficiency, 3-alpha hydroxyacyl-CoA dehydrogenase deficiency, 3-hydroxyisobutyric aciduria, 3-methylcrotonyl-CoA carboxylase deficiency, 3-methylglutaconyl-CoA hydratase deficiency (AUH defect), 5-oxoprolinase deficiency, 6-pyruvoyl-tetrahydropterin synthase deficiency, abdominal obesity metabolic syndrome, abetalipoproteinemia, acatalasemia, aceruloplasminemia, acetyl CoA acetyltransferase 2 deficiency, acetyl-carnitine deficiency, acrodermatitis enteropathica, adenine phosphoribosyltransferase deficiency, adenosine deaminase deficiency, adenosine monophosphate deaminase 1 deficiency, adenylosuccinase deficiency, adrenomyeloneuropathy, adult polyglucosan body disease, albinism deafness syndrome, alkaptonuria, Alpers syndrome, alpha-1 antitrypsin deficiency, alpha-ketoglutarate dehydrogenase deficiency, alpha-mannosidosis, aminoacylase 1 deficiency, anemia sideroblastic and spinocerebellar ataxia, arginase deficiency, argininosuccinic aciduria, aromatic L-amino acid decarboxylase deficiency, arthrogryposis renal dysfunction cholestasis syndrome, Arts syndrome, aspartylglycosaminuria, atypical Gaucher disease due to saposin C deficiency, autoimmune polyglandular syndrome type 2, autosomal dominant optic atrophy and cataract, autosomal erythropoietic protoporphyria, autosomal recessive spastic ataxia 4, Barth syndrome, Bartter syndrome, Bartter syndrome antenatal type 1, Bartter syndrome antenatal type 2, Bartter syndrome type 3, Bartter syndrome type 4, Beta ketothiolase deficiency, biotinidase deficiency, Bjornstad syndrome, carbamoyl phosphate synthetase 1 deficiency, carnitine palmitoyl transferase 1A deficiency, carnitine-acylcarnitine translocase deficiency, carnosinemia, central diabetes insipidus, cerebral folate deficiency, cerebrotendinous xanthomatosis, ceroid lipofuscinosis neuronal 1, Chanarin-Dorfman syndrome, Chediak-Higashi syndrome, childhood hypophosphatasia, cholesteryl ester storage disease, chondrocalcinosisc, chylomicron retention disease, citrulline transport defect, congenital bile acid synthesis defect, type 2, Crigler Najjar syndrome, cytochrome c oxidase deficiency, D-2-hydroxyglutaric aciduria, D-bifunctional protein deficiency, D-glycericacidemia, Danon disease, dicarboxylic aminoaciduria, dihydropteridine reductase deficiency, dihydropyrimidinase deficiency, diabetes insipidus, dopamine beta hydroxylase deficiency, Dowling-Degos disease, erythropoietic uroporphyria associated with myeloid malignancy, Familial chylomicronemia syndrome, Familial HDL deficiency, Familial hypocalciuric hypercalcemia type 1, Familial hypocalciuric hypercalcemia type 2, Familial hypocalciuric hypercalcemia type 3, Familial LCAT deficiency, Familial partial lipodystrophy type 2, Fanconi Bickel syndrome, Farber disease, fructose-1,6-bisphosphatase deficiency, gamma-cystathionase deficiency, Gaucher disease, Gilbert syndrome, Gitelman syndrome, glucose transporter type 1 deficiency syndrome, glutamine deficiency, congenital, Glutaric acidemia. glutathione synthetase deficiency, glycine N-methyltransferase deficiency, Glycogen storage disease hepatic lipase deficiency, homocysteinemia, Hurler syndrome, hyperglycerolemia, Imerslund-Grasbeck syndrome, iminoglycinuria, infantile neuroaxonal dystrophy, Kearns-Sayre syndrome, Krabbe disease, lactate dehydrogenase deficiency, Lesch Nyhan syndrome, Menkes disease, methionine adenosyltransferase deficiency, mitochondrial complex deficiency, muscular phosphorylase kinase deficiency, neuronal ceroid lipofuscinosis, Niemann-Pick disease type A, Niemann-Pick disease type B, Niemann-Pick disease type C1, Niemann-Pick disease type C2, ornithine transcarbamylase deficiency, Pearson syndrome, Perrault syndrome, phosphoribosylpyrophosphate synthetase superactivity, primary carnitine deficiency, hyperoxaluria, purine nucleoside phosphorylase deficiency, pyruvate carboxylase deficiency, pyruvate dehydrogenase complex deficiency, pyruvate dehydrogenase phosphatase deficiency, yruvate kinase deficiency, Refsum disease, diabetes mellitus, Scheie syndrome, Sengers syndrome, Sialidosis Sjogren-Larsson syndrome, Tay-Sachs disease, transcobalamin 1 deficiency, trehalase deficiency, Walker-Warburg syndrome, Wilson disease, Wolfram syndrome, and Wolman disease.

### Non-Transitory Computer Readable Medium

Also provided herein is a computer readable medium comprising computer executable instructions configured to implement any of the methods described herein. In various embodiments, the computer readable medium is a non-transitory computer readable medium. In some embodiments, the computer readable medium is a part of a computer system (e.g., a memory of a computer system). The computer readable medium can comprise computer executable instructions for performing methods disclosed herein, such as methods for building, maintaining, implementing, and providing standardized solutions (e.g., DMSs and TSPs).

### Computing Device

The methods described above, including the methods of building, maintaining, implementing, and providing standardized solutions (e.g., DMSs and TSPs) are, in some embodiments, performed on a computing device. Examples of a computing device can include a personal computer, desktop computer laptop, server computer, a computing node within a cluster, message processors, hand-held devices, multi-processor systems, microprocessor-based or programmable consumer electronics, network PCs, minicomputers, mainframe computers, mobile telephones, PDAs, tablets, pagers, routers, switches, and the like.

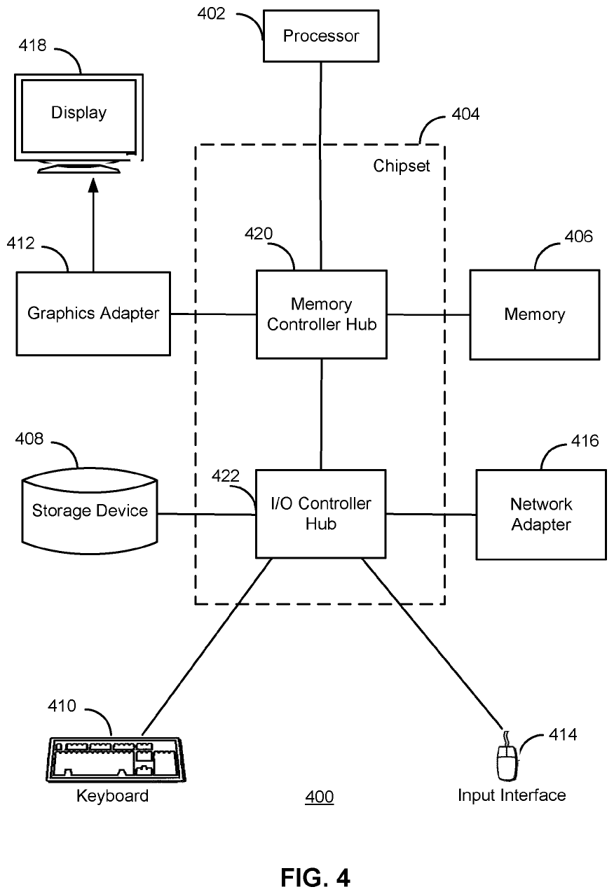

FIG. 4 illustrates an example computing device 400 for implementing system and methods described in FIGS. 1A-1B, 2A-2F, and 3A-3C. In some embodiments, the computing device 400 includes at least one processor 402 coupled to a chipset 404. The chipset 404 includes a memory controller hub 420 and an input/output (I/O) controller hub 422. A memory 406 and a graphics adapter 412 are coupled to the memory controller hub 420, and a display 418 is coupled to the graphics adapter 412. A storage device 408, an input interface 414, and network adapter 416 are coupled to the I/O controller hub 422. Other embodiments of the computing device 400 have different architectures.

The storage device 408 is a non-transitory computer-readable storage medium such as a hard drive, compact disk read-only memory (CD-ROM), DVD, or a solid-state memory device. The memory 406 holds instructions and data used by the processor 402. The input interface 414 is a touch-screen interface, a mouse, track ball, or other type of input interface, a keyboard, or some combination thereof, and is used to input data into the computing device 400. In some embodiments, the computing device 400 may be configured to receive input (e.g., commands) from the input interface 414 via gestures from the user. The graphics adapter 412 displays images and other information on the display 418. As an example, the display 418 can show a catalog of standardized solutions (e.g., DMSs and/or TSPs). The network adapter 416 couples the computing device 400 to one or more computer networks.

The computing device 400 is adapted to execute computer program modules for providing functionality described herein. As used herein, the term “module” refers to computer program logic used to provide the specified functionality. Thus, a module can be implemented in hardware, firmware, and/or software. In one embodiment, program modules are stored on the storage device 408, loaded into the memory 406, and executed by the processor 402.

The types of computing devices 400 can vary from the embodiments described herein. For example, the computing device 400 can lack some of the components described above, such as graphics adapters 412, input interface 414, and displays 418. In some embodiments, a computing device 400 can include a processor 402 for executing instructions stored on a memory 406.

In various embodiments, the different entities depicted in FIGS. 1A and/or FIG. 1B may implement one or more computing devices to perform the methods described above. For example, the digital solution system 130, third party entity 110A, and third party entity 110B may each employ one or more computing devices. As another example, one or more of the modules of the digital solution system 130 (e.g., asset module 140, target solution profile module 145, digital measurement solution module 150, life-cycle management module 155, disease characterization module 160, and marketplace module 165) may be implemented by one or more computing devices to perform the methods described above.

The methods of building, maintaining, implementing, and providing TSPs and/or DMSs can be implemented in hardware or software, or a combination of both. In one embodiment, a non-transitory machine-readable storage medium, such as one described above, is provided, the medium comprising a data storage material encoded with machine readable data which, when using a machine programmed with instructions for using said data, is capable of perform the methods disclosed herein including methods of building, maintaining, implementing, and providing TSPs and/or DMSs. Embodiments of the methods described above can be implemented in computer programs executing on programmable computers, comprising a processor, a data storage system (including volatile and non-volatile memory and/or storage elements), a graphics adapter, an input interface, a network adapter, at least one input device, and at least one output device. A display is coupled to the graphics adapter. Program code is applied to input data to perform the functions described above and generate output information. The output information is applied to one or more output devices, in known fashion. The computer can be, for example, a personal computer, microcomputer, or workstation of conventional design.

Each program can be implemented in a high level procedural or object oriented programming language to communicate with a computer system. However, the programs can be implemented in assembly or machine language, if desired. In any case, the language can be a compiled or interpreted language. Each such computer program is preferably stored on a storage media or device (e.g., ROM or magnetic diskette) readable by a general or special purpose programmable computer, for configuring and operating the computer when the storage media or device is read by the computer to perform the procedures described herein. The system can also be considered to be implemented as a computer-readable storage medium, configured with a computer program, where the storage medium so configured causes a computer to operate in a specific and predefined manner to perform the functions described herein.

The signature patterns and databases thereof can be provided in a variety of media to facilitate their use. “Media” refers to a manufacture that contains the signature pattern information of the present invention. The databases of the present invention can be recorded on computer readable media, e.g., any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. One of skill in the art can readily appreciate how any of the presently known computer readable mediums can be used to create a manufacture comprising a recording of the present database information. “Recorded” refers to a process for storing information on computer readable medium, using any such methods as known in the art. Any convenient data storage structure can be chosen, based on the means used to access the stored information. A variety of data processor programs and formats can be used for storage, e.g., word processing text file, database format, etc.

### ADDITIONAL EMBODIMENTS

Disclosed herein is a method for characterizing a disease of a subject, the method comprising: obtaining a measurement of interest from the subject; selecting a target solution profile from a plurality of target solution profiles; and applying the target solution profile to the obtained measurement of interest to characterize the disease for the subject, wherein the target solution profile comprises: a measurement definition defining one or more subject changes relevant to the disease; an instrumentation asset that transforms the measurement of interest captured according to the measurement definition to a dataset, the instrumentation asset being device-agnostic and is thereby interchangeable across different target solution profiles, wherein the instrumentation asset is validated; and an interpretation asset aligned with the measurement definition, the interpretation asset configured to interpret the dataset from the instrumentation asset and characterize the disease of the subject.

In various embodiments, the target solution profile is previously validated by implementing one or more qualification protocols used to establish equivalency of solutions across the plurality of target solution profiles.

Additionally disclosed herein is a method for building a target solution profile for characterizing a disease, the method comprising: generating a measurement definition of the target solution profile that defines one or more subject changes relevant to the disease; selecting an instrumentation asset that transforms data captured according to the measurement definition to a dataset, the instrumentation asset being device-agnostic and is thereby interchangeable across different target solution profiles; and generating an interpretation asset of the target solution profile aligned with the measurement definition, the interpretation asset configured to interpret the dataset from the instrumentation asset and characterize the disease. In various embodiments, the method disclosed herein further comprises implementing a qualification protocol to validate the target solution profile, the qualification protocol used to establish equivalency of solutions across the plurality of target solution profiles. In various embodiments, the measurement definition and interpretation asset are fixed for the target solution profile and specific for the disease. In various embodiments, the instrumentation asset is interchangeable across different target solution profiles for characterizing different diseases. In various embodiments, the instrumentation asset is specific for a class of devices. In various embodiments, the class of devices comprises wearable devices (e.g., wrist-worn device), ingestibles, image and voice based devices, touchless sensors, and sensor based smart devices (e.g., scales, thermometers, and respirators). In various embodiments, the instrumentation asset comprises a machine learning model that transforms data captured according to the measurement definition to the dataset.

In various embodiments, the qualification protocol is implemented to validate equivalency of solutions across different classes of devices. In various embodiments, the target solution profile represents a standardized digital measurement solution for characterizing the disease. In various embodiments, the target solution profile comprises a measurement stack comprising a plurality of layers. In various embodiments, each of the measurement definition, the instrumentation asset, and the interpretation asset are represented by one or more layers in the plurality of layers. In various embodiments, an order of the plurality of layers comprises one or more layers of the measurement definition, one or more layers of the instrumentation asset, and one or more layers of the interpretation asset.

In various embodiments, a layer of the measurement definition interfaces with a layer of the instrumentation asset, and a layer of the instrumentation asset interfaces with the interpretation asset. In various embodiments, the one or more layers of the measurement definition comprise one or more of a hypothesis of the disease and a measurable concept of interest. In various embodiments, the one or more layers of the instrumentation asset comprise one or more of a measurement method, health data, and a machine learning algorithm. In various embodiments, the one or more layers of the interpretation asset comprise one or more of an analytical validation and a clinical interpretation. In various embodiments, the one or more layers of the measurement definition comprise a hypothesis of the disease and a measurable concept of interest, wherein the one or more layers of the instrumentation asset comprise a measurement method, health data, and a machine learning algorithm, and wherein the one or more layers of the interpretation asset comprise an analytical validation and a clinical interpretation.

In various embodiments, the disease is dementia. In various embodiments, the hypothesis comprises an intervention that slows progression of dementia. In various embodiments, the measurable concept of interest comprises one or more of attention, language, or executive functioning. In various embodiments, the measurement method comprises a method for capturing speech. In various embodiments, the health data comprises raw speech data captured from a subject or magnetic resonance imaging data. In various embodiments, the machine learning algorithm comprises one or more of a natural language processing algorithm, a clustering algorithm, and a clinical variable predictor. In various embodiments, the analytical validation comprises evidence validating performance of the machine learning algorithm. In various embodiments, the clinical interpretation comprises evidence supporting characterization of the disease according to a clinical dementia rating.

In various embodiments, the disease is Parkinson's Disease. In various embodiments, the hypothesis comprises an intervention that reduces tremor. In various embodiments, the measurable concept of interest comprises ability to perform daily activities of moderate intensity. In various embodiments, the measurement method comprises methods for capturing physiological data using a biosensor. In various embodiments, the health data comprises raw physiological data captured using a biosensor. In various embodiments, the machine learning algorithm transforms the health data to the dataset. In various embodiments, the analytical validation comprises evidence validating performance of the machine learning algorithm. In various embodiments, the clinical interpretation comprises evidence supporting characterization of the disease in a Parkinson's Disease patient population

Additionally disclosed herein is a method for providing one or more target solution profiles useful for characterizing one or more diseases, the method comprising: providing a catalogue comprising a plurality of target solution profiles, wherein each of one or more of the target solution profiles comprises: a measurement definition of the target solution profile defining one or more subject changes relevant to a disease of the one or more diseases; an instrumentation asset that transforms data captured according to the measurement definition to a dataset, the instrumentation asset being device-agnostic and is thereby interchangeable across different target solution profiles, wherein the instrumentation asset is validated by implementing one or more qualification protocols used to establish equivalency of solutions across a plurality of instrumentation assets; and an interpretation asset aligned with the measurement definition, the interpretation asset configured to interpret the dataset from the interchangeable instrumentation asset and characterize the disease of the subject; receiving, from a third party, a selection of one or more of the target solution profiles; providing the selected one or more target solution profiles to the third party.

In various embodiments, the target solution profile comprises a measurement stack comprising a plurality of layers. In various embodiments, each of the measurement definition, the instrumentation asset, and the interpretation asset are represented by one or more layers in the plurality of layers. In various embodiments, an order of the plurality of layers comprises one or more layers of the measurement definition, one or more layers of the instrumentation asset, and one or more layers of the interpretation asset. In various embodiments, a layer of the measurement definition interfaces with a layer of the instrumentation asset, and a layer of the instrumentation asset interfaces with the interpretation asset. In various embodiments, the one or more layers of the measurement definition comprise one or more of a hypothesis of the disease and a measurable concept of interest. In various embodiments, the one or more layers of the instrumentation asset comprise one or more of a measurement method, health data, and a machine learning algorithm. In various embodiments, the one or more layers of the interpretation asset comprise one or more of an analytical validation and a clinical interpretation. In various embodiments, the one or more layers of the measurement definition comprise a hypothesis of the disease and a measurable concept of interest, wherein the one or more layers of the instrumentation asset comprise a measurement method, health data, and a machine learning algorithm, and wherein the one or more layers of the interpretation asset comprise an analytical validation and a clinical interpretation.

In various embodiments, methods disclosed herein further comprise: receiving, from the third party, a search query; for each of one or more target solution profiles in the plurality of target solution profiles, evaluating the target solution profile to determine whether the target solution profile satisfies the query; and returning a list of target solution profiles that satisfy the query. In various embodiments, evaluating the target solution profile comprises: evaluating one or more layers of the measurement definition for a concept of interest that satisfies the query. In various embodiments, methods disclosed herein further comprise: in response to a request from the third party, replacing the instrumentation asset of the target solution profile with a second instrumentation asset to generate a revised target solution profile; and providing the revised target solution profile in the catalogue comprising the plurality of target solution profiles.

Additionally disclosed herein is a non-transitory computer readable medium comprising instructions that, when executed by a processor, cause the processor to obtain a measurement of interest from the subject; select a target solution profile from a plurality of target solution profiles; and apply the target solution profile to the obtained measurement of interest to characterize the disease for the subject, wherein the target solution profile comprises: a measurement definition defining one or more subject changes relevant to the disease; an instrumentation asset that transforms the measurement of interest captured according to the measurement definition to a dataset, the instrumentation asset being device-agnostic and is thereby interchangeable across different target solution profiles, wherein the instrumentation asset is validated; and an interpretation asset aligned with the measurement definition, the interpretation asset configured to interpret the dataset from the instrumentation asset and characterize the disease of the subject.

In various embodiments, the target solution profile is previously validated by implementing one or more qualification protocols used to establish equivalency of solutions across the plurality of target solution profiles.

Additionally disclosed herein is a non-transitory computer readable medium for building a target solution profile for characterizing a disease comprising instructions that, when executed by a processor, cause the processor to: generate a measurement definition of the target solution profile that defines one or more subject changes relevant to the disease; select an instrumentation asset that transforms data captured according to the measurement definition to a dataset, the instrumentation asset being device-agnostic and is thereby interchangeable across different target solution profiles; and generate an interpretation asset of the target solution profile aligned with the measurement definition, the interpretation asset configured to interpret the dataset from the instrumentation asset and characterize the disease.

In various embodiments, the non-transitory computer readable medium further comprises instructions that when executed by the processor, cause the processor to implement a qualification protocol to validate the target solution profile, the qualification protocol used to establish equivalency of solutions across the plurality of target solution profiles. In various embodiments, the measurement definition and interpretation asset are fixed for the target solution profile and specific for the disease. In various embodiments, the instrumentation asset is interchangeable across different target solution profiles for characterizing different diseases. In various embodiments, the instrumentation asset is specific for a class of devices. In various embodiments, the class of devices comprises wearable devices, ingestibles, image and voice based devices, touchless sensors, and sensor based smart devices (e.g., scales, thermometers, and respirators). In various embodiments, the instrumentation asset comprises a machine learning model that transforms data captured according to the measurement definition to the dataset.

In various embodiments, the qualification protocol is implemented to validate equivalency of solutions across different classes of devices. In various embodiments, the target solution profile represents a standardized digital measurement solution for characterizing the disease. In various embodiments, the target solution profile comprises a measurement stack comprising a plurality of layers. In various embodiments, each of the measurement definition, the instrumentation asset, and the interpretation asset are represented by one or more layers in the plurality of layers. In various embodiments, an order of the plurality of layers comprises one or more layers of the measurement definition, one or more layers of the instrumentation asset, and one or more layers of the interpretation asset.

In various embodiments, a layer of the measurement definition interfaces with a layer of the instrumentation asset, and a layer of the instrumentation asset interfaces with the interpretation asset. In various embodiments, the one or more layers of the measurement definition comprise one or more of a hypothesis of the disease and a measurable concept of interest. In various embodiments, the one or more layers of the instrumentation asset comprise one or more of a measurement method, health data, and a machine learning algorithm. In various embodiments, the one or more layers of the interpretation asset comprise one or more of an analytical validation and a clinical interpretation. In various embodiments, the one or more layers of the measurement definition comprise a hypothesis of the disease and a measurable concept of interest, wherein the one or more layers of the instrumentation asset comprise a measurement method, health data, and a machine learning algorithm, and wherein the one or more layers of the interpretation asset comprise an analytical validation and a clinical interpretation.

In various embodiments, the disease is dementia. In various embodiments, the hypothesis comprises an intervention that slows progression of dementia. In various embodiments, the measurable concept of interest comprises one or more of attention, language, or executive functioning. In various embodiments, the measurement method comprises a method for capturing speech. In various embodiments, the health data comprises raw speech data captured from a subject or magnetic resonance imaging data. In various embodiments, the machine learning algorithm comprises one or more of a natural language processing algorithm, a clustering algorithm, and a clinical variable predictor. In various embodiments, the analytical validation comprises evidence validating performance of the machine learning algorithm. In various embodiments, the clinical interpretation comprises evidence supporting characterization of the disease according to a clinical dementia rating.

In various embodiments, the disease is Parkinson's Disease. In various embodiments, the hypothesis comprises an intervention that reduces tremor. In various embodiments, the measurable concept of interest comprises ability to perform daily activities of moderate intensity. In various embodiments, the measurement method comprises methods for capturing physiological data using a biosensor. In various embodiments, the health data comprises raw physiological data captured using a biosensor. In various embodiments, the machine learning algorithm transforms the health data to the dataset. In various embodiments, the analytical validation comprises evidence validating performance of the machine learning algorithm. In various embodiments, the clinical interpretation comprises evidence supporting characterization of the disease in a Parkinson's Disease patient population

Additionally disclosed herein is a non-transitory computer readable medium for providing one or more target solution profiles useful for characterizing one or more diseases, the non-transitory computer readable medium comprising instructions that, when executed by a processor, cause the processor to: provide a catalogue comprising a plurality of target solution profiles, wherein each of one or more of the target solution profiles comprises: a measurement definition of the target solution profile defining one or more subject changes relevant to a disease of the one or more diseases; an instrumentation asset that transforms data captured according to the measurement definition to a dataset, the instrumentation asset being device-agnostic and is thereby interchangeable across different target solution profiles, wherein the instrumentation asset is validated by implementing one or more qualification protocols used to establish equivalency of solutions across a plurality of instrumentation assets; and an interpretation asset aligned with the measurement definition, the interpretation asset configured to interpret the dataset from the interchangeable instrumentation asset and characterize the disease of the subject; receive, from a third party, a selection of one or more of the target solution profiles; provide the selected one or more target solution profiles to the third party. In various embodiments, the target solution profile comprises a measurement stack comprising a plurality of layers. In various embodiments, each of the measurement definition, the instrumentation asset, and the interpretation asset are represented by one or more layers in the plurality of layers. In various embodiments, an order of the plurality of layers comprises one or more layers of the measurement definition, one or more layers of the instrumentation asset, and one or more layers of the interpretation asset. In various embodiments, a layer of the measurement definition interfaces with a layer of the instrumentation asset, and a layer of the instrumentation asset interfaces with the interpretation asset. In various embodiments, the one or more layers of the measurement definition comprise one or more of a hypothesis of the disease and a measurable concept of interest. In various embodiments, the one or more layers of the instrumentation asset comprise one or more of a measurement method, health data, and a machine learning algorithm. In various embodiments, the one or more layers of the interpretation asset comprise one or more of an analytical validation and a clinical interpretation. In various embodiments, the one or more layers of the measurement definition comprise a hypothesis of the disease and a measurable concept of interest, wherein the one or more layers of the instrumentation asset comprise a measurement method, health data, and a machine learning algorithm, and wherein the one or more layers of the interpretation asset comprise an analytical validation and a clinical interpretation.

In various embodiments, the non-transitory computer readable medium further comprises instructions that, when executed by the processor, cause the processor to: receive, from the third party, a search query; for each of one or more target solution profiles in the plurality of target solution profiles, evaluate the target solution profile to determine whether the target solution profile satisfies the query; and return a list of target solution profiles that satisfy the query. In various embodiments, the instructions that cause the processor to evaluate the target solution profile further comprise instructions that, when executed by the processor, cause the processor to: evaluate one or more layers of the measurement definition for a concept of interest that satisfies the query. In various embodiments, the non-transitory computer readable medium further comprises instructions that, when executed by the processor, cause the processor to: in response to a request from the third party, replace the instrumentation asset of the target solution profile with a second instrumentation asset to generate a revised target solution profile; and provide the revised target solution profile in the catalogue comprising the plurality of target solution profiles.

### EXAMPLES

### Example 1: Target Solution Profile and Digital Measurement Solutions for Atopic Dermatitis

The measurement stack is divided into nine individual layers. Each components includes unique information that can stand alone and offer additional value combined with the other components. These components have been categorized into three sub-stacks (otherwise referred to as assets): the definition-, instrumentation- and validation sub-stacks. The definition sub-stack describes the condition, meaningful aspect of health, and concepts of interest. Regulatory acceptance of this sub-stack is valuable for pursuing a study and should be aligned early on. The instrumentation sub-stack includes components for collecting, analyzing, and interpreting data. These layers include both specific solutions and generically described classes of solutions. Finally, the validation sub-stack describes the validation studies and regulatory approval to complete the solutions. Although stacks can be built starting from every component, the process often begins with available instrumentation or a medical condition. Atopic dermatitis (AD) is showcased here to walk through the measurement stack and an example target solution profile.

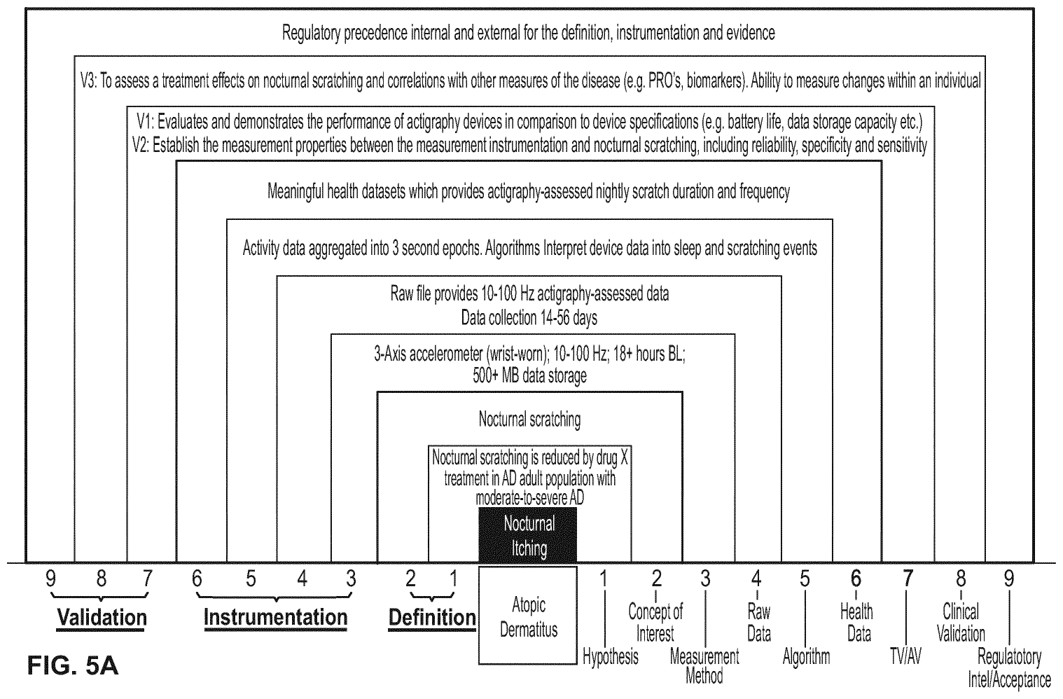

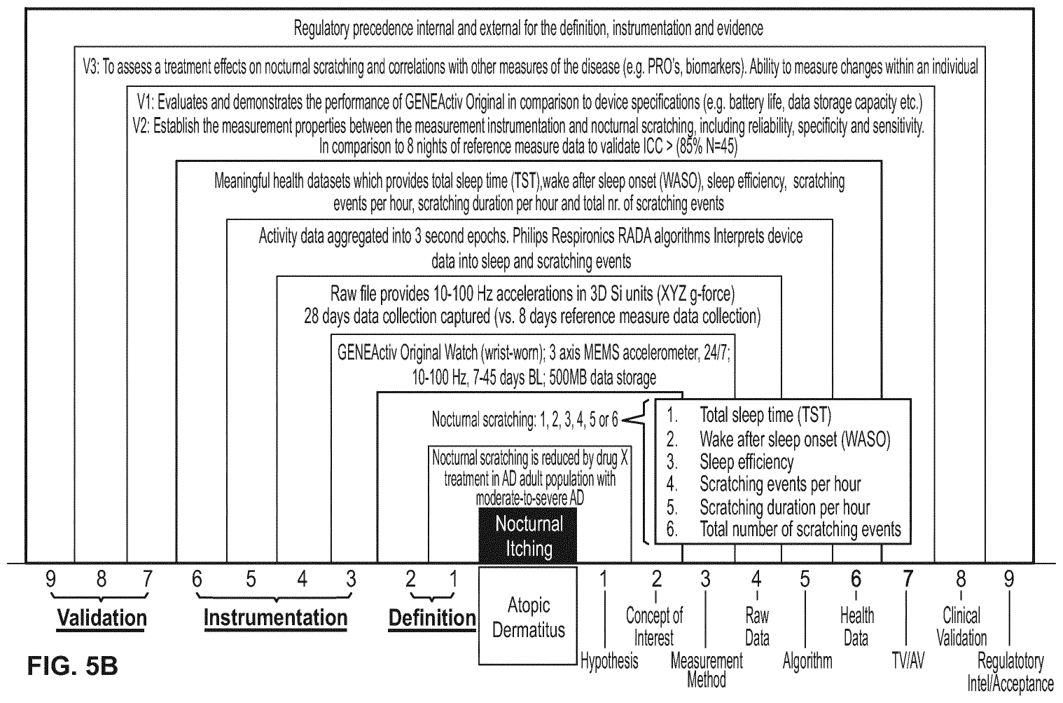

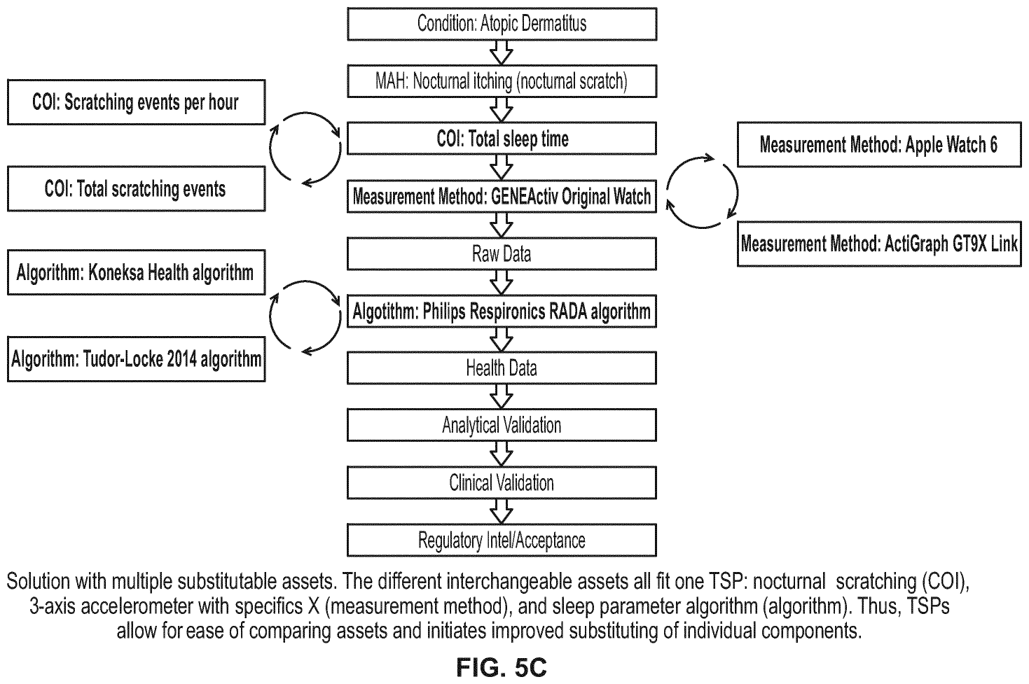

FIG. 5A depicts an example target solution profile for atopic dermatitis. The condition describes the medical condition of the patient population. The meaningful aspect of health (MAH) defines an aspect of this condition which a patient: (1) does not want to become worse, (2) wants to improve, or (3) wants to prevent. Here, the condition is atopic dermatitis and the meaningful aspect of health is nocturnal itching. Referring to the hypothesis component (e.g., component 1 shown in FIG. 5A), it describes the goal of the meaning aspect of health. For example, here, the hypothesis is that nocturnal itching is decreased due to a drug X in an adult population with moderate-to-severe AD.

Component 2 in the measurement stack refers to the concept of interest. Here, the concept of interest (COI) describes how this specific meaningful aspect of health will be measured. For example, if the goal is to measure nocturnal scratching, the concept of interest can be any of a measure of total sleep time, scratching events per hour, or total scratching events. This COI is practically measured using an outcome to measure (OTM). For example, the outcome to measure includes the specific, measurable characteristics of the condition that evaluate the MAH described by the COI. Thus, the outcome to measure reveals whether the treatment of the MAH is beneficial. For example, reduction in scratching events after X weeks. Although not explicitly shown in FIG. 5A, a single condition can have multiple MAHs; one MAH can have multiple COIs; one COI can have multiple outcomes to measure.

Component 3 in the measurement stack refers to the measurement method, which is part of the instrumentation asset. The measurement method includes hardware, software, or firmware solutions. Examples are wearable devices, mobile applications, and sensors. Additionally, complete software solutions can be a measurement method (e.g., speech batteries) as long as the method can reliably measure the OTM. As a specific example, a measurement method is a watch with a 3-axis MEMS accelerometer (10-100 Hz). This device captures accelerations 24/7 for 7 to 45 days (dependent on the set frequency).

Component 4 in the measurement stack refers to the raw data. This component represents the raw datasets which are the outcomes of the measurement method. Generally, raw data does not yet deliver meaningful health data. However, raw data is an individual layer as each of these datasets can be leveraged by multiple algorithms for different purposes. Here, the raw data layer is introduced as, for example, a raw file that provides 10-100 Hz accelerations. This is captured in 3D SI units (XYZ g-force) with 28 days of continuous data collection. In short, this example describes what raw data is captured (accelerations in 3D SI units), its frequency (10-100 Hz), and the amount of data that is captured (28 days).

Component 5 in the measurement stack refers to the algorithm. The algorithm represents a method in transforming raw data into meaningful health datasets. Thus, meaningful health datasets are readily analyzable and interpreted. Algorithms can be integrated into the complete instrumentation, or individual algorithms can be leveraged to transform (individual) raw datasets. For example, Philips Respironics RADA algorithm interprets measurement device data into sleep and scratching events.

Component 6 in the measurement stack refers to the health data. The health data component describes the health dataset generated by the algorithm from the initial raw datasets. Health datasets can be assessed as individual datasets for multiple purposes. Therefore, health datasets are considered a standalone asset and are included in the stack as an individual layer. For example, meaningful health datasets provide total sleep time, scratching events per hour, and the total number of scratching events.

Component 7 in the measurement stack refers to the technical and analytical validations. Further, component 8 in the measurement stack refers to clinical validation. The different validations ensure that digital measures are valid and therefore, can be recognized as eligible for clinical trial approval. Instrumentation outputs are evaluated both in silico and in vitro at the sample level. Specifically, technical validation (referred to as V1 in FIG. 5A) of the digital asset assures that the instrumentation captured the fundamental analog data accordingly. The technical validation verifies whether captured data resulted in the generation of appropriate output data. For example, the technical validation ensures that the instrumentation matches the specifications of the device used to capture the data (e.g., battery life, data storage, and available measure frequencies).

The analytical validation (referred to as V2 in FIG. 5A) evaluates the complete instrumentation, often in vitro. The analytical validation includes validating device specifics, processing algorithms, and reliable health data output. For example, the analytical validation validates the ability to measure, detect and predict physiological or behavioral metrics against an appropriate measurement standard. The specific example in FIG. 5A shows the analytical validation of establishing the measurement properties between the measurement instrumentation and nocturnal scratching, including reliability, specificity, and sensitivity. In comparison to 8 nights of reference measure data to validate ICC>85% (N=45). The ICC validates whether the novel digital instrumentation is better or at least comparable to the reference measure (e.g., infrared observation to monitor nocturnal scratching).

The clinical validation (referred to as V3 in FIG. 5A) evaluates whether the solution identifies, measures, and predicts the meaningful clinical, biological, physical, functional state, or experience in the specific context of use (COU). This is stated by its study design. Clinical validation is the in vivo validation and includes the specific target population and study-specific digital endpoints. The clinical validation allows for a meaningful interpretation of outcomes and evaluates whether the solution is valid to answer clinical questions related to the instrumentation- and definition sub-stack. For example, the clinical validation assesses treatment effects on nocturnal scratching and correlations with other measures of the disease (e.g., PROs, biomarkers) and the ability to measure changes within an individual.

Component 9 of the measurement stack refers to regulatory validation. Here, regulatory qualifications by health authorities are of importance for the acceptance and adoption of digital measures. The regulatory validation assesses all layers of the measurement stack, but predominantly is applied for the definition- and validation components. For example, regulatory precedence internal and external for the definition, instrumentation, and evidence.

As shown in FIG. 5A, the target solution profile for adult Atopic Dermatitis population includes a nocturnal scratching-endpoint based on actigraphy. The TSP represents a generic, solution-agnostic stack. Here, all layers of the instrumentation asset are generically described. The measurement method is a 3-axis wrist-worn accelerometer with a range of specifications. Also, the raw data-, algorithm- and health data layers are generically described, often with a certain scope (e.g., 14-56 days of data collection instead of a specific number of days). Furthermore, the COIs are generically described as nocturnal scratching. Many DMSs, such as the DMS shown in FIG. 5B, are of a class that is represented by this TSP.

FIG. 5B depicts an example digital measurement solution (DMS) for atopic dermatitis. Here, the DMS is populated with a nocturnal scratching endpoint based on actigraphy. The meaningful aspect of health is nocturnal itching, and a total of 6 different concepts of interest have been identified (e.g., total sleep time, wake after sleep onset, sleep efficiency, scratching events per hour, scratching duration per hour, and total number of scratching events). Each of the different concepts of interest can be selected for a particular DMS. Thus, here, the 6 different concepts of interests correspond to 6 different DMSs for atopic dermatitis.

Component 3 (measurement method) in the DMS identifies the particular device that is used for capturing raw data. Specifically, as shown in FIG. 5B, the GENEActiv Original watch captures the raw data. Furthermore, component 5 (Algorithm) in the DMS identifies the particular algorithm that is used to transform the raw data (component 4) into the health data (component 6). Here, the algorithm in this DMS is the Philips Respironics RADA algorithm that interprets device data into sleep and scratching events.

FIG. 5C depicts the interchangeability of assets of different digital measurement solutions for atopic dermatitis. Here, the interchangeability of assets enables the rapid development of numerous digital measurement solutions incorporating the different assets. For example, the center measurement stack in FIG. 5C refers to the DMS shown in FIG. 5B. The GENEActiv Original Watch is included in component 3 (measurement method). However, other devices such as the Apple Watch (e.g., Apple Watch 6), Fitbit, and ActiGraph (e.g., ActiGraph GT9X Link) can be alternatively included in component 3 in place of the GENEActiv Watch. These additional DMSs are also of the class that is represented by a TSP. Furthermore, the Philips Respironics RADA algorithm can be interchangeable with other types of algorithms, such as the Koneska health algorithm or the Tudor-Locke 2014 algorithm. Furthermore, the particular concept of interest in the measurement stack (e.g., total sleep time) can be interchangeable with other concepts of interests (e.g., scratching events per hour or total scratching events).

### Example 2: Target Solution Profile and Digital Measurement Solutions for Pulmonary Arterial Hypertension

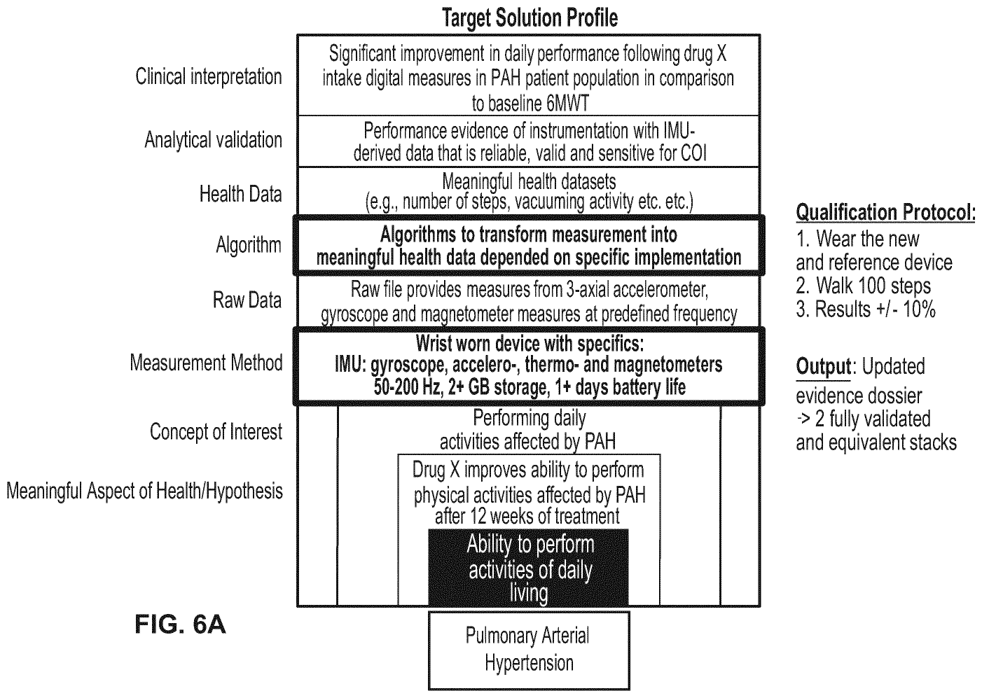

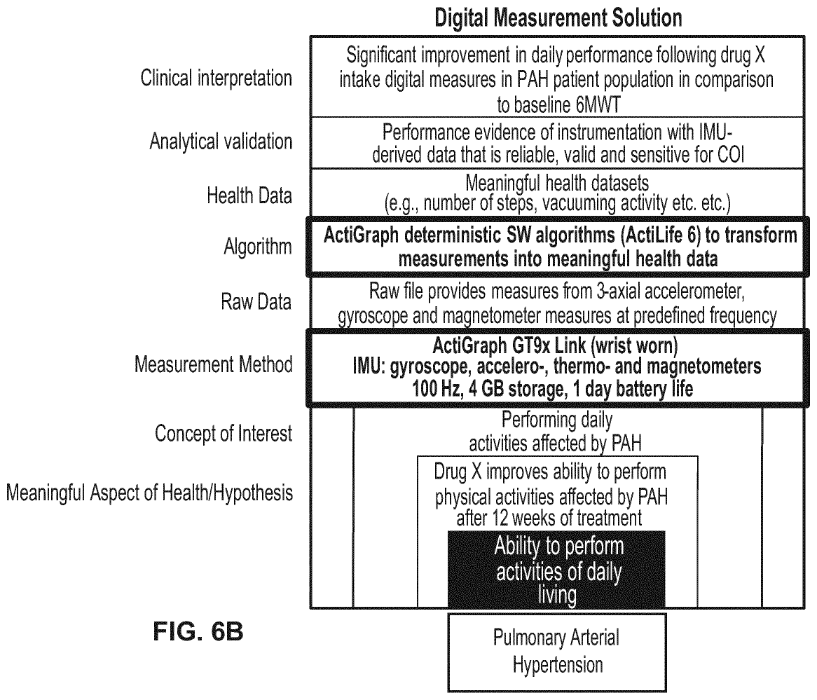

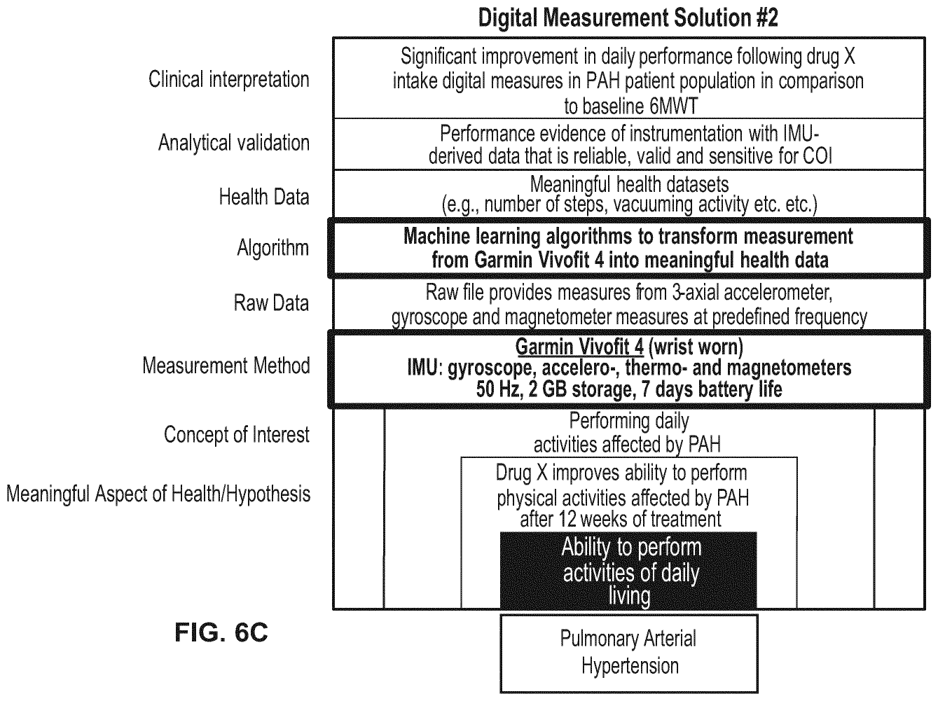

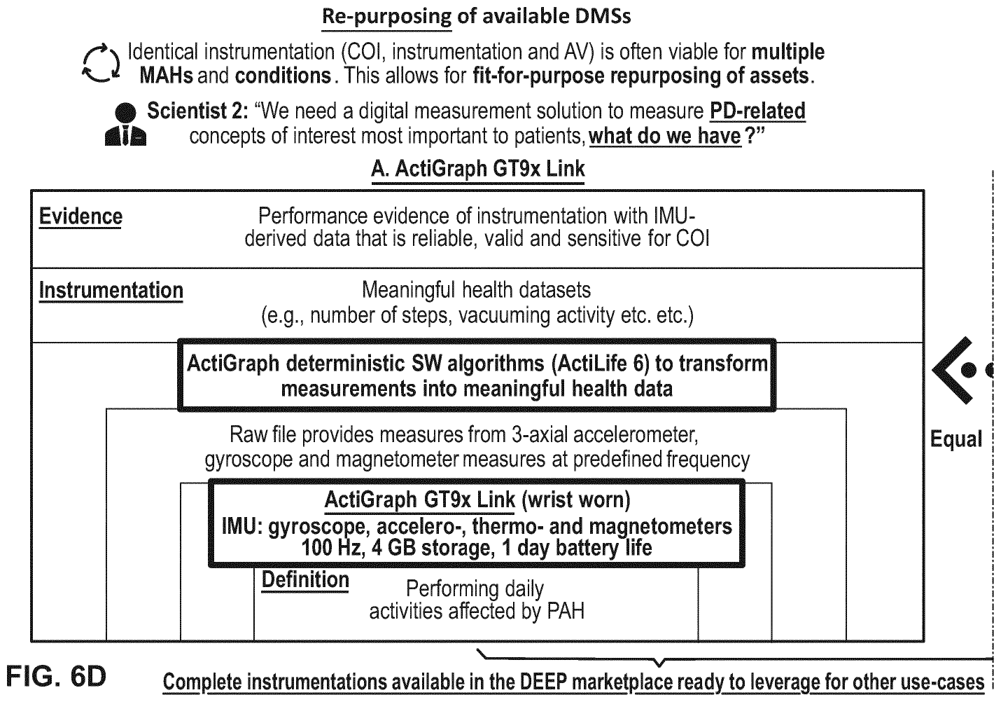

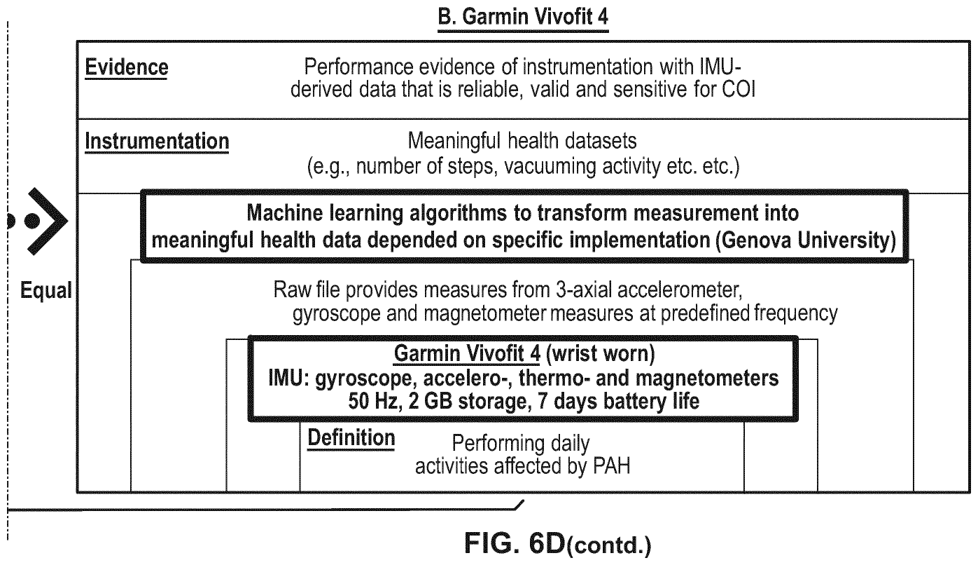

FIG. 6A depicts an example target solution profile for pulmonary arterial hypertension. Here, the meaningful aspect of health is the ability to perform activities of daily living and the corresponding hypothesis is that a particular therapeutic intervention (e.g., drug X) improves ability to perform physical activities. The concept of interest that is to be measured is the ability to perform daily activities while being affected by pulmonary arterial hypertension.

In the instrumentation asset, the measurement method generically identifies a wrist worn device with device specifications. Here, the measurement method is device-technology agnostic and does not identify a particular device nor a particular device-software. The raw data component includes the raw data file that is captured using the measurement method (e.g., wrist worn device). The algorithm component identifies algorithms that transform the raw data file captured using the measurement method into meaningful health data. The health data component includes the meaningful health dataset transformed by the algorithm of the preceding component. As shown in FIG. 6A, examples of meaningful health data for pulmonary arterial hypertension include measures of daily activity such as number of steps, vacuuming activity, and others).

The analytical validation component ensures that the meaningful health dataset is reliable, valid, and sensitive for the concept of interest. The clinical interpretation identifies a significant improvement in daily performance in PAH patients following the drug X intervention.

FIG. 6B depicts a first example digital measurement solution for pulmonary arterial hypertension. Additionally, FIG. 6C depicts a second example digital measurement solution for pulmonary arterial hypertension. Here, each of the digital measurement solution shown in FIGS. 6B and 6C are a common class represented by the target solution profile shown in FIG. 6A.

Referring to the DMS shown in FIG. 6B, the measurement method component specifies a particular wrist worn device (e.g., ActiGraph GT9x Link). Thus, the ActiGraph GT9X Link device captures data which is represented in a raw data file. The algorithm component transforms the raw data file into a meaningful health dataset. As shown in FIG. 6B, ActiGraph deterministic algorithms (e.g., ActiLife 6) is implemented as an algorithm to transform the raw data file into a meaningful health dataset. Altogether, in comparison to the TSP shown in FIG. 6A, the DMS shown in FIG. 6B specifies the particular device of the measurement method component and the particular algorithm of the algorithm component.

Referring to the DMS shown in FIG. 6C, the measurement method component specifies a particular wrist worn device (e.g., Garmin Vivofit 4) that differs from the device specified in FIG. 6B. Thus, the Garmin Vivofit 4 device captures data which is represented in a raw data file. The algorithm component transforms the raw data file into a meaningful health dataset. As shown in FIG. 6C, machine learning algorithms are implemented that transform the measurements captured by the Garmin Vivofit 4 into a meaningful health dataset. Altogether, in comparison to the TSP shown in FIG. 6A, the DMS shown in FIG. 6C specifies the particular device of the measurement method component and the particular algorithm of the algorithm component.

Each digital measurement solution shown in FIGS. 6B and 6C undergoes validation via a qualification protocol to ensure that the respective DMS generates comparable results. An example qualification protocol includes the following:
1) Participants wear the device of the DMS as well as a reference device of a DMS that had previously been successfully validated
2) Participants walk X steps (e.g., 100 steps)
3) Total number of steps measured by the device of the DMS is compared to the total number of steps measured by the reference device. If the difference is less than a threshold number (e.g., 10%), then the device of the DMS is successfully validated.

FIG. 6D depicts the repurposing of at least the instrumentation asset of digital measurement solutions. Here, FIG. 6D shows the target instrumentation profile (e.g., concept of interest component, the instrumentation asset, and analytical validation component) for each DMS shown in FIGS. 6B and 6C. Notably, the target instrumentation profiles of each DMS are interchangeable and viable for other meaningful aspects of health and conditions. Thus, the stack of assets that make up a target instrumentation profile were readily leveraged and incorporated into a different DMS. As a specific example, a digital measurement solution was needed for measuring Parkinson's Disease related concepts of interest. Thus, these target instrumentation profiles that were used for the pulmonary arterial hypertension condition were repurposed for measuring Parkinson's Disease.

### Example 3: Target Solution Profile and Digital Measurement Solutions for Parkinson's Disease

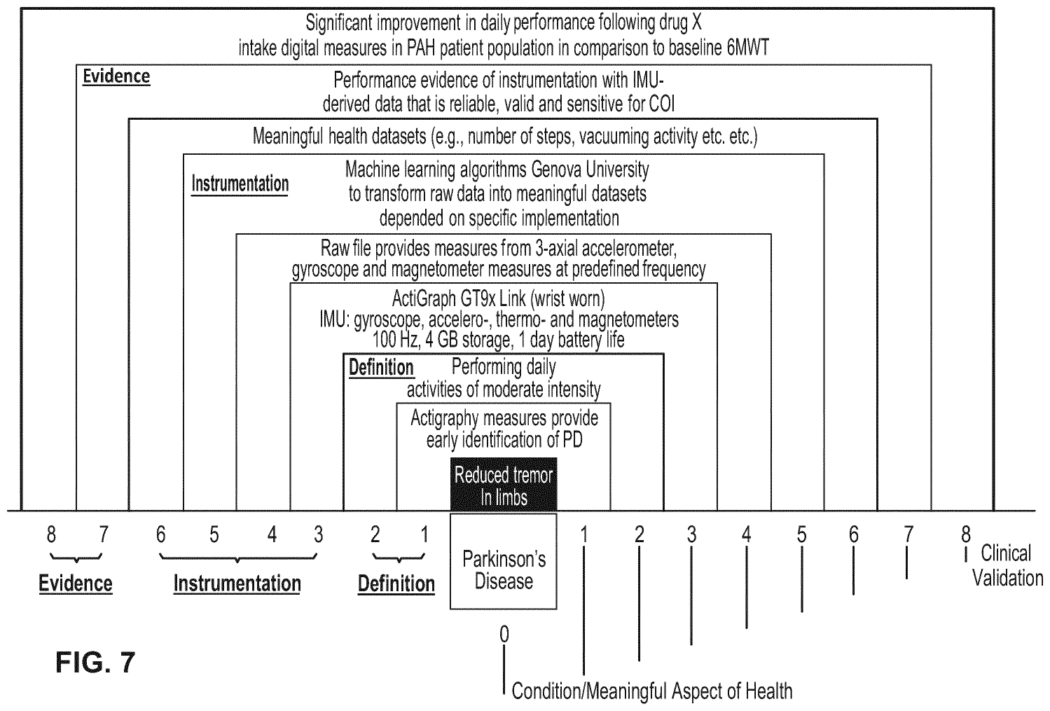

FIG. 7 depicts an example digital measurement solution for Parkinson's Disease. Here, the DMS for Parkinson's Disease incorporates a target instrumentation profile that was repurposed from a DMS of the pulmonary arterial hypertension condition. For example, the target instrumentation profile (which includes components 2 to 7) is identical to components 2 to 7 of a DMS for pulmonary arterial hypertension.

As shown in FIG. 7, the DMS for Parkinson's Disease identifies a specific wrist worn device (e.g., ActiGraph GT9X Link) in the measurement method component. Furthermore, the DMS for Parkinson's Disease identifies a specific algorithm (e.g., ActiGraph deterministic algorithm (e.g., ActiLife 6)) which transforms the raw data file captured by the ACtiGraph GT9X Link to a meaningful health dataset. Here, these components are identical to the corresponding components of the DMS for pulmonary arterial hypertension shown in FIG. 6B. Altogether, even though the condition (Parkinson's Disease) and meaningful aspect of health (reduced tremor in limbs) differs in the DMS shown in FIG. 7 in comparison to the DMS shown in FIG. 6B, components of the instrumentation asset are identical.

### Example 4: Standardized Solutions for Improved Regulatory Acceptance

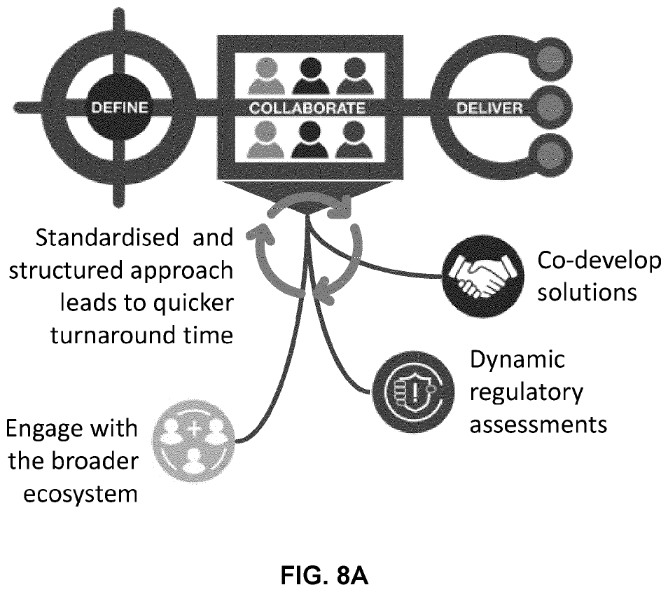

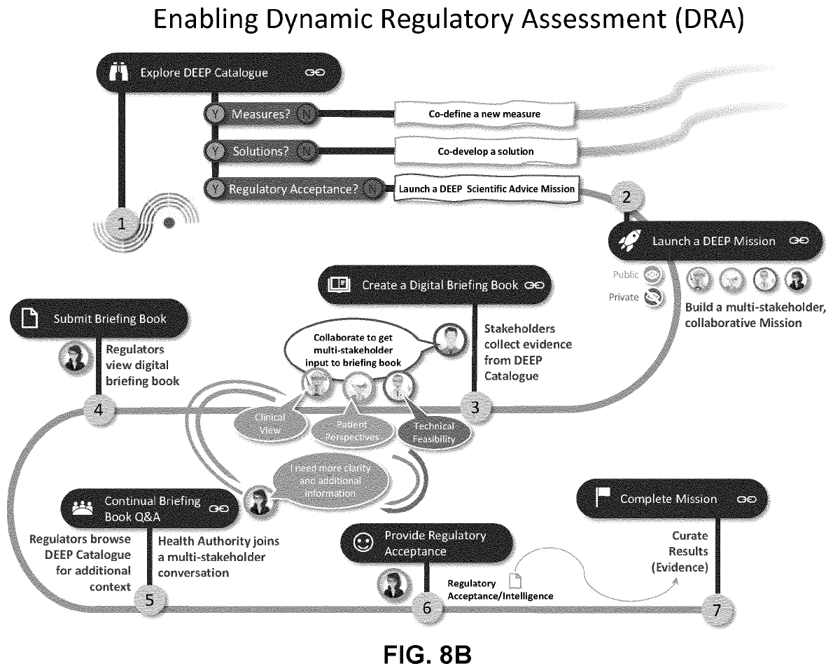

FIG. 8A depicts a high level overview involving collaborative efforts for developing standardized solutions. Here, multiple parties are involved in a collaborative effort for co-developing standardized solutions. These parties engage in a standardized and structured approach, which leads to quicker turnaround and development time. These standardized solutions undergo dynamic regulatory assessments by involving regulators at an early stage during development. Developed solutions are then delivered (e.g., to customers).

FIG. 8B depicts an example flow process involving various parties for enabling dynamic regulatory assessment of standardized solutions. The flow process begins at step 1 involving the Digital Endpoints Ecosystem and Protocols (DEEP) Catalogue. Here, additional digital measures and/or digital solutions are developed and furthermore, these digital solutions can undergo a dynamic regulatory acceptance. Dynamic regulatory acceptance (DRA) involves launching a DEEP mission, which involves multiple stakeholders that collaborate together on a common Mission. Specifically, at step 3, the stakeholders collaborate to develop a digital briefing book that includes the digital solution. As shown in FIG. 8B, the various stakeholders can provide various input into the creation of the briefing book, such as a clinical view, patient perspectives, and/or technical feasibility. At step 4, the briefing book is submitted for regulatory approval such that regulators access the briefing book including the digital solution.

The interaction between regulators and the stakeholders can be as follows: at step 5, the regulator logs in, browses the DEEP catalogue to further understand the digital solution with additional context, and views the public questions as well as the private background materials from both sponsor companies with their private questions. If needed, the regulators can re-engage with the stakeholders to obtain additional clarity and information. The regulator sees that patient and clinician input has already been incorporated. However, the regulator still has questions e.g., it appears important that for severe forms of the disease a comprehensive assessment is made about scratch activity. Thus, regulator asks for additional context regarding patient behavior such as where the patients scratch, how do they scratch, the hours of scratching, etc. The regulators also want to ask patients with less severe disease and understand if their scratch activity is different. The stakeholders (e.g., patient representatives) provide this feedback. Here, patients with severe forms of the disease scratch everywhere and also use both hands and even their feet to scratch. Alternatively, patients with the less severe form of the disease report that usually their itch flares up in one area and they end up scratch just that one spot.

The regulator then wants to ask the patients with a severe form of the disease about the camera solution. In light of their disease, would they tolerate the use of such a solution for periods of time? The patient representative responds that yes, their disease is already burdensome so that they would be willing to do this in order to help find a solution. They do however express concerns about doing this for an extended period of time. The regulator considers all this input and then formulates their response to the questions. They agree that scratch indeed is very meaningful and can be used as a key endpoint.

Regarding the two solutions envisioned, the regulator sees good applicability for both in different kinds of trials. They both sound plausible, but evidence of their performance to detect scratch activity is required and they also recommend developing evidence to better understand how much change in this measure is going to result in a meaningful benefit to patients. Also, the impact on sleep and next day sleepiness should be explored.

Regarding the private questions, the regulator recommends to Pharma company A (severe disease) to consider using both envisioned solutions in their trials. A study could be designed with periods of camera observation as well as wearable device use. It could be studied if the wearable solution could be a suitable surrogate for the more robust video measures. Thus, if the additional value of the video solution can be better understood, better guidance can be provided in the future. An ideal solution could be to use both in studies with severe forms of the disease, balancing scientific value with the burden on patients.

For pharma company B, the regulator foresees that the single wearable device solution could be sufficient to measure these isolated scratching flares. The regulator however recommends that the company also works to understand how well the video and wearable measures correlate and then make an informed choice in their trial design. The regulator recommends the sponsor comes back for more advice when more evidence is available and then discuss specific trial designs again.

Thus, if the regulator sees sufficient evidence, at step 6, the regulator provides regulatory acceptance of the digital solution. At step 7, the collaborative mission involving the multiple stakeholders is completed. The regulatory feedback is curated and connected with the catalogue (MAH, COI and TSP). Stakeholders involved in this regulatory process now have clear direction about next steps. Both solutions have their uses for different purposes and both sponsor companies are already starting to plan for solution development missions. Pharma company A wants to invest in both options, Pharma company B is interested in both, but clearly wants to prioritize the wearable solution development first.

### Example 5: Example Development of a Standardized Solution Involving Multiple Stakeholders

Assume that Pharmaceutical company A and Pharmaceutical Company B have generated their respective measurement definitions for atopic dermatitis and are interested in measuring number of nighttime scratching events. The remaining question is how to capture these measurements. Pharmaceutical company A would like to develop the right solution for measuring their endpoint. Thus Pharmaceutical company A accesses and searches the DEEP catalogue to identify the solutions that are already in existence.

For example, Pharmaceutical company A types into the asset search box: “scratch”, which results in discovering sensor devices, algorithms and relevant datasets for this use case. Thus, Pharmaceutical A can find the building blocks needed for their solution.

Here, Pharmaceutical company A can create a new mission seeking the services of a Custodian that can assemble a digital solution and maintain it. Pharmaceutical company A will fully fund this work, sets the access rights to Pharmaceutical company A fully owning the solution, but granting an operating license to the Custodian for a period of 3 years, with the Custodian being responsible for maintaining documentation of the solution, including any component upgrades during the licensing period.

The custodian assembles the solution from the components in the catalogue and connects the solution to the measurement definition already established. Pharmaceutical company A can now access the solution they need in their clinical trial.

Additionally, Pharmaceutical company B can now also see the solution being available for licensing from the Custodian (they get a notification that DMS is available for the TSP they are following/subscribed to). The solution performance looks promising and the licensing conditions appears fair. Pharmaceutical company B also decides to license the solution from the Custodian.

Altogether, multiple stakeholders can rapidly adopt solutions through more efficient pathways that are provided through standardized solutions.

### Tables

TABLE 1
Example Conditions of a Measurement Stack

| | |
|---|---|
| Occupant of heavy transport vehicle injured in | Systemic atrophies primarily affecting central nervous |
| collision with pedal cycle | system in diseases classified elsewhere |
| Diseases of capillaries | Other localized connective tissue disorders |
| Elevated erythrocyte sedimentation rate and | Pedal cycle rider injured in collision with other pedal |
| abnormality of plasma viscosity | cycle |
| Vascular syndromes of brain in cerebrovascular | Intracranial laceration and hemorrhage due to birth |
| diseases | injury |
| Obstructed labor due to malposition and | Unspecified fall |
| malpresentation of fetus | |
| Other specified types of T/NK-cell lymphoma | Transient cerebral ischemic attacks and related |
| | syndromes |
| Maternal infectious and parasitic diseases classifiable | Other and unspecified noninfective gastroenteritis and |
| elsewhere but complicating pregnancy, childbirth and | colitis |
| the puerperium | |
| Urethritis and urethral syndrome | Vitamin A deficiency |
| Other acantholytic disorders | Crushing injury of neck |
| Encounter for fitting and adjustment of other devices | Female pelvic inflammatory disorders in diseases |
| | classified elsewhere |
| Unspecified protein-calorie malnutrition | Explosion and rupture of other specified pressurized |
| | devices |
| Ophthalmic devices associated with adverse incidents | Encounter for prophylactic surgery |
| Contact with other mammals | Other diseases of inner ear |
| Retention of urine | Other disorders of binocular movement |
| Irritable bowel syndrome | Nonrheumatic mitral valve disorders |
| Exposure to other nonionizing radiation | Encounter for other aftercare and medical care |
| Congenital malformations of lung | Other headache syndromes |
| Yellow fever | Exfoliation due to erythematous conditions according |
| | to extent of body surface involved |
| Viral meningitis | Duodenal ulcer |
| Other degenerative diseases of basal ganglia | Other congenital malformations, not elsewhere |
| | classified |
| Abnormalities of forces of labor | Cicatricial alopecia [scarring hair loss] |
| Cataract in diseases classified elsewhere | Diaphragmatic hernia |
| Dietary zinc deficiency | Unspecified viral hepatitis |
| Congenital musculoskeletal deformities of head, face, | Burn and corrosion of respiratory tract |
| spine and chest | |
| Benign neoplasm of other and unspecified sites | Adult and child abuse, neglect and other |
| | maltreatment, suspected |
| Scarlet fever | Microcephaly |
| Intracranial and intraspinal abscess and granuloma | Embedded and impacted teeth |
| Open wound of knee and lower leg | Other disorders of pancreatic internal secretion |
| Opioid related disorders | Ill-defined and unknown cause of mortality |
| Fistulae involving female genital tract | Complications of anesthesia during pregnancy |
| Gastroenterology and urology devices associated with | Rheumatic tricuspid valve diseases |
| adverse incidents | |
| Disorder of patella | Lupus erythematosus |
| Malignant neoplasm of other and ill-defined sites | Other disorders of breast and disorders of lactation |
| | associated with pregnancy and the puerperium |
| Frostbite with tissue necrosis | Pemphigoid |
| Cholesteatoma of middle ear | Motorcycle rider injured in collision with two- or |
| | three-wheeled motor vehicle |
| Exposure to noise | Contact with hot air and other hot gases |
| Other viral diseases, not elsewhere classified | Mononeuropathy in diseases classified elsewhere |
| Hydatidiform mole | Open wound of shoulder and upper arm |
| Intraoperative and postprocedural complications and | Car occupant injured in noncollision transport |
| disorders of genitourinary system, not elsewhere | accident |
| classified | |
| Absent, scanty and rare menstruation | Glaucoma in diseases classified elsewhere |
| Fecal incontinence | Nocardiosis |
| Allergic contact dermatitis | Inhalant related disorders |
| Acute poliomyelitis | Pedal cycle rider injured in collision with two- or |
| | three-wheeled motor vehicle |
| Problems related to upbringing | Hemiplegia and hemiparesis |
| Superficial injury of hip and thigh | Fall from chair |
| Other salmonella infections | Generalized hyperhidrosis |
| Family history of primary malignant neoplasm | Specific developmental disorder of motor function |
| Malignant neoplasm of trachea | Acute nasopharyngitis [common cold] |
| Other helminthiases | Cataclysmic storm |
| Parapsoriasis | Malignant neoplasm of stomach |
| Benign neoplasm of soft tissue of retroperitoneum and | Aspergillosis |
| peritoneum | |
| Complications specific to multiple gestation | Other spirochetal infections |
| Pedal cycle rider injured in collision with pedestrian | Secondary parkinsonism |
| or animal | |
| Injury of nerves at wrist and hand level | Other and unspecified effects of other external causes |
| Malignant neoplasm of vulva | Other and unspecified soft tissue disorders, not |
| | elsewhere classified |
| Other immunodeficiencies | Drowning and submersion due to accident to |
| | watercraft |
| Phlebitis and thrombophlebitis | Injury of blood vessels of thorax |
| Nonvenereal syphilis | Paraphilias |
| Carcinoma in situ of breast | Abnormal findings on diagnostic imaging of lung |
| Presence of cardiac and vascular implants and grafts | Disorders of endocrine glands in diseases classified |
| | elsewhere |
| Contact with steam and other hot vapors | Nail disorders in diseases classified elsewhere |
| Primary disorders of muscles | Down syndrome |
| Congenital malformations of trachea and bronchus | Burns classified according to extent of body surface |
| | involved |
| Unspecified arthropod-borne viral fever | Failed attempted termination of pregnancy |
| Symptoms and signs concerning food and fluid intake | Erysipelas |
| Typhoid and paratyphoid fevers | Encounter for mental health services for victim and |
| | perpetrator of abuse |
| Shigellosis | Cushing's syndrome |
| Pre-existing hypertension with pre-eclampsia | Ankylosing spondylitis |
| Unspecified disorder of psychological development | Congenital ichthyosis |
| Abnormal findings on antenatal screening of mother | Carcinoma in situ of skin |
| Unspecified intellectual disabilities | Cytomegaloviral disease |
| Malignant neoplasm of adrenal gland | Other noninflammatory disorders of uterus, except |
| | cervix |
| Contact with other powered hand tools and household | Pedal cycle rider injured in noncollision transport |
| machinery | accident |
| Unspecified nephritic syndrome | Accidental rifle, shotgun and larger firearm discharge |
| | and malfunction |
| Disorders of autonomic nervous system | Gonococcal infection |
| Superficial injury of knee and lower leg | Foreign body in ear |
| Symptoms and signs involving appearance and | Problems related to employment and unemployment |
| behavior | |
| Other conduction disorders | Persons encountering health services for specific |
| | procedures and treatment, not carried out |
| Orchitis and epididymitis | Esophagitis |
| Rubella [German measles] | Do not resuscitate status |
| Toxic effect of soaps and detergents | Spontaneous rupture of synovium and tendon |
| Toxic effect of other noxious substances eaten as food | Anemia in chronic diseases classified elsewhere |
| Malignant neoplasm of pyriform sinus | Motorcycle rider injured in collision with heavy |
| | transport vehicle or bus |
| Disorders of lipoprotein metabolism and other | Complications of the puerperium, not elsewhere |
| lipidemias | classified |
| Periprosthetic fracture around internal prosthetic joint | Other disorders of ear, not elsewhere classified |
| Infections of genitourinary tract in pregnancy | Carcinoma in situ of oral cavity, esophagus and |
| | stomach |
| Impetigo | Influenza due to unidentified influenza virus |
| Lactose intolerance | Assault by bodily force |
| Hyperparathyroidism and other disorders of | Hookworm diseases |
| parathyroid gland | |
| Omphalitis of newborn | General hospital and personal-use devices associated |
| | with adverse incidents |
| Disorders of glycoprotein metabolism | Poisoning by, adverse effect of and underdosing of |
| | other systemic anti-infectives and antiparasitics |
| Other rickettsioses | Necrotizing enterocolitis of newborn |
| Actinomycosis | Other heart disorders in diseases classified elsewhere |
| Methemoglobinemia | Malignant neoplasm of other endocrine glands and |
| | related structures |
| Assault by other and unspecified firearm and gun | Disorders of gallbladder, biliary tract and pancreas in |
| discharge | diseases classified elsewhere |
| Congenital malformations of esophagus | Occupant of three-wheeled motor vehicle injured in |
| | collision with pedal cycle |
| Trichuriasis | Chromomycosis and pheomycotic abscess |
| Other benign neoplasms of uterus | Other disorders of gingiva and edentulous alveolar |
| | ridge |
| Other diseases of pancreas | Car occupant injured in collision with fixed or |
| | stationary object |
| Failure of sterile precautions during surgical and | Severe intellectual disabilities |
| medical care | |
| Unspecified sexually transmitted disease | Brucellosis |
| _DEEP transform is not an official code | Bus occupant injured in collision with two- or three- |
| | wheeled motor vehicle |
| Gender identity disorders | Polyhydramnios |
| Obstructed labor due to maternal pelvic abnormality | Anencephaly and similar malformations |
| Blood type | Other protozoal diseases, not elsewhere classified |
| Pedestrian conveyance accident | Lichen planus |
| Dentofacial anomalies [including malocclusion] | Intracranial and intraspinal abscess and granuloma in |
| | diseases classified elsewhere |
| Pneumothorax and air leak | Benign neoplasm of mesothelial tissue |
| Lymphoid leukemia | Vitamin D deficiency |
| Effects of heat and light | Pedestrian injured in collision with car, pick-up truck |
| | or van |
| Burn and corrosion of trunk | Encounter for fitting and adjustment of external |
| | prosthetic device |
| Foreign body in genitourinary tract | Acute appendicitis |
| Congenital malformations of anterior segment of eye | Spotted fever [tick-borne rickettsioses] |
| Erysipeloid | Superficial frostbite |
| Other and unspecified injuries of elbow and forearm | Viral and other specified intestinal infections |
| Schizotypal disorder | Contact with rodent |
| Dislocation and sprain of joints and ligaments at neck | Herpesviral [herpes simplex] infections |
| level | |
| Dermatitis due to substances taken internally | Urethral disorders in diseases classified elsewhere |
| Superficial injury of thorax | Other disorders of optic [2nd] nerve and visual |
| | pathways |
| Pediculosis and phthiriasis | Other tetanus |
| Contact with sharp glass | Ventral hernia |
| Poisoning by, adverse effect of and underdosing of | Female infertility |
| drugs primarily affecting the autonomic nervous | |
| system | |
| Pressure ulcer | Enterobiasis |
| Retinal disorders in diseases classified elsewhere | Other diseases of pericardium |
| Pneumoconiosis associated with tuberculosis | Crushing injury of lower leg |
| Retained foreign body fragments | Soft tissue disorders related to use, overuse and |
| | pressure |
| Family history of mental and behavioral disorders | Plasmodium vivax malaria |
| Military operations | Striking against or struck by sports equipment |
| Intraoperative and postprocedural complications and | Abnormalities of breathing |
| disorders of nervous system, not elsewhere classified | |
| Acute kidney failure | Other bacterial foodborne intoxications, not elsewhere |
| | classified |
| Malignant neoplasm of bladder | Peritonitis |
| Other disorders of penis | Unspecified viral hemorrhagic fever |
| Striking against or struck by other objects | Hyperfunction of pituitary gland |
| Ovarian dysfunction | Unspecified cause of accidental drowning and |
| | submersion |
| Hemolytic disease of newborn | Labor and delivery complicated by abnormality of |
| | fetal acid-base balance |
| Deficiency of other nutrient elements | Dislocation and sprain of joints and ligaments of |
| | elbow |
| Toxic effect of alcohol | Burn and corrosion of wrist and hand |
| Other complications of surgical and medical care, not | Encounter for procreative management |
| elsewhere classified | |
| Superficial injury of neck | Specific developmental disorders of scholastic skills |
| Other superficial mycoses | Pulmonary hemorrhage originating in the perinatal |
| | period |
| Osteomyelitis | Viral agents as the cause of diseases classified |
| | elsewhere |
| Disorders of aromatic amino-acid metabolism | Complications of transplanted organs and tissue |
| Other maternal diseases classifiable elsewhere but | Other and unspecified nontraumatic intracranial |
| complicating pregnancy, childbirth and the | hemorrhage |
| puerperium | |
| Other specified malaria | Contact with other heat and hot substances |
| Other congenital malformations of respiratory system | Other disorders involving the immune mechanism, |
| | not elsewhere classified |
| Congenital obstructive defects of renal pelvis and | Amebiasis |
| congenital malformations of ureter | |
| Disorders of vitreous body | Abnormal findings on diagnostic imaging of central |
| | nervous system |
| Nontraumatic subarachnoid hemorrhage | Salpingitis and oophoritis |
| Secondary malignant neoplasm of other and | Hypospadias |
| unspecified sites | |
| Varicose veins of lower extremities | Systemic lupus erythematosus (SLE) |
| Other conditions of integument specific to newborn | Fracture of shoulder and upper arm |
| Other disorders of synovium and tendon | Fracture of foot and toe, except ankle |
| Complications of other internal prosthetic devices, | Occupant of three-wheeled motor vehicle injured in |
| implants and grafts | collision with railway train or railway vehicle |
| Other intestinal helminthiases, not elsewhere | Encounter for immunization |
| classified | |
| Other disorders of peritoneum | Other benign neoplasms of connective and other soft |
| | tissue |
| Other mononeuropathies | Fracture of forearm |
| Chronic tubulo-interstitial nephritis | Chronic rhinitis, nasopharyngitis and pharyngitis |
| Adult osteomalacia | Malignant neoplasm of spinal cord, cranial nerves and |
| | other parts of central nervous system |
| Other psychotic disorder not due to a substance or | Unspecified abdominal hernia |
| known physiological condition | |
| Contact with powered lawn mower | Reduction defects of upper limb |
| Toxic effect of carbon monoxide | Reactions and intoxications due to drugs administered |
| | to newborn |
| Syndactyly | Other sepsis |
| Respiratory tuberculosis | Enlarged lymph nodes |
| Nail disorders | Persons encountering health services for other |
| | counseling and medical advice, not elsewhere |
| | classified |
| Caught, crushed, jammed or pinched in or between | Cranial nerve disorders in diseases classified |
| objects | elsewhere |
| Occlusion and stenosis of cerebral arteries, not | Other symptoms and signs involving the digestive |
| resulting in cerebral infarction | system and abdomen |
| Polyuria | Congenital malformations of ear causing impairment |
| | of hearing |
| Placental disorders | Occupant of pick-up truck or van injured in |
| | noncollision transport accident |
| Other paralytic syndromes | Chancroid |
| Fall from non-moving wheelchair, nonmotorized | Scoliosis |
| scooter and motorized mobility scooter | |
| Myiasis | Neoplasm of uncertain behavior of endocrine glands |
| Diphyllobothriasis and sparganosis | Abnormal results of function studies |
| Mononeuropathies of lower limb | Congenital malformations of cardiac chambers and |
| | connections |
| Age-related physical debility | Gestational [pregnancy-induced] hypertension |
| | without significant proteinuria |
| Pemphigus | Bronchitis, not specified as acute or chronic |
| Contact with nonvenomous amphibians | Pneumonitis due to solids and liquids |
| Pedal cycle rider injured in collision with railway | Overweight and obesity |
| train or railway vehicle | |
| Conductive and sensorineural hearing loss | Other osteochondrodysplasias |
| Malignant neoplasm of nasopharynx | Other bullous disorders |
| Effects of other deprivation | Poisoning by, adverse effect of and underdosing of |
| | diuretics and other and unspecified drugs, |
| | medicaments and biological substances |
| Contact with other sharp objects | Injury of muscle, fascia and tendon at lower leg level |
| Other disorders of bone | Osteonecrosis |
| Abnormal findings in other body fluids and | Contact with hypodermic needle |
| substances | |
| Localized swelling, mass and lump of skin and | Unspecified mycosis |
| subcutaneous tissue | |
| Bacterial sepsis of newborn | Toxic effect of corrosive substances |
| Other hypothyroidism | Occupant of heavy transport vehicle injured in |
| | collision with car, pick-up truck or van |
| Malignant neoplasm of other and ill-defined digestive | Plague |
| organs | |
| Cleft palate with cleft lip | General- and plastic-surgery devices associated with |
| | adverse incidents |
| Unspecified pneumoconiosis | False labor |
| Urethral discharge | Other nonscarring hair loss |
| Cholecystitis | Malignant neoplasm of vagina |
| Abnormalities of gait and mobility | Abuse of non-psychoactive substances |
| _DEEP transform is not an official code | Trisomy 18 and Trisomy 13 |
| Other congenital malformations of heart | Certain current complications following ST elevation |
| | (STEMI) and non-ST elevation (NSTEMI) |
| | myocardial infarction (within the 28 day period) |
| Occupant of heavy transport vehicle injured in other | Injury of muscle, fascia and tendon at forearm level |
| and unspecified transport accidents | |
| Transitory disorders of carbohydrate metabolism | Other and unspecified disorders of Eustachian tube |
| specific to newborn | |
| Other epidermal thickening | Intentional self-harm by crashing of motor vehicle |
| Rheumatic fever with heart involvement | Occupant of heavy transport vehicle injured in |
| | collision with fixed or stationary object |
| Unspecified protozoal disease | Other aneurysm |
| Complications of bariatric procedures | Malignant neoplasm of cervix uteri |
| Small kidney of unknown cause | Carcinoma in situ of cervix uteri |
| Eustachian salpingitis and obstruction | Functional dyspepsia |
| Injury of blood vessels at abdomen, lower back and | Malignant neoplasm of gum |
| pelvis level | |
| Age-related cataract | Traumatic amputation of elbow and forearm |
| Toxic effect of other and unspecified substances | Strongyloidiasis |
| Occupant of three-wheeled motor vehicle injured in | Fissure and fistula of anal and rectal regions |
| collision with two- or three-wheeled motor vehicle | |
| Malignant neoplasm of rectosigmoid junction | Other strabismus |
| Malignant neoplasm of hypopharynx | Problems related to lifestyle |
| Chronic nephritic syndrome | Hypoparathyroidism |
| Abnormal findings in specimens from male genital | Hypothermia of newborn |
| organs | |
| Superficial injury of abdomen, lower back, pelvis and | Superficial injury of ankle, foot and toes |
| external genitals | |
| Problems related to certain psychosocial | Benign neoplasm of major salivary glands |
| circumstances | |
| Perineal laceration during delivery | Malignant neoplasm of bone and articular cartilage of |
| | other and unspecified sites |
| Pedal cycle rider injured in collision with other | Flatulence and related conditions |
| nonmotor vehicle | |
| Somatoform disorders | Q fever |
| Other leukemias of specified cell type | Immunization not carried out and underimmunization |
| | status |
| Acute laryngitis and tracheitis | Chronic respiratory disease originating in the perinatal |
| | period |
| Thiamine deficiency | Exposure to ignition or melting of nightwear |
| Other disorders of breast | Reduction defects of lower limb |
| Motorcycle rider injured in collision with pedestrian | Injury of nerves and spinal cord at neck level |
| or animal | |
| Birth injury to scalp | Malignant neoplasm of uterus, part unspecified |
| Disseminated intravascular coagulation [defibrination | Cardiomyopathy in diseases classified elsewhere |
| syndrome] | |
| Balanced rearrangements and structural markers, not | Malignant neoplasm of accessory sinuses |
| elsewhere classified | |
| Lichen simplex chronicus and prurigo | Dengue fever [classical dengue] |
| Other disorders of white blood cells | Bacterial meningitis, not elsewhere classified |
| Other diseases of hard tissues of teeth | Chronic ischemic heart disease |
| Coalworker's pneumoconiosis | Pedal cycle rider injured in other and unspecified |
| | transport accidents |
| Anthrax | Congenital deformities of feet |
| Intentional self-harm by rifle, shotgun and larger | Testicular dysfunction |
| firearm discharge | |
| Fracture at wrist and hand level | Granuloma inguinale |
| Carcinoma in situ of other and unspecified digestive | Other injury due to accident on board watercraft, |
| organs | without accident to watercraft |
| Osteoporosis without current pathological fracture | Contact with other hot fluids |
| Meningococcal infection | Encounter for other prophylactic measures |
| Other bacterial diseases, not elsewhere classified | Encounter for procedures for purposes other than |
| | remedying health state |
| Acquired absence of limb | Exposure to other animate mechanical forces |
| Other and unspecified infectious diseases | Pneumoconiosis due to other inorganic dusts |
| Taeniasis | Hemorrhage from respiratory passages |
| Visual disturbances | Other and unspecified disorders of circulatory system |
| Adrenogenital disorders | Persons encountering health services in other |
| | circumstances |
| Fracture of cervical vertebra and other parts of neck | Bitten or stung by nonvenomous insect and other |
| | nonvenomous arthropods |
| Hypertensive chronic kidney disease | Assault by smoke, fire and flames |
| Other congenital malformations of male genital | Other congenital malformations of intestine |
| organs | |
| Other specified transport accidents | Phakomatoses, not elsewhere classified |
| Premature rupture of membranes | Thoracic, thoracolumbar, and lumbosacral |
| | intervertebral disc disorders |
| Cerebrovascular disorders in diseases classified | Rifle, shotgun and larger firearm discharge, |
| elsewhere | undetermined intent |
| Diverticular disease of intestine | Other noninflammatory disorders of vulva and |
| | perineum |
| Other arthropod-borne viral fevers, not elsewhere | Dyslexia and other symbolic dysfunctions, not |
| classified | elsewhere classified |
| Malignant neoplasm of thymus | Neoplasms of unspecified behavior |
| Malignant neoplasm of pancreas | Intraoperative and postprocedural complications and |
| | disorders of eye and adnexa, not elsewhere classified |
| Sequelae of leprosy | Esophageal varices |
| Personal history of certain other diseases | Keratitis |
| Intraoperative complications of endocrine system | Inflammatory disorders of male genital organs, not |
| | elsewhere classified |
| Injury of nerves and spinal cord at thorax level | Other infestations |
| Other disturbances of temperature regulation of | Other disorders of brain in diseases classified |
| newborn | elsewhere |
| Alcohol related disorders | Congenital lens malformations |
| Other stimulant related disorders | Benign neoplasm of other and unspecified female |
| | genital organs |
| Acute hepatitis A | Rat-bite fevers |
| Unspecified mental disorder due to known | Poisoning by, adverse effect of and underdosing of |
| physiological condition | systemic antibiotics |
| Bus occupant injured in collision with heavy transport | Foreign body or object entering through skin |
| vehicle or bus | |
| Fall on and from playground equipment | Foreign body on external eye |
| Other chromosome abnormalities, not elsewhere | Chronic viral hepatitis |
| classified | |
| Multiple valve diseases | Other viral infections characterized by skin and |
| | mucous membrane lesions, not elsewhere classified |
| Aortic aneurysm and dissection | Other diseases of intestine |
| Suppurative and unspecified otitis media | Other hereditary hemolytic anemias |
| Miliary tuberculosis | Sequelae of hyperalimentation |
| Fall on and from stairs and steps | Assault by steam, hot vapors and hot objects |
| Other complications of labor and delivery, not | Delusional disorders |
| elsewhere classified | |
| Persistent mood [affective] disorders | Other rheumatic heart diseases |
| Bartonellosis | Burn and corrosion of lower limb, except ankle and |
| | foot |
| Leprosy [Hansen's disease] | Other congenital malformations of brain |
| Urethral stricture | Foreign body in respiratory tract |
| Dislocation and sprain of joints and ligaments of | Congenital malformations of larynx |
| thorax | |
| Enthesopathies, lower limb, excluding foot | Occupational exposure to risk factors |
| Obstructive and reflux uropathy | Other sex chromosome abnormalities, male |
| | phenotype, not elsewhere classified |
| Crushing injury of shoulder and upper arm | Handgun discharge, undetermined intent |
| Complications of artificial openings of the digestive | Fall, jump or diving into water |
| system | |
| Paralytic ileus and intestinal obstruction without | Hypotension |
| hernia | |
| Unspecified severe protein-calorie malnutrition | Other and unspecified injuries of wrist, hand and |
| | finger(s) |
| Erythema multiforme | Pain and other conditions associated with female |
| | genital organs and menstrual cycle |
| Other diseases of esophagus | Maternal care for other conditions predominantly |
| | related to pregnancy |
| Hydrocele and spermatocele | Syncope and collapse |
| Sequelae of cerebrovascular disease | Occupant of railway train or railway vehicle injured in |
| | transport accident |
| Car occupant injured in collision with heavy transport | Occupant of heavy transport vehicle injured in |
| vehicle or bus | collision with pedestrian or animal |
| Cystitis | Pleural effusion, not elsewhere classified |
| Flood | Other disorders of kidney and ureter, not elsewhere |
| | classified |
| Disorders of retroperitoneum | Other congenital malformations of peripheral vascular |
| | system |
| Poisoning by, adverse effect of and underdosing of | Streptococcal sepsis |
| antiepileptic, sedative- hypnotic and | |
| antiparkinsonismdrugs | |
| Lack of expected normal physiological development | Hepatic failure, not elsewhere classified |
| in childhood and adults | |
| Injury of intra-abdominal organs | Pneumonia due to Streptococcus pneumoniae |
| Other disorders of adrenal gland | Facial nerve disorders |
| Contact with hot tap-water | Cerebral palsy |
| African trypanosomiasis | Convulsions, not elsewhere classified |
| Portal vein thrombosis | Migraine |
| Benign neoplasm of meninges | Malnutrition in pregnancy, childbirth and the |
| | puerperium |
| Neoplasm of uncertain behavior of female genital | Disorders of lacrimal system |
| organs | |
| Other respiratory conditions originating in the | Exposure to controlled fire in building or structure |
| perinatal period | |
| Hypofunction and other disorders of the pituitary | Other puerperal infections |
| gland | |
| Hormone sensitivity malignancy status | Monocytic leukemia |
| Other female pelvic inflammatory diseases | Diseases of tongue |
| Glycosuria | Toxic effect of halogen derivatives of aliphatic and |
| | aromatic hydrocarbons |
| Polyp of female genital tract | Encounter for administrative examination |
| Other conditions originating in the perinatal period | Other abnormalities of plasma proteins |
| Umbilical hernia | Other and unspecified injuries of lower leg |
| Disturbances of skin sensation | Iodine-deficiency related thyroid disorders and allied |
| | conditions |
| Other congenital malformations of digestive system | Donors of organs and tissues |
| Fall from bed | Other inflammation of eyelid |
| Injury of blood vessels at shoulder and upper arm | Problems related to life management difficulty |
| level | |
| Unspecified parasitic disease | Pain in throat and chest |
| Encephalitis, myelitis and encephalomyelitis in | Arthropathies in other diseases classified elsewhere |
| diseases classified elsewhere | |
| Staphylococcal scalded skin syndrome | Exposure to ignition of highly flammable material |
| Problems related to housing and economic | Excessive, frequent and irregular menstruation |
| circumstances | |
| Polyneuropathy in diseases classified elsewhere | Motorcycle rider injured in collision with pedal cycle |
| Occupant of pick-up truck or van injured in collision | Benign mammary dysplasia |
| with railway train or railway vehicle | |
| Adult and child abuse, neglect and other | Labor and delivery complicated by umbilical cord |
| maltreatment, confirmed | complications |
| Eclampsia | Chlamydial lymphogranuloma (venereum) |
| Acquired deformities of fingers and toes | Influenza due to certain identified influenza viruses |
| Encounter for other special examination without | Exposure to excessive natural heat |
| complaint, suspected or reported diagnosis | |
| Immunodeficiency associated with other major | Fall from tree |
| defects | |
| Radiodermatitis | Impulse disorders |
| Pleural effusion in conditions classified elsewhere | Assault by pushing or placing victim in front of |
| | moving object |
| Malignant neoplasm of other connective and soft | Femoral hernia |
| tissue | |
| Assault by explosive material | Sequelae of inflammatory diseases of central nervous |
| | system |
| Birth injury to peripheral nervous system | Paroxysmal tachycardia |
| Vascular disorders of intestine | Occupant of three-wheeled motor vehicle injured in |
| | collision with fixed or stationary object |
| Myasthenia gravis and other myoneural disorders | Postpolio syndrome |
| Superficial injury of shoulder and upper arm | Complications of anesthesia during labor and delivery |
| Other lack of coordination | Cervical disc disorders |
| Other and unspecified disorders of prostate | Superficial injury of wrist, hand and fingers |
| Anesthesiology devices associated with adverse | Malignant neoplasm of heart, mediastinum and pleura |
| incidents | |
| Accidental hit, strike, kick, twist, bite or scratch by | Dislocation and sprain of joints and ligaments of |
| another person | lumbar spine and pelvis |
| Exposure to other inanimate mechanical forces | Congenital malformations of musculoskeletal system, |
| | not elsewhere classified |
| Feeding problems of newborn | Spontaneous abortion |
| Dengue hemorrhagic fever | Complications of internal orthopedic prosthetic |
| | devices, implants and grafts |
| Other human herpesviruses | Other acquired deformities of limbs |
| Other problems related to primary support group, | Dissociative and conversion disorders |
| including family circumstances | |
| Malignant neoplasm of gallbladder | Dislocation and sprain of joint and ligaments of hip |
| Liver disorders in diseases classified elsewhere | Intentional self-harm by sharp object |
| Malignant neoplasm of floor of mouth | Disorders of puberty, not elsewhere classified |
| Tick-borne viral encephalitis | Benign neoplasm of thyroid gland |
| Poisoning by, adverse effect of and underdosing of | Malignant neoplasm of other and unspecified parts of |
| agents primarily affecting the cardiovascular system | tongue |
| Other symptoms and signs involving general | Exposure to uncontrolled fire in building or structure |
| sensations and perceptions | |
| Disorders of tooth development and eruption | Measles |
| Perforation of tympanic membrane | Injury of other and unspecified intrathoracic organs |
| Thyrotoxicosis [hyperthyroidism] | Congenital syphilis |
| Nutritional marasmus | Follicular lymphoma |
| Osteopathies in diseases classified elsewhere | Asphyxiation |
| Other and unspecified medical devices associated | Intentional self-harm by other specified means |
| with adverse incidents | |
| Atheroembolism | Immunodeficiency with predominantly antibody |
| | defects |
| Open wound of ankle, foot and toes | Retinal vascular occlusions |
| Other disorders of pigmentation | Nontraffic accident of specified type but victim's |
| | mode of transport unknown |
| Explosion and rupture of boiler | Neuromuscular dysfunction of bladder, not elsewhere |
| | classified |
| Other noninflammatory disorders of vagina | Arterial embolism and thrombosis |
| Diphtheria | Contact with nonvenomous plant thorns and spines |
| | and sharp leaves |
| Other disorders of urethra | Other congenital malformations of tongue, mouth and |
| | pharynx |
| Zygomycosis | Combined immunodeficiencies |
| Edema, not elsewhere classified | Unspecified viral infection characterized by skin and |
| | mucous membrane lesions |
| Typhus fever | Struck by thrown, projected or falling object |
| Poisoning by, adverse effect of and underdosing of | Encounter for observation and evaluation of newborn |
| narcotics and psychodysleptics [hallucinogens] | for suspected diseases and conditions ruled out |
| Unspecified contracted kidney | Disorders of other cranial nerves |
| Drowning and submersion, undetermined intent | Acute pharyngitis |
| Anemia due to enzyme disorders | Encounter for screening for infectious and parasitic |
| | diseases |
| Sequelae of tuberculosis | Varicose veins of other sites |
| Explosion and rupture of pressurized tire, pipe or hose | Other endocrine disorders |
| Otalgia and effusion of ear | Fibroblastic disorders |
| Dislocation and sprain of joints and ligaments at wrist | Burn and corrosion of head, face, and neck |
| and hand level | |
| Gangrene, not elsewhere classified | Bladder disorders in diseases classified elsewhere |
| Infectious mononucleosis | Calcification and ossification of muscle |
| Orthopedic devices associated with adverse incidents | Family history of certain disabilities and chronic |
| | diseases (leading to disablement) |
| Disorders of iris and ciliary body in diseases classified | Kaposi's sarcoma |
| elsewhere | |
| Malignant neoplasm of other and unspecified female | Acute myocardial infarction |
| genital organs | |
| Trichomoniasis | Tuberculosis of other organs |
| Assault by other specified means | Unspecified transport accident |
| Fracture of femur | Encounter for maternal postpartum care and |
| | examination |
| Other congenital infectious and parasitic diseases | Recurrent pregnancy loss |
| Malignant neoplasm of parotid gland | Unspecified jaundice |
| Myocarditis in diseases classified elsewhere | Chagas' disease |
| Intracranial nontraumatic hemorrhage of newborn | Newborn affected by maternal conditions that may be |
| | unrelated to present pregnancy |
| Intraoperative and postprocedural complications and | Long term (current) drug therapy |
| disorders of musculoskeletal system, not | |
| elsewhereclassified | |
| Hypothermia | Intraoperative and postprocedural complications and |
| | disorders of digestive system, not elsewhere classified |
| Other disorders of fluid, electrolyte and acid-base | Accidental discharge and malfunction from other and |
| balance | unspecified firearms and guns |
| Acute obstructive laryngitis [croup] and epiglottitis | Congenital malformations of ovaries, fallopian tubes |
| | and broad ligaments |
| Acute myocarditis | Venous complications and hemorrhoids in the |
| | puerperium |
| Other congenital malformations of circulatory system | Other disturbances of cerebral status of newborn |
| Benign neoplasm of other and unspecified endocrine | Vascular dementia |
| glands | |
| Other pleural conditions | Open wound of head |
| Dislocation and sprain of joints and ligaments at | Rosacea |
| ankle, foot and toe level | |
| Other obstructed labor | Turner's syndrome |
| Other acute viral hepatitis | Crushing injury of wrist, hand and fingers |
| Rash and other nonspecific skin eruption | Nonadministration of surgical and medical care |
| Relapsing fevers | Problems related to medical facilities and other health |
| | care |
| Other enthesopathies | Other disorders of bladder |
| Encounter for pregnancy test and childbirth and | Inflammatory disease of cervix uteri |
| childcare instruction | |
| Benign neoplasm of breast | Toxic effect of noxious substances eaten as seafood |
| Other effects of reduced temperature | Burn and corrosion of other internal organs |
| Acute nephritic syndrome | Problems related to social environment |
| Cystic kidney disease | Open wound of elbow and forearm |
| Myositis | Marasmic kwashiorkor |
| Other nonpsychotic mental disorders | Pedestrian injured in other and unspecified transport |
| | accidents |
| Congenital pneumonia | Other mycoses, not elsewhere classified |
| Unspecified contact dermatitis | Other congenital malformations of skull and face |
| | bones |
| Assault by rifle, shotgun and larger firearm discharge | Other disorders of skin and subcutaneous tissue |
| | related to radiation |
| Infectious gastroenteritis and colitis, unspecified | Physical medicine devices associated with adverse |
| | incidents |
| Injury of cranial nerve | Epidermolysis bullosa |
| Influenza due to other identified influenza virus | Dietary selenium deficiency |
| Other congenital malformations of spinal cord | Congenital malformations of spine and bony thorax |
| Malignant neoplasm of ureter | Encounter for contraceptive management |
| Contact with hot household appliances | Hyperaldosteronism |
| Candidiasis | Pneumonia due to other infectious organisms, not |
| | elsewhere classified |
| Disseminated intravascular coagulation of newborn | Exposure to other specified smoke, fire and flames |
| Viral warts | Malignant neoplasm of meninges |
| Other sexually transmitted chlamydial diseases | Acute hepatitis B |
| Motor- or nonmotor-vehicle accident, type of vehicle | Non-follicular lymphoma |
| unspecified | |
| Traumatic amputation of shoulder and upper arm | Leptospirosis |
| Earthquake | Malignant neoplasm of nasal cavity and middle ear |
| Congenital malformations of aortic and mitral valves | Subclinical iodine-deficiency hypothyroidism |
| Other diseases of stomach and duodenum | Contact with hot heating appliances, radiators and |
| | pipes |
| Other mosquito-borne viral fevers | Other diseases caused by chlamydiae |
| Problems related to other psychosocial circumstances | Malignant neoplasm of ovary |
| Rheumatic chorea | Hordeolum and chalazion |
| Malignant neoplasm of retroperitoneum and | Acute and subacute endocarditis |
| peritoneum | |
| Other local infections of skin and subcutaneous tissue | Infections of breast associated with pregnancy, the |
| | puerperium and lactation |
| Disorders of myoneural junction and muscle in | Other and unspecified dorsopathies, not elsewhere |
| diseases classified elsewhere | classified |
| Foreign body in alimentary tract | Injury of muscle, fascia and tendon at shoulder and |
| | upper arm level |
| Melanoma in situ | Abnormal findings in specimens from other organs, |
| | systems and tissues |
| Contaminated medical or biological substances | Osteoarthritis of first carpometacarpal joint |
| Other symptoms and signs involving cognitive | Occupant of special vehicle mainly used in agriculture |
| functions and awareness | injured in transport accident |
| Other specified congenital malformation syndromes | Toxic effect of aflatoxin and other mycotoxin food |
| affecting multiple systems | contaminants |
| Family history of other specific disorders | Nonrheumatic pulmonary valve disorders |
| Other arthritis | Other acute skin changes due to ultraviolet radiation |
| Specific personality disorders | Metabolic acidemia in newborn |
| Neutropenia | Contact with blunt object, undetermined intent |
| Chorioretinal inflammation | Functional disorders of polymorphonuclear |
| | neutrophils |
| Neurological devices associated with adverse | Occupant of pick-up truck or van injured in collision |
| incidents | with car, pick-up truck or van |
| Cannabis related disorders | Gastritis and duodenitis |
| Dislocation and sprain of joints and ligaments of | Car occupant injured in other and unspecified |
| shoulder girdle | transport accidents |
| Mastoiditis and related conditions | Newborn affected by intrauterine (fetal) blood loss |
| Other joint disorder, not elsewhere classified | Poisoning by, adverse effect of and underdosing of |
| | topical agents primarily affecting skin and |
| | mucousmembrane and by ophthalmological, |
| | otorhinorlaryngological and dental drugs |
| Fall on same level from slipping, tripping and | Legal intervention |
| stumbling | |
| Zoster [herpes zoster] | Other zoonotic bacterial diseases, not elsewhere |
| | classified |
| Hereditary ataxia | Systemic disorders of connective tissue in diseases |
| | classified elsewhere |
| Nosocomial condition | Hypertensive heart and chronic kidney disease |
| Other transitory neonatal endocrine disorders | Delirium due to known physiological condition |
| Malignant neoplasm of liver and intrahepatic bile | Nasal polyp |
| ducts | |
| Other medical procedures as the cause of abnormal | Other sex chromosome abnormalities, female |
| reaction of the patient, or of later complication, | phenotype, not elsewhere classified |
| withoutmention of misadventure at the time of the | |
| procedure | |
| Noninflammatory disorders of testis | Disorders of sclera |
| Exposure to controlled fire, not in building or | Presence of other devices |
| structure | |
| Atherosclerosis | Injury of muscle, fascia and tendon at neck level |
| Burn and corrosion confined to eye and adnexa | Inguinal hernia |
| Hereditary and idiopathic neuropathy | Nonspecific lymphadenitis |
| Dorsalgia | Acute sinusitis |
| Late syphilis | Skin changes due to chronic exposure to nonionizing |
| | radiation |
| Symptoms and signs involving emotional state | Labor and delivery complicated by intrapartum |
| | hemorrhage, not elsewhere classified |
| Blastomycosis | Chronic sinusitis |
| Injury of blood vessels at lower leg level | Nerve root and plexus disorders |
| Other transitory neonatal electrolyte and metabolic | Pyoderma gangrenosum |
| disturbances | |
| Unspecified malaria | Disorders of newborn related to long gestation and |
| | high birth weight |
| Intentional self-harm by handgun discharge | Pre-eclampsia |
| Estrogen receptor status | Abnormality of red blood cells |
| Disorders of newborn related to slow fetal growth and | Rheumatic mitral valve diseases |
| fetal malnutrition | |
| Other disorders of adult personality and behavior | Pneumoconiosis due to asbestos and other mineral |
| | fibers |
| Septic arterial embolism | Unspecified kidney failure |
| Brief psychotic disorder | Newborn affected by complications of placenta, cord |
| | and membranes |
| Other psychoactive substance related disorders | Other congenital malformations of ear |
| Hereditary factor IX deficiency | Fall from cliff |
| Other nutritional anemias | Other disorders of conjunctiva |
| Mental and behavioral disorders associated with the | Chronic diseases of tonsils and adenoids |
| puerperium, not elsewhere classified | |
| Acute pericarditis | Bus occupant injured in other and unspecified |
| | transport accidents |
| Open wound of hip and thigh | Crushing injury of thorax, and traumatic amputation |
| | of part of thorax |
| Motorcycle rider injured in other and unspecified | Injury of nerves at hip and thigh level |
| transport accidents | |
| Aphagia and dysphagia | Conduct disorders |
| Other aplastic anemias and other bone marrow failure | Malignant immunoproliferative diseases and certain |
| syndromes | other B-cell lymphomas |
| Osteitis deformans [Paget's disease of bone] | Listeriosis |
| Chronic laryngitis and laryngotracheitis | Inconclusive laboratory evidence of human |
| | immunodeficiency virus [HIV] |
| Early syphilis | Sexual dysfunction, unspecified |
| Injury of lumbar and sacral spinal cord and nerves at | Other specified diseases with participation of |
| abdomen, lower back and pelvis level | lymphoreticular and reticulohistiocytic tissue |
| Outcome of delivery | Disorders of external ear in diseases classified |
| | elsewhere |
| Contact with explosive material, undetermined intent | Diseases of Bartholin's gland |
| Abnormal serum enzyme levels | Convulsions of newborn |
| Disorders of purine and pyrimidine metabolism | Hydrops fetalis due to hemolytic disease |
| Nonrheumatic aortic valve disorders | Other necrotizing vasculopathies |
| Chorioretinal disorders in diseases classified | Bacterial infection of unspecified site |
| elsewhere | |
| Other specified and unspecified injuries of neck | Malignant neoplasm of oropharynx |
| Malignant neoplasm of peripheral nerves and | Other and unspecified injuries of thorax |
| autonomic nervous system | |
| Mononeuropathies of upper limb | Coccidioidomycosis |
| Toxoplasmosis | Other noninflammatory disorders of cervix uteri |
| Injury of blood vessels at wrist and hand level | Malignant neoplasm of other and unspecified parts of |
| | mouth |
| Mental disorder, not otherwise specified | Overexertion and strenuous or repetitive movements |
| Echinococcosis | Crushing injury of head |
| Pedestrian injured in collision with heavy transport | Avalanche, landslide and other earth movements |
| vehicle or bus | |
| Injury of muscle, fascia and tendon at hip and thigh | Multiple gestation |
| level | |
| Exposure to man-made visible and ultraviolet light | Atrioventricular and left bundle-branch block |
| Congenital absence, atresia and stenosis of small | Neonatal aspiration |
| intestine | |
| Filariasis | Disorders of vestibular function |
| Bipolar disorder | Sequelae of malnutrition and other nutritional |
| | deficiencies |
| Motorcycle rider injured in noncollision transport | Cleft lip |
| accident | |
| Renal tubulo-interstitial disorders in diseases | Other erythematous conditions |
| classified elsewhere | |
| Terrorism | Other misadventures during surgical and medical care |
| Abnormal findings on diagnostic imaging of other | Other specific joint derangements |
| body structures | |
| Exposure to uncontrolled fire, not in building or | Irritant contact dermatitis |
| structure | |
| Injury of eye and orbit | Diseases of vocal cords and larynx, not elsewhere |
| | classified |
| Sequelae of other and unspecified infectious and | Benign neoplasm of urinary organs |
| parasitic diseases | |
| Injury of nerves at ankle and foot level | Falling, jumping or pushed from a high place, |
| | undetermined intent |
| Occupant of heavy transport vehicle injured in | Eating disorders |
| collision with other nonmotor vehicle | |
| Neonatal jaundice due to other excessive hemolysis | Other neonatal hemorrhages |
| Myelodysplastic syndromes | Other disorders of lens |
| Pregnant state | Congenital malformations of breast |
| Other sexual disorders | Other predominantly sexually transmitted diseases, |
| | not elsewhere classified |
| Folate deficiency anemia | Common variable immunodeficiency |
| Leukemia of unspecified cell type | Occupant of heavy transport vehicle injured in |
| | noncollision transport accident |
| Acute upper respiratory infections of multiple and | Abnormal findings in cerebrospinal fluid |
| unspecified sites | |
| Pyogenic arthritis | Other and unspecified arthropathy |
| Other disorders of eyelid | Car occupant injured in collision with other nonmotor |
| | vehicle |
| Other perinatal hematological disorders | Congenital malformations of great veins |
| Encounter for follow-up examination after completed | Encounter for general examination without complaint, |
| treatment for conditions other than malignant | suspected or reported diagnosis |
| neoplasm Medical surveillance following completed | |
| treatment | |
| Disturbances of smell and taste | Apocrine sweat disorders |
| Yaws | Iridocyclitis |
| Vasculitis limited to skin, not elsewhere classified | Encounter for adjustment and management of |
| | implanted device |
| Other disorders of central nervous system | Intraoperative and postprocedural complications of |
| | the spleen |
| Illness, unspecified | Abnormalities of heart beat |
| Malignant neoplasm without specification of site | Other venous embolism and thrombosis |
| Fall on and from ladder | Encephalocele |
| _DEEP transform is not an official code | Other renal tubulo-interstitial diseases |
| Contact with dog | Congenital malformations of posterior segment of eye |
| Certain early complications of trauma, not elsewhere | Synovitis and tenosynovitis |
| classified | |
| Occupant of heavy transport vehicle injured in | Inflammatory disorders of breast |
| collision with heavy transport vehicle or bus | |
| Injury of nerves at lower leg level | Biomechanical lesions, not elsewhere classified |
| Acute pancreatitis | Toxic encephalopathy |
| Pedal cycle rider injured in collision with car, pick-up | Other inflammatory liver diseases |
| truck or van | |
| Tubulo-interstitial nephritis, not specified as acute or | Unspecified urinary incontinence |
| chronic | |
| Otosclerosis | Complications following infusion, transfusion and |
| | therapeutic injection |
| Pruritus | Exposure to other specified electric current |
| Cellulitis and acute lymphangitis | Otorhinolaryngological devices associated with |
| | adverse incidents |
| Sequelae of poliomyelitis | Problems related to education and literacy |
| Superficial injury of head | Other congenital musculoskeletal deformities |
| Other and unspecified polyneuropathies | Benign neoplasm of brain and other parts of central |
| | nervous system |
| Exposure to electric transmission lines | Benign neoplasm of other and ill-defined parts of |
| | digestive system |
| Spinal muscular atrophy and related syndromes | Complications of genitourinary prosthetic devices, |
| | implants and grafts |
| Other disorders of middle ear and mastoid in diseases | Other congenital malformations of nervous system |
| classified elsewhere | |
| Complications of anesthesia during the puerperium | Assault by pushing from high place |
| Pedestrian injured in collision with two- or three- | Other degenerative disorders of nervous system in |
| wheeled motor vehicle | diseases classified elsewhere |
| Venous complications and hemorrhoids in pregnancy | Other disorders of ear in diseases classified elsewhere |
| Intentional self-harm by jumping or lying in front of | Anophthalmos, microphthalmos and macrophthalmos |
| moving object | |
| Cleft palate | Carrier of infectious disease |
| Shoulder lesions | Purpura and other hemorrhagic conditions |
| Toxic liver disease | Maternal care for disproportion |
| Granulomatous disorders of skin and subcutaneous | Occupant of three-wheeled motor vehicle injured in |
| tissue | other and unspecified transport accidents |
| Seborrheic keratosis | Resistance to antimicrobial drugs |
| Contact with other nonvenomous reptiles | Systemic sclerosis [scleroderma] |
| Malignant neoplasm of corpus uteri | Pulmonary edema |
| Cutaneous abscess, furuncle and carbuncle | Disorders of esophagus in diseases classified |
| | elsewhere |
| Disorders of sphingolipid metabolism and other lipid | Somnolence, stupor and coma |
| storage disorders | |
| Obstetrical tetanus | Other fetal stress complicating labor and delivery |
| Malignant neoplasm of lip | Dysplasia of cervix uteri |
| Corneal scars and opacities | _DEEP transform is not an official code |
| Contact with and (suspected) exposure to | Personal risk factors, not elsewhere classified |
| communicable diseases | |
| Counseling related to sexual attitude, behavior and | Other disorders of external ear |
| orientation | |
| Pneumonia, unspecified organism | Abnormal findings on neonatal screening |
| Disorders of social functioning with onset specific to | Other disorders of skin and subcutaneous tissue, not |
| childhood and adolescence | elsewhere classified |
| Intraoperative and postprocedural complications and | Amyloidosis |
| disorders of respiratory system, not | |
| elsewhereclassified | |
| Failure in dosage during surgical and medical care | Inflammatory diseases of prostate |
| Malignant neoplasm of other and unspecified parts of | Other vitamin deficiencies |
| biliary tract | |
| Other slipping, tripping and stumbling and falls | Other diseases of jaws |
| Exposure to smoke, fire and flames, undetermined | Malignant neoplasm of small intestine |
| intent | |
| Assault by crashing of motor vehicle | Hypertrichosis |
| Poisoning by, adverse effect of and underdosing of | Other congenital malformations of face and neck |
| anesthetics and therapeutic gases | |
| Indeterminate sex and pseudohermaphroditism | Spondylosis |
| Injury of muscle, fascia and tendon at wrist and hand | Heartburn |
| level | |
| Malignant neoplasm of other and unspecified urinary | Kernicterus |
| organs | |
| Headache | Abscess of anal and rectal regions |
| Exposure to other forces of nature | Drowning and submersion due to accident on board |
| | watercraft, without accident to watercraft |
| Congenital malformations of gallbladder, bile ducts | Encounter for care involving renal dialysis |
| and liver | |
| Abnormal involuntary movements | Exposure to other specified factors |
| Abnormality in fetal heart rate and rhythm | Occupant of pick-up truck or van injured in collision |
| complicating labor and delivery | with fixed or stationary object |
| Failed induction of labor | Hemorrhoids and perianal venous thrombosis |
| Encounter for antenatal screening of mother | Artificial opening status |
| Mild intellectual disabilities | Contact with hot drinks, food, fats and cooking oils |
| Retained placenta and membranes, without | Occupant of three-wheeled motor vehicle injured in |
| hemorrhage | collision with other nonmotor vehicle |
| Leishmaniasis | Hypertensive heart disease |
| Intentional self-harm by steam, hot vapors and hot | Neoplasm of uncertain behavior of other and |
| objects | unspecified sites |
| Obstetric and gynecological devices associated with | Acute tonsillitis |
| adverse incidents | |
| Abnormal findings in specimens from respiratory | Retarded development following protein-calorie |
| organs and thorax | malnutrition |
| Assault by blunt object | Encounter for follow-up examination after completed |
| | treatment for malignant neoplasm |
| Other and unspecified malignant neoplasms of | Plasmodium falciparum malaria |
| lymphoid, hematopoietic and related tissue | |
| Radiological devices associated with adverse | Other diseases of gallbladder |
| incidents | |
| Malignant neoplasm of esophagus | Anogenital herpesviral [herpes simplex] infections |
| Other neoplasms of uncertain behavior of lymphoid, | Excessive vomiting in pregnancy |
| hematopoietic and related tissue | |
| Stillbirth | Secondary hypertension |
| Occupant of pick-up truck or van injured in collision | Other and unspecified injuries of abdomen, lower |
| with other nonmotor vehicle | back, pelvis and external genitals |
| Noninflammatory disorders of ovary, fallopian tube | Other disorders of brain |
| and broad ligament | |
| Malignant neoplasm of testis | Ascariasis |
| Other and unspecified metabolic disorders | Asymptomatic human immunodeficiency virus [HIV] |
| | infection status |
| Other disorders of eye and adnexa | Disorders of peritoneum in infectious diseases |
| | classified elsewhere |
| Other spondylopathies | Discharge of firework |
| Intentional self-harm by blunt object | Meningitis due to other and unspecified causes |
| Rapidly progressive nephritic syndrome | Personal history of other diseases and conditions |
| Occupant of pick-up truck or van injured in collision | Profound intellectual disabilities |
| with pedal cycle | |
| Erythema nodosum | Alcoholic liver disease |
| Other congenital malformations of limb(s) | Intentional self-harm by other and unspecified firearm |
| | and gun discharge |
| Car occupant injured in collision with two- or three- | Exposure to sunlight |
| wheeled motor vehicle | |
| Other disorders of cornea | Otitis media in diseases classified elsewhere |
| Acute pyelonephritis | Sexual dysfunction not due to a substance or known |
| | physiological condition |
| Hypertrophic disorders of skin | Protein-calorie malnutrition of moderate and mild |
| | degree |
| Other and unspecified syphilis | Placenta previa |
| Hemorrhage, not elsewhere classified | Viral conjunctivitis |
| Melanocytic nevi | Other congenital malformations of kidney |
| Accident to powered aircraft causing injury to | Bus occupant injured in collision with railway train or |
| occupant | railway vehicle |
| Enteropathic arthropathies | Sunburn |
| Other disorders of carbohydrate metabolism | Malaise and fatigue |
| Disorders of male genital organs in diseases classified | Other specified events, undetermined intent |
| elsewhere | |
| Gastrojejunal ulcer | Other deforming dorsopathies |
| Open wound of neck | Other acute ischemic heart diseases |
| Encounter for supervision of normal pregnancy | Congenital malformations of uterus and cervix |
| Occupant of heavy transport vehicle injured in | Disorders of orbit |
| collision with two- or three-wheeled motor vehicle | |
| Paracoccidioidomycosis | Stomatitis and related lesions |
| Pedestrian injured in collision with other nonmotor | Other and unspecified injuries of head |
| vehicle | |
| Abdominal and pelvic pain | Dementia in other diseases classified elsewhere |
| Cysticercosis | Pulmonary embolism |
| Other trisomies and partial trisomies of the | Rheumatic aortic valve diseases |
| autosomes, not elsewhere classified | |
| Hemorrhage in early pregnancy | Encounter for cesarean delivery without indication |
| Leiomyoma of uterus | Encounter for examination and observation for other |
| | reasons |
| Peptic ulcer, site unspecified | Other behavioral and emotional disorders with onset |
| | usually occurring in childhood and adolescence |
| Motorcycle rider injured in collision with fixed or | Polyarteritis nodosa and related conditions |
| stationary object | |
| Other specified and unspecified types of non-Hodgkin | Conjunctivitis |
| lymphoma | |
| Malignant neoplasm of colon | Non-pressure chronic ulcer of lower limb, not |
| | elsewhere classified |
| Open wound of wrist, hand and fingers | Perpetrator of assault, maltreatment and neglect |
| Complications following ectopic and molar pregnancy | Specific developmental disorders of speech and |
| | language |
| Malignant neoplasm of base of tongue | Other viral hemorrhagic fevers, not elsewhere |
| | classified |
| Pericarditis in diseases classified elsewhere | Other protozoal intestinal diseases |
| Obsessive-compulsive disorder | Onchocerciasis |
| Chlamydia psittaci infections | Fracture of lumbar spine and pelvis |
| Occupant of pick-up truck or van injured in collision | Injury of blood vessels at hip and thigh level |
| with pedestrian or animal | |
| Hair color and hair shaft abnormalities | Voice and resonance disorders |
| Assault by drowning and submersion | Osteochondrodysplasia with defects of growth of |
| | tubular bones and spine |
| Other and unspecified dermatitis | Other postprocedural states |
| Other cerebrovascular diseases | Malignant neoplasm of placenta |
| Cysts of oral region, not elsewhere classified | Transitory neonatal disorders of calcium and |
| | magnesium metabolism |
| Maternal care for known or suspected fetal | Kyphosis and lordosis |
| abnormality and damage | |
| Maternal care for other fetal problems | Contact with lifting and transmission devices, not |
| | elsewhere classified |
| Nonsuppurative otitis media | Seborrheic dermatitis |
| Nontraumatic intracerebral hemorrhage | Personal history of malignant neoplasm |
| Pneumoconiosis due to dust containing silica | Hemorrhagic disease of newborn |
| Other diseases of digestive system | Other anemias |
| Accidental handgun discharge and malfunction | Paralytic strabismus |
| Newborn affected by noxious substances transmitted | Pedal cycle rider injured in collision with heavy |
| via placenta or breast milk | transport vehicle or bus |
| Exposure to other man-made environmental factors | Papulosquamous disorders in diseases classified |
| | elsewhere |
| Iron deficiency anemia | Other follicular disorders |
| Other congenital malformations of urinary system | Scabies |
| Congenital malformation syndromes due to known | Acute posthemorrhagic anemia |
| exogenous causes, not elsewhere classified | |
| Male erectile dysfunction | Isolated proteinuria with specified morphological |
| | lesion |
| Hypertrophy of breast | Fracture of skull and facial bones |
| Malignant neoplasm of brain | Falling, lying or running before or into moving object, |
| | undetermined intent |
| Fall while being carried or supported by other persons | Other symptoms and signs involving the nervous and |
| | musculoskeletal systems |
| Drug- and heavy-metal-induced tubulo-interstitial and | Other and unspecified disorders of nose and nasal |
| tubular conditions | sinuses |
| Other bacterial intestinal infections | Problems related to care provider dependency |
| Dietary calcium deficiency | Fall from, out of or through building or structure |
| Accidental drowning and submersion while in | Erythema in diseases classified elsewhere |
| swimming-pool | |
| Malignant neoplasm of kidney, except renal pelvis | Bus occupant injured in collision with pedal cycle |
| Bus occupant injured in noncollision transport | Operations of war |
| accident | |
| Ascites | Contact with birds (domestic) (wild) |
| Acute bronchitis | Congenital malformations of cardiac septa |
| Hemangioma and lymphangioma, any site | Spondylopathies in diseases classified elsewhere |
| Other functional intestinal disorders | Occupant of pick-up truck or van injured in collision |
| | with heavy transport vehicle or bus |
| Long labor | Other specified health status |
| Effects of air pressure and water pressure | Localized adiposity |
| Other disorders of amino-acid metabolism | Schistosomiasis [bilharziasis] |
| Birth injury to skeleton | Unspecified lump in breast |
| Personality and behavioral disorders due to known | Other disorders of kidney and ureter in diseases |
| physiological condition | classified elsewhere |
| Occupant of three-wheeled motor vehicle injured in | Direct infections of joint in infectious and parasitic |
| collision with heavy transport vehicle or bus | diseases classified elsewhere |
| Acquired pure red cell aplasia [erythroblastopenia] | Other disorders of teeth and supporting structures |
| Neoplasm of uncertain behavior of male genital | Volume depletion |
| organs | |
| Crohn's disease [regional enteritis] | Nondiabetic hypoglycemic coma |
| Other peripheral vascular diseases | Vitamin B12 deficiency anemia |
| Other bacterial agents as the cause of diseases | Other specified cause of accidental non-transport |
| classified elsewhere | drowning and submersion |
| Other congenital malformations of eye | Malignant neoplasm of other and ill-defined sites in |
| | the lip, oral cavity and pharynx |
| Other cataract | Carcinoma in situ of middle ear and respiratory |
| | system |
| Other congenital malformations of integument | Occupant of special construction vehicle injured in |
| | transport accident |
| Disorders of globe | Unspecified chronic bronchitis |
| Occupant of powered streetcar injured in transport | Sarcoidosis |
| accident | |
| Interstitial emphysema and related conditions | Follicular cysts of skin and subcutaneous tissue |
| originating in the perinatal period | |
| Pyothorax | Meningitis in bacterial diseases classified elsewhere |
| Other disorders of thyroid | Poisoning by, adverse effect of and underdosing of |
| | nonopioid analgesics, antipyretics and antirheumatics |
| Toxic effect of other gases, fumes and vapors | Secondary malignant neoplasm of respiratory and |
| | digestive organs |
| Tuberculosis of nervous system | Animal-rider or occupant of animal-drawn vehicle |
| | injured in transport accident |
| Avulsion and traumatic amputation of part of head | Diseases of pulp and periapical tissues |
| Other and unspecified diseases of spinal cord | Arenaviral hemorrhagic fever |
| Other and unspecified diseases of blood and blood- | Other disorders of peripheral nervous system |
| forming organs | |
| Hematuria | Bus occupant injured in collision with car, pick-up |
| | truck or van |
| Carcinoma in situ of other and unspecified sites | Intracranial and intraspinal phlebitis and |
| | thrombophlebitis |
| Bus occupant injured in collision with other nonmotor | Hodgkin lymphoma |
| vehicle | |
| Radiation sickness, unspecified | Calculus of urinary tract in diseases classified |
| | elsewhere |
| Symptoms and signs specifically associated with | Burn and corrosion, body region unspecified |
| systemic inflammation and infection | |
| Superficial injury of elbow and forearm | Other diseases of biliary tract |
| Hereditary factor VIII deficiency | Contact with agricultural machinery |
| Malignant neoplasm of palate | Monosomies and deletions from the autosomes, not |
| | elsewhere classified |
| Motorcycle rider injured in collision with railway | Acne |
| train or railway vehicle | |
| Pain, unspecified | Other disorders of cartilage |
| Manic episode | Pneumonia in diseases classified elsewhere |
| Other contact with and (suspected) exposures | Deficiency of other B group vitamins |
| hazardous to health | |
| Sequelae of complication of pregnancy, childbirth, | Unspecified viral encephalitis |
| and the puerperium | |
| Exposure to ionizing radiation | Malignant neoplasm of tonsil |
| Abnormal findings in specimens from digestive | Tularemia |
| organs and abdominal cavity | |
| Other disorders of amniotic fluid and membranes | Accidental drowning and submersion while in bath- |
| | tub |
| Activity codes | Intentional self-harm by drowning and submersion |
| Reaction to severe stress, and adjustment disorders | Encounter for medical observation for suspected |
| | diseases and conditions ruled out |
| Other disorders of arteries and arterioles | Other and unspecified injuries of hip and thigh |
| Vasomotor and allergic rhinitis | Other benign neoplasms of skin |
| Intentional self-harm by smoke, fire and flames | Moderate intellectual disabilities |
| Pain associated with micturition | Other inflammation of vagina and vulva |
| Unspecified mood [affective] disorder | _DEEP transform is not an official code |
| Other papulosquamous disorders | Other abdominal hernia |
| Other disorders of bone density and structure | Polydactyly |
| Ascorbic acid deficiency | Eccrine sweat disorders |
| Trachoma | Benign neoplasm of male genital organs |
| Family history of other conditions | Volcanic eruption |
| Gastric ulcer | Injury of heart |
| Assault by unspecified means | Diseases of thymus |
| Exposure to high and low air pressure and changes in | Fall due to ice and snow |
| air pressure | |
| Optic neuritis | Thalassemia |
| Contact with crocodile or alligator | Unspecified maternal hypertension |
| Emphysema | Disorders of newborn related to short gestation and |
| | low birth weight, not elsewhere classified |
| Other viral encephalitis, not elsewhere classified | Injury of nerves at shoulder and upper arm level |
| Rabies | Exfoliative dermatitis |
| Neoplasm of uncertain behavior of brain and central | Place of occurrence of the external cause |
| nervous system | |
| Sedative, hypnotic, or anxiolytic related disorders | Other disorders of veins |
| Contact with nonvenomous marine animal | Intraoperative and postprocedural complications and |
| | disorders of ear and mastoid process, not |
| | elsewhereclassified |
| Open wound of thorax | Disorders of refraction and accommodation |
| Cholelithiasis | Other noninfective disorders of lymphatic vessels and |
| | lymph nodes |
| Unspecified psychosis not due to a substance or | Obstetric embolism |
| known physiological condition | |
| Injury of nerves at forearm level | Malignant neoplasm of bronchus and lung |
| Bus occupant injured in collision with pedestrian or | Other intestinal obstruction of newborn |
| animal | |
| Unspecified viral infection of central nervous system | Encounter for other postprocedural aftercare |
| Other disorders of blood and blood-forming organs in | Amnestic disorder due to known physiological |
| diseases classified elsewhere | condition |
| Encounter for screening for malignant neoplasms | Benign neoplasm of bone and articular cartilage |
| Other infections specific to the perinatal period | Malignant neoplasm of bone and articular cartilage of |
| | limbs |
| Fibrosis and cirrhosis of liver | Encounter for screening for other diseases and |
| | disorders |
| Prolonged stay in weightless environment | Newborn affected by other complications of labor and |
| | delivery |
| Other fluke infections | Assault by handgun discharge |
| Other specified air transport accidents | Poisoning by, adverse effect of and underdosing of |
| | primarily systemic and hematological agents, not |
| | elsewhere classified |
| Varicella [chickenpox] | Puerperal sepsis |
| Unspecified acute lower respiratory infection | Cardiovascular devices associated with adverse |
| | incidents |
| Kwashiorkor | Other diseases of liver |
| Pneumocystosis | Calculus of lower urinary tract |
| Congenital deformities of hip | Mature T/NK-cell lymphomas |
| Alopecia areata | Other diseases of pulmonary vessels |
| Niacin deficiency [pellagra] | Shared psychotic disorder |
| Juvenile osteochondrosis of hip and pelvis | Bullous disorders in diseases classified elsewhere |
| Mumps | Erosion and ectropion of cervix uteri |
| External cause status | Malignant neoplasm of other and ill-defined sites in |
| | the respiratory system and intrathoracic organs |
| Pedestrian injured in collision with pedal cycle | Disorder of continuity of bone |
| Tetanus neonatorum | Nystagmus and other irregular eye movements |
| Tic disorder | Premature separation of placenta [abruptio placentae] |
| Whooping cough | Cardiovascular disorders originating in the perinatal |
| | period |
| Dental caries | Crashing of motor vehicle, undetermined intent |
| Bronchiectasis | Malignant neoplasm of renal pelvis |
| Transepidermal elimination disorders | Other symptoms and signs involving the circulatory |
| | and respiratory system |
| Surgical operation and other surgical procedures as | Psychological and behavioral factors associated with |
| the cause of abnormal reaction of the patient, or of | disorders or diseases classified elsewhere |
| later complication, without mention of misadventure | |
| at the time of the procedure | |
| Complications peculiar to reattachment and | Fracture of lower leg, including ankle |
| amputation | |
| Vulvovaginal ulceration and inflammation in diseases | Occupant of three-wheeled motor vehicle injured in |
| classified elsewhere | collision with car, pick-up truck or van |
| Motorcycle rider injured in collision with other | Malignant neoplasm of eye and adnexa |
| nonmotor vehicle | |
| Benign neoplasm of colon, rectum, anus and anal | Pedal cycle rider injured in collision with fixed or |
| canal | stationary object |
| Otitis externa | Sporotrichosis |
| Occupant of heavy transport vehicle injured in | Disorders of porphyrin and bilirubin metabolism |
| collision with railway train or railway vehicle | |
| Other and unspecified disorders of male genital | Benign neoplasm of mouth and pharynx |
| organs | |
| Unspecified misadventure during surgical and | Gingivitis and periodontal diseases |
| medical care | |
| Other disorders of nervous system in diseases | Maternal care for malpresentation of fetus |
| classified elsewhere | |
| Other diseases of anus and rectum | Other and unspecified symptoms and signs involving |
| | the genitourinary system |
| Disorders of trigeminal nerve | Other and unspecified water transport accidents |
| Benign prostatic hyperplasia | Subsequent ST elevation (STEMI) and non-ST |
| | elevation (NSTEMI) myocardial infarction |
| Intraoperative and postprocedural complications of | Postpartum hemorrhage |
| skin and subcutaneous tissue | |
| Essential (primary) hypertension | Cardiac arrest |
| Malignant neoplasm of rectum | Intentional self-harm by explosive material |
| Traumatic amputation of ankle and foot | Polycythemia vera |
| Urticaria | Disorders of prepuce |
| Dislocation and sprain of joints and ligaments of knee | Umbilical hemorrhage of newborn |
| Pinta [carate] | Other perinatal digestive system disorders |
| Neonatal jaundice from other and unspecified causes | Cardiomyopathy |
| Proteinuria | Encounter for plastic and reconstructive surgery |
| | following medical procedure or healed injury |
| Abscess of lung and mediastinum | Vertiginous syndromes in diseases classified |
| | elsewhere |
| Chronic kidney disease (CKD) | Congenital iodine-deficiency syndrome |
| Exposure to excessive natural cold | Corns and callosities |
| Polyglandular dysfunction | Mycetoma |
| Congenital viral diseases | Other disorders of iris and ciliary body |
| Allergy status to drugs, medicaments and biological | Other congenital malformations of skin |
| substances | |
| Malignant neoplasm of other and unspecified male | Newborn affected by maternal complications of |
| genital organs | pregnancy |
| Poisoning by, adverse effect of and underdosing of | Corrosions classified according to extent of body |
| agents primarily acting on smooth and skeletal | surface involved |
| muscles and the respiratory system | |
| Occupant of three-wheeled motor vehicle injured in | Crushing injury and traumatic amputation of |
| noncollision transport accident | abdomen, lower back, pelvis and external genitals |
| Cardiac murmurs and other cardiac sounds | Other juvenile osteochondrosis |
| Genetic susceptibility to disease | Car occupant injured in collision with car, pick-up |
| | truck or van |
| Reduction defects of unspecified limb | Disorders of muscle in diseases classified elsewhere |
| Hypertensive crisis | Neoplasm of uncertain behavior of middle ear and |
| | respiratory and intrathoracic organs |
| Nephrotic syndrome | Malignant neoplasm of penis |
| Fall from other furniture | Other and unspecified firearm discharge, |
| | undetermined intent |
| Airway disease due to specific organic dust | Retinal detachments and breaks |
| Other injury due to accident to watercraft | Complications of procedures, not elsewhere classified |
| Motorcycle rider injured in collision with car, pick-up | Other congenital malformations of female genitalia |
| truck or van | |
| Bacterial pneumonia, not elsewhere classified | Other abnormal products of conception |
| Dizziness and giddiness | Streptococcus, Staphylococcus, and Enterococcus as |
| | the cause of diseases classified elsewhere |
| Congenital malformations of great arteries | Contact with nonpowered hand tool |
| Deformity and disproportion of reconstructed breast | Male infertility |
| Acute lymphadenitis | Hepatomegaly and splenomegaly, not elsewhere |
| | classified |
| Meningitis in other infectious and parasitic diseases | Ectopic pregnancy |
| classified elsewhere | |
| Occupant of three-wheeled motor vehicle injured in | Toxic effect of contact with venomous animals and |
| collision with pedestrian or animal | plants |
| Encephalitis, myelitis and encephalomyelitis | Encounter for full-term uncomplicated delivery |
| Phobic anxiety disorders | Other abnormal uterine and vaginal bleeding |
| Fracture of rib(s), sternum and thoracic spine | Postinfective and reactive arthropathies |
| Exposure to ignition or melting of other clothing and | Crushed, pushed or stepped on by crowd or human |
| apparel | stampede |
| Benign neoplasm of middle ear and respiratory | Female genital prolapse |
| system | |
| Car occupant injured in collision with railway train or | _DEEP transform is not an official code |
| railway vehicle | |
| Gestational [pregnancy-induced] edema and | Occupant of special all-terrain or other off-road motor |
| proteinuria without hypertension | vehicle, injured in transport accident |
| Peritonsillar abscess | Explosion and rupture of gas cylinder |
| Respiratory conditions due to other external agents | Neoplasm of uncertain behavior of urinary organs |
| Maternal care for abnormality of pelvic organs | Emotional disorders with onset specific to childhood |
| Acquired absence of organs, not elsewhere classified | Calculus of kidney and ureter |
| Poisoning by, adverse effect of and underdosing of | Infection due to other mycobacteria |
| psychotropic drugs, not elsewhere classified | |
| Other disorders of middle ear mastoid | Traffic accident of specified type but victim's mode of |
| | transport unknown |
| Injury of blood vessels at forearm level | Crushing injury of hip and thigh |
| Smallpox | Malignant melanoma of skin |
| Intraoperative and postprocedural complications and | Unspecified intestinal parasitism |
| disorders of circulatory system, not elsewhere | |
| classified | |
| Occlusion and stenosis of precerebral arteries, not | Hallucinogen related disorders |
| resulting in cerebral infarction | |
| DEEP transform is not an official code | Internal derangement of knee |
| Hereditary nephropathy, not elsewhere classified | Burn and corrosion of ankle and foot |
| Pulmonary eosinophilia, not elsewhere classified | Other abnormal findings of blood chemistry |
| Other congenital malformations of upper alimentary | test-condition for testcases |
| tract | |
| Occupant of special vehicle mainly used on industrial | Other and unspecified malignant neoplasm of skin |
| premises injured in transport accident | |
| Diseases of salivary glands | Malignant neoplasms of breast |
| Secondary and unspecified malignant neoplasm of | Multiple myeloma and malignant plasma cell |
| lymph nodes | neoplasms |
| Other diseases of upper respiratory tract | Carcinoma in situ of other and unspecified genital |
| | organs |
| Endometriosis | Sickle-cell disorders |
| Other osteochondropathies | Diabetes mellitus due to underlying condition |
| Other problems with newborn | Type 1 diabetes mellitus |
| Atrophic disorders of skin | Type 2 diabetes mellitus |
| Injury of urinary and pelvic organs | Other specified diabetes mellitus |
| Pilonidal cyst and sinus | Cystic fibrosis |
| Abnormal blood-pressure reading, without diagnosis | Unspecified dementia |
| Benign neoplasm of other and unspecified | Other mental disorders due to known physiological |
| intrathoracic organs | condition |
| Histoplasmosis | Schizophrenia |
| Fall on and from scaffolding | Major depressive disorder, recurrent |
| Pedestrian injured in collision with railway train or | Sleep disorders not due to a substance or known |
| railway vehicle | physiological condition |
| Disorders of arteries, arterioles and capillaries in | Pervasive developmental disorders |
| diseases classified elsewhere | |
| Other viral infections of central nervous system, not | Huntington's disease |
| elsewhere classified | |
| Acute bronchiolitis | Parkinson's disease |
| Unspecified behavioral syndromes associated with | Dystonia |
| physiological disturbances and physical factors | |
| Androgenic alopecia | Other extrapyramidal and movement disorders |
| Malignant neoplasm of other and unspecified major | Alzheimer's disease |
| salivary glands | |
| Other male sexual dysfunction | Other degenerative diseases of nervous system, not |
| | elsewhere classified |
| Diseases of spleen | Multiple sclerosis |
| Mosquito-borne viral encephalitis | Other acute disseminated demyelination |
| Nicotine dependence | Other demyelinating diseases of central nervous |
| | system |
| Cocaine related disorders | Epilepsy and recurrent seizures |
| Presence of other functional implants | Sleep disorders |
| Injury of unspecified body region | Pain, not elsewhere classified |
| Disorders resulting from impaired renal tubular | Glaucoma |
| function | |
| Dermatophytosis | Other and unspecified hearing loss |
| Osteoporosis with current pathological fracture | Angina pectoris |
| Other nutritional deficiencies | Other pulmonary heart diseases |
| Exposure to excessive cold of man-made origin | Atrial fibrillation and flutter |
| Congenital malformations of nose | Other cardiac arrhythmias |
| Other disorders of skin and subcutaneous tissue in | Heart failure |
| diseases classified elsewhere | |
| Anuria and oliguria | Complications and ill-defined descriptions of heart |
| | disease |
| Renal agenesis and other reduction defects of kidney | Cerebral infarction |
| Vitiligo | Viral pneumonia, not elsewhere classified |
| Unspecified appendicitis | Other chronic obstructive pulmonary disease |
| Other birth injuries to central nervous system | Asthma |
| Other appendicitis | Respiratory conditions due to inhalation of chemicals, |
| | gases, fumes and vapors |
| Cryptococcosis | Other interstitial pulmonary diseases |
| Endocarditis, valve unspecified | Gastro-esophageal reflux disease |
| Other respiratory disorders | Ulcerative colitis |
| Postprocedural endocrine and metabolic | Intestinal malabsorption |
| complications and disorders, not elsewhere classified | |
| Trichinellosis | Psoriasis |
| Thyroiditis | Autoinflammatory syndromes |
| Abnormal and inconclusive findings on diagnostic | Rheumatoid arthritis with rheumatoid factor |
| imaging of breast | |
| Glomerular disorders in diseases classified elsewhere | Other rheumatoid arthritis |
| Other inflammatory spondylopathies | Osteoarthritis of hip |
| Hypersensitivity pneumonitis due to organic dust | Osteoarthritis of knee |
| Complications of cardiac and vascular prosthetic | Other and unspecified osteoarthritis |
| devices, implants and grafts | |
| Other hyperalimentation | Other systemic involvement of connective tissue |
| Inflammatory disease of uterus, except cervix | Other disorders of urinary system |
| Occupant of pick-up truck or van injured in other and | Menopausal and other perimenopausal disorders |
| unspecified transport accidents | |
| Spina bifida | Cough |
| Other retinal disorders | Other skin changes |
| Monkeypox | Speech disturbances, not elsewhere classified |
| Malignant neoplasm of prostate | Fever of other and unknown origin |
| Other obstetric trauma | Cachexia |
| Sequelae of inflammatory and toxic polyneuropathies | Elevated blood glucose level |
| Crushing injury of ankle and foot | Abnormal tumor markers |
| Body mass index (BMI) | Traumatic amputation of lower leg |
| Acquired hemolytic anemia | Poisoning by, adverse effect of and underdosing of |
| | hormones and their synthetic substitutes and |
| | antagonists, not elsewhere classified |
| Diaper dermatitis | Orthopedic aftercare |
| Occupant of pick-up truck or van injured in collision | Transplanted organ and tissue status |
| with two- or three-wheeled motor vehicle | |
| Respiratory failure, not elsewhere classified | Cholera |
| Major depressive disorder, single episode | Atopic dermatitis |
| Glanders and melioidosis | Dependence on enabling machines and devices, not |
| | elsewhere classified |
| Injuries involving multiple body regions | Late pregnancy |
| Toxic effect of metals | Other crystal arthropathies |
| Shock, not elsewhere classified | Neoplasm of uncertain behavior of oral cavity and |
| | digestive organs |
| Other and unspecified myopathies | Benign neoplasm of eye and adnexa |
| Dracunculiasis | Antepartum hemorrhage, not elsewhere classified |
| Contact with steam, hot vapors and hot objects, | Other fall from one level to another |
| undetermined intent | |
| Blindness and low vision | Contact with other hot metals |
| Simple and mucopurulent chronic bronchitis | Malignant neoplasm of thyroid gland |
| Rheumatic fever without heart involvement | Mesothelioma |
| Pneumonia due to Hemophilus influenzae | Atypical virus infections of central nervous system |
| Crushing injury of elbow and forearm | Acute respiratory distress syndrome |
| Injury of blood vessels at neck level | Undescended and ectopic testicle |
| Intracranial injury | Endocarditis and heart valve disorders in diseases |
| | classified elsewhere |
| Other diseases of appendix | Unspecified renal colic |
| Other acquired deformities of musculoskeletal system | Unspecified disorder of adult personality and behavior |
| and connective tissue | |
| Polyosteoarthritis | Disorders of mineral metabolism |
| Car occupant injured in collision with pedal cycle | Evidence of alcohol involvement determined by blood |
| | alcohol level |
| Other disorders of tympanic membrane | Accidental striking against or bumped into by another |
| | person |
| Respiratory disorders in diseases classified elsewhere | Disorders of muscle tone of newborn |
| Other birth injuries | Other disorders of choroid |
| Attention-deficit hyperactivity disorders | Adverse effects, not elsewhere classified |
| Other and unspecified injuries of shoulder and upper | Nerve root and plexus compressions in diseases |
| arm | classified elsewhere |
| Malignant neoplasm of anus and anal canal | Other abnormal immunological findings in serum |
| Benign lipomatous neoplasm | Dermatopolymyositis |
| Juvenile arthritis | Schizoaffective disorders |
| Disorders of glycosaminoglycan metabolism | Traumatic amputation of hip and thigh |
| Other fall on same level due to collision with another | Diabetes mellitus in pregnancy, childbirth, and the |
| person | puerperium |
| Acanthosis nigricans | Congenital malformations of pulmonary and tricuspid |
| | valves |
| Other intellectual disabilities | Contact with hot engines, machinery and tools |
| Disorders of branched-chain amino-acid metabolism | Myeloid leukemia |
| and fatty-acid metabolism | |
| Extrapyramidal and movement disorders in diseases | Other and unspecified abnormal findings in urine |
| classified elsewhere | |
| Car occupant injured in collision with pedestrian or | Malignant neoplasm of larynx |
| animal | |
| Other diseases of lip and oral mucosa | Preterm labor |
| Abnormal findings in specimens from female genital | Other coagulation defects |
| organs | |
| Supervision of high risk pregnancy | Other bursopathies |
| Genetic carrier | Injury of muscle and tendon at ankle and foot level |
| Other disorders of nervous system not elsewhere | Injury of blood vessels at ankle and foot level |
| classified | |
| Spinal osteochondrosis | Other cestode infections |
| Gout | Personal history of medical treatment |
| Toxic effect of other inorganic substances | Pre-existing hypertension complicating pregnancy, |
| | childbirth and the puerperium |
| Other and unspecified injuries of ankle and foot | Benign neoplasm of ovary |
| Traumatic amputation of wrist, hand and fingers | Congenital absence, atresia and stenosis of large |
| | intestine |
| Other anxiety disorders | Liveborn infants according to place of birth and type |
| | of delivery |
| Contact with sharp object, undetermined intent | Other disorders of psychological development |
| Bus occupant injured in collision with fixed or | Burn and corrosion of shoulder and upper limb, |
| stationary object | except wrist and hand |
| Contact with other and unspecified machinery | Complications following (induced) termination of |
| | pregnancy |
| Intentional self-harm by jumping from a high place | Chronic hepatitis, not elsewhere classified |
| Complications associated with artificial fertilization | Nonrheumatic tricuspid valve disorders |
| Recurrent and persistent hematuria | Plasmodium malariae malaria |
| Accident to nonpowered aircraft causing injury to | Congenital hydrocephalus |
| occupant | |
| Nausea and vomiting | Inflammatory polyneuropathy |
| Keratoderma in diseases classified elsewhere | Neoplasm of uncertain behavior of meninges |
| Open wound of abdomen, lower back, pelvis and | Findings of drugs and other substances, not normally |
| external genitals | found in blood |
| Toxic effect of organic solvents | Accidental drowning and submersion while in natural |
| | water |
| Poisoning by, adverse effect of and underdosing of | Toxic effect of pesticides |
| agents primarily affecting the gastrointestinal system | |
| Dislocation and sprain of joints and ligaments of head | Assault by sharp object |
| Pityriasis rosea | Respiratory distress of newborn |
| Drug or chemical induced diabetes mellitus | Paraplegia (paraparesis) and quadriplegia |
| | (quadriparesis) |
| Other general symptoms and signs | Explosion of other materials |
| Encounter for attention to artificial openings | Exposure to excessive heat of man-made origin |
| Other nontoxic goiter | Hydrocephalus |
| Congenital malformations of eyelid, lacrimal | Pleural plaque |
| apparatus and orbit | |
| Human immunodeficiency virus [HIV] disease | Other disorders of muscle |
| Viral infection of unspecified site | |

TABLE 2
Example Meaningful Aspects of Health (MAH) of a Measurement Stack.
Certain descriptions of example MAHs are left on purpose.

| Example MAH | Description |
|---|---|
| Language Impairment | Language impairment in Alzheimer's disease primarily occurs because of decline in |
| | semantic and pragmatic levels of language processing |
| Fatigue | A condition characterized by a lessened capacity for work and reduced efficiency of |
| | accomplishment, usually accompanied by a feeling of weariness and tiredness. |
| Syncope | Syncope is a temporary loss of consciousness usually related to insufficient blood |
| | flow to the brain. It's also called fainting or ‘passing out.’” |
| Chest pressure | Chest pressure is the sensation of a squeezing, tightening, crushing or pressing in the |
| | chest area, with or without pain. It is sometimes described as a feeling of a band |
| | tightening around your chest or of something heavy sitting on your chest. |
| Heart palpitations | Heart palpitations are feelings of having a fast-beating, fluttering or pounding heart. |
| Apnea | Apnea. A sleep disorder in which breathing repeatedly stops and starts |
| Anxiety | Anxiety: A feeling of worry, nervousness, or unease about something with an |
| | uncertain outcome |
| Insomnia | Trouble sleeping |
| Palpitations | Heart palpitations are feelings of having a fast-beating, fluttering or pounding heart. |
| Gait Impairment | Abnormal gait or a walking abnormality is when a person is unable to walk in the |
| | usual way. This may be due to injuries, underlying conditions, or problems with the |
| | legs and feet. |
| Neuro-Psychiatric | ICHOM: Includes anxiety, depression, behavior, apathy, and psychosis. Tracked via |
| | the Neuropsychiatric Inventory (NPI). |
| Skin Color | Skin can appear red in patches |
| Skin Texture | Skin can appear bumpy |
| Social | Includes community affairs and relationships. |
| Daily Living | ICHOM: Includes instrumental and basic activities of daily living. Tracked via the |
| | Bristol Activity Daily Living Scale (BADLS). |
| Cognition | ICHOM: Includes anxiety, depression, behavior, apathy, and psychosis. Tracked via |
| | the Neuropsychiatric Inventory (NPI). |
| Quality of Life & | Includes finance, enjoyment of activities, pain, and side effects of medication. |
| Wellbeing (ICHOM) | Tracked via the Quality of Life-AD (QOL-AD) and Quality of Wellbeing Scale-Self |
| | Administered (QWB-SA). |
| Overall survival | Overall survival |
| Hospital admissions | Hospital admissions |
| Disease progression | Tracked via the Clinical Dementia Rating (CDR) |
| Falls | Falls |
| Time to full-time care | Time to full-time care |
| Carer quality of life | Tracked via the EuroQol-5D (EQ-5D). |
| Hand and Feet Dexterity | Ability to move hand and feet without pain |
| Early Detection of Skin | Early detection of skin Melanoma to prevent spread and cure/remove early and avoid |
| Melanoma | more serious consequences |
| skin deformity | |
| Receive correct | |
| treatment for Prostate | |
| Cancer | |
| psychometric | personality attributes and cognitive capability |
| Early Diagnosis | Identification of patients with risk of developing pulmonary hypertension |
| Sleep Quality | Information regarding sleep/wake episodes |
| Wound Status | Measurement of wound type, circumference, progress etc. |
| Mobility | Mobility is an aspect of health Parkinson's Disease patients struggle with |
| Limb movements | Nearly irresistible urge to move the limbs is an aspect of health Restless Legs |
| | Syndrome patients struggle with. |
| Sleep disturbance | Sleep disturbance is an aspect of health, Chronic Insomnia patients struggle with. |
| Sleep disturbance | Sleep disturbance is an aspect of health Insomnia Disease patients struggle with. |
| Heart Rate Variability | Heart Rate Variability is an aspect of health Cardiomyopathies patients struggle with |
| Mobility | Mobility is a very important aspect of health patients who had lower limb amputation |
| | struggle with |
| Intraocular pressure | IOP fluctuation is an aspect of health Glaucoma Disease patients struggle with |
| (IOP) fluctuation | |
| Weight loss | Weight loss is an aspect of health Cachexia Disease patients struggle with |
| Pain | Pain is an aspect of health Diabetic Peripheral Neuropathy Disease patients struggle |
| | with. |
| Pain | Pain is an aspect of health Knee Osteoarthritis Disease patients struggle with |
| Sleep disturbance | Sleep disturbance is an aspect of health Alzheimer's Disease patients struggle with. |
| Hyperglycaemia | Hyperglycemia is an aspect of health Diabetes Mellitus patients struggle with. |
| Dyspnoea | Dyspnoea is an aspect of health Angina pectoris (Chronic Stable Angina) Disease |
| | patients struggle with |
| Medication adherence | Medication adherence refers to whether patients take their medications as prescribed |
| | (e.g., twice daily), as well as whether they continue to take a prescribed medication. |
| Glucose Variability | Glucose Variability is an aspect of health Diabetes Mellitus Type 2 patients struggle |
| | with |
| Cough Count | Cough Count is an aspect of health Chronic Cough patients struggle with |
| Heart Rate Variability | Heart Rate Variability is an aspect of health Heart Failure patients struggle with |
| Airflow limitation | Airflow limitation is an aspect of health Chronic obstructive pulmonary Disease |
| | patients struggle with |
| Cognition | Cognition in Parkinson's Disease |
| Blood Volume Pulse | Blood Volume Pulse Variations is an aspect of health Hearing Loss patients struggle |
| Variations | with |
| Pain | Pain is an aspect of health Chronic Pain Disease patients struggle with |
| Skin itch | |
| Glucose Variability | Glucose Variability is an aspect of health Type I Diabetes patients struggle with |
| WASO (Wake After | WASO (Wake After Sleep Onset) Count is an aspect of health Sleep Wake Disorders |
| Sleep Onset) Count | patients struggle with |
| Skin condition | Skin can be dry and cracked in AD. |
| Pain | Pain is an aspect of health Osteoarthritis of hip Disease patients struggle with |
| Tremor Count | Tremor Count is an aspect of health Parkinson's Disease patients struggle with |
| Gait speed | Gait speed is an aspect of health Cerebral infarction patients struggle with |
| Blinking Activity | Blinking Activity is an important aspect of Blepharospasm |
| Airway remodeling- | Medication adherence can be defined as the extent to which a patient's behavior |
| Medication Adherence | corresponds with the prescribed medication dosing regime, including time, dosing and |
| | interval of medication intake. Poor asthma management can lead to airway |
| | remodeling |
| Tremor | Resting, postural, kinetic, and lateral wing beating tremor on both sides |
| Cardinal Parkinson's | The four cardinal motor symptoms are: bradykinesia: slow movement. rigidity: |
| Disease Motor Signs | stiffness of the arms, legs, or neck. tremor. postural instability: balance issues. |
| Sedentary behavior | Sedentary behavior is an independent predictor of diabetic foot ulcer development |
| Functional wrist range | The wrist is often severely affected in rheumatoid arthritis. Rheumatoid arthritis (RA) |
| of motion | causes pain, limited range of motion (ROM) of joints, that seriously impacts patients' |
| | psychological and physical, well-being |
| Physical activity | Physical activity makes it easier to control the blood glucose (blood sugar) level of the |
| | people suffering from Diabetes Type 1. Exercise benefits people with type 1 because |
| | it increases their insulin sensitivity. |
| Sleep duration and | Sleep disruption may negatively affect disease progression and development of |
| quality | complications in people with type 1 diabetes. Sleep may be disrupted as a result of |
| | both behavioral and physiological aspects of diabetes and its management. |
| Heart Rate Variability | People with foot ulcers and diabetes are showing more cardiovascular risk factors, |
| | such as high blood pressure, and are more likely to die from cardiovascular causes. |
| Mobility | Impaired mobility is a frequently serious side effect of surgery |
| Pulmonary function | how well one person is able to breathe and how effectively the lungs send oxygen to |
| | the rest of the body |
| Fever | Fever is an aspect of health Covid 19 patients struggle with |
| Airflow limitation | Airflow limitation is an aspect of health Chronic obstructive pulmonary Disease |
| | patients struggle with |
| Insomnia | Insomnia is an aspect of health Major Depressive Disorder patients struggle with |
| Seizures | Seizure is an aspect of health Rett's Syndrome patients struggle with |
| Medication adherence | Medication adherence refers to whether patients take their medications as prescribed |
| | (e.g., twice daily), as well as whether they continue to take a prescribed medication. |
| Respiratory disturbance | Respiratory disturbance is an aspect of health Cystic Fibrosis Disease patients struggle |
| | with |
| Fatigue | Fatigue is an aspect of health Chronic Heart Failure With Reduced Ejection Fraction |
| | patients struggle with |
| Sleep disturbance | Sleep is often affected in AD patients. |
| Sleep disturbance | Sleep disturbance is an aspect of health Irregular Sleep-Wake Rhythm Disorder |
| | patients struggle with. |
| Change in Moderate to | Increased physical activity improves the quality of life in people with interstitial lung |
| Vigorous Physical | disease (PH-ILD) who are at risk of pulmonary hypertension, |
| Activity (MVPA) | |
| Cognitive impairment | Cognitive impairment is an aspect of health Major Depressive Disorder patient |
| | struggle with |
| Physical activity | physical activity and exercise is an effective non-pharmacological intervention to |
| | improve diabetic foot related outcomes |
| Tissue oxygenation in | Adequate tissue oxygenation is an essential factor during wound healing in patients |
| lower extremities | with diabetic foot ulcer. |
| Gait speed | Patients with diabetes frequently exhibit a conservative gait strategy where there is |
| | slower walking speed, wider base of gait, and prolonged double support time. |
| Foot complications | Foot complications is an aspect of health Other specified diabetes mellitus with |
| | diabetic chronic kidney disease patients struggle with |
| Dry eyes | As a result of the mucous membranes and moisture-secreting glands, the eyes are |
| | usually affected - resulting in decreased tears. |
| Dry mouth | As a result of the mucous membranes and moisture-secreting glands, the mouth is |
| | usually affected - resulting in decreased saliva. |
| Balance and postural | Diabetic foot ulcer patients are often affected by balance and postural sway |
| sway | impairment. |
| Abdominal cramping | As a result of a chronic inflammatory bowel |
| Physical activity | Impaired physical activity is a frequent serious side effect of surgery |
| Soreness/pain | AD patches can result in pain experiences by patients. Not (yet) measurable by digital |
| | health technologies. |
| Mental health | Fear for symptoms and anxieties can influence a patients QoL. Not (yet) measurable |
| | by digital health technologies. |
| Sleep disturbance | Sleep disturbances are common in patients after surgery and produce harmful effects |
| | on postoperative recovery |
| Chorea | Chorea is a movement disorder that causes involuntary, irregular, unpredictable |
| | muscle movements |
| Physical activity | Physical activity can be an effective intervention to reduce symptoms associated with |
| | peripheral neuropathy. |
| Motor activity | Motor activity is an aspect of health Parkinson's Disease patients struggle with |
| Atrial Fibrillation | Atrial Fibrillation Burden is an aspect of health Atrial Fibrillation patients struggle |
| Burden | with |
| WASO (Wake After | WASO (Wake After Sleep Onset) is an aspect of health Restless Legs Syndrome |
| Sleep Onset) | patients struggle with |
| Sleep Duration | Sleep Duration is an aspect of health Restless Legs Syndrome patients struggle with |
| Facial Task | Facial Task Performance is an aspect of health Huntington Disease patients struggle |
| Performance | with |
| Motor Performance | Motor Performance is an aspect of health Huntington Disease patients struggle with |
| Energy levels | Energy levels can be significantly lower in patients suffering COVID-19 |
| Pruritus | Skin itch is a prevalent symptom in patients with Atopic Dermatitis. |
| Change in skin | Change in skin roughness is an aspect of health Changes in Skin Texture patients |
| roughness | struggle with |
| Change in skin wrinkle | Change in skin wrinkle is an aspect of health Changes in Skin Texture patients |
| | struggle with |
| Change in skin age | Change in skin age is an aspect of health Changes in Skin Texture patients struggle |
| | with |
| Physical Activity | Physical Activity is an aspect of health Breast Cancer patients struggle with |
| Disease control | Self-empowerment and disease control in Rheumatoid arthritis |
| Physical Activity | Physical Activity is an aspect of health Mild Cognitive Impairment patients struggle |
| | with |
| Sleep disturbance | Sleep Disturbance is an aspect of health Reflux Esophagitis Disease patients struggle |
| | with |
| Pain | Pain is an aspect of health Neuromyelitis Optica Spectrum Disorder patients struggle |
| | with |
| Pain | Pain is an aspect of health Transverse Myelitis patients struggle with |
| Pain | Pain is an aspect of health Multiple Sclerosis Disease patients struggle with |
| Cognition | |
| Exercise Tolerance | Exercise Tolerance is an aspect of health Heart Failure Disease patients struggle with |
| Dyspnea | Dyspnea is an aspect of health Pulmonary Hypertension Disease patients struggle with |
| Glucose Variability | Glucose Variability is an aspect of health Glucose Intolerance patients struggle with |
| Sleep Efficiency | Sleep Efficiency is an aspect of health Glucose Intolerance patients struggle with |
| Sleep Midpoint | Sleep Midpoint is an aspect of health Glucose Intolerance patients struggle with |
| Glucose Intolerance | Glucose Intolerance is an aspect of health Short Bowel Syndrome patients struggle |
| | with |
| Feeding Patterns | Feeding Patterns is an aspect of health Short Bowel Syndrome patients struggle with |
| Sleep Quality | Sleep Quality is an aspect of health Short Bowel Syndrome patients struggle with |
| Motor activity | Motor activity can be significantly reduced in patients suffering MDD |
| Early diagnosis of | |
| epilepsis attacks | |
| Spikes | |
| Word Recognition Rate | Word Recognition Rate is an aspect of health Speech Disorder patients struggle with |
| Prosodic tone | Prosodic tone intelligibility is an aspect of health Speech Disorder patients struggle |
| intelligibility | with |
| Word Recognition Rate | Word Recognition Rate is an aspect of health Speech Disorder patients struggle with |
| Prosodic tone | Prosodic tone intelligibility is an aspect of health Speech Disorder patients struggle |
| intelligibility | with |
| Sleep Efficiency | Sleep Efficiency is an aspect of health Sleep Disturbance patients struggle with |
| Glucose Variability | Glucose Variability is an aspect of health Kidney Transplant patients struggle with |
| Medication adherence | Medication adherence refers to whether patients take their medications as prescribed |
| | (e.g., twice daily), as well as whether they continue to take a prescribed medication. |
| Physical activity | Physical activity reduces the risk of heart disease by lowering blood pressure. |
| Tremor | Tremors are unintentional trembling or shaking movements in one or more parts of |
| | the body. |
| Excessive daytime | inappropriate and undesirable sleepiness during waking hours and is a common non- |
| sleepiness (EDS) | motor symptom in Parkinson's disease, affecting up to 50% of patients. |
| Overnight pulse | Nocturnal oxygen saturation variance can affect the sleep quality of sickle cell |
| oximetry variance | disease (SCD) patients. |
| Sleep disturbance | Sleep disturbance is common in patients with sickle cell disease (SCD) |
| Physical activity | Sickle cell disease (SCD) affects the level of physical activity of the patients |
| Glycemic Variability | Glycemic variability (GV) refers to fluctuations in blood glucose levels |
| Nocturnal Activity | Sleep disturbances are common during menopause |
| Nocturnal Activity | Nocturnal worsening of asthma symptoms is common in Asthma patients |
| Fatigue | |
| Early diagnosis of heart | |
| disease | |
| Early diagnosis | Early diagnosis of tumors |
| Energy levels | Energy levels can be significantly lower in patients suffering MS |
| Vitality | |
| Fever | |
| Daily physical activities | Allergic asthma symptoms can worsen or be triggered by physical activity |
| Cardiac monitoring | Continuously monitoring of cardiac patients |
| Poisoning | Poisoning by glucocorticoids overdosing |
| Low energy levels | Low energy levels in Diabetes |
| Sleep quality | Sleep quality in RSV |
| Minimize adverse | Minimize adverse effect in atrial fibrillation |
| effects | |
| Behavioral disorders | |
| Infection | To ensure a positive/negative infection |
| Vital signs | Vital signs in RSV |
| Respiratory decline | |
| Motor activity | Motor activity in autism spectrum disorder (ASD) |
| Epilepsis monitoring | To alert caregivers upon seizures |
| Loss of verbal fluency | Loss of verbal fluency is impacting patients suffering from Alzheimer's disease. |
| | Individuals may stutter, halt or find it difficult to finish sentences. |
| Motor activity | |
| Sleep disturbance | Sleep disturbance in heart disease |
| Motor activity | Motor activity in heart disease |
| Sleep disturbance | Sleep disturbance in PD. |
| Sleep disturbance | Sleep is often affected in MDD patients. |
| Motor activity | Motor activities in RSV |
| Sleep Efficiency | Sleep Efficiency is an aspect of health Sleep Disturbance patients struggle with |
| Vital signs | Vital signs in cardiac arrhythmia |
| Cognitive engagement | Cognitive engagement is an important aspect of Cognitive Impairment |
| Cognitive engagement | Cognitive engagement is an important aspect of Cognitive Impairment |
| Burning/tingling | The feeling like the skin is on fire is a common symptom in AD. |
| sensation | |
| Motor activity | Exercise Tolerance is an aspect of health Pulmonary Arterial Hypertension patients |
| | struggle with |
| Cardiac health | Cardiac health is an important aspect of Autoinflammatory syndrome |
| Negative symptoms | |
| Negative symptoms | Negative symptoms in schizophrenia |
| Early diagnosis | Early diagnosis in lung cancer |
| Early diagnosis | Early diagnosis in multiple myeloma |
| Sleep disturbance | Sleep disturbance in PH |
| Cognitive decline | Cognitive decline in BPSD |
| Motor activity | Motor activity in BPSD |
| Verbal disturbance | Verbal disturbance in BPSD |
| Emotional decline | Emotional decline in BPSD |
| Vegetative decline | Vegetative decline in BPSD |
| BPSD monitoring | BPSD monitoring in BPSD |
| Pain | Pain in Psoriasis. Pain is defined as “an unpleasant sensory and emotional experience |
| | associated with actual or potential tissue damage, or described in terms of such |
| | damage”. |
| Pain | Pain in Ulcerative Colitis. Symptoms: Persistent diarrhea, Abdominal pain, Rectal |
| | bleeding/bloody stools, Weight loss, Fatigue, Painful and Swollen joints (arthritis), |
| | Mouth ulcers, Areas of painful, red and swollen skin Irritated and red eyes. |
| Pain | Pain in rheumatoid arthritis. RA mainly attacks the joints, usually many joints at once. |
| | RA commonly affects joints in the hands, wrists, and knees. In a joint with RA, the |
| | lining of the joint becomes inflamed, causing damage to joint tissue. |
| Pain | Pain in Atopic Dermatitis |
| Pain | The hallmark symptom of osteoarthritis (OA) is pain. This symptom drives |
| | individuals to seek medical attention, and contributes to functional limitations and |
| | reduced QoL |
| Involuntary Urine Loss | |
| test mah-Shig | |
| Rigidity | Stiff or inflexible muscles |
| Bradykinesia | Slowness of movement |
| Clonic Seizure | ‘Clonus’” (KLOH-nus) means fast stiffening and relaxing of a muscle that happens |
| | repeatedly. In other words |
| Dyskinesia | Dyskinesia is a movement disorder that often appears as uncontrolled shakes, tics, or |
| | tremors. Often, the condition occurs in people with Parkinson's disease due to the |
| | overstimulation of their dopamine receptors from medications that increase this |
| | neurotransmitter in the brain. One common example of a Parkinson's medication that |
| | can have this effect is levodopa. Another type of dyskinesia is tardive dyskinesia, |
| | which occurs when a person takes certain dopamine receptor-blocking medications. |
| | The term “tardive” means delayed, and doctors use the term because this dyskinesia |
| | type usually occurs after the long-term use of such medications. |

TABLE 3
Example Concepts of Interest (COI) of a Measurement Stack. Certain
descriptions of example COIs are left blank on purpose.

| Example COIs | Description |
|---|---|
| Lower extremity balance | The ability to maintain balance in lower extremities |
| Blacking out | Suddenly Fainting |
| Dizziness | Feeling dizzy |
| Drowsiness | Feeling drowsy or groggy |
| Unsteadiness | Unsteadiness |
| Impaired Vision | Changes in vision, such as seeing spots or having tunnel vision |
| Shortness of breath | Feeling like one cannot breath |
| Nausea | A feeling of sickness with an inclination to vomit |
| Sweating | Excessive sweating |
| Lower extremity strength | The ability to generate force in the lower extremity muscles |
| Tremor | Trembling in hands, arms, legs, jaw, or head. |
| Total sleep time (TST) | Total sleep time (TST): The amount of time that a person spends |
| | actually sleeping during a planned sleep episode. TST is the sum of all |
| | REM and NREM sleep in a sleep episode. |
| Wake after sleep onset (WASO) | Normal adult mean sleep latency is between 10 and 20 min. |
| | Pathologic sleepiness is defined as a mean sleep latency <5 min and |
| | this has been associated with impaired performance. According to the |
| | AASM, a sleep latency of <8 min is diagnostic of sleepiness. |
| Sleep efficiency | Normal sleep efficiency is considered to be 80% or greater. For |
| | example, if a person spends 8 hours in bed (from 10 p.m. to 6 a.m), at |
| | least 6.4 hours or more should be spent sleeping to achieve an 80% or |
| | greater sleep efficiency. |
| Scratching events per hour | Number of times per hour that the patient scratches |
| Scratching duration per hour | Length of time of the scratching event |
| Number of scratching events | Count of the number of scratching events during a specific time period |
| Memory | |
| Speech | |
| Visuospatial/Executive | |
| Naming | |
| Attention | |
| Abstraction | |
| Delayed recall | |
| Orientation | |
| Anxiety | |
| Depression | |
| Behavior | |
| Apathy | |
| Psychosis | |
| Community affairs | |
| Relationships | |
| Finance | |
| Enjoyment of activities | |
| Pain | |
| Side effects of medication | |
| VDHmodified Sharp score on XRAYs | |
| Suspicious Skin Lession Detection | Identify potentially malignant skin deformities. Post Identification, |
| | further investigation or intervention (Excision, Biopsy, expert |
| | assessment) may be needed. |
| Early identification of PDL1 status | PDL1 gene mutation is a strong indicator of CPRC (Castration |
| | Resistant Prostate Cancer) |
| dyspepsia | upset stomach pain: gastrointestinal (GI) organs, primarily stomach, |
| | first small intestinal part, and occasionally esophagus, function |
| | abnormally |
| Probability of Developing PH | |
| Sleep Latency | |
| Total Sleep Time | |
| Wake After Sleep Onset | |
| Sleep Efficiency | |
| Skin Moisture Level | Amount of moisture the skin holds |
| Wound Circumference | Size of the wound |
| Wound Progress | Is wound growing, stable or shrinking. |
| Processing speed | Listening and understanding speach |
| Executive function | |
| digiterra coi | digiterra coi |
| Periodic limb movements | |
| Sleep efficiency | |
| Sleep efficiency | |
| Sleep efficiency | |
| Physical Activity | |
| IOP reduction | |
| Physical activity: walking | |
| Physical activity: walking | |
| Physical activity: walking | |
| Sleep efficiency | |
| Glycemic variability | |
| Physical activity: maximal exertion | |
| Medication intake | |
| Glucose Variability | |
| Cough Activity | |
| Heart Rate Variability | |
| Heart Rate Variability | |
| Physical activity | |
| Blood Volume Pulse Variations | |
| Physical activity | |
| Reduction in skin lesions | |
| Glucose Variability | |
| Sleep Activity | |
| Physical activity | |
| Tremor | |
| Gait metrics | |
| Medication Adherance | |
| Tremor | A tremor is an involuntary quivering movement or shake |
| Balance | Postural instability. It appears as a tendency to be unstable when |
| | standing, as PD affects the reflexes that are necessary for maintaining |
| | an upright position |
| Gait | Parkinsonian gait is characterized by small shuffling steps and a |
| | general slowness of movement (hypokinesia), or even the total loss of |
| | movement (akinesia) in the extreme cases. |
| Pulmonary Function | how well one person is able to breathe and how effectively the lungs |
| | send oxygen to the rest of the body |
| EASI score | An EASI score is a tool used to measure the extent (area) and severity |
| | of atopic eczema (Eczema Area and Severity Index). |
| Nocturnal scratch | Patients with atopic dermatitis experience increased nocturnal pruritus |
| | which leads to scratching and sleep disturbances. |
| Physical activity | |
| Gait | |
| Language/speech | |
| Sleep efficiency | |
| Episodic memory | |
| Cognitive flexibility | |
| Wrist range of motion | The wrist is often severely affected in rheumatoid arthritis. |
| | Rheumatoid arthritis (RA) causes pain, limited range of motion |
| | (ROM) of joints, that seriously impacts patients' psychological and |
| | physical, well-being |
| Physical activity | Physical activity makes it easier to control the blood glucose (blood |
| | sugar) level of the people suffering from Diabetes Type 1. Exercise |
| | benefits people with type 1 because it increases their insulin |
| | sensitivity. |
| Physical activity | Physical activity makes it easier to control the blood glucose (blood |
| | sugar) level of the people suffering from Diabetes Type 1. Exercise |
| | benefits people with type 1 because it increases their insulin |
| | sensitivity. |
| Sleep duration | Sleep disruption may negatively affect disease progression and |
| | development of complications in people with type 1 diabetes. Sleep |
| | may be disrupted as a result of both behavioral and physiological |
| | aspects of diabetes and its management. |
| Body Temperature | |
| Physical activity | |
| Seizure actvity | |
| Physical Activity | |
| Medication intake | |
| Cognitive assesment | |
| Physical activity | |
| Sleep efficiency | |
| Physical Activity | |
| Physical Activity | |
| Sleep efficiency | |
| Physical activity | Increased physical activity improves the quality of life in people with |
| | interstitial lung disease (PH-ILD) who are at risk of pulmonary |
| | hypertension, |
| Physical activity | physical activity and exercise is an effective non-pharmacological |
| | intervention to improve diabetic foot related outcomes |
| Attention | |
| lower extremeties oxygen saturation | Adequate tissue oxygenation is an essential factor during wound |
| | healing in patients with diabetic foot ulcer. |
| kinematics of lower body | Foot ulcers can affect the kinematics of lower body in patients |
| | suffering from diabetes. |
| Physical Activity | |
| Balance | Diabetic foot ulcer patients are often affected by balance and postural |
| | sway imairment. |
| Total Mayo score (compound score) | Combines clinical disease features, physician global assessment and |
| | mucosal disease burden |
| Sedentary behaviour | Sedentary behaviour is an independent predictor of diabetic foot ulcer |
| | development |
| Heart Rate Variability | People with foot ulcers and diabetes are showing more cardiovascular |
| | risk factors, such as high blood pressure, and are more likely to die |
| | from cardiovascular causes. |
| Wound status | Status of the wounds initiated by extensive skin scratching |
| Irregular Heart Rythm | |
| Sleep Efficiency | |
| Facial Movement | |
| Movement Detection | |
| Nocturnal Activity | Sleep disturbances are common in patients after surgery and produce |
| | harmful effects on postoperative recovery |
| Mobility | Impaired mobility is a frequently serious side effect of surgery |
| Mobility | Impaired mobility is a frequently serious side effect of surgery |
| Physical activity | Impaired physical activity is a frequent serious side effect of surgery |
| Chorea | Chorea is a movement disorder that causes involuntary, irregular, |
| | unpredictable muscle movements |
| Physical activity | Physical activity can be an effective intervention to reduce symptoms |
| | associated with peripheral neuropathy. |
| Physical activity | |
| Glucose Variability | |
| Skin Aging | |
| Physical Activity | |
| Physical activity | |
| Sleep | |
| Sleep efficiency | |
| Sleep efficiency | |
| Physical activity | |
| Sleep efficiency | |
| Physical Activity | |
| Sleep efficiency | |
| Physical Activity | |
| Sleep Efficiency | |
| Physical Activity | |
| Glucose Variability | |
| Sleep Activity | |
| Glucose Variability | |
| Blood oxygen saturation (SpO2) | Pulse Oxygenation Level is an aspect of health Covid 19 patients |
| | strugle with |
| Glucose Variability | |
| Sleep Activity | |
| Working memory | |
| Walking | |
| Energy levels | |
| Posture | Body position of a patient during the day |
| Sleep patency | Sleep patency is often affected in MDD patients. |
| Sleep efficiency | Sleep is often affected in MDD patients. |
| Ambient light | |
| Physical activity | Daily life physical activity |
| Word Recognition | |
| Prosody Recognition | |
| Cardiac signs | |
| Prosody Recognition | |
| Sleep Activity | |
| Glucose Variability | |
| Medication Intake | |
| Physical activity | Allergic asthma symptoms can worsen or be triggered by physical |
| | activity |
| Physical activity | Physical activity reduces the risk of heart disease by lowering blood |
| | pressure. |
| Nocturnal scratch | Nocturnal scratching is one of the factors causing sleep disturbance in |
| | AD patients |
| Skin color | The skin color can become red as a result of Atopic Dermatitis |
| Swelling | Swelling is typically the result of inflammation or a buildup of fluid. |
| Bleeding | In more severe cases of AD, patches of dry skin can bleed. |
| Tremor | Tremors are unintentional trembling or shaking movements in one or |
| | more parts of the body. |
| Light to vigorous physical activity | Physical activity reduces the risk of heart disease by lowering blood |
| | pressure. |
| Bradykinsesia | Bradykinesia means slowness of movement and is one of the cardinal |
| | manifestations of Parkinson's disease |
| Dyskinesia | Dyskinesias are involuntary, erratic, writhing movements of the face, |
| | arms, legs or trunk |
| Daytime somnolence | inappropriate and undesirable sleepiness during waking hours and is a |
| | common non-motor symptom in Parkinson”s disease, affecting up to |
| | 50% of patients. |
| Overnight pulse oximetery variance | Nocturnal oxygen saturation variance can affect the sleep quality of |
| | sickle cell disease (SCD) patients. |
| Sleep Efficacy | Sleep disturbance is common in patients with sickle cell disease |
| | (SCD) |
| Nocturnal Activity | Sleep disturbance is common in patients with sickle cell disease |
| | (SCD) |
| Physical activity | Sickle cell disease (SCD) affects the level of physical activity of the |
| | patients |
| Glycemic Variability | Glycemic variability (GV) refers to fluctuations in blood glucose |
| | levels |
| Nocturnal Activity | Sleep disturbances are common during menopause |
| Nocturnal Activity | Nocturnal worsening of asthma symptoms is common in Asthma |
| | patients |
| Psychomotor function | |
| Dry mouth | As a result of the mucous membranes and moisture-secreting glands, |
| | the mouth is usually affected - resulting in decreased saliva. |
| Dry eyes | As a result of the mucous membranes and moisture-secreting glands, |
| | the eyes are usually affected - resulting in decreased tears. |
| Cognitive function | |
| Processing speed | |
| Sleep efficiency | Sleep efficiency in RSV |
| Physical activity | |
| Brain wave abnormalities | |
| Heart activity | |
| Muscle activity | |
| Cardiac signs | To measure early diagnosis of heart disease in heart disease |
| Respiratory rate | To measure early diagnosis of heart disease in heart disease |
| Meta-analysis | Meta-analysis of tumor markers for early diagnosis of tumors |
| Typing behaviour | To measure energy levels in typing behaviour in MS |
| Cardiac signs | |
| Body temperature | To measure early diagnosis of heart disease in heart disease |
| Physical activity | To measure early diagnosis of heart disease in heart disease |
| Cumulative worsening score (CWS) | For trials in some diseases, it may be most important to document |
| | ANY GC toxicity that occurs. The CWS represents cumulative |
| | toxicity, both permanent AND transient. The CWS serves as a record |
| | of worsening toxicity. |
| Aggregate Improvement Score (AIS) | With AIS, baseline toxicities that resolve are removed from the score. |
| | Newly-occurring toxicities are added to the score. The AIS serves as a |
| | record of improved toxicity. |
| Glycemic variability | |
| Social skills | Social skills in autism spectrum disorder (ASD) |
| Repetitive behaviour | Repetitive behaviour in autism spectrum disorder (ASD) |
| Difficulty communicating | Difficulty communicating in autism spectrum disorder (ASD) |
| Episodic memory | Episodic memory in MDD |
| Inflammation levels | To measure inflammation levels in infection in coronavirus infection |
| Skin/body temperature | To measure skin/body temperature in vital signs in RSV |
| Heart rate | To measure heart rate in vital signs in RSV |
| Respiratory rate | To measure respiratory rate in vital signs in RSV |
| Walking | To measure walking in daily physical activities in RSV |
| Physical activity | To measure walking in daily physical activities in RSV |
| Pulse oximetry | To measure pulse oxygen levels in vital signs in RSV |
| Apnea | To measure apnea in sleep quality in RSV |
| Atrial fibrillation (early detection) | Atrial fibrillation detection to lower adverse clinical cardiovascular |
| | outcomes in AF patients |
| Cough | Cough in respiratory decline |
| Wheezing | Wheezing in respiratory decline |
| Lung sounds | Lung sounds in respiratory decline |
| Depression | Depression in cognitive impairment in MDD |
| Physical activity | PA in motor activity in ASD |
| Cardiac signs | Cardiac signs in motor activity in ASD |
| Body condition | Body condition in motor activity in ASD |
| Seizures | Seizures in epilepsis monitoring |
| Language features | Loss of verbal fluency is impacting patients suffering from |
| | Alzheimer's disease. Speech data can serve as a window into cognitive |
| | functioning and can be used to screen for early signs of AD. |
| Daily life physical activities | DLPA |
| Cough | Cough in RSV during sleep |
| Environmental conditions | The environmental conditions during sleep in RSV patients (in |
| | relation to outbursts) |
| Physical activity | Physical activity in motor activity in heart disease |
| Blood pressure | Blood pressure in cardiac monitoring in heart disease |
| Sleep efficiency | Sleep efficiency in insomnia in heart disease |
| Cardiac signs | Cardiac signs in vital signs in cardiac arrhytmia |
| Skin Conductance | Skin conductance is an indicative of sweat used in measuring |
| | cognitive engagement |
| Sleep efficiency | Sleep efficiency is an important aspect of patients with Parkinson's |
| | disease |
| Skin sensitivity | Skin sensitivity is important in the condition of the surface of your |
| | skin. Ideally, the skin is smooth, soft, and supple, but it can be uneven |
| | or dull due to dry skin, blemishes, loss of collagen from aging, sun |
| | damage, or lack of exfoliation. |
| Concentration | Lack of concentration in mental health in Atopic Dermatitis. |
| Depression | Depression in mental health in Atopic Dermatitis. |
| Heat | Heat in burning/tingling sensation in Atopic Dermatitis |
| Blisters/welts | Blister/welts in soreness/pain in Atopic Dermatitis |
| Cracking/fissuring | Cracking/fissuring in soreness/pain in Atopic Dermatitis |
| Executive function | Executive functions are a set of cognitive processes that are necessary |
| | for the cognitive control of behavior: selecting and successfully |
| | monitoring behaviors that facilitate the attainment of chosen goals |
| Pulse wave velocity | Pulse wave velocity is an important aspect of cardiac health in |
| | Autoinflammatory syndrome |
| Blink Count | Blink Count is a an important measurement in Blepharospasm |
| Self-empowerment | Self-empowerment in disease control in Reumatoid Arthritis |
| Treatment adherence | Treatment adherence in negative symptoms in schizophrenia |
| Vital signs | Vital signs in negative symptoms in schizophrenia |
| Identify patients with high risk to | Identify patients with high risk to develop lung cancer in lung cancer |
| develop lung cancer | |
| Progression free survival | Progression free survival in early diagnosis in multiple myeloma |
| Sleep efficiency | Sleep efficiency in insomnia in PH |
| Physical capacity | Physical capacity in fatigue in PH |
| Syncope | Syncope in fatigue in PH |
| Palpitations | Palpitations in fatigue in PH |
| Dyspnea | Dyspnea in fatigue in PH |
| Posture | Posture in motor activity in PD. |
| Essential tremor | Essential tremor is a nervous system (neurological) disorder that |
| | causes involuntary and rhythmic shaking |
| Working memory | Working memory in AD. |
| Hallucinations | Hallucinations in cognitive decline in BPSD |
| Repetitive movements | Repetitive movements in motor activity in BPSD. ‘The indoor |
| | wandering patterns according to repetitive movements are analyzed |
| | and classified using the machine learning technique.’” |
| Apathy | Apathy in emotional decline in BPSD. (1) ‘Apathy is defined as lack |
| | of motivation with motivation being the set of behaviors and cognitive |
| | activities that transform the intention of doing something into a |
| | concluded action. Is characterized by diminished motivation and by |
| | emotional blunting (ie restricted emotional display). |
| Physical aggression | Physical aggression in motor activity in BPSD. ‘Physically aggressive |
| | behavior was defined as “an overt act in-volving delivery of a noxious |
| | stimulus to another person which wasclearly not accidental. Physically |
| | aggressive behavior is re-lated to depression and impairment in |
| | activities of daily living.’” |
| Verbal aggression/vocally disruptive | Verbal aggression/vocally disruptive behavior (VDB) in verbal |
| behavior (VDB) | disturbance in BPSD. Several tools have been developed to measure |
| | BPSD, but none is intended exclusively for the assessment of VA. |
| Delusions | Delusions in cognitive decline in BPSD. ‘For the patient the presence |
| | of delusions can result in increased aggression, agitation, wandering, |
| | insomnia, and distress. |
| Language impairment | Language impairment in verbal disturbance in BPSD. ‘Vocal features |
| | extracted from the audio recorded in a controlled environment during |
| | performance of simple vocal tasks enable quite accurate classification |
| | of healthy and demented subjects’”. |
| Falling | Falling in motor activity in BPSD. ‘There is a need for simple clinical |
| | tools that can objectively assess the fall risk in people with dementia. |
| | Wearable sensors seem to have the potential for fall prediction.’” |
| Anxiety | Anxiety in emotional decline in BPSD. ‘Anxiety is a pervasive |
| | disorder that increases the symptom burden for persons with dementia. |
| | Diagnosing anxiety in those experiencing dementia is often difficult |
| | because of overlapping symptoms with their primary |
| | neurodegenerative condition, other neuropsychiatric symptoms, |
| | severity of dementia, reliance on caregiver reports, and the lack of |
| | agreed-upon diagnostic criteria specific to dementia. |
| Depression | Depression in emotional decline in BPSD. ‘Depression: a period of at |
| | least 2 weeks characterized by sad mood and loss of interest and |
| | pleasure in almost all aspects of life with concomitants such as |
| | dysphoric symptoms (eg., helplessness, hopelessness, and feelings of |
| | guilt), appetite disorders, insomnia, and low energy |
| Eating behavior | Eating behavior in vegetative decline in BPSD. ‘Appetite/eating |
| | impairment is one of the most common and intense findings because |
| | of the general decline of physiological systems in the elderly’ |
| Wandering/pacing | Wandering/pacing in motor activity in BPSD. (1) ‘Wandering is |
| | reported to be one of the most challenging care burden issues and is |
| | also a safety issue because it is associated with increased risk of falls. |
| | The definition of wandering is mostly subjective description of |
| | ambulation in people with dementia (PWD) often characterized by |
| | “aimless” or “purposeless” ambulation. Wandering develops as |
| | cognitive function deteriorates. |
| Skin condition | IBD patients have dry skin features and frequently develop pruritus. |
| | These symptoms need to be recognized as clinical complications in |
| | IBD patients.’” |
| Sleep efficiency | Sleep efficiency in vegetative disturbance in BPSD. Significant sleep |
| | disruption is often observed in patients with dementia and is believed |
| | to be related to the neurodegenerative process. Less is known about |
| | the sleep of cognitively intact older adults and its relationship to |
| | subsequent cognitive decline. |
| Involuntary muscle movement | Involuntary movements compose a group of uncontrolled movements |
| | that may manifest as a tremor, tic, myoclonic jerk, chorea, athetosis, |
| | dystonia or hemiballism. |
| Physical activity | Physical activity in motor activity in BPSD. (1) ‘Self-reported data |
| | suggest that older adults with dementia are inactive. The purpose was |
| | to objectively assess the physical activity (PA) levels of |
| | communitydwelling and institutionalized ambulatory patients with |
| | dementia and to compare with the PA levels of cognitive healthy older |
| | adults. |
| Remote patient monitoring | Remote patient monitoring (RPM) in BPSD monitoring in BPSD. (1) |
| | Key statistics related to PCL Health care: 70% of care home residents |
| | suffer from some form of Dementia. PCL”s technology comes in |
| | specifically useful in managing such residents. (https://pcl- |
| | health.com/monitoring-devices/). (2) Study explores functional and |
| | psychological needs of people with dementia using participatory user- |
| | centered design methods that produced a rich understanding of their |
| | experiences. (Tiersen at al. 2021: |
| | https://aging.jmir.org/2021/3/e27047). (3) RPM has also been shown |
| | to increase patient safety in the home, with researchers noting |
| | medication reminders, wandering avoidance tracking, and caregiver |
| | education support as examples of how telehealth technologies promote |
| | patient safety. (4) insight into factors impacting adoption and use of |
| | Remote Monitoring Technologies in Dementia Care (Snyder et al. |
| | 2020: https://nsuworks.nova.edu/tgr/vol25/iss5/5/). |
| Tenderness and swelling | A hallmark feature of psoriatic arthritis (PsA) is the presence of |
| | inflammatory arthritis, characterized by tenderness and or swelling due |
| | to synovial inflammation. Joints are palpated for the purpose of |
| | determining if they are tender and/or swollen, the latter implying the |
| | presence of active synovitis, and both implying the presence of |
| | inflammation. |
| Fatigue | Fatigue is a symptom defined as a feeling of exhaustion, as well as |
| | reduced physical and mental capacity. The condition of chronic |
| | inflammation associated with psoriatic arthritis ca be regarded as a |
| | potential factor affecting development of fatigue. Fatigue is often |
| | present in chronic inflammatory skin and joint diseases. Fatigue is a |
| | common symptom in chronic inflammatory diseases of joints and |
| | skin, even though it is rarely subjected to an evaluation in everyday |
| | clinical practice. There are no objective methods of measurement of |
| | the severity of fatigue associated with chronic diseases, and all |
| | available measuring instruments are based on self-assessment. |
| Sleep disturbance | Sleep disturbances are particularly common and troublesome in |
| | people with OA. ‘Recent publications on OA pain and sleep |
| | disturbances stress the central importance of sleep in the well-being of |
| | patients. They also underline that sleep should be systematically |
| | assessed in those OA patients with an optimal management of pain to |
| | achieve synergistic improvements in quality of life.’” |
| Physical activity | 1. Pain is a prevalent and debilitating symptom in arthritis. Pain |
| | assessment is part of the Outcome Measures in Rheumatology Clinical |
| | Trials core domain set and one of the three PROs in the ACR response |
| | indices. It is an outcome measure that is uniformly collected in PsA |
| | RCTs and longitudinal studies. 2. Inflammation of joints leads to pain |
| | and loss of function, and structural damage resulting from PsA has |
| | been well recognized. Many patients with PsA experience significant |
| | joint damage and disability over time. For these reasons, physical |
| | function is an important outcome in PsA and is one of the core |
| | domains to be monitored. (Orbai et al. 2016; PRO; |
| | https://www.ncbi.nlm.nih.gov/pmc/articles/PMC4853652/). 3. Flares |
| | in axial spondyloarthritis (axSpA) may influence physical activity, |
| | are important for patients since they contribute to the unpredictability |
| | of the disease. Furthermore, due to the link between inflammation and |
| | structural degradation, flares are important to assess for disease |
| | management. (Gossec et al. 2018: https://sci- |
| | hub.se/10.1002/acr.23768). |
| Sleep disturbance | There might be an association between sleep disturbance and the |
| | severity of AD. Regardless of the cause, disturbed sleep can have |
| | many negative consequences, including impaired motor and cognitive |
| | function and changes in mood. 4 Patients and families also report a |
| | lower quality of life that is related to problems with sleep. Sleep |
| | disorders have a wide range of effects on both children and adults with |
| | AD. ADHD, Attention deficit hyperactivity disorder; QoL, quality of |
| | life. |
| Obesity | Obesity is associated with an increasing prevalence of musculoskeletal |
| | complaints and pain. Obesity is a major risk factor for osteoarthritis |
| | (OA), and pain can manifest in load-bearing and nonload-bearing |
| | joints. |
| Physical activity | Joint pain: Arthralgia = pain of the joints. Is the most common |
| | extraintestinal manifestation of inflammatory bowel disease (IBD) and |
| | occurs in up to one-third of patients. Joint pain involves smaller joints, |
| | such as the wrists, knees, or ankles, and is usually symmetric and less |
| | often involves large joints and is usually unilateral (a single knee or |
| | shoulder might be inflamed and swollen). Flares: Flares in axial |
| | spondyloarthritis (axSpA) may influence physical activity, are |
| | important for patients since they contribute to the unpredictability of |
| | the disease. Furthermore, due to the link between inflammation and |
| | structural degradation, flares are important to assess for disease |
| | management. Muscle weakness: Higher levels of chronic inflammation |
| | markers are associated with decreased muscle strength, lower muscle |
| | mass and disability. |
| Itch | Inflammatory bowel disease (IBD) is often complicated by |
| | extraintestinal manifestations. We frequently encounter IBD patients |
| | with pruritus; however, clinical evidence for the association of these |
| | conditions is lacking. |
| Scars | AD does not directly cause scars. However, if you scratch your skin so |
| | much that it bleeds, you”ll cause an open wound, which can lead to a |
| | scar-a normal part of the healing process. |
| Anxiety | Anxiety in mental health in Atopic Dermatitis |
| Itch | Itch is defined as “an unpleasant sensation that provokes the desire to |
| | scratch.” The definition provides 2 approaches to measurement, that |
| | of itch itself and the behavioral response, scratch. Although itch |
| | sensation is an inherently subjective phenomenon, recent advances in |
| | technology have allowed for the quantification of itch through |
| | measurement of itch threshold. The measurement of scratching |
| | through physical manifestations of scratching, video surveillance, |
| | actigraphy, and acoustic devices also provides valuable objective data |
| | about itch intensity. ‘Pruritus is a common complaint among patients |
| | with psoriasis of the chronic plaque type. The presence and intensity |
| | of itching has been found to be independent of age, gender, family |
| | history of psoriasis or atopy, alcohol or smoking habits, duration of |
| | the disease, as well as duration of the last outbreak of psoriasis |
| Skin sensitivity | Skin sensivity in Psoriasis. PSI was developed in patients with |
| | psoriasis and PsA and consists of a single NRS (0-10, “no itch” to |
| | “worst imaginable itch”) for itch with an assessment over the past 24 |
| | hours) |
| Sleep disturbance | Poor sleep quality in patients with PsA or Ps is a common symptom. |
| | Sleep disorders are more frequent in patients with PsA than in those |
| | with psoriasis. Both PsA and Ps, are associated with deterioration of |
| | social functioning and with psychological problems and is important |
| | to be evaluated. The regularity and severity of sleep disorders may be |
| | associated with inflammation, chronic pain and pruritus, decreased |
| | quality of life caused by the disease, emotional disorders such as |
| | anxiety and depressive reactions and adverse reactions to current |
| | medication. 1. There is evidence that PsA is associated with sleep |
| | disturbance and patients prioritized this impact in the EULAR PsAID |
| | measure, yet sleep is rarely assessed in PsA research or clinical care. |
| | The Medical Outcomes Study Sleep Scale has been used in a study of |
| | psoriasis and fibromyalgia but not specifically in PsA. The PsAID |
| | questionnaire is the only PsA specific PRO assessing sleep |
| | disturbance as one of its domains. Further studies are needed to |
| | address optimal PROs for sleep in PsA. |
| Depression | Depression is correlated with severe pruritus, pain, sensitiveness, |
| | weakness and botheration by contact with water and affects the quality |
| | of life in many patients. |
| Swelling and tenderness | Arthritis = inflamed joints or synovium. Arthritis is the most common |
| | complication of UC. The articular disease is characterized by subacute |
| | episodes, closely related to exacerbations of the intestinal symptoms, |
| | and only rarely does it persist and lead to permanent joint damage and |
| | chronic arthritis. Regional ileitis is probably associated with a similar |
| | type of arthritis, although the limited number of observations makes |
| | this a tentative opinion. |
| Inflammatory skin | The skin is one of the most commonly affected organ systems in |
| | patients who suffer from IBD. A disturbance of the equilibrium |
| | between host defense and tolerance, and the subsequent over-activity |
| | of certain immune pathways are responsible for the cutaneous |
| | disorders seen so frequently in IBD patients. 1. Specific cutaneous |
| | manifestations or granulomatous cutaneous lesions with the same |
| | histological features as the underlying bowel disease 2. Reactive |
| | cutaneous manifestation of IBD with immunological mechanisms |
| | triggered by common antigens shared by gut bacteria and skin 3. |
| | Cutaneous disorders or dermatosis associated with IBD 4. Secondary |
| | cutaneous manifestations either due to complications of IBD or |
| | adverse effects of IBD treatments. |
| Stifness | Persons with knee osteoarthritis demonstrate a reduction in knee joint |
| | excursion during loading response which is often coupled with a |
| | reduction in the moment acting to flex the knee. Higher muscle |
| | activity aimed at reducing the knee flexion and pain with movement |
| | would also result in higher dynamic joint stiffness. |
| Inflammatory eyes | Evaluation of the eye should be a routine component in the care of |
| | patients with IBD. Clinicians must be aware of the spectrum of ocular |
| | symptoms and know that these complaints may precede a diagnosis of |
| | ulcerative colitis (UC) or Crohn's disease (CD). Clinical |
| | manifestations include blurred vision, teary, burning or itchy |
| | eyes, ocular pain, photophobia, conjunctival or scleral hyperemia, loss |
| | of visual acuity, and possible blindness. The visual system is affected |
| | by inflammatory disorders such as episcleritis, uveitis and scleritis. |
| Mouth ulcers | Aphthous stomatitis or ulcers are: minor aphthous ulcers - small, |
| | shallow, round to oval shaped, have a grayish base, and can be |
| | painful, but heal within 2 weeks without scarring or major recurrent |
| | ulcers - larger, can last for 6 weeks, and frequently scar. |
| Abdominal cramping | Reversible causes of abdominal pain in IBD include strictures, |
| | abscesses, fistulae, and small intestinal bacterial overgrowth. |
| | Abdominal pain that persists beyond flares, despite optimal treatment |
| | of the gut disease, presents a common, disabling, and unresolved |
| | problem, affecting patients” quality of life (QoL) and psychological |
| | well-being and posing challenges for management. |
| Tenderness | By using longitudinal MRI and clinical data from the Oslo hand OA |
| | cohort, we demonstrated significant associations between |
| | increasing/incident synovitis and BMLs and incident joint tenderness, |
| | supporting the validity of synovitis and BMLs as sources of pain in |
| | hand OA. |
| Bone spurs | Hand OA is the second clinical condition leading to bone spur |
| | formation along the peripheral joints. |
| Fatigue | Fatigue in OA is associated with pain, sleep disturbance and depressed |
| | mood. |
| Aching | Characterized by diffuse aching, burning pain in joints that is usually |
| | moderately severe, and usually intermittent with exacerbations and |
| | remissions. It is cons' people with RA also often use descriptors that |
| | are more characteristic of neuropathic pain such as ‘burning’ or |
| | ‘shooting’ suggesting possible nerve damage. ‘“idered to be recurrent |
| | and chronic in nature. It is characterized by the following components: |
| | physiologic, affective, sensory-discriminative, and cognitive. |
| Stifness | Our data point to several potential reasons for the stiffness-pain |
| | interdependence, including the following: the experience of pain is |
| | overwhelming, with stiffness considerably overshadowed; stiffness |
| | and pain experiences are tightly connected, whereby separation is |
| | meaningless; and stiffness is poorly understood by other people in |
| | comparison to pain, such that reports of pain have more usefulness for |
| | people living with RA |
| Fatigue | RA fatigue can be influenced by numerous factors, such as |
| | inflammation, pain, disability, and psychosocial factors (mood, |
| | beliefs, behavior) 8-10. Although chronic inflammation may cause |
| | fatigue, in RA it has been shown that pain, rather than inflammation, |
| | is associated with fatigue severity. |
| Physical activity | People with RA often reduce their physical activity levels, due to the |
| | mechanical drive to pain during movement and weight bearing, and |
| | fear that activity might induce further pain, or flare of inflammatory |
| | disease activity. Muscle weakness: Muscle weakness is generally |
| | attributed to a reflex response to pain, joint deformation or disuse, |
| | extra-articular manifestations of the disease and/or psychological |
| | factors. Cachexia: rheumatoid cachexia leads to muscle weakness and |
| | a loss of functional capacity, and is believed to accelerate morbidity |
| | and mortality in rheumatoid arthritis. |
| Sleep disturbance | Furthermore, they(RA Patients) commonly describe more widespread |
| | pain associated with sleep disturbance, fatigue, and low mood. These |
| | ‘fibromyalgic’ symptoms suggest abnormalities of central pain |
| | processing. |
| Depression | There is also growing evidence that antidepressant medication”“ and |
| | cognitive behaviour therapy” 3 can help decrease levels of self- |
| | reported pain improve functional status and improve quality of life in |
| | this population. Depression and anxiety were highly correlated with |
| | several measures of arthritisrelated pain and functional impairment. |
| | Patients with depression as well as arthritis tend to report increased |
| | functional disability and increased levels of arthritis-related pain |
| | compared to individuals with arthritis alone. |
| Itch | Itching has been defined as an unpleasant sensation that provokes the |
| | desire to scratch. 10 It is so unbearable that patients often find that they |
| | must scratch until the itch is replaced by pain. Itch and pain are |
| | separate phenomena Both sensations may be experienced |
| | simultaneously. When C-fibers are stimulated electrically, some |
| | transmit pain, and others transmit itch. Increasing the intensity of |
| | stimulation does not change the quality of the sensation in any given |
| | fiber. (Pain is always pain, and itch is always itch. |
| Depression | Skin pain, particularly severe pain, was associated with increased AD |
| | severity, poor sleep, depressive symptoms and poorer QOL. Atopic |
| | dermatitis (AD) is a common disease associated with an |
| | underappreciated increased risk of depression and suicidality. |
| Physical activity | Exercise therapy has slightly positive benefits in people with OA; self- |
| | reported physical function and pain improved approximately 6% on |
| | average, whereas depression decreased by an average of 2.4%. Muscle |
| | strengthening programs that include combinations of strength, |
| | flexibility, and aerobic exercises had more benefits for pain and |
| | physical function than general activity. |
| Obesity | Patients with improved RA disease activity may be more likely to lose |
| | weight through mechanisms such as improved quality of life, less |
| | pain, increased physical activity, and healthier diet. Severe obesity is |
| | associated with a more rapid progression of disability in RA. Weight |
| | loss is also associated with worsening disability, possibly due to it |
| | being an indication of chronic illness and the development of age- |
| | related or disease-related frailty. |
| Synovitis | Synovitis in Osteoarthritis |
| Leakage of Urine | |
| coi-sh | |
| Myoclonus discharge | Myoclonus is sudden, brief, jerky, shock-like, involuntary movements |
| | arising from the central nervous system and involving extremities, |
| | face, and trunk. |
| Rhythmicity of consecutive myoclonus | Myoclonus is sudden, brief, jerky, shock-like, involuntary movements |
| discharges | arising from the central nervous system and involving extremities, |
| | face, and trunk. |
| Spread of myoclonus among different | Myoclonus is sudden, brief, jerky, shock-like, involuntary movements |
| muscles | arising from the central nervous system and involving extremities, |
| | face, and trunk. |
| Amplitude and frequency of myoclonus | Myoclonus is sudden, brief, jerky, shock-like, involuntary movements |
| | arising from the central nervous system and involving extremities, |
| | face, and trunk. |
| Daytime scratch | Scratching events measured during daytime activities |

TABLE 4
Example Target Solution Profiles (TSP). Certain descriptions of example TSPs are left blank on purpose.

| Example TSP | Description |
|---|---|
| Actigraphy | A TSP that makes use of a (actigraphy), to determine (total sleep time), to |
| | address (nocturnal itching) for Atopic Dermatitis true |
| Early CRPC patient identification | The use of noninvasive PET CT based radiomics to predict Tumor PDL1 |
| through PDL1 status prediction | status as a proxy for CRPC occurance |
| based on PETCT | |
| Test TSP | |
| Radio Frequency | A TSP that makes use of a (radio frequency sensor), to determine (wake |
| | after sleep onset), to address (nocturnal itching) for Atopic Dermatitis |
| Actigraphy | A TSP that makes use of a (actigraphy), to determine (wake after sleep |
| | onset), to address (nocturnal itching) for Atopic Dermatitis |
| Multiple Sensor Device | A TSP that makes use of a (multiple sensors), to determine (wake after |
| | sleep onset), to address (nocturnal itching) for Atopic Dermatitis |
| Skin Measurement Devices | Devices to measure skin conditions such as humidity, integrity. |
| Radio Frequency | A TSP that makes use of a (radio frequency), to determine (total sleep |
| | time), to address (nocturnal itching) for Atopic Dermatitis true |
| Automated SVDH hand and feet | Automated solution to translate patient XRAYs into a structured Van der |
| XRAYs scoring | Heide modified Sharp score for Rheumatoid Arthritis |
| Crossplatform mobile application for | A android and apple compatible mobile application that can use |
| automated malignant lession | automated imaging analysis locally on the phone on pictures taken by the |
| detection in skin cancer | native phone camera to predict whether a patient needs to go to a |
| | specialist for a specific lession for follow up. |
| High Fidelity and magnified | Using dedicated special mangnifiers and a selective breed of smartphones |
| specialised mobile application for | with HighRes and stabilizing photo equipment with dedicated software |
| automated Malignant Lession | predict risk of skin lessions in relation to skin cancer for further follow up |
| detection in skin cancer in highrisk | |
| patient groups | |
| Actigraphy | |
| Radio Frequency | |
| Radio Frequency | |
| Actigraphy | |
| Actigraphy | Devices which determine sleep efficiency by actigraphy measures |
| Radio Frequency | Devices which determine sleep efficiency by radio frequency |
| Actigraphy | Devices which determine sleep efficiency by actigraphy measures |
| Radio Frequency | Devices which determine sleep efficiency by radio frequency |
| Multiple Sensor Device | A TSP that makes use of a (multiple sensors), to determine (total sleep |
| | time), to address (nocturnal itching) for Atopic Dermatitis |
| Eye Movement Tracking | |
| Face Recognition | A TSP that make use of a (facial recognition) to measure the (memory), |
| | to infer information about the patient's (cognition) in (Alzheimers) |
| Skin Analysis Software | Software to classify skin diseases, analyze the wound types etc. |
| eCOA cognitive batteries | |
| eCOA cognitive batteries | Cognitive assessments: 25-30 minutes; no professional necessary |
| Skin Analysis Software | Software to classify skin diseases, analyze the wound types etc. |
| tsp: test | |
| Radio frequency | |
| Multiple Sensor Device | A TSP that makes use of a (multiple sensors), to determine (nocturnal |
| | scratching), to address (nocturnal itching) for Atopic Dermatitis |
| TSP: Radio Frequency | A TSP that make use of a (radio frequency) to measure the (nocturnal |
| | itching events), to determine (total number of events), to address |
| | (nocturnal itching) |
| Cognitive battery: AI analysis of | |
| assessed speech recordings in | |
| episodic memory | |
| Automated speech modalities | A TSP that make use of a (speech recording) to measure the (memory), to |
| | infer information about the patient's (cognition) in (Alzheimers) |
| Cognitive battery: AI analysis of | A TSP that make use of a (a recording device or app) to measure the |
| assessed speech recordings in | (speech), to address (cognition: memory) in (Alzheimers) |
| cognitive flexibility | |
| Face recognition in episodic memory | A TSP that make use of a (facial recognition) to measure the (memory), |
| | to infer information about the patient's (cognition) in (Alzheimers) |
| Cognitive battery: AI analysis of | |
| assessed speech recordings in | |
| naming | |
| eCOA cognitive batteries | |
| Cognitive battery: AI analysis of | Cognitive assessments: 25-30 minutes; no professional necessary |
| assessed speech recordings in | |
| processing speed | |
| Cognitive battery: AI analysis of | |
| assessed speech recordings in | |
| language/speech | |
| TSP: Accelerometery | A TSP that make use of a (Accelerometery) to measure the (hours of |
| | sleep time), to determine (total sleep time), to address (nocturnal itching) |
| Echocardiogram: image analysis | Identify patients for echocardiographic probabilty of PH |
| software in early diagnosis of PH | |
| Actigraphy | A TSP that makes use of a (Activity Monitor) to measure the (steps per |
| | day), to determine (walking capacity), to address (fatigue). |
| Cognitive battery: AI analysis of | |
| assessed speech recordings in | |
| working memory | |
| tsp: test1 | tsp: test |
| Actigraphy: sleep time in sleep | Measuring sleep activity and waso (wake after sleep onset) count in Sleep |
| efficiency | Wake Disorders |
| Actigraphy: movement detection in | Measuring movements in periodic limv events in Restless Legs |
| periodic limb events | Syndrome. |
| Actigraphy: movement detection, | Measuring nocturnal scratch and night itch in Atopic Dermatitis |
| degree, intensity and duration in | |
| nocturnal scratch | |
| Actigraphy: movement detection, | |
| degree, intensity and duration in | |
| sleep efficiency | |
| Actigraphy: movement detection in | Measuring sleep efficiency and sleep disturbance in Chronic Insomnia |
| sleep efficiency | Disease |
| Actigraphy: movement detection in | Measuring sleep efficiency and sleep disturbance in Insomnia Disease |
| sleep efficiency | |
| Activity monitor: triaxial inertial | Measuring physical activity and mobility in Lower Limb Amputation |
| measurement in physical activity | (knee level) |
| walking | |
| Tonometry: corneoscleral area | Measuring IOP and IOP fluctuation in Glaucoma Disease |
| changes in IOP reduction. | |
| Accelerometry: physical movement | Measuring physical activity and weight loss in Cachexia Disease |
| measurements and intensity | |
| detection in physical activity | |
| (walking) | |
| Actigraphy: physical movement | Measuring physical activity and pain in Diabetic Peripheral Neuropathy |
| measurements in physical activity | Disease |
| (walking) | |
| Actigraphy: movement detection in | Measuring sleep efficiency and sleep disturbance in Alzheimer's Disease |
| sleep efficiency | in Actigraphy: movement detection in sleep efficiency |
| CGM: continuous measurement of | Measuring glycemic variability and hyperglycemia in Diabetes Mellitus |
| glucose levels in glycemic | Disease. |
| variability | |
| Accelerometry: movement detection | Measuring Physical Activity and Airflow Limitation in Chronic |
| in physical activity | Obstructive Pulmonary Disease |
| Actigraphy: Exercise tolerance and | Measuring physical activity and exercise tolerance in Pulmonary Arterial |
| walking distance measurement in | Hypertension |
| physical activity. | |
| Cough monitor: recorded sounds | Measuring cough activity and cough count in Chronic Cough |
| from the lungs and trachea by chest | |
| contact sensor and ambient sounds | |
| by lapel microphone in coughs per | |
| hour | |
| Wearable Defibrillator: Heart Rate | Measuring heart rate variability in Cardiomyopathies |
| Monitor Enhanced Treatment | |
| Optimization by Wearable | |
| Cardioverter Defibrilator in Heart | |
| Rate Variability | |
| Wearable Defibrillator: Heart Rate | Measuring heart rate variability in Heart Failure |
| Monitor Enhanced Treatment | |
| Optimization by Wearable | |
| Cardioverter Defibrilator in Heart | |
| Rate Variability | |
| Pulse oximeter: blood volume by | Measuring blood volume pulse variations in Hearing Loss |
| photoplethysmographic (PPG) | |
| sensor in blood volume pulse | |
| Activity monitor: inertial | Mearuring balance in Parkinson's Disease |
| measurement unit sensor data in | |
| physical activity | |
| CGM: glucose values by continuous | Measuring glucose variability in Type I Diabetes |
| glucose monitor in glucose | |
| variability | |
| Live image analysis: analyze and | Leprosy\| unspecified - skin itch - reduction in skin lesions |
| quantify skin lesions using AI/ML | |
| Actigraphy: physical movement | Measuring Physical activity and Pain in Osteoarthritis of Hip Disease |
| measurements in walking activity | |
| Accelerometry: physical movement | Measuring physical activity and pain in Knee Osteoarthritis Disease |
| measurements in walking activity | |
| Decision Support Tool: glucose | Measuring glucose variability in Type I Diabetes |
| patterns by algorithm in glucose | |
| variability | |
| Actigraphy: physical movement | Measuring Physical Activity and Airflow Limitation in Chronic |
| measurements in physical activity | Obstructive Pulmonary Disease |
| Digital Therapeutics: patient | Measuring gait metrics and gait speed in Cerebral infarction |
| application and sensors in gait | |
| metrics | |
| Clip-on sensor: records vial | Measuring medication adherence in Asthma |
| open/close in medication adherence | |
| Activity monitor: motor test | Measuring movement in Parkinson's Disease |
| measurements in tremor | |
| Activity monitor: inertial | Mearuring tremor in Parkinson's Disease |
| measurement unit sensor data in | |
| tremor | |
| PSG | Polysomnography |
| Activity monitor: inertial | Measuring gait in Parkinson's Disease |
| measurement unit sensor data in gait | |
| Spirometry: pre-bronchodilator | Assesing the pulmonary function by measuring pre-bronchodilator forced |
| forced expiratory volume measure in | expiratory volume in COPD Patients |
| pulmonary function | |
| Activity monitor: Movement degrees | Measuring wrist range of motion in Rheumatoid arthritis patients |
| measurement in wrist range of | |
| motion | |
| Activity monitor: movement | Measuring physical activity in patients suffering from Diabetes Type 1 |
| detection in physical activity | |
| TSP: AA Test Actigraphy Skin on | AA Test Actigraphy Skin on Skin Contact Nocturnal Scratch |
| Skin Contact Nocturnal Scratch | |
| Actigraphy: physical movement | Measuring skin itch and nighttime scratch in Atopic Dermatitis Disease |
| measurements in nocturnal scratch | |
| (test) | |
| Infrared | A TSP used to monitor nocturnal itching using infrared technology |
| CGM: glucose values by glucose | Measuring glucose variability in Diabetes Mellitus Type 2 |
| monitoring device in glucose | |
| variability | |
| System: physical movement | Measuring lower extremity gait and mobility in Parkinson's Disease |
| measurements in gait | |
| Accelerometer: tremor count by | Measuring tremor and tremor count in Parkinson's Disease |
| accelerometry in Performance of a | |
| wearable accelerometer in detecting | |
| dyskinesia (17019) | |
| Actigraphy: movements degree and | Measuring Physical Activity and Dyspnoea in Angina pectoris (Chronic |
| intensity detection in daily physical | Stable Angina) |
| activity | |
| Actigraphy: physical movement | Measuring Physical Activity and Pain in Chronic Pain Disease |
| measurements in walking activity | |
| CGM: continuous measurement of | Measuring glucose variability in Type I Diabetes |
| glucose levels in glycemic | |
| variability | |
| Activity monitor: movement | Measuring sleep efficiency in Type 1 Deabetes |
| detection in sleep efficiency | |
| Thermometer: temperature by non- | Measuring body temperature and fever in Covid 19 |
| contact infrared thermometer in time | |
| to sustained absence of fever | |
| Actigraphy: physical movement | Measuring Physical Activity and Airflow Limitation in Chronic |
| measurements in physical activity | Obstructive Pulmonary Disease |
| Actigraphy: physical movement | Measuring Insomnia and Sleep Efficiency in Major Depressive Disorder |
| measurements in walking activity | |
| The constantly fluctuating changes | Measuring Seizures and Seizure Activity in Rett's Syndrome |
| in certain electrical properties of the | |
| skin: Seizure activity (prediction) by | |
| Electrodermal activity | |
| Accelerometry: movement detection | Measuring Physical Activity and Seizures in Rett's Syndrome |
| in physical activity | |
| Heart rate variability: | Measuring Seizures and Seizure Activity in Rett's Syndrome |
| Photoplethysmographic Heart Rate | |
| in Seizure Activity (seizure | |
| prediction) | |
| Skin Temperature variability: | Measuring Seizures and Seizure Activity in Rett's Syndrome |
| peripheral skin temperature in | |
| Seizure Activity (seizure prediction) | |
| Actigraphy: movement detection in | Measuring Physical activity and Respiratory Disturbance in Cystic |
| physical activity | Fibrosis Disease |
| Actigraphy: movement detection in | Measuring Sleep Efficiency and Respiratory Disturbance in Cystic |
| sleep efficiency | Fibrosis Disease |
| Accelerometry: activity monitoring | Measuring Physical Activity and Foot Complications in Other specified |
| of physical activity providing | diabetes mellitus with diabetic chronic kidney disease |
| feedback about gait speed | |
| Patient adherence: system for | Measuring Medication Adherence and Medication Intake in HIV Disease |
| continuous monitoring in medication | |
| intake | |
| Accelerometry: activity monitoring | Measuring Physical Activity and Foot Complications in Other specified |
| of physical activity providing | diabetes mellitus with diabetic chronic kidney disease |
| feedback about body sway | |
| Actigraphy: sleep movement | To measure TST in sleep quality in AD. |
| detections in sleep quality | |
| Activity monitor: Balance and | Measuring balance in diabetic foot ulcer patients |
| postural sway in balance | |
| Actigraphy: movements degree and | Measuring Physical Activity and Sleep Disturbance in Irregular Sleep- |
| intensity detection in physical | Wake Rhythm Disorder |
| activity | |
| Actigraphy: movements degree and | Measuring Physical Activity and Sleep Disturbance in Irregular Sleep- |
| intensity detection in sleep | Wake Rhythm Disorder |
| efficiency | |
| Activity monitor: Wearable activity | Measuring physical activity in patients suffering from Pulmonary |
| monitor (actigraph) by activity | hypertension associated with interstitial lung disease (PH-ILD) |
| counts | |
| Activity monitor: movement | Measuring physical activity in patients with diabetic foot ulcer |
| detection in physical activity | |
| Wrist-worn watch: cognitive | Measuring Working Memory and Cognitive Impairment in Major |
| performance variability with | Depressive Disorder |
| assessment tests in working memory | |
| Pulse oximetry: non-invasive | measuring tissue oxygenation of lower extremities in patients with |
| oxygenation measurement in lower | diabetic foot ulcer |
| extremeties oxygen saturation | |
| Wrist-worn watch: cognitive | Measuring Attention and Cognitive Impairment in Major Depressive |
| performance variability with | Disorder |
| assessment tests in attention | |
| Activity monitor: Gait speed in | Measuring gait speed in Diabetic Foot Ulcers Patients |
| kinematics of lower body | |
| Video: automated endoscopy image | Scoring system to determine MES 0-3 by ML analyzing video images |
| MES in abdominal pain and | |
| cramping | |
| Activity monitor: Sensor for | Measuring sedentary behaviour in diabetic foot ulcer patients |
| continuous remote monitoring in | |
| sedentary behaviour | |
| Heart Rate Monitor: Sensor | Measuring heart rate variability in diabetic foot ulcer patients |
| measuring physiological signs in | |
| Heart Rate Variability | |
| Biosensor: direct trans-epidermic | To measure skin skin texture in skin condition in Atopic Dermatitis |
| water loss (TEWL) and stratum | |
| corneu hydration (SCH) measures in | |
| skin condition | |
| Electrical Impedance Spectroscopy | To measure skin skin texture in skin condition in Atopic Dermatitis |
| (EIS): analyzes precise electrical | |
| measurements applied to the skin in | |
| skin condition | |
| Skin analysis application | To measure wound status in skin condition in AD. |
| Activity monitor: movement | Measuring nocturnal activity in postoperative recovery |
| detection in nocturnal activity | |
| Radio frequency: ambient RF | To measure TST in sleep quality in AD. |
| measures in sleep quality | |
| ActiGraph: Accelerometer | measuring mobility in postoperative recovery |
| measuring mobility | |
| Activity monitor: Activity counts in | Measuring physical activity in postoperative recovery |
| physical activity | |
| Activity monitor: Movement | Measuring chorea in Huntington Disease patients |
| detection in chorea | |
| Wireless motion sensor: motor test | Measuring Physical Activity and Motor Activity in Parkinson's Disease |
| measurements in tremor, | |
| bradykinesia, dyskinesia | |
| Pulse oximetry: blood oxygen level | Measuring pulse oxygenation and pulse oxygenation level in Covid 19 |
| measurements in oxygen saturation | |
| (SpO2) | |
| Patient adherence: system for | Measuring Medication Intake and Medication Adherence in Tuberculosis |
| continuous monitoring in medication | Disease |
| intake | |
| Actigraphy: sleep movement | Measuring Insomnia and Sleep Efficiency in Major Depressive Disorder |
| detections in sleep efficiency | |
| Mobile application: skin analysis in | To measure wound status in skin condition in AD. |
| wound status | |
| Wireless motion sensor: motor | Measuring Physical Activity and Motor Activity in Parkinson's Disease |
| symptoms recording by continuous | |
| measurement in tremor, slowness, | |
| dyskinesia and mobility | |
| ECG: Electrocardiogram by | Measuring irregular heart rythm and atrial fibrillation burden in Atrial |
| submersible arrhythmia monitor | Fibrillation |
| (ECG wearable patch) in Irregular | |
| Heart Rythm | |
| Actigraphy: Sleep WASO by | Measuring sleep efficiency and waso (wake after sleep onset) in Restless |
| Actigraphy in Sleep Efficiency | Legs Syndrome |
| Actigraphy: Sleep Duration by | Measuring sleep efficiency and sleep duration in Restless Legs Syndrome |
| Actigraphy in Sleep Efficiency | |
| Audio recordings: voice tone tension | |
| analysis in depression | |
| CGM: AID insulin dosing in glucose | AID insulin dosing and Measuring glucose variability in Diabetes |
| variability | Mellitus |
| Radio frequency: ambient RF | To measure EASI in pruritas in AD. |
| measures in EASI score | |
| Infrared: ambient IR measures in | To measure EASI in pruritas in AD. |
| EASI score | |
| Actigraphy: physical movement | Actigraphy is a non-invasive method of monitoring human rest/activity |
| measurements in EASI | cycles. A small actigraph unit, also called an actimetry sensor, is worn to |
| | measure gross motor activity. |
| Motion sensor: facial recognition by | Measuring facial movement and facial task performance in Huntington |
| 3-D optical motion capture system in | Disease |
| facial movement | |
| Motion sensor: physical activity by | Measuring movement detection and motor performance in Huntington |
| motion capture system in movement | Disease |
| detection | |
| Camera: Changes in skin roughness | Measuring skin aging and change in skin roughness in Changes in Skin |
| by high resolution b/w video sensor | Texture |
| in Skin Aging | |
| Camera: Change in skin wrinkle by | Measuring skin aging and change in skin wrinkle in Changes in Skin |
| high resolution b/w video sensor in | Texture |
| Skin Aging | |
| Camera: Change in skin age by | Measuring skin aging in Changes in Skin Texture |
| Facial Photo Capture in Skin Aging | |
| Actigraphy: Activity counts in | Measuring physial activity in Breast Cancer |
| physical activity | |
| Activity Monitor: activity count by | Measuring physical activity and sleep in Mild Cognitive Impairment |
| wearable wireless sensors in | |
| physical activity and sleep | |
| Activity Monitor: activity count by | Measuring physical activity and sleep in Mild Cognitive Impairment |
| wearable wireless sensors in | |
| physical activity and sleep | |
| Actigraphy: movement detection in | Measuring Sleep Efficiency and Sleep Disturbance in Reflux Esophagitis |
| sleep efficiency | Disease |
| Actigraphy: movement detection in | Measuring Sleep Efficiency and Pain in Neuromyelitis Optica Spectrum |
| sleep efficiency | Disorder |
| Actigraphy: physical movement | Measuring Physical Activity and Pain in Neuromyelitis Optica Spectrum |
| measurements in Physical Activity | Disorder |
| Actigraphy: movement detection in | Measuring Sleep Efficiency and Pain in Transverse Myelitis Disease |
| sleep efficiency | |
| Actigraphy: physical movement | Measuring Physical Activity and Pain in Transverse Myelitis Disease |
| measurements in Physical Activity | |
| Actigraphy: physical movement | Measuring Physical Activity and Pain in Multiple Sclerosis Disease |
| measurements in Physical Activity | |
| Actigraphy: movement detection in | Measuring Sleep Efficiency and Pain in Multiple Sclerosis Disease |
| sleep efficiency | |
| Actigraphy: physical movement | Measuring Physical Activity and Exercise Tolerance in Chronic Heart |
| measurements in physical activity | Failure With Reduced Ejection Fraction Disease |
| Actigraphy: movement detection and | Measuring Sleep Efficiency and Exercise Tolerance in Chronic Heart |
| intensity in sleep efficiency | Failure With Reduced Ejection Fraction Disease |
| Accelerometry: physical movement | Measuring Physical Activity and Exercise Tolerance in Heart Failure |
| measurements in physical activity | With Preserved Ejection Fraction Disease |
| Accelerometry: physical movement | Measuring Physical Activity and Dyspnea in Pulmonary Hypertension |
| measurements in physical activity | Disease |
| Actigraphy: physical activity | To measure physical activity measurements in nocturnal scratch in |
| measurements in nocturnal scratch | pruritas in AD. |
| Radio frequency: ambient RF | To measure nocturnal scratch in pruritas in AD. |
| measures in nocturnal scratch | |
| Infrared: ambient IR measures in | To measure nocturnal scratch in pruritas in AD. |
| EASI score | |
| Radio frequency: ambient RF | To measure TSO in sleep quality in AD. |
| measures in sleep quality | |
| Radio frequency: ambient RF | To measure WASO in sleep quality in AD. |
| measures in sleep quality | |
| Actigraphy: sleep movement | To measure TSO in sleep quality in AD. |
| detections in sleep quality | |
| Actigraphy: sleep movement | To measure WASO in sleep quality in AD. |
| detections in sleep quality | |
| CGM: glucose values by continuous | Measuring glucose variability in Glucose Intolerance |
| glucose monitor in glucose | |
| variability | |
| Actigraphy: physical movement in | Measuring sleep activity and sleep efficiency in Glucose Intolerance |
| sleep efficiency | |
| Actigraphy: physical movement in | Measuring sleep activity and sleep midpoint in Glucose Intolerance |
| sleep efficiency | |
| CGM: glucose values by continuous | Measuring glucose variability and glucose intolerance in Short Bowel |
| glucose monitor in glucose | Syndrom |
| variability | |
| CGM: glucose values by continuous | Measuring glucose variability and feeding patterns in Short Bowel |
| glucose monitor in glucose | Syndrom |
| variability | |
| CGM: glucose values by continuous | Measuring glucose variability and sleep quality in Short Bowel Syndrom |
| glucose monitor in glucose | |
| variability | |
| Actigraphy: physical movement | Measuring Physical Activity and Exercise Tolerance in Pulmonary |
| measurements in physical activity | Arterial Hypertension Disease |
| Assessment tools | |
| Actigraphy: physical movement in | Measuring sleep activity and glucose intolerance in Short Bowel |
| sleep efficiency | Syndrome |
| Actigraphy: physical movement in | Measuring sleep activity and feeding patterns in Short Bowel Syndrom |
| sleep efficiency | |
| Actigraphy: physical movement in | Measuring sleep activity and sleep quality in Short Bowel Syndrom |
| sleep efficiency | |
| Actigraphy: physical movement | To measure DLPA in |
| measurements in DLPA | |
| Actigraphy: physical movement | To measure DLPA in physical activity in MDD |
| measurements in DLPA | |
| Actigraphy: physical movement | To measure DLPA in physical activity in MDD |
| measurements in DLPA | |
| Actigraphy: physical movement | To measure energy levels in physical activity in MDD |
| measurements in energy levels | |
| Actigraphy: physical movement | To measure posture in physical activity in MDD |
| measurements in posture | |
| Actigraphy: sleep movement | To measure sleep patency in sleep quality in MDD |
| detections in sleep patency | |
| Actigraphy: sleep movement | To measure TST in sleep quality in MDD |
| detections in TST | |
| Actigraphy: sleep movement | To measure WASO in sleep quality in MDD |
| detections in WASO | |
| Actigraphy: sleep movement | To measure ambient light in sleep quality in MDD |
| detections in relation to ambient | |
| light | |
| System: physical movement | |
| measurements in posture | |
| System: physical movement | To measure PA in motor activity in Parkinson's disease |
| measurements in physical activity | |
| Audio recordings: electrical signals | Measuring word recognition rate in Speech Disorder |
| recorded non-invasively by | |
| Augmentative and Alternative | |
| Communication device in Word | |
| Recognition Rate, Prosody | |
| Recognition Rate, Prosodic tone and | |
| break index and Prosodic sEMG- | |
| Acoustic Correlation | |
| Audio recordings: electrical signals | Measuring word recognition rate in Speech Disorder |
| recorded non-invasively by | |
| Augmentative and Alternative | |
| Communication device in Word | |
| Recognition Rate, Prosody | |
| Recognition Rate, Prosodic tone and | |
| break index and Prosodic sEMG- | |
| Acoustic Correlation | |
| Audio recordings: electrical signals | Measuring word recognition rate in Speech Disorder |
| recorded non-invasively by | |
| Augmentative and Alternative | |
| Communication device in Word | |
| Recognition Rate, Prosody | |
| Recognition Rate, Prosodic tone and | |
| break index and Prosodic sEMG- | |
| Acoustic Correlation | |
| Audio recordings: electrical signals | Measuring word recognition rate in Speech Disorder |
| recorded non-invasively by | |
| Augmentative and Alternative | |
| Communication device in Word | |
| Recognition Rate, Prosody | |
| Recognition Rate, Prosodic tone and | |
| break index and Prosodic sEMG- | |
| Acoustic Correlation | |
| Actigraphy: Activity counts in | Measuring physical activity in patients with Diabetic Peripheral |
| physical activity | Neuropathic pain |
| Audio recordings: electrical signals | Measuring word recognition rate in Speech Disorder |
| recorded non-invasively from speech | |
| muscles by Augmentative and | |
| Alternative Communication device | |
| in Word Recognition Rate, Prosody | |
| Recognition Rate, Prosodic tone and | |
| break index and Prosodic sEMG- | |
| Acoustic Correlation | |
| Audio recordings: electrical signals | Measuring prosody recognition and prosodic tone intelligibility in Speech |
| recorded non-invasively from speech | Disorder |
| muscles by Augmentative and | |
| Alternative Communication device | |
| in Word Recognition Rate, Prosody | |
| Recognition Rate, Prosodic tone and | |
| break index and Prosodic sEMG- | |
| Acoustic Correlation | |
| Audio recordings | Measuring word recognition rate in Speech Disorder |
| Audio recordings | Measuring prosody recognition and prosodic tone intelligibility in Speech |
| | Disorder |
| CGM: glucose values by continuous | Measuring glucose variability in Diabetes Mellitus Type 2 |
| glucose monitor in glucose | |
| variability | |
| CGM: glucose values by continuous | Measuring glucose variability in Kidney Transplant |
| glucose monitor in glucose | |
| variability | |
| Patient adherence: nebulizer to | Measuring Medication Intake and Medication Adherence in Pulmonary |
| monitor medication intake | Hypertension |
| Activity monitor: Movement | Measuring physical activity in allergic asthma patients |
| detection in Physical Activity | |
| Activity monitor: sensors measuring | Measuring nocturnal scratch in Atopic Dermatitis patients |
| scratch duration | |
| Actigraphy: scratch duration in | Measuring nocturnal scratch in Atopic Dermatitis patients |
| Nocturnal Scratch | |
| Activity monitor: wireless sensor | measuring motion in tremor |
| measuring motion in Tremor | |
| Accelerometry: movement detection | Measuring light to vigorous physical activity in heart failure patients |
| in light to vigorous physical activity | |
| Activity monitor: movement | measuring movement detection in PD patients |
| detection in tremor | |
| Activity monitor: movement | measuring movement in PD patient |
| detection in bradykinsesia | |
| Activity monitor: movement | Measuring movement in Parkinson's Disease |
| detection in bradykinsesia | |
| Activity monitor: movement | Measuring movement in Parkinson's Disease |
| detection in dyskinesia | |
| Activity monitor: movement | Measuring movement in Parkinson's Disease patients |
| detection in daytime somnolence | |
| Activity monitor: movement | Measuring movement in Parkinson's Disease patients |
| detection in daytime somnolence | |
| Activity monitor: movement | Measuring movement in Parkinson's Disease patients |
| detection in daytime somnolence | |
| Activity monitor: movement | Measuring movement in Parkinson's Disease patients |
| detection in daytime somnolence | |
| Activity monitor: movement | |
| detection in daytime somnolence | |
| Wearable patch: direct skin-on-patch | |
| measurements via patch in skin/body | |
| temperature | |
| Actigraphy: physical movement | To measure heart rate in physical activity in MDD |
| measurements in cardiac signs | |
| Accelerometry: Movement detection | Measuring physical activity in heart failure patients |
| in Physical Activity (20220) | |
| Pulse oximetry: Oxygenation | measuring overnight pulse oximetery variance in Sickle cell disease |
| measurement in overnight pulse | (SCD) patients |
| oximetery variance | |
| Actigraphy: movement detection in | measuring movement detection in Sickle cell disease (SCD) patients |
| sleep efficacy | |
| Actigraphy: movement detection in | Measuring nocturnal activity in Sickle cell disease (SCD) patients |
| nocturnal activity | |
| Actigraphy: activity count in | Measuring physical activity in Sickle cell disease (SCD) patients |
| physical activity | |
| CGM: continuous measurement of | Measuring glucose variability in Diabetes |
| glucose levels in glycemic | |
| variability | |
| Actigraphy: Movement detection in | measuring nocturnal activity in menopausal depression |
| nocturnal activity | |
| Actigraphy: Movement detection in | measuring nocturnal activity in asthma |
| nocturnal activity | |
| Chest Contact Sensor: cough count | measuring cough activity in chronic cough |
| in cough activity | |
| Chest Contact Sensor: Awake cough | measuring cough activity in chronic cough |
| frequency in cough activity | |
| Cognitive battery: AI analysis of | Cognitive assessments: 25-30 minutes; no professional necessary |
| assessed speech recordings in | |
| executive function | |
| Cognitive battery: AI analysis of | |
| assessed speech recordings in | |
| depression | |
| Cognitive battery: AI analysis of | |
| assessed speech recordings in | |
| depression | |
| Cognitive battery: AI analysis of | |
| assessed speech recordings in | |
| psychomotor function | |
| Cognitive battery: AI analysis of | |
| assessed speech recordings in | |
| psychomotor function | |
| Cognitive battery: AI analysis of | |
| assessed speech recordings in | |
| cognitive function | |
| Cognitive battery: AI analysis of | |
| assessed speech recordings in | |
| processing speed | |
| Cognitive battery: AI analysis of | |
| assessed speech recordings in 11 | |
| COIs | |
| Cognitive battery: AI analysis of | |
| assessed speech recordings in | |
| psychomotor function | |
| Bodysensensor(s): behavorial | |
| symptoms measures in social skills | |
| ECG: heart activity measure in early | |
| diagnosis of epilepsis | |
| EMG: muscle activity measure in | |
| early diagnosis of epilepsis | |
| Actigraphy: physical movement | |
| measurements in early diagnosis of | |
| epilepsis | |
| EEG: electrical brain waves activity | |
| in early diagnosis of epilepsis | |
| Audio recordings: automated speech | |
| analysis | |
| Patient adherence: recordings of vial | To measure executive function in cognition in Alzheimer's Disease |
| openings for medication intake | |
| PPG: recordings of cardiac signs in | To measure cardiac signs in early diagnosis of heart disease |
| early diagnosis of heart disease | |
| PPG: recordings of respiratory rate | To measure early diagnosis of heart disease in heart disease |
| in early diagnosis of heart disease | |
| PPG: recordings of skin temperature | |
| in early diagnosis of heart disease | |
| Big data: AI meta-analysis in tumor | To measure early diagnosis of tumor (markers) via AI meta-analysis |
| diagnosis | |
| Cognitive battery: touchscreen finger | To measure energy levels in typing behaviour in MS |
| movement detection in typing | |
| behaviour | |
| ECG: heart activity measure in | To measure cardiac signs in vitality in PH patients |
| cardiac signs | |
| ECG: heart activity measure in | |
| cardiac signs | |
| Actigraphy: sleep movement | Measuring Sleep Efficiency and Exercise Tolerance in Pulmonary |
| detections in sleep efficiency | Arterial Hypertension Disease |
| Medical exam kit: remote diagnosis | |
| via medical tools in vitality | |
| Chest patch wearable: electrial | |
| signal cardiac sign recordings via | |
| patch in physical activity | |
| Chest patch wearable: electrial | |
| signal cardiac sign recordings via | |
| patch in cardiac signs | |
| Chest patch wearable: electrial | |
| signal cardiac sign recordings via | |
| patch in respiratory rate | |
| Chest patch wearable: electrial | |
| signal cardiac sign recordings via | |
| patch in body temperature | |
| Medical device(system): brain health | Measuring Cognitive Assessment and Cognition in Alzheimer's Disease |
| measurement by cognitive assesment | |
| in 10 minutes | |
| GTI: calculation of CWS in | |
| glucocorticoid toxicity | |
| GTI: calculation of AIS in | |
| glucocorticoid toxicity | |
| CGM: glucose levels measured | |
| directly under the skin in glucose | |
| variation | |
| Bodysensensor(s): behavorial | |
| symptoms measures in repetitive | |
| behaviour | |
| Bodysensensor(s): behavorial | |
| symptoms measures in difficulty | |
| communicating | |
| Software application: word list recall | To measure episodic memory in cognitive impairment in MDD |
| tests in verbal episodic memory | |
| Microfluidics: photonics | |
| quantification of single blood drops | |
| in infections | |
| Software application system: | |
| assessments and HRV measures in | |
| heart rate variability | |
| Wearable patch: ambient patch | |
| measures in sleep apnea | |
| Wearable patch: direct skin-on-patch | |
| measurements via patch in vital | |
| signs | |
| Wearable patch: direct skin-on-patch | |
| measurements via patch in vital | |
| signs | |
| Wearable patch: direct skin-on-patch | |
| measurements via patch in vital | |
| signs | |
| Wearable patch: direct skin-on-patch | |
| measurements via patch in vital | |
| signs | |
| Wearable patch: direct skin-on-patch | |
| measurements via patch in heart rate | |
| Wearable patch: direct skin-on-patch | |
| measurements via patch in | |
| respiratory rate | |
| Wearable patch: direct skin-on-patch | |
| measurements via patch in pulse | |
| oximetry | |
| Wearable patch: direct skin-on-patch | |
| measurements via patch in walking | |
| Wearable patch: continuous vital | |
| sign monitoring via patch in physical | |
| activity | |
| ECG: heart activity measure in atrial | |
| fibbrilation | |
| Wearable patch: lung sound capture | |
| in cough | |
| Wearable patch: lung sound capture | |
| in wheezing | |
| Wearable patch: lung sound capture | |
| in lung sounds | |
| Audio recordings: voice tone tension | |
| analysis in energy levels | |
| Audio recordings: voice tone tension | |
| analysis in depression | |
| Audio recordings: sd | |
| sdsd | |
| Actigraphy: physical movement | |
| measurements in motor activity | |
| Actigraphy: cardiac sign | |
| measurements in motor activity | |
| Actigraphy: body condition | |
| measurements in motor activity | |
| Actigraphy: detects possible | |
| convulsive seizures in epilepsy | |
| Microphone: Speech assessment in | measuring language features in Alzheimer Disease patients |
| language features | |
| Actigraphy: physical movement | To measure DLPA in motor activity in Diabetes |
| measurements in daily life physical | |
| activity | |
| Audio recordings: ambient measures | |
| in nocturnal respiratory rate | |
| Audio recordings: ambient measures | |
| in nocturnal cough | |
| Audio recordings: ambient measures | |
| in nocturnal environmental | |
| conditions | |
| Audio recordings: ambient measures | |
| in nocturnal cough | |
| sss | |
| Audio recordings: ambient measures | |
| in nocturnal cough | |
| Audio recordings: ambient measures | |
| in nocturnal environmental | |
| conditions | |
| EEG: electrical brain waves activity | |
| in sleep efficiency | |
| Wearable patch: continuous vital | |
| sign monitoring via patch in sleep | |
| efficiency | |
| Wearable patch: continuous vital | |
| sign monitoring via patch in | |
| respiratory rate | |
| Wearable patch: continuous vital | |
| sign monitoring via patch in heart | |
| rate | |
| Wearable patch: continuous vital | |
| sign monitoring via patch in | |
| skin/body temperature | |
| Activity monitoring: motion and | Measuring motion and muscle activity in sleep activity in Parkinson's |
| muscle activity biometrics | Disease |
| measurement in sleep activity | |
| EOG: measuring the corneo-retinal | |
| standing potential in sleep efficiency | |
| EMG: measures the electrical | |
| activity of muscle in sleep efficiency | |
| ECG: records the electrical signals in | |
| the heart in sleep efficiency | |
| EEG: electrical brain waves activity | To measure sleep efficiency in insomnia in MDD |
| in sleep efficiency | |
| Actigraphy: oscillometric | |
| measurements via inflatable cuff in | |
| blood pressure variability | |
| Actigraphy: oscillometric | |
| measurements via inflatable cuff in | |
| blood pressure variability | |
| Actigraphy: movement | |
| measurements via inflatable cuff in | |
| physical activity | |
| Actigraphy: sleep movement | |
| measurements in sleep efficiency | |
| Biosensor: single-lead ECG | |
| outcomes in cardiac arrhytmias | |
| Microsample: urine-, saliva- or | |
| blood collection in research settings | |
| Activity monitoring: nanomembrane | Measuring blink count using nanomembrane electrodes in |
| electrodes measurement in blink | Blepharospasm |
| count | |
| EDA biosensor: skin conductance | Measuring skin conductance in cognitive engagement in cognitive |
| measurement in cognitive | impairment |
| engagement | |
| System: cognitive assessments in | |
| executive function | |
| System: cognitive assessments in | |
| executive function | |
| Heart rate monitor: pulse detection | Measuring pulse wave velocity in Autoinflammatory Syndrome |
| in pulse wave velocity | |
| Heart rate monitor: blood pressure | Measuring blood pressure variability in cardiac health in |
| variability measuring in cardiac | Autoinflammatory syndrome |
| health | |
| Heart rate monitor: | Measuring heart rate by photoplethysmography (PPG) in |
| photoplethysmography (PPG) in | Autoinflammatory syndrome |
| heart rate | |
| ECG: myocardial electrial pattern | |
| recognition in early diagnosis of PH | |
| Mobile application: measures | |
| disease progression in self- | |
| empowerment | |
| Mobile application: skin analysis in | |
| skin sensitivity | |
| Mobile application: measures | |
| disease progression in treatment | |
| adherence | |
| Mobile application: measures | |
| disease progression in vital signs | |
| Patient enrollment: AI model to | |
| identify patients with high-risks to | |
| develop lung cancer | |
| AI analysis: AI model to assess PFS | |
| in multiple myeloma | |
| Wearable patch: direct skin-on-patch | |
| measurements via patch in physical | |
| activity | |
| Actigraphy: sleep movement | |
| detections in sleep efficiency | |
| Actigraphy: sleep movement | |
| detections in sleep efficiency | |
| Activity monitoring: motion and | Measuring motion and muscle activity in sleep activity in Parkinson's |
| muscle activity biometrics | Disease |
| measurement in sleep efficiency | |
| Activity monitor: inertial | |
| measurement unit sensor data in | |
| posture | |
| Face recognition in executive | |
| function | |
| 21 PDI scoring | The 21-item Peters et al Delusions Inventory (PDI): |
| Assessment tools | |
| Computerized tool: MRI image | Analysis of whole-brain 3-T high-resolution brain magnetic resonance |
| analysis | imaging was used to determine the gray matter density changes across |
| | groups and their relations to eating behaviors. |
| Assessment tools | |
| Wandering scoring | Scales administered by caregivers: one measures wandering as a part of |
| | BPSD and the other measures wandering itself |
| Device: Monitoring system | |
| Device: Motion sensor | |
| Smart home | The proposed system used ambient sensors instead of wearable sensors or |
| | cameras to let the elders feel more comfortable when they are being |
| | monitored. |
| Mini-mental state | |
| Psychogeriatric scale | The psychogeriatric dependency rating scale |
| General medical health rating | |
| Actigraphy | |
| Audio recordings: vocal biomarkers | Vocal biomarkers extracted from the audio recorded in a controlled |
| | environment during performance of simple vocal tasks |
| Actigraphy | |
| Actigraphy | |
| Multimodal sensor system | To provide relevant quantitative evaluation of apathy close to real life |
| | situation by means of a multimodal sensor system integrated. |
| Geriatric Anxiety Invetory scale | |
| Assessment tools | |
| Penn State Worry Questionnaire | |
| RAID scale | Rating Anxiety in Dementia (RAID) scale: The RAID has the highest |
| | sensitivity for anxiety disorders, includes a caregiver interview, and was |
| | specifically designed for those experiencing dementia. |
| Actigraphy | |
| Computerized tool: MRI image | |
| analysis | |
| Video surveillance | Video Surveillance: enables direct observation of scratch activity, |
| | allowing for the calculation of total scratching time (TST) or the sum of |
| | the duration of all the scratching bouts (Scratch activity). |
| Assessment tools | |
| Device: blood pressure | |
| Device: blood pressure | |
| Patch: temperature | integrated Celsium's continuous temperature monitoring Medical Grade |
| | sensor. The sensor comes with adhesive patches that help fix temperature |
| | monitor to patient's body allowing for unniterrupted temperature |
| | measurement flow. |
| Device: weight scale | PCL connect's medical grade Scale can help pick up slightest change in |
| | weight. Readings captured from our Scale device are automatically |
| | synced with PCL's platform and made accessible to authorised parties be |
| | it carer, doctor or a family member. |
| Actigraphy | |
| RPPG | What is remote Photoplethysmography? Remote photoplethysmography |
| | (rPPG) is a noncontact video-based method that monitors the change in |
| | blood volume by capturing pixel intensity changes from the skin to |
| | measure pulse rate. |
| Mobile application: measures self- | Potential Applications of Digital Technology in Assessment, Treatment, |
| help, treatment and assessments | and Self-help for Hallucinations |
| Computerized tool: Strasbourg | “Strasbourg Visual Scale (SVS),” a novel computerized tool that allows |
| Visual Scale | us to explore and capture the subjective experience of visual |
| | hallucinations |
| Acoustic devices | Acoustic Devices: a wrist-worn sound detector has also been recently |
| | developed to objectively quantify scratching behavior, detect bone- |
| | conducted scratching sounds, which are transmitted to a piezoelectric |
| | sensor on the wrist. |
| Actigraphy | Wrist actigraphy, or the measure of body movement over time by a wrist- |
| | worn device with a microaccelerometer, has recently been used to |
| | measure the objective correlate of itch, scratch. (Physical activity, |
| | Nighttime activity) |
| Actigraphy | Physical activity including everyday walking as well as aerobic exercise |
| | may be objectively and longitudinally assessed using connected activity |
| | trackers. |
| Transepidermal water loss (TEWL) | |
| Stratum corneum (SC) hydration | |
| Device: soft tissue analysis | |
| Computer-adaptive testing (CAT) | CAT for fatigue in RA is not yet available, but will be developed in the |
| | near future. Potentially, this promising technology contributes to better |
| | measurements of fatigue in RA and the effects of treatment can be |
| | demonstrated more clear in the future |
| Dynamometer | 1. Maximum non-dominant handgrip strength (HGS) was evaluated using |
| | a hand dynamometer. Maximal quadriceps strength (QS) was measured in |
| | the non-dominant leg with an electro-mechanic chair dynamometer. |
| Chronometer | Lower-extremity functional performance was assessed by measures of |
| | ability to rise from a chair and walking velocity: A digital chronometer |
| | was used to measure the time spent on each of the two tests. 1. Sit-up test |
| | (ST). 2. Gait speed (GS). |
| Dynamometer | Muscle strength assessments |
| Bio-electrical impedance analysis | — |
| (BIA) | |
| DEXA | Total body DEXA technology (dual energy X-ray absorptiometry). |
| Assessment tools | Self-reported- Sleep Scale questionnaire |
| Treadmill | Instrumented treadmill with dual force plates |
| Pad-based fluid monitoring system | Pad based monitoring system that can detect leakage and register time of |
| | day and duration |
| Parkinson's tremor involuntary | A profile for the measurement of various aspects of involuntary |
| movement electromyography and | movement associated with Tremor in Parkinson's Disease using |
| accelerometry | electromyography and accelerometry |

TABLE 5
Example Digital Measurement Solutions (DMS). Certain descriptions
of example DMSs are left blank on purpose.

| Example DMS | Description |
|---|---|
| Pedometer | A DMS that measures (steps per day) to address TSP 2502 - Pulmonary |
| | hypertension -Fatigue-Walking capacity-Steps per day: Motion Detector |
| Mu et Al Algorithm based | |
| solution to predict CRPC by | |
| PDL1 status based on PETCT | |
| scans | |
| RF Wall mounted sensor | This device measures (hours of sleep time) to address TSP L209 - Atopic |
| device | Dermatitis - Nocturnal Itching - Total Sleep Time- hours of sleep: Radio |
| | Frequency |
| AA Apple Watch | AA Apple Watch |
| KI Elements Algorithm | |
| Modality Conversational AI | |
| system | |
| Neurotrack Eye Tracking | |
| Technology | |
| J&G DMS | J&G DMS |
| RA2 DREAM Automated | CNN for automated labeling and individual extremeties scoring |
| XRAY analysis for RA | |
| ActiGraph CentrePoint | |
| Insight Watch | |
| ActiGraph GT9X Link | |
| GENEActiv Original Watch | GENEActiv Original Watch (wrist-worn) |
| Emerald | Emerald (wall mounted RF sensor) |
| GPSkin | Simultaneously scans the Trans-Epidermic Water Loss (TEWL) and Stratum |
| | Corneum Hydration (SCH) levels of your skin. |
| Swift Skin and Wound | Automatically capture length, width and surface area accurately with fiducial |
| | marker, HealX. |
| Infrared camera ImageIR ® | Infrared camera ImageIR ® 9400 hp from InfraTec. Includes: Cooled FPA |
| 9400 hp from InfraTec. | photon detector with (1,280 × 1,024) IR pixels; Opto-mechanical MicroScan |
| | with (2,560 × 2,048) IR pixels; Extremely short integration times in the |
| | microsecond range |
| ActiGraph: Actisleep | |
| Activity monitor | Measuring physical activity and mobility in Lower Limb Amputation (knee |
| | level) in Activity monitor: triaxial inertial measurement in physical activity |
| | walking |
| Scibase: NeviSense | SciBase's Electrical Impedance Spectroscopy (EIS) |
| InfraTec: Infrared monitor | A DMS created for the use of the ImageIR Infrared monitor |
| ImageIR ® 9400 hp Infrared | |
| camera ImageIR ® 9400 hp | |
| Action: Magic band 2 (mock- | Measure your blood oxygen level with a revolutionary sensor and app. Take an |
| up) | ECG anytime, anywhere. See your fitness metrics at a glance with the enhanced |
| | Always-On Retina display. |
| Signant Health: Smartsignals | Cognitive assessments: 25-30 minutes |
| Clario: Opal V2C system | Measuring lower extremity gait and mobility in Parkinson's Disease in |
| | Actigraphy: physical movement measurements in lower extremity gait |
| CogState: Brief battery | eCOA cognitive battery |
| Cambridge Cognition: | Cognitive assessments: 20-30 minutes |
| CANTAB insight | |
| KI:Elements: AI speech | A DMS that measures episodic memory - Alzheimers |
| battery | |
| Winterlight Labs: AI speech | Speech analysis algorithm with recorded MP3 audiofile |
| battery | |
| Samsung: Galaxy Fit 2 | |
| ActiGraph: Actisleep | Measuring Nocturnal Scratch and Night Itch in Atopic Dermatitis in Actigraphy: |
| | movement detection, degree, intensity and duration in nocturnal scratch. |
| Device (placeholder) | Measuring periodic limb movements in Restless Legs Syndrome in Actigraphy: |
| | movement detection in periodic limb events. |
| Device (placeholder) | Sleep efficiency and sleep disturbane in Chronic Insomnia Disease in |
| | Actigraphy: movement detection in sleep efficiency |
| Philips: Actiwatch Spectrum | Sleep efficiency and sleep disturbance in Insomnia Disease in Actigraphy: |
| | movement detection in sleep efficiency |
| Bodymedia: Sensewear | Measuring sleep efficiency and sleep disturbance in Insomnia in Actigraphy: |
| | movement detection in sleep efficiency |
| Sensimed: SENSIMED | Measuring IOP and IOP fluctuation in Glaucoma Disease in Tonometry: |
| Triggerfish | corneoscleral area changes in IOP reduction. |
| Apple: Apple watch 7 | Wrist-worn device that contains an accelerometric sensor |
| Activity monitor | Measuring physical activity and mobility in Lower Limb Amputation (knee |
| | level) in Activity monitor: triaxial inertial measurement in physical activity |
| | walking |
| Janssen: US2.AI Algorithm | |
| Abbott: Freestyle Libre 14- | Measuring glucose variability in Type 2 Diabetes in CGM: glucose values by |
| day system | continuous glucose monitor in glucose variability |
| Device | Measuring Physical Activity and Respiratory Disturbance in Cystic Fibrosis |
| | Disease in Actigraphy: movement detection in physical activity |
| Photoplethysmographic | Measuring blood volume pulse variations in Hearing Loss in Pulse oximeter: |
| (PPG) sensor | blood volume by photoplethysmographic (PPG) sensor in blood volume pulse |
| DexCom: G6 Continuous | Measuring glucose variability in Type I Diabetes in CGM: glucose values by |
| Glucose Monitor | continuous glucose monitor in glucose variability |
| Canfield Scientific: VISIA | Leprosy\| unspecified - skin itch - reduction in skin lesions - Live image analysis: |
| Skin Analysis | analyze and quantify skin lesions using AI/ML |
| Actigraph | Measuring sleep activity and waso (wake after sleep onset) count in Sleep Wake |
| | Disorders in Actigraphy: sleep time in sleep efficiency |
| Device | Measuring Physical Activity and Pain in Osteoarthritis of Hip Disease in |
| | Actigraphy: physical movement measurements in walking activity |
| Bodymedia: SenseWear | Measuring Physical Activity and Airflow Limitation in Chronic Obstructive |
| Armband Gecko | Pulmonary Disease in Actigraphy: physical movement measurements in |
| | physical activity |
| SOMNOmedics: | Measuring Physical Activity and Airflow Limitation in Chronic Obstructive |
| SOMNOwatch Plus | Pulmonary Disease in Actigraphy: physical movement measurements in |
| | physical activity |
| OncoDNA: OncoKDM | |
| Philips Respironics: Actical | Measuring physical activity and pain in Diabetic Peripheral Neuropathy Disease |
| | in Actigraphy: physical movement measurements in physical activity (walking) |
| Biosensics: PAMSys | Measuring physical activity and pain in Knee Osteoarthritis Disease in |
| | Accelerometry: physical movement measurements in walking |
| Device (placeholder) | Sleep efficiency and sleep disturbance in Alzheimer's Disease in Actigraphy: |
| | movement detection in sleep efficiency |
| Abbott: FreeStyle Navigator | Glycemic variability and hyperglycemia in Diabetes Mellitus Disease in |
| | CGM: continuous measurement of glucose levels in glycemic variability |
| Proteus Digital Health: | Measuring Medication Intake and Medication Adherence in Tuberculosis |
| Proteus ® Digital Health | Disease in Patient adherence: ingestibles for continuous monitoring in |
| Feedback Device | medication intake |
| ActiGraph: wGT3X-BT | Measuring Physical Activity and Dyspnoea in Angina Pectoris (Chronic Stable |
| | Angina) in Actigraphy: movements degree and intensity detection in daily |
| | physical activity |
| McRoberts: Dynaport | Measuring Physical Activity and Airflow Limitation in Chronic Obstructive |
| MoveMonitor | Pulmonary Disease in Accelerometry: movement detection in physical activity |
| Bodymedia: SenseWear | Measuring Physical Activity and Airflow Limitation in Chronic Obstructive |
| Armband Gecko | Pulmonary Disease in Actigraphy: physical movement measurements in |
| | physical activity |
| ActiGraph: GT9x Link watch | Measuring Physical Activity and Airflow Limitation in Actigraphy: physical |
| | movement measurements in physical activity |
| Garmin: Vivofit 2 | Measuring Physical Activity and Airflow Limitation in Chronic Obstructive |
| | Pulmonary Disease in Actigraphy: physical movement measurements in |
| | physical activity |
| Device | Measuring Physical Activity and Pain in Knee Osteoarthritis Disease in |
| | Actigraphy: physical movement measurements in walking activity |
| ZOLL: LifeVest | Measuring heart rate variability in Heart Failure in Wearable Defibrillator: Heart |
| | Rate Monitor Enhanced Treatment Optimization by Wearable Cardioverter |
| | Defibrilator in Heart Rate Variability |
| Great Lakes | Measuring movement in Parkinson's Disease by Activity monitor: motor test |
| NeuroTechnologies: Kinesia | measurements in tremor |
| One Device | |
| GSK: Ellipta | Measuring medication adherence in Asthma in Clip-on sensor: records vial |
| | open/close in medication adherence |
| VitaloGraph: VitaloJAK ™ | Measuring cough activity and cough count in Chronic Cough in Cough monitor: |
| cough monitor | recorded sounds from the lungs and trachea by chest contact sensor and ambient |
| | sounds by lapel microphone in coughs per hour |
| DreaMed: Advisor PRo | Measuring glucose variability in Type I Diabetes in Decision Support Tool: |
| | glucose patterns by algorithm in glucose variability |
| Abbott: FreeStyle Libre Flash | Measuring glucose variability in Diabetes Mellitus Type 2 in CGM: glucose |
| Glucose Monitor | values by flash glucose monitor device in glucose variability |
| ZOLL: LifeVest | Measuring heart rate variability in Cardiomyopathies in Wearable Defibrillator: |
| | Heart Rate Monitor Enhanced Treatment Optimization by Wearable Cardioverter |
| | Defibrilator in Heart Rate Variability |
| MedRhythms: MR-010 | Measuring gait metrics and gait speed in Cerebral infarction in Digital |
| | Therapeutics: patient application and sensors in gait metrics |
| Wearable accelerometer | Measuring tremor and tremor count in Parkinson's Disease in Accelerometer: |
| | tremor count by accelerometry in Performance of a wearable accelerometer in |
| | detecting dyskinesia |
| ActiGraph: CentrePoint | Measuring physical activity: walking and activity count in Pulmonary Arterial |
| Insight Watch | Hypertension in Actigraphy: Walking distance measurement in physical activity |
| | walking. |
| Roche: Galaxy S3 mini | Roche: Galaxy S3 mini(Samsung, Seoul, South Korea) provided with a single, |
| provided with a single, | preinstalled custom application (Roche PD Mobile Application v1; Roche, |
| preinstalled custom | Basel, Switzerland) |
| application | |
| ActiGraph: GT9x Link watch | Measuring Physical Activity and Airflow Limitation in Asthma Disease in |
| | Actigraphy: physical movement measurements in physical activity |
| Device | Measuring Sleep Efficiency and Respiratory Disturbance in Cystic Fibrosis |
| | Disease in Actigraphy: movement detection in sleep efficiency |
| Device | Measuring Physical Activity and Sleep Disturbance in Irregular Sleep-Wake |
| | Rhythm Disorder in Actigraphy: movements degree and intensity detection in |
| | physical activity |
| BetaBionics: iLet ™′ Insulin | Measuring glucose variability in Type I Diabetes by CGM: continuous |
| Pump System with a | measurement of glucose levels in glycemic variability |
| Continuous Glucose | |
| Monitoring (CGM) device | |
| Philips: Actiwatch Spectrum | Measuring Physical Activity and Exercise Tolernace in Chronic Heart Failure |
| | With Reduced Ejection Fraction in Actigraphy: physical movement |
| | measurements in physical activity |
| Roche: Galaxy S3 mini | Roche: Galaxy S3 mini(Samsung, Seoul, South Korea) provided with a single, |
| provided with a single, | preinstalled custom application (Roche PD Mobile Application v1; Roche, |
| preinstalled custom | Basel, Switzerland) |
| application | |
| Sprometry device | Spirometry is the most common type of pulmonary function or breathing test. |
| (placeholder) | This test measures how much air the patient can breathe in and out of the lungs, |
| | as well as how easily and fast the patient can the blow the air out of the lungs. |
| Empatica: E4 watch | Measuring Seizures and Seizure Activity in Rett's Syndrome in Heart rate |
| | variability: Photoplethysmographic Heart Rate in Seizure Activity (seizure |
| | prediction) |
| Empatica: E4 watch | Measuring Seizures and Seizure Activity in Rett's Syndrome in Skin |
| | Temperature variability: peripheral skin temperature in Seizure Activity (seizure |
| | prediction) |
| Empatica: E4 watch | Measuring Physical Activity and Seizures in Rett's Syndrome in Accelerometry: |
| | movement detection in physical activity |
| Proteus Digital Health: | Measuring Medication Adherence and Medication Intake in HIV Disease in |
| Proteus ® Digital Health | Patient adherence: ingestibles for continuous monitoring in medication intake |
| Feedback Device | |
| Camntech: MotionWatch8 | Measuring Physical Activity and Exercise Tolerance in Chronic Heart Failure |
| | With Reduced Ejection Fraction in Actigraphy: physical movement |
| | measurements in physical activity |
| Device | Measuring Sleep Efficiency and Sleep Disturbance in Irregular Sleep-Wake |
| | Rhythm Disorder in Actigraphy: movements degree and intensity detection in |
| | sleep efficiency |
| Device | Measuring Insomnia and Sleep Efficiency in Major Depressive Disorder in |
| | Actigraphy: physical movement measurements in walking activity |
| Device | Measuring Insomnia and Sleep Efficiency in Major Depressive Disorder in |
| | Actigraphy: physical movement detection and measurements in nighttime |
| | activity |
| Philips: Actiwatch Spectrum | Measuring Sleep Efficiency and Exercise Tolerance in Chronic Heart Failure |
| | With Reduced Ejection Fraction Disease in Actigraphy: movement detection and |
| | intensity in sleep efficiency |
| Bodymedia: SenseWear | Measuring Physical Activity and Airflow Limitation in Asthma Disease in |
| Armband Gecko | Actigraphy: physical movement measurements in physical activity |
| Garmin: Vivofit 2 | Measuring Physical Activity and Airflow Limitation in Asthma Disease in |
| | Actigraphy: physical movement measurements in physical activity |
| SOMNOmedics: | Measuring Physical Activity and Airflow Limitation in Asthma Disease in |
| SOMNOwatch Plus | Actigraphy: physical movement measurements in physical activity |
| GSK: PARADE APP | GSK: PARADE APP (installed on iPhone) - PARADE app developed using |
| (installed on iPhone) | ResearchKit platform(Apple Inc, Cupertino, CA, USA) |
| Fitbit/ActiGraph: Actigraphy | Measuring physical activity in patients suffering from Diabetes Type 1 |
| Novo Nordisk: Tresiba ® | Measuring glucose variability in Type I Diabetes |
| FlexTouch ® Insulin Pen | |
| Sanofi: LANTUS ® | Measuring glucose variability in Type I Diabetes |
| SOLOSTAR ® INSULIN | |
| PEN | |
| DexCom: G6 Continuous | Measuring glucose variability in Type I Diabetes in CGM: glucose values by |
| Glucose Monitor | continuous glucose monitor in glucose variability |
| Medtronic MiniMed: | Measuring glucose variability in Type I Diabetes |
| MINIMED ™ 670G | |
| SYSTEM Insulin Pump | |
| BetaBionics: iLet ™′ Insulin | Measuring glucose variability in Type I Diabetes by CGM: continuous |
| Pump System with a | measurement of glucose levels in glycemic variability |
| Continuous Glucose | |
| Monitoring (CGM) device | |
| FitBit (no specific device) | Measuring sleep efficiency in Type 1 Deabetes by Activity monitor: movement |
| | detection in sleep efficiency |
| Hand-held pulse oximeter | Measuring pulse oxygenation and pulse oxygenation level in Covid 19 in Pulse |
| | oximeter: oxygen levels by pulse oximetry in oxygenation improvement |
| No-contact thermometer | Measuring body temperature and fever in Covid 19 in Thermometer: temperature |
| | by non-contact infrared thermometer in time to sustained absence of fever |
| Janssen: ML automated | To measure MES and total mayo score in abdominal pain and cramping in |
| scoring algorithm | Ulcerative Colitis |
| Janssen: ML automated | To measure MES and total mayo score in abdominal pain and cramping in |
| scoring algorithm | Ulcerative Colitis |
| Apple: Apple Watch 1 | Measuring Working Memory and Cognitive Impairment in Major Depressive |
| | Disorder in Wearable watch: testing the cognitive performance variability with |
| | assessment tests in working memory |
| GPSkin: GPSkin barrier | Simultaneously scans the Trans-Epidermic Water Loss (TEWL) and Stratum |
| | Corneum Hydration (SCH) levels of your skiS. |
| ActivInsights: GENEactiv | To measure WASO in sleep quality in AD. |
| Original Watch | |
| Emerald innovations: | To measure WASO in sleep quality in AD. |
| Emerald | |
| Pebble Smart Warables: | Measuring chorea in Huntington Disease patients by Activity monitor: |
| Pebble Pace Watch | Movement detection in chorea |
| Janssen: Ordinal multi- | To measure MES and total mayo score in abdominal pain and cramping in |
| instance learning | Ulcerative Colitis |
| Actigraph | Measuring sleep efficiency and waso (wake after sleep onset) in Restless Legs |
| | Syndrome in Actigraphy: Sleep WASO by Actigraphy in Sleep Efficiency |
| BioSensics: LegSys ™ | Measuring Physical Activity and Foot Complications in Other specified diabetes |
| | mellitus with diabetic chronic kidney disease in Accelerometry: activity |
| | monitoring of physical activity providing feedback about gait speed and body |
| | sway |
| BioSensics: LegSys ™ | Measuring Physical Activity and Foot Complications in Other specified diabetes |
| | mellitus with diabetic chronic kidney disease in Accelerometry: activity |
| | monitoring of physical activity providing feedback about body sway |
| Great Lakes | Measuring Physical Activity and Motor Activity in Parkinson's Disease in |
| NeuroTechnologies: Kinesia- | Wireless motion sensor: motor test measurements in tremor, bradykinesia, |
| ONE | dyskinesia |
| Great Lakes | Measuring Physical Activity and Motor Activity in Parkinson's Disease in |
| NeuroTechnologies: Kinesia- | Wireless motion sensor: motor symptoms recording by continuous measurement |
| 360 | in tremor, slowness, dyskinesia and mobility |
| Device [placeholder #1]: | Measuring Nocturnal Scratch and Pruritus in Atopic Dermatitis Disease in |
| device (no specific brand) | Actigraphy: physical movement measurements in nocturnal scratch |
| Actigraph | Measuring sleep efficiency and sleep duration in Restless Legs Syndrome in |
| | Actigraphy: Sleep Duration by Actigraphy in Sleep Efficiency |
| Apple: Apple Watch 1 | Measuring Attention and Cognitive Impairment in Major Depressive Disorder |
| | in Wrist-worn watch: cognitive performance variability with assessment tests in |
| | attention |
| Omnipod: Omnipod | Measuring glucose variability in Diabetes Mellitus in CGM: AID insulin dosing |
| 5/Horizon HCL system | in glucose variability |
| Avazzia: Tennant | Measuring physical activity in patients with diabetic foot ulcer by Activity |
| Biomodulator | monitor: movement detection in physical activity |
| Kent Imaging: SnapshotNIR | measuring tissue oxygenation of lower extremities in patients with diabetic foot |
| | ulcer by Pulse oximetry: non-invasive oxygenation measurement in lower |
| | extremeties oxygen saturation |
| BioSensics: LEGSys | Measuring gait speed in Diabetic Foot Ulcers Patients by Activity monitor: Gait |
| | speed in kinematics of lower body |
| BioSensics: BalanSens | Measuring balance in diabetic foot ulcer patients by Activity monitor: Balance |
| | and postural sway in balance |
| BioSensics: PAMSys | Measuring sedentary behaviour in diabetic foot ulcer patients by Activity |
| (physical activity monitoring | monitor: Sensor for continuous remote monitoring in sedentary behaviour |
| system) | |
| MC10: BioStamp nPoint | Measuring heart rate variability in diabetic foot ulcer patients by Heart Rate |
| | Monitor: Sensor measuring physiological signs in Heart Rate Variability |
| ActiGraph: Accelerometer | Measuring nocturnal activity in postoperative recovery by activity monitor: |
| measuring nocturnal activity | movement detection in nocturnal activity |
| ActiGraph | Measuring mobility in postoperative recovery by ActiGraph: Accelerometer |
| | measuring mobility |
| ActiGraph: Accelerometer | Measuring physical activity in postoperative recovery by Activity monitor: |
| measuring physical activity | Activity counts in physical activity |
| Motion Analysis | Measuring facial movement and facial task performance in Huntington Disease |
| Corporation: Motion Capture | in Motion sensor: facial recognition by 3-D optical motion capture system in |
| Systems | facial movement |
| Xsens Technologies: Xsens | Measuring movement detection and motor performance in Huntington Disease in |
| MVN Awinda motion capture | Motion sensor: physical activity by motion capture system in movement |
| system | detection |
| Courage + Khazaka: | Measuring skin aging and change in skin roughness in Changes in Skin Texture |
| VisioScan VC98 | in Camera: Changes in skin roughness by high resolution b/w video sensor in |
| | Skin Aging |
| Swift: Skin and wound | To measure wound status in skin condition in AD. |
| mobile application | |
| Actigraph: | Measuring physical activity and exercise tolerance in Pulmonary Arterial |
| | Hypertension in Actigraphy: Activity count by actigraphy in physical activity |
| Actigraph | Measuring physial activity in Breast Cancer in Actigraphy: Activity counts in |
| | physical activity |
| Continuous Glucose Sensor | Measuring glucose variability and glucose intolerance in Short Bowel Syndrom |
| | in CGM: glucose values by continuous glucose monitor in glucose variability |
| Device | Measuring Physical Activity and Pain in Transverse Myelitis Disease in |
| | Actigraphy: movement detection in sleep efficiency |
| Device | Measuring Physical Activity and Pain in Transverse Myelitis Disease in |
| | Actigraphy: physical movement measurements in Physical Activity |
| Device | Measuring Sleep Efficiency and Pain in Multiple Sclerosis Disease in |
| | Actigraphy: movement detection in sleep efficiency |
| Device | Measuring Physical Activity and Pain in Multiple Sclerosis Disease in |
| | Actigraphy: movement detection in physical activity |
| Continuous Glucose Sensor | Measuring glucose variability in Glucose Intolerance in CGM: glucose values by |
| | continuous glucose monitor in glucose variability |
| Actigraph | Measuring sleep activity and sleep efficiency in Glucose Intolerance in |
| | Actigraphy: physical movement in sleep efficiency |
| Actigraph | Measuring sleep activity and sleep midpoint in Glucose Intolerance in |
| | Actigraphy: physical movement in sleep efficiency |
| WestRock: MEMS Cap | |
| Action: Magic watch 5 | Beyond actigraphy, this watch outputs tri-axial, raw accelerometery data with |
| (mock-up) | environmental light and temperature measurements. It is robust in objectively |
| | monitoring physical activity, sleep and everyday living behaviours reliably. |
| Nonin: Pulse oximetry | To measure blood oxygen saturation in COVID-19 |
| Actigraph | Measuring sleep activity and feeding patterns in Short Bowel Syndrom in |
| | Actigraphy: physical movement in sleep efficiency |
| Actigraph: GT9X Link | Wrist-worn watch Actigraph device |
| Actigraph: CentrePoint | Wrist-worn watch Actigraph device |
| Insight Watch | |
| Actigraph: wGT3X-BT | Digital filtering technology; Wear time sensor Ambient light sensors; Bluetooth |
| | Smart technology, USB; BL 25 days; Memory 4GB; Dynamic range 8G; 30-100 |
| | Hz; 19 grams; Dimensions 4.6 × 3.3 × 1.5 cm; Data storage 180 days; Water |
| | resistance 1 meter 30 min |
| Philips: Actiwatch 2 | Actigraphy: 3-axis accelerometer (solid state piezoelectric) |
| Philips: Actiwatch spectrum | Actigraphy: 3-axis accelerometer (MEMS) |
| Philips: Health band | Actirgaphy: 3-axis accelerometer (MEMS) |
| Samsung: Galaxy Watch 3 | |
| TytoCare: TytoCare medical | |
| exam kit | |
| VitalConnect: VitalPatch | |
| RTM | |
| Device | Measuring Sleep Efficiency and Sleep Disturbance in Reflux Esophagitis |
| | Disease in Actigraphy: movement detection in sleep efficiency |
| Device | Measuring Sleep Efficiency and Pain in Neuromyelitis Optica Spectrum |
| | Disorder in Actigraphy: movement detection in sleep efficiency |
| Device | Measuring Physical Activity And Pain in Neuromyelitis Optica Spectrum |
| | Disorder in Actigraphy: physical movement measurements in Physical Activity |
| MC10: BioStamp | Measuring physical activity and sleep in Mild Cognitive Impairment in Activity |
| | Monitor: activity count by wearable wireless sensors in physical activity and |
| | sleep |
| Device (18980) | Measuring Physical Activity and Exercise Tolerance in Heart Failure With |
| | Preserved Ejection Fraction in Accelerometry: physical movement |
| | measurements in physical activity |
| ActiGraph: GT9x Link watch | Measuring Sleep Efficiency and Exercise Tolerance in Pulmonary Arterial |
| | Hypertension Disease in Actigraphy: movement detection in sleep efficiency |
| Canfield Scientific: VISIA- | Measuring skin aging in Changes in Skin Texture in Camera: Change in skin age |
| CR | by Facial Photo Capture in Skin Aging |
| Actigraph | Measuring physial activity in Breast Cancer in Actigraphy: Activity counts in |
| | physical activity |
| Actigraph | Measuring sleep activity and glucose intolerance in Short Bowel Syndrom in |
| | Actigraphy: physical movement in sleep efficiency |
| Continuous Glucose Sensor | Measuring glucose variability and sleep quality in Short Bowel Syndrom in |
| | CGM: glucose values by continuous glucose monitor in glucose variability |
| Actigraph | Measuring sleep activity and sleep quality in Short Bowel Syndrom in |
| | Actigraphy: physical movement in sleep efficiency |
| PHQ9 scale | — |
| MINIMED: 670G or 640G | AID insulin dosing and Measuring glucose variability in Diabetes Mellitus type |
| insulin pump with guardian | 1 |
| sensor | |
| EPILOG: AI EEG analysis | To measure EEG trends for early diagnosis of epilepsis in epilepsis patients |
| Dexcom: G6 CGM System | Measuring glucose variability in Diabetes Mellitus Type 2 in CGM: glucose |
| (20132) | values by continuous glucose monitor in glucose variability |
| Parkinson's KinetiGraph - | measuring movement detection in PD patients by Activity monitor: movement |
| Global Kinetics: PKG ® | detection in tremor |
| system - Watch (20147) | |
| Bayer: Breelib nebulizer | Measuring Medication Intake and Medication Adherence in Pulmonary |
| (+Breeconnect app) | Hypertension in Patient adherence: nebulizer to monitor medication intake |
| Device: Actigraph device | |
| (no specific brand) | |
| Device: Chest Contact | Cough recording digital wearable monitoring |
| Sensor (no specific brand) | |
| Byteflies: Sensor Dot | |
| EPILOG: AI EEG analysis | To measure brain wave abnormalities for early diagnosis of epilepsis in epilepsis |
| | patients |
| Amazon: Halo band | |
| Biobeat: Wrist monitor | |
| Biobeat: Chest monitor | |
| NVIDIA: AI HCP (DGX | |
| A10) | |
| OncoDNA: AI HCP | |
| Neurokeys: Neurokeys | |
| smartphone application | |
| McRobrets: MoveMonitor | device: MoveMonitor |
| (20146) | |
| Dexcom: G6 CGM System | Measuring glucose variability in Kidney Transplant in CGM: glucose values by |
| | continuous glucose monitor in glucose variability |
| Apple: Watch 2 | Measuring Physical Activity and Dyspnea in Pulmonary Hypertension Disease |
| | in Accelerometry: physical movement measurements in physical activity |
| Apple: iPhone 6s and | Measuring Physical Activity and Dyspnea in Pulmonary Hypertension Disease |
| application | in Accelerometry: physical movement measurements in physical activity |
| Device [placeholder #2]: | Measuring nocturnal scratch in Atopic Dermatitis patients by Actigraph device |
| Actigraph device (no specific | (no specific brand) |
| brand) | |
| PKG ® system - Watch | Parkinson's KinetiGraph - Global Kinetics: PKG ® system - Watch |
| Dexcom: CGM G6 | Measuring glucose variability in Diabetes by CGM: continuous measurement of |
| | glucose levels in glycemic variability |
| PKG ® system - Watch | Parkinson's KinetiGraph - Global Kinetics: PKG ® system - Watch |
| Device: Activity monitor (no | device: Wearable Physical Activity and Sleep Monitor |
| specific brand) | |
| Device: Actigraph device | measuring nocturnal activity in asthma by Actigraphy: Movement detection in |
| (no specific brand) | nocturnal activity |
| Device: Chest Contact | measuring cough activity in chronic cough by Chest Contact Sensor: Awake |
| Sensor (no specific brand) | cough frequency in cough activity |
| Omnipod Horizon ™: | Measuring glucose variability in Type I Diabetes by CGM: continuous |
| Automated Glucose Control | measurement of glucose levels in glycemic variability |
| System | |
| ActiGraph: ActiGraph GT9X | Measuring physical activity in patients with Diabetic Peripheral Neuropathic |
| Link | pain by Actigraphy: Activity counts in physical activity |
| DEEM: Dreem 2 Headband | device: Dreem 2 Headband |
| Great Lakes | measuring motion in tremor by Activity monitor: wireless sensor measuring |
| NeuroTechnologies: Kinesia | motion in Tremor |
| One Device | |
| Device: pulse oximeter | measuring overnight pulse oximetery variance in Sickle cell disease (SCD) |
| device (no specific brand) | patients by Pulse oximetry: Oxygenation measurement in overnight pulse |
| | oximetery variance |
| Device: Actigraph device | Measuring sleep efficacy in Sickle cell disease (SCD) patients by Actigraphy: |
| (no specific brand) | movement detection in sleep efficacy |
| Actigraphy: movement | Measuring nocturnal activity in Sickle cell disease (SCD) patients by |
| detection in nocturnal activity | Actigraphy: movement detection in nocturnal activity |
| Actigraphy: activity count in | Measuring physical activity in Sickle cell disease (SCD) patients by Actigraphy: |
| physical activity | activity count in physical activity |
| ActiGraph: Actiwatch 2 | measuring nocturnal activity in menopausal depression by Actigraphy: |
| | Movement detection in nocturnal activity |
| MyoVoice | |
| Steritas: GTI platform | |
| Janssen: JAKE | |
| Janssen: Revere | |
| 1DROP diagnostics: 1DROP | |
| Achu Health: Achu | |
| Sibel Health: ANNE Sleep | |
| Janssen: Heartline | Application: Heartline (paired with iPhone/Apple watch) |
| Janssen: Heartline | |
| Strados Labs: Strados RESP | |
| PhysIQ: AccelerateIQ | |
| Medical PST: MIMOSYS | |
| Empatica: E4 wristband | |
| Empatica: Embrace | |
| ActivInsights: Band | |
| BreatheOx: Albus Health | |
| Actigraphy device | Measuring Sleep Efficiency in Major Depressive Disorder in Actigraphy: |
| | physical movement detection and measurements in nighttime activity |
| Cyma: StepWatch Activity | Measuring Physical Activity in motor activity in Parkinson's disease in Activity |
| Monitor (SAM) | monitoring: intensity changing and number of steps/day in walking activity |
| Activity monitor | Measuring Physical Activity in Cystic Fibrosis Disease in Actigraphy: |
| | movement detection in physical activity |
| Advanced Brain Monitoring: | |
| SleepProfiler | |
| IM Systems: DigiTrac | |
| EDA biosensor | Measuring skin conductance in cognitive engagement in cognitive impairment in |
| | EDA biosensor: skin conductance measurement in cognitive engagement |
| Pal: ActivPAL | Measuring Physical Activity and Weight Loss in Cachexia Disease in |
| | Accelerometry: physical movement measurements and intensity detection in |
| | physical activity (walking) |
| Owkin: AI solutions | |
| MC10: BioStamp digital | Measuring Physical and Sleep Activity in motor activity and sleep efficiency in |
| wearable device | Parkinson's disease |
| Actigraphy device | Measuring ambulatory activity in Actigraphy: movement detection in sleep |
| | efficiency |
| BioIntelliSense: Biosticker | |
| BioIntelliSense: Biosticker | |
| IMEC: Health patch | |
| The Siesta Group: EEG | |
| device | |
| Fitbit: Charge 5 | |
| Omron: HeartGuide | |
| Tasso Inc.: Tasso+ | Placeholder in coronavirus (!) |
| Continuous Glucose Monitor | Measuring glucose variability in Diabetes Mellitus Type 2 in CGM: glucose |
| [steakholder] | values by continuous glucose monitor in glucose variability |
| AliveCor: Kardia ECG | |
| Mobile | |
| Continuous Glucose Monitor | Measuring glucose variability in Diabetes Mellitus Type 2 in CGM: glucose |
| [steakholder] | values by continuous glucose monitor in glucose variability |
| Koneksa Health: Koneksa | |
| Actigraphy device | Measuring Physical Activity in Chronic Heart Failure in Actigraphy: physical |
| | movement measurements in physical activity |
| Neoteryx: Mitra with VAMS | |
| Neoteryx: Mitra with VAMS | |
| Neoteryx: Mitra with VAMS | |
| Empatica: E4 watch | Measuring Seizures and Seizure Activity in Rett's Syndrome in The constantly |
| | fluctuating changes in certain electrical properties of the skin: Seizure activity |
| | (prediction) by Electrodermal activity |
| Altoida: System = | Measuring Cognitive Assessment and Cognition in Alzheimer's Disease in |
| Microphone (audio | Medical device(system): brain health measurement by cognitive assessment in |
| recordings) + Smartphone | 10 minutes |
| App | |
| PSG: WATCH-PD system | The Parkinson Study Group (PSG) comprises physicians and medical centers |
| | across the US and Canada who are committed in finding new therapeutics for |
| | those with Parkinson's. |
| Skintronics: wearable device | Measuring blink count using nanomembrane electrodes in Blepharospasm in |
| | Activity monitoring: nanomembrane electrodes measurement in blink count |
| Whoop: wearable device | Measuring heart rate by photoplethysmography (PPG) in Autoinflammatory |
| | syndrome in Heart rate monitor: photoplethysmography (PPG) in heart rate |
| Withings: BPM Core | Measuring pulse wave velocity and blood pressure variability in |
| | Autoinflammatory Syndrome in Heart rate monitor: pulse detection in pulse |
| | wave velocity |
| FitBit (no specific device) | Fitbit devices use a 3-axis accelerometer to count steps. This sensor also allows |
| | your device to determine the frequency, duration, intensity, and patterns of your |
| | movement. |
| Roche: Galaxy S3 mini | Roche: Galaxy S3 mini(Samsung, Seoul, South Korea) provided with a single, |
| provided with a single, | preinstalled custom application (Roche PD Mobile Application v1; Roche, |
| preinstalled custom | Basel, Switzerland) |
| application | |
| Bellerophon Pulse | Measuring physical activity in patients suffering from Pulmonary hypertension |
| Technologies: INOpulse | associated with interstitial lung disease (PH-ILD) by Activity monitor: Wearable |
| device | activity monitor (actigraph) by activity counts |
| Preventice Solutions: | Measuring irregular heart rythm and atrial fibrillation burden in Atrial |
| BodyGuardian ® MINI PLus | Fibrillation in ECG: Electrocardiogram by submersible arrhythmia monitor |
| | (ECG wearable patch) in Irregular Heart Rythm |
| Continuous Glucose Sensor | Measuring glucose variability and feeding patterns in Short Bowel Syndrom in |
| | CGM: glucose values by continuous glucose monitor in glucose variability |
| Janssen: Corvista Health | |
| propriatory ML algorithm | |
| Science37: Skin imaging | |
| analysis | |
| HUMA: Huma application | |
| Janssen: AI Model (LCFRP) | Identify patients with high risk to develop lung cancer in lung cancer |
| Janssen: AI Model (IMWG) | |
| RheumaBuddy: Mobile | |
| application | |
| ActiGraph: GT9x Link watch | Measuring Physical Activity and Exercise Tolerance in Pulmonary Arterial |
| | Hypertension Disease in Actigraphy: physical movement measurements in Daily |
| | Life Physical Activity (daily time spent in non-sedentary activity) |
| Janssen: JAKE | |
| Coping with Voices | Online self-management program |
| AVATAR therapy | Combination of digital image and speech modulation software |
| IC Tag | Integrated Circuit Tag Monitoring System: The IC Tag monitoring system with |
| | Powertags was used for monitoring (Matrix Int, Osaka, Japan) |
| IC Tag | Integrated Circuit Tag Monitoring System: The IC Tag monitoring system with |
| | Powertags was used for monitoring (Matrix Int, Osaka, Japan) |
| Physilog | The Physilog system (BioAGM, CH): a motion sensor attached to the chest with |
| | an elastic belt. |
| PT-RFID | The Power Tag system is able to monitor the whereabouts of a subject and the |
| | rhythm of daily activities such as walking distance per day and frequency of |
| | toileting: |
| Actical | |
| CamNTech: Actiwatch | The current study quantified dementia patients' daily PA levels, characterized |
| | their PA patterns, and derived estimates of relative PA intensity based on |
| | actigraphy. |
| Empatica E4 | Measurement of quantity of movement through the use of an actigraph in |
| | participants with apathy and depression compared to people with dementia not |
| | affected with these disorders. |
| MOVE MOVISENS | |
| Observer NOLDUS | |
| Observer NOLDUS | |
| CamNTech: Actiwatch mini | |
| Sleep-Watch-O | Actigraphy provide a reliable estimate of sleep/wake activity |
| Mini Nutritional Assessment | |
| short-form (MNA-SF) | |
| SNAQ& CNAQ- appetite | |
| assessment tools predicting | |
| weight loss | |
| SNAQ& CNAQ- appetite | |
| assessment tools predicting | |
| weight loss | |
| Mini Nutritional | |
| Assessment ® (MNA ®, | |
| Société des Produits Nestlé, | |
| S.A., Vevey, Switzerland) | |
| Appetite and Eating Habits | |
| Questionnaire (APEHQ) | |
| [placeholder[ | |
| Actigraphy | |
| Geriatric Anxiety Invetory | |
| scale | |
| Penn State Worry | |
| Questionnaire | |
| RAID scale | Rating Anxiety in Dementia (RAID) scale: The RAID has the highest sensitivity |
| | for anxiety disorders, includes a caregiver interview, and was specifically |
| | designed for those experiencing dementia. |
| CMAI scale | CMAI scale which includes 8 of 29 items that assess the frequency of VA |
| Cornell scale | |
| Mini-mental state | |
| Psychogeriatric scale | |
| General medical health rating | |
| 21 PDI scoring | The 21-item Peters et al Delusions Inventory (PDI) |
| Eppendorf itch questionnaire | https://link.springer.com/article/10.1007/s12016-010-8230-2#Sec9 |
| DRP3 scoring | The DRP3 screen, comprising three yes/no questions, is a content-valid tool for |
| | detecting H + D in dementia |
| VAS scoring | 100-mm horizontal visual analogue scale (VAS), which allows the patient to |
| | determine the severity by indicating the space between outermost points, where |
| | 0 indicates no fatigue, and 100 complete lack of strength and energy. |
| SF-36 scoring | 2. 36-Item Short Form Health Survey (SF-36) Vitality Scale: do not include all |
| | aspects of fatigue. |
| SF-36 scoring | 36-Item Short Form Health Survey (SF-36) Vitality Scale: do not include all |
| | aspects of fatigue. |
| MAF scoring | MAF (Multidimensional Assessment of Fatigue) contains questions to study four |
| | dimensions of fatigue: severity, mental fatigue, frequency and impact on |
| | everyday activity. |
| FACIT-F scoring | Functional Assessment of Chronic Illness Therapy - Fatigue Scale (FACIT-F), |
| | consisting of questions on general, physical, mental fatigue and the will to live. |
| FACIT-F scoring | Functional Assessment of Chronic Illness Therapy - Fatigue Scale (FACIT-F), |
| | consisting of questions on general, physical, mental fatigue and the will to live. |
| FSS scoring | Fatigue Severity Scale (FSS), consisting of questions on the influence of fatigue |
| | in everyday function. |
| NRS scoring | NRS - an 11 point numerical rating scale. |
| HAQ DI scoring | Health Assessment Questionnaire disability index (HAQ DI): adapted for use in |
| | PsA; 20 items assessing 8 domains: dressing, rising, eating, walking, hygiene, |
| | reach, grip, and usual activities, with scores ranging from 0 = none to 3 = |
| | maximum disability. |
| DLQI scoring | ‘Dermatology Life Quality Index (DLQI): 10-item questionnaire which |
| | ascertains the impact of skin disease on work and leisure activities |
| WINR scale | Worst Itch Numerical Rating Scale |
| WINR scale | Worst Itch Numerical Rating Scale |
| WINR scale | Worst Itch Numerical Rating Scale |
| CamNTech: Actiwatch | |
| CamNTech: Actiwatch plus | |
| IM Systems: ActiTrac | |
| PSI scaling | Psoriasis Symptom Inventory (PSI): s a patient-reported outcome instrument that |
| | measures the severity of psoriasis signs and symptoms. |
| PSQI scoring | Pittsburgh Sleep Quality Index (PSQI) - assesses seven domains: subjective |
| | sleep quality, sleep latency, sleep duration, habitual sleep efficiency, sleep |
| | disturbances, use of sleeping medications and daytime dysfunctions |
| ISI scaling | Insomnia severity index (ISI) |
| Iso Teste: electromechanic | |
| chair dynamometer | |
| MOS-SS scaling | Medical outcomes study sleep scale (MOS-SS) |
| GSDS scaling | General sleep disturbance scale (GSDS) |
| Withings: Activité Pop watch | Withings ® Activité Pop watch - activity tracker, records the number of steps per |
| | minute. |
| SQUASH scoring | Physical activity was assessed using the validated Short Questionnaire to Assess |
| | Health-enhancing physical activity (SQUASH) |
| IBD-SS scale | Inflammatory Bowel Disease Symptom Severity (IBD-SS) scale |
| VSI scale | Visceral Sensitivity Index (VSI) |
| McGill Pain Questionnaire | The McGill Pain Questionnaire is also not specific for abdominal pain but |
| | provides information about pain intensity and also a qualitative description of the |
| | pain (eg, burning vs stabbing). |
| VA scale | General pain intensity scales: Visual Analog Scale (VAS) |
| NR scale | General pain intensity scales: Visual: Numeric Rating Scale (NRS) |
| JAMAR: Hydraulic hand | |
| dynamometer | |
| Elastic modulus pen | Portable pen-sized instrumentation to measure stiffness of soft tissues. |
| Kenko sport timer: | |
| Chronometer | |
| Delfin technologies: | |
| VapoMeter | |
| Delfin technologies: | |
| MoistureMeterSC compact | |
| RAPS | Development of an instrument to measure pain in rheumatoid arthritis: |
| | Rheumatoid Arthritis Pain Scale (RAPS) |
| IPRS Mediquipe: Isokinetic | Knee extension and flexion isokinetic assessment of the dominant thigh- |
| dynamometer | isokinetic biodex system 4-muscle testing and rehabilitation isokinetic |
| | dynamometer (IPRS Mediquipe Ltd, Little Blakenham, Suffolk, UK) |
| BDI scale | Beck Depression Inventory (BDI) scale II questionnaire |
| Electronic diaries | |
| Multidimensional itch | |
| assessment | |
| Unidimensional itch intensity | |
| scale | |
| WOMAC scale | Self-reported -Western Ontario and McMaster Universities Osteoarthritis Index |
| | (WOMAC) |
| Scanco Medical: XtremeCT | High-resolution peripheral QCT was performed with an XtremeCT scanner |
| scanner | (Scanco Medical), ll images were analyzed using open-source DICOM viewer |
| | software (OsiriX V4.0) |
| Bertec: instrumented split- | |
| belt treadmill | |
| Camera motion analysis | Three-dimensional kinematic data was recorded at 60 Hz from 23 reflective |
| system | markers using a six camera Motion Analysis system (Santa Rosa, CA, USA). |
| Tena Idenfiti Sensor Wear | A 72-hour capturing method using an electrical logger attached to a specified |
| | patch |
| Tena Idenfiti Sensor Wear | A 72-hour capturing method using an electrical logger attached to a specified |
| | patch |
| Tena Identify Sensor Wear | external logger enabled smart-patch with 72 hour fluid and time stamps |
| MDPI standalone module | MDPI standard detection kit that is deployable in any diaper/patch |
| applyable on any diaper | |
| Adamant Health | A measurement solution that measures various aspects of myoclonus using a |
| Measurement and Analysis | combination of electromyography and accelerometry. |
| Service | |

What is claimed is:

1. A method for characterizing a disease of a subject, the method comprising:
obtaining a measurement of interest from the subject;
selecting a digital measurement solution from a plurality of digital measurement solutions, wherein the plurality of digital measurement solutions are of a common class that is represented by a target solution profile; and
applying the selected digital measurement solution to the obtained measurement of interest to characterize the disease for the subject,
wherein the digital measurement solution comprises:
a measurement definition defining one or more concepts of interest relevant to the disease;
an instrumentation asset that transforms the measurement of interest captured according to the measurement definition to a dataset that is informative for characterizing the disease, wherein the instrumentation asset of the digital measurement solution is specific for a device used to capture the measurement of interest; and
optionally, an evidence asset for performing one or more validations on the dataset generated by the instrumentation asset,
wherein the target solution profile is unchanged over time and enables efficient life-cycle management of the plurality of digital measurement solutions.

2. The method of claim 1, wherein the target solution profile represents a generalization of the plurality of digital measurement solutions, wherein an instrumentation asset of the target solution profile is device technology agnostic.

3. The method of claim 1, wherein performing the one or more validations comprises performing one or more of a technical validation, an analytical validation, or a clinical validation.

4. The method of claim 3, wherein performing the technical validation comprises comparing the dataset generated by the instrumentation asset to specifications of one or more devices used to capture the measurement of interest.

5. The method of claim 3, wherein performing the analytical validation comprises: determining any of reliability, specificity, or sensitivity metrics for the dataset; and
comparing the reliability, specificity, or sensitivity metrics to a threshold value.

6. The method of claim 3, wherein performing the clinical validation comprises:
assessing treatment effects on measurements of interest for the disease.

7. The method of claim 1, wherein the digital measurement solution is previously validated by implementing one or more qualification protocols used to establish comparability of solutions across the digital measurement solutions of the target solution profile.

8. The method of claim 7, wherein a qualification protocol comprises steps of:
a) recruiting a N member participant group;
b) capturing measurements of interest across the N member participant group according to a specification of the digital measurement solution;
c) transforming the measurements of interest into a dataset according to the specification; and
d) validating the dataset to determine whether the digital measurement solution achieves comparable solutions of the target solution profile.

9. The method of claim 8, wherein validating the dataset comprises:
determining whether a characteristic of the dataset satisfies a threshold value of the target solution profile; and
responsive to the determination that the characteristic of the dataset satisfies the threshold value, validating the digital measurement solution as achieving comparability of solutions.

10. The method of claim 9, wherein validating the dataset further comprises responsive to determining that the digital measurement solution achieves comparability of solutions, storing an indication of a successful validation in metadata of the digital measurement solution.

11. The method of claim 10, wherein the metadata of the digital measurement solution is stored in a catalog accessible for inspection by third party users.

12. The method of claim 8, wherein the specification of the digital measurement solution represents an upgraded capability in comparison to a prior version of the digital measurement solution.

13. The method of claim 12, wherein the specification of the digital measurement solution represents an upgraded capability included in a newly released device used to capture the measurement of interest.

14. The method of claim 13, wherein the upgraded capability is one of an upgraded battery, upgraded data storage, upgraded acquisition frequency, or upgraded data collection algorithm.

15. The method of f claim 1, wherein the common class of the plurality of digital measurement solutions represents a common method of measuring activity from an individual.

16. The method of claim 15, wherein the common method of measuring activity uses a class of devices comprising one or more of wearable devices, devices including accelerometers, devices including gyroscopes, ingestibles, image and voice based devices, touchless sensors, and sensor based smart devices (e.g., scales, thermometers, and respirators).

17. The method of claim 1, wherein the instrumentation asset comprises a machine learning algorithm that transforms data captured according to the measurement definition to the dataset.

18. A method for building a digital measurement solution for characterizing a disease, the method comprising:
generating a measurement definition of a target solution profile, the measurement definition defining one or more concepts of interest relevant to the disease;
generating or selecting an instrumentation asset for the target solution profile, the instrumentation asset configured to transform data captured according to the measurement definition to a dataset, the instrumentation asset being device technology agnostic and is thereby interchangeable across different target solution profiles;
generating an evidence asset of the target solution profile for performing one or more validations on the dataset generated by the instrumentation asset;
generating a digital measurement solution by at least specifying a device for the instrumentation asset of target solution profile, wherein the digital measurement solution is of a common class that is represented by the target solution profile,
wherein the target solution profile is unchanged over time and thereby enables efficient life-cycle management of the plurality of digital measurement solutions.
